(19) United States
(12) Patent Application Publication
(10) Pub. No.: US 2022/0073531 A1
(43) Pub. Date: Mar. 10, 2022

(54) COMPOUNDS FOR ELECTRONIC DEVICES

(71) Applicant: Merck Patent GmbH, Darmstadt (DE)

(72) Inventors: Amir Hossain PARHAM, Frankfurt am Main (DE); Jens ENGELHART, Darmstadt (DE); Christian EICKHOFF, Mannheim (DE); Christian EHRENREICH, Darmstadt (DE); Jonas Valentin KROEBER, Frankfurt am Main (DE)

(21) Appl. No.: 17/298,552

(22) PCT Filed: Nov. 29, 2019
(86) PCT No.: PCT/EP2019/083011

§ 371 Date: May 29, 2021

(30) Foreign Application Priority Data
Nov. 30, 2018 (EP) 18209649.5

(51) Int. Cl. C07D 487/16; C07D 487/22; C07D 519/00; H01L 51/00

(52) U.S. Cl. CPC C07D 487/16; H01L 51/0072; C07D 519/00; C07D 487/22

(57) ABSTRACT

The present invention relates to condensed N-heteroaromatic compounds, to processes for preparing these compounds, and to electronic devices containing said compounds.

The present application relates to particular N-heteroaromatic compounds. The compounds are suitable for use in electronic devices.

Electronic devices in the context of this application are understood to mean what are called organic electronic devices, which contain organic semiconductor materials as functional materials. More particularly, these are understood to mean OLEDs (organic electroluminescent devices). The term OLEDs is understood to mean electronic devices which have one or more layers comprising organic compounds and emit light on application of electrical voltage. The construction and general principle of function of OLEDs are known to those skilled in the art.

In electronic devices, especially OLEDs, there is great interest in an improvement in the performance data, especially lifetime, efficiency, operating voltage and color purity. In these aspects, it has not yet been possible to find any entirely satisfactory solution. There is also a need for novel, alternative compounds for use in electronic devices, especially in OLEDs.

A great influence on the performance data of electronic devices is possessed by emitting layers, especially phosphorescent emitting layers. Novel compounds are also being sought for use in these layers, especially compounds that can serve as matrix material in an emitting layer. For this purpose, there is a search particularly for compounds that have a high glass transition temperature T_{G} and high oxidation stability and thermal stability, especially high oxidation stability and thermal stability.

The prior art discloses a multitude of heteroaromatic compounds for use as matrix material for phosphorescent emitting layers, for example carbazole derivatives and triazine derivatives.

However, there is still a need for alternative compounds suitable for use in electronic devices, especially for compounds having one or more of the abovementioned advantageous properties. In particular, there is a need for alternatives to triazine derivatives as matrix materials for phosphorescent emitters. There is still a need for improvement in the performance data achieved when the compounds are used in electronic devices, especially in respect of lifetime, operating voltage, efficiency and color purity of the devices.

It has been found that particular N-heteroaromatic compounds are of excellent suitability for use in electronic devices, especially for use in OLEDs, even more especially for use therein as matrix materials for phosphorescent emitters. The compounds lead to high lifetime, high efficiency, low operating voltage and high color purity of the devices.

Further preferably, the compounds have a high glass transition temperature T_{G} and high oxidation stability and thermal stability.

The present application provides an electronic device comprising a compound containing a structural element of a formula (I)

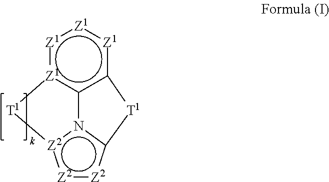

where:

Z¹ is the same or different at each instance and is C, CR¹ or N;

Z² is the same or different at each instance and is C, CR² or N;

T¹ is the same or different at each instance and is selected from (C═O)(NAr¹)—, —(C═S)(NAr¹)—, —(SO₂)(NAr¹)—, and —(C═O)O—;

Ar¹ is selected from aromatic ring systems which have 6 to 40 aromatic ring atoms and are substituted by one or more R³ radicals, and heteroaromatic ring systems which have 5 to 40 aromatic ring atoms and are substituted by one or more R³ radicals;

R¹ is the same or different at each instance and is selected from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form a ring; where the alkyl, alkoxy, alkenyl and alkynyl groups mentioned and the aromatic ring systems and heteroaromatic ring systems mentioned are each substituted by R⁴ radicals; and where one or more CH₂ groups in the alkyl, alkoxy, alkenyl and alkynyl groups mentioned may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂;

R² is the same or different at each instance and is selected from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where the alkyl, alkoxy, alkenyl and alkynyl groups mentioned and the aromatic ring systems and heteroaromatic ring systems mentioned are each substituted by R⁴ radicals; and where one or more CH₂ groups in the alkyl, alkoxy, alkenyl and alkynyl groups mentioned may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂;

R³ is the same or different at each instance and is selected from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups mentioned and the aromatic ring systems and heteroaromatic ring systems mentioned are each substituted by R⁴ radicals; and where one or more CH₂ groups in the alkyl, alkoxy, alkenyl and alkynyl groups mentioned may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂;

R⁴ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;

R⁵ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁵ radicals may be joined to one another and may form a ring; and where the alkyl, alkoxy, alkenyl and alkynyl groups, aromatic ring systems and heteroaromatic ring systems mentioned may be substituted by one or more radicals selected from F and CN;

k is 0 or 1, where, in the case that k=1, the Z¹ and Z² groups that bind to the T¹ group in question are C and are bonded to one another via the T¹ group, and where, in the case that k=0, the T¹ group in question is absent, and the Z¹ and Z² groups in question are not bonded to one another.

A circle within a six- or five-membered ring in the context of the present invention means that the ring in question is aromatic or heteroaromatic on account of pi electrons of double bonds or of heteroatoms.

The definition Z¹═C is understood to mean that the structural element of the formula (I) is part of a larger fused ring system, for example in that a benzene ring is fused thereto and to an adjacent Z¹. The same applies to Z²═C.

The divalent T¹ group may thus be the same or different at each instance and may be in either of the two possible orientations, i.e. in the case of —(C═O)(NAr¹)—, for example, as —(C═O)(NAr¹)— or as —(NAr¹)(C═O)—.

The definitions which follow are applicable to the chemical groups that are used in the present application. They are applicable unless any more specific definitions are given.

An aryl group in the context of this invention is understood to mean either a single aromatic cycle, i.e. benzene, or a fused aromatic polycycle, for example naphthalene, phenanthrene or anthracene. A fused aromatic polycycle in the context of the present application consists of two or more single aromatic cycles fused to one another. Fusion between cycles is understood here to mean that the cycles share at least one edge with one another. An aryl group in the context of this invention contains 6 to 40 aromatic ring atoms of which none is a heteroatom.

A heteroaryl group in the context of this invention is understood to mean either a single heteroaromatic cycle, for example pyridine, pyrimidine or thiophene, or a fused heteroaromatic polycycle, for example quinoline or carbazole. A fused heteroaromatic polycycle in the context of the present application consists of two or more single aromatic or heteroaromatic cycles that are fused to one another, where at least one of the aromatic and heteroaromatic cycles is a heteroaromatic cycle. Fusion between cycles is understood here to mean that the cycles share at least one edge with one another. A heteroaryl group in the context of this invention contains 5 to 40 aromatic ring atoms of which at least one is a heteroatom. The heteroatoms of the heteroaryl group are preferably selected from N, O and S.

An aryl or heteroaryl group, each of which may be substituted by the abovementioned radicals, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo[1,2-a]benzimidazole, naphthimidazole, phenanthroimidazole, pyridoimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention is a system which does not necessarily contain solely aryl groups, but which may additionally contain one or more non-aromatic rings fused to at least one aryl group. These non-aromatic rings contain exclusively carbon atoms as ring atoms. Examples of groups covered by this definition are tetrahydronaphthalene, fluorene and spirobifluorene. In addition, the term “aromatic ring system” includes systems that consist of two or more aromatic ring systems joined to one another via single bonds, for example biphenyl, terphenyl, 7-phenyl-2-fluorenyl, quaterphenyl and 3,5-diphenyl-1-phenyl. An aromatic ring system in the context of this invention contains 6 to 40 carbon atoms and no heteroatoms in the ring system. The definition of “aromatic ring system” does not include heteroaryl groups.

A heteroaromatic ring system conforms to the abovementioned definition of an aromatic ring system, except that it must contain at least one heteroatom as ring atom. As is the case for the aromatic ring system, the heteroaromatic ring system need not contain exclusively aryl groups and heteroaryl groups, but may additionally contain one or more non-aromatic rings fused to at least one aryl or heteroaryl group. The nonaromatic rings may contain exclusively carbon atoms as ring atoms, or they may additionally contain one or more heteroatoms, where the heteroatoms are preferably selected from N, O and S. One example of such a heteroaromatic ring system is benzopyranyl. In addition, the term “heteroaromatic ring system” is understood to mean systems that consist of two or more aromatic or heteroaromatic ring systems that are bonded to one another via single bonds, for example 4,6-diphenyl-2-triazinyl. A heteroaromatic ring system in the context of this invention contains 5 to 40 ring atoms selected from carbon and heteroatoms, where at least one of the ring atoms is a heteroatom. The heteroatoms of the heteroaromatic ring system are preferably selected from N, O and S.

The terms “heteroaromatic ring system” and “aromatic ring system” as defined in the present application thus differ from one another in that an aromatic ring system cannot have a heteroatom as ring atom, whereas a heteroaromatic ring system must have at least one heteroatom as ring atom. This heteroatom may be present as a ring atom of a non-aromatic heterocyclic ring or as a ring atom of an aromatic heterocyclic ring.

In accordance with the above definitions, any aryl group is covered by the term “aromatic ring system”, and any heteroaryl group is covered by the term “heteroaromatic ring system”.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be replaced by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethenylthio, propynylthio, butynylthio, pentenylthio, hexenylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical binds to the position to which the hydrogen atom was bonded, forming a ring.

T¹ is preferably —(C═O)(NAr¹)—.

Preferably, Ar¹ is the same or different at each instance and is selected from monovalent groups derived from benzene, biphenyl, terphenyl, quaterphenyl, triphenylene, naphthalene, fluorene, benzofluorene, spirobifluorene, indenofluorene, indenocarbazole, dibenzofuran, dibenzothiophene, benzocarbazole, carbazole, benzofuran, benzothiophene, indole, benzimidazole, quinazoline, quinoxaline, quinoline, pyridine, pyrimidine, pyrazine, pyridazine and triazine, where the monovalent groups may each be substituted by one or more R³ radicals. Alternatively, Ar¹ may preferably be the same or different at each instance and may be selected from combinations of groups derived from benzene, biphenyl, terphenyl, quaterphenyl, triphenylene, naphthalene, fluorene, especially 9,9′-dimethylfluorene and 9,9′-diphenylfluorene, benzofluorene, spirobifluorene, indenofluorene, indenocarbazole, dibenzofuran, dibenzothiophene, carbazole, benzofuran, benzothiophene, indole, benzimidazole, quinazoline, quinoxaline, quinoline, pyridine, pyrimidine, pyrazine, pyridazine and triazine, where the groups may each be substituted by one or more R³ radicals.

More preferably, Ar¹ is the same or different at each instance and is selected from phenyl, biphenyl, terphenyl, quaterphenyl, triphenylene, naphthyl, fluorenyl, dibenzofuranyl, dibenzothiophenyl, carbazolyl, benzofuranyl, benzothiophenyl, triazinyl, pyrimidyl, pyridyl, quinazoline, quinoxaline and quinoline, where the groups mentioned may each be substituted by one or more R³ radicals.

Preferably not more than one Z¹ group per formula is N, and the other Z¹ groups are selected from C and CR¹. Z¹ is preferably the same or different at each instance and is selected from C and CR¹.

Preferably not more than one Z² group per formula is N, and the other Z² groups are selected from C and CR². Z² is preferably the same or different at each instance and is selected from C and CR².

Preferably, k is 0.

Preferably, R¹ is the same or different at each instance and is selected from H, D, F, CN, Si(R⁴)₃, N(R⁴)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁴ radicals; and where one or more CH₂ groups in the alkyl or alkoxy groups mentioned may be replaced by —C≡C—, —R⁴C═CR⁴—, Si(R⁴)₂, C═O, C═NR⁴, —NR⁴—, —O—, —S—, —C(═O)O— or —C(═O)NR⁴—. More preferably, R¹ is the same or different at each instance and is selected from H, aromatic ring systems having 6 to 40 aromatic ring atoms, and heteroaromatic ring systems having 5 to 40 aromatic ring atoms; where the aromatic ring systems mentioned and the heteroaromatic ring systems mentioned are each substituted by R⁴ radicals.

Preferably, R² is the same or different at each instance and is selected from H, D, F, CN, Si(R⁴)₃, N(R⁴)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁴ radicals; and where one or more CH₂ groups in the alkyl or alkoxy groups mentioned may be replaced by —C≡C—, —R⁴C═CR⁴—, Si(R⁴)₂, C═O, C═NR⁴, —NR⁴—, —O—, —S—, —C(═O)O— or —C(═O)NR⁴—. More preferably, R² is the same or different at each instance and is selected from H, aromatic ring systems having 6 to 40 aromatic ring atoms, and heteroaromatic ring systems having 5 to 40 aromatic ring atoms; where the aromatic ring systems mentioned and the heteroaromatic ring systems mentioned are each substituted by R⁴ radicals.

Preferably, R³ is the same or different at each instance and is selected from H, D, F, CN, Si(R⁴)₃, N(R⁴)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁴ radicals; and where one or more CH₂ groups in the alkyl or alkoxy groups mentioned may be replaced by —C≡C—, —R⁴C═CR⁴—, Si(R⁴)₂, C═O, C═NR⁴, —NR⁴—, —O—, —S—, —C(═O)O— or —C(═O)NR⁴—. More preferably, R³ is the same or different at each instance and is selected from H, aromatic ring systems having 6 to 40 aromatic ring atoms, and heteroaromatic ring systems having 5 to 40 aromatic ring atoms; where the aromatic ring systems mentioned and the heteroaromatic ring systems mentioned are each substituted by R⁴ radicals.

Preferably, R⁴ is the same or different at each instance and is selected from H, D, F, CN, Si(R⁵)₃, N(R⁵)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁵ radicals; and where one or more CH₂ groups in the alkyl or alkoxy groups mentioned may be replaced by —C≡C—, —R⁵C═CR⁵—, Si(R⁵)₂, C═O, C═NR⁵, —NR⁵—, —O—, —S—, —C(═O)O— or —C(═O)NR⁵—.

Preferably, R⁵ is H.

Formula (I) preferably conforms to one of the following formulae:

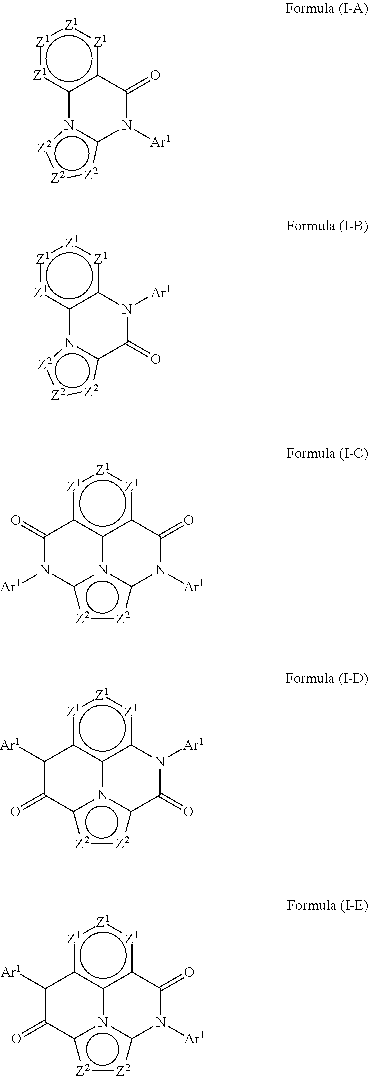

where the symbols that occur are as defined above. In a preferred embodiment, Z¹ is the same or different at each instance and is C or CR¹. In an alternative preferred embodiment, exactly one Z¹ group per formula is N, and the remaining Z¹ groups are the same or different at each instance and are C or CR¹. In a further preferred embodiment, Z² is the same or different at each instance and is C or CR². In an alternative preferred embodiment, exactly one Z² group per formula is N, and the remaining Z² groups are the same or different at each instance and are C or CR².

In a preferred embodiment, Z¹ is the same or different at each instance and is C or CR¹, and Z² is the same or different at each instance and is C or CR². In an alternative preferred embodiment, Z¹ is the same or different at each instance and is C or CR¹, and exactly one Z² group per formula is N, and the remaining Z² groups are the same or different at each instance and are C or CR².

It is preferable that the compound containing a structural unit of formula (I) conforms to one of the following formulae:

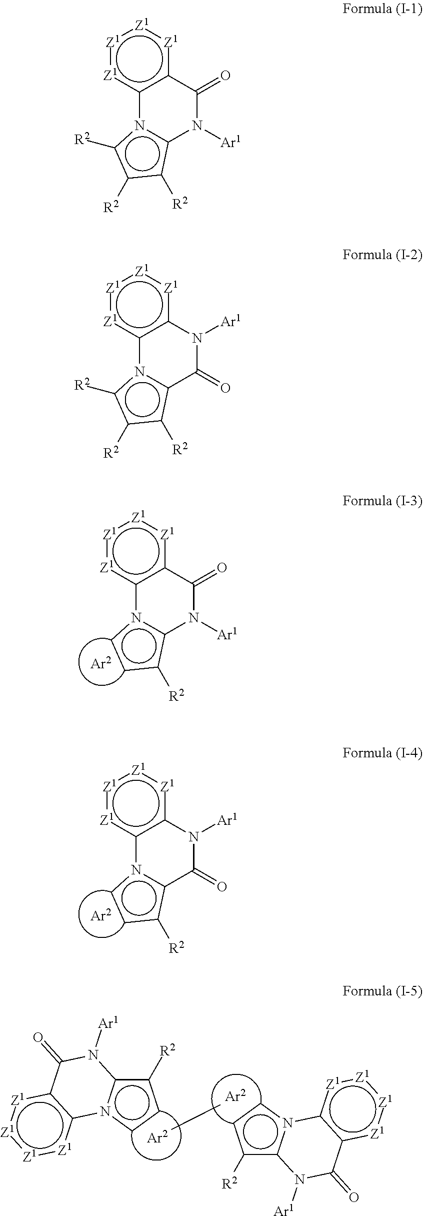

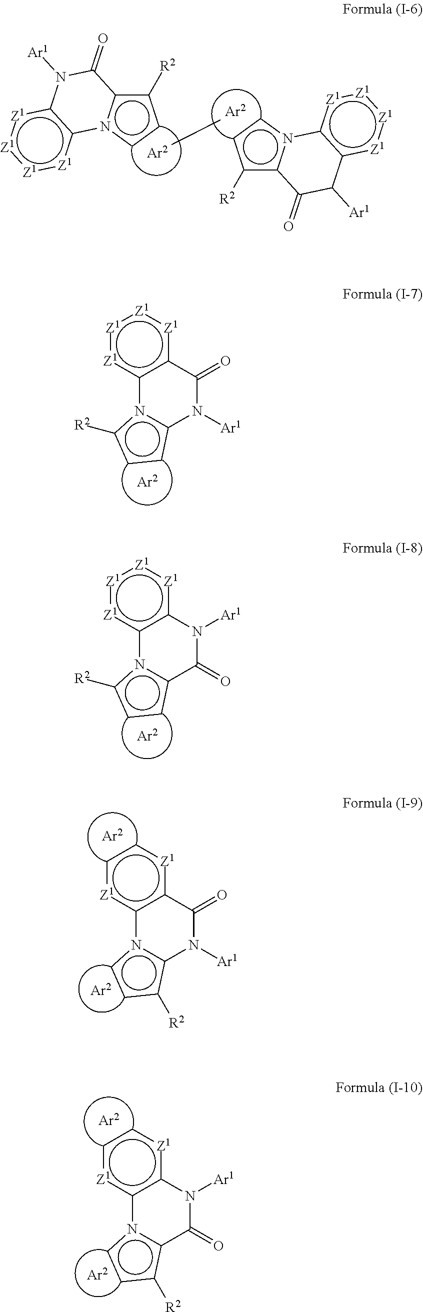

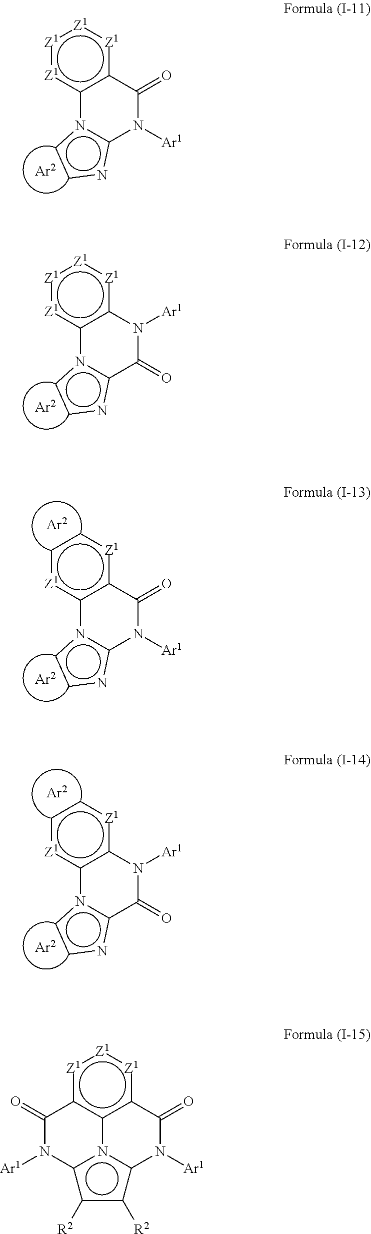

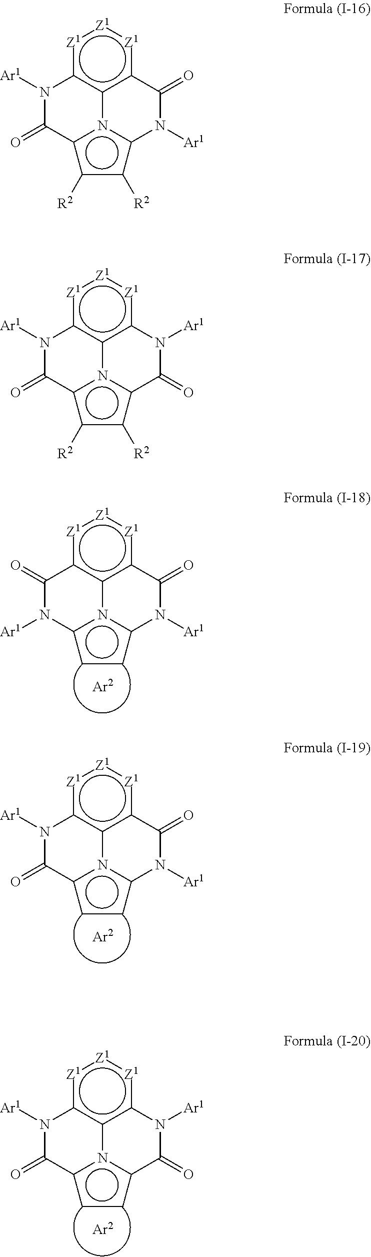

where Ar² is selected from aromatic ring systems which have 4 to 40 aromatic ring atoms and are substituted by R^{A} radicals and heteroaromatic ring systems which have 3 to 40 aromatic ring atoms and are substituted by R^{A} radicals, and where Z¹ is the same or different at each instance and is CR¹ or N, and where the other symbols are as defined above.

Preferably, Ar² is an aromatic ring system which has 4 aromatic ring atoms and is substituted by R^{A} radicals; in other words, fused-on benzene substituted by R^{A} radicals.

The Ar² group shown in the above formulae (I-3) to (I-14) and (I-18) to (I-20) is fused onto the five-membered ring or six-membered ring in exactly the same way as a benzene group is fused onto another benzene group in an ethylene group, in that the two benzene rings share two carbon atoms and the bond between them.

One example of this is the following embodiment covered by formula (I-3):

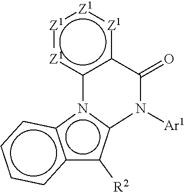

in which Ar² is a C₄H₄ unit derived from a benzene ring.

R^{A} is the same or different at each instance and is selected from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R^{A} radicals may be joined to one another and may form a ring; where the alkyl, alkoxy, alkenyl and alkynyl groups mentioned and the aromatic ring systems and heteroaromatic ring systems mentioned are each substituted by R⁴ radicals; and where one or more CH₂ groups in the alkyl, alkoxy, alkenyl and alkynyl groups mentioned may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂.

Preferably, R^{A} is the same or different at each instance and is selected from H, D, F, CN, Si(R⁴)₃, N(R⁴)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and heteroaromatic ring systems having 5 to 40 aromatic ring atoms, where the alkyl and alkoxy groups mentioned, the aromatic ring systems mentioned and the heteroaromatic ring systems mentioned are each substituted by R⁴ radicals.

Preferably, Z¹ in the above formulae is CR¹. In an alternative preferred embodiment, exactly one Z¹ group per formula is N, and the remaining Z² groups are CR¹.

Preferably, in the above formulae (I-1) to (I-20), there is at least one R¹, R², R³ or R^{A} group selected from
aromatic ring systems which have 6 to 40 aromatic ring atoms and are substituted by R⁴ radicals (radical A),
heteroaromatic ring systems which have 5 to 40 aromatic ring atoms and are substituted by R⁴ radicals (radical B),
N(R⁴)₂, where R⁴ groups in N(R⁴)₂ groups are preferably the same or different at each instance and are selected from aromatic ring systems which have 6 to 40 aromatic ring atoms and are substituted by R⁵ radicals, and heteroaromatic ring systems which have 5 to 40 aromatic ring atoms and are substituted by R⁵ radicals (radical C).

Corresponding compounds are provided by the present application.

Radicals A are preferably the same or different at each instance and are selected from phenyl, biphenyl, terphenyl, quaterphenyl, triphenylene, naphthyl, fluorenyl and spirobifluorenyl, each substituted by R^{A} radicals.

Radicals B are preferably the same or different at each instance and are selected from dibenzofuranyl, dibenzothiophenyl, carbazolyl, benzofuranyl, benzothiophenyl, triazinyl, pyrimidyl, pyridyl, quinazolinyl, quinoxalinyl and quinolinyl, each substituted by R^{A} radicals.

Preferably, in the above formulae (I-1) to (I-20), there is exactly one R¹, R², R³ or R^{A} group selected from the abovementioned radicals A, B and C. More preferably, in this case, the other R¹, R², R³ or R^{A} groups present are H.

Among the abovementioned formulae, particular preference is given to the formulae (I-1) to (I-4), (I-11), (I-12) and (I-15).

Particularly preferred embodiments of the compound conform to the following formulae:

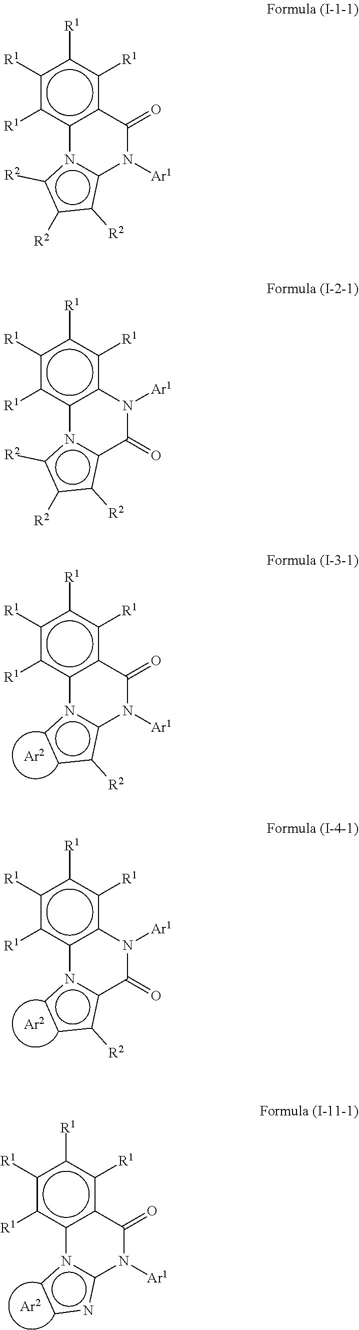

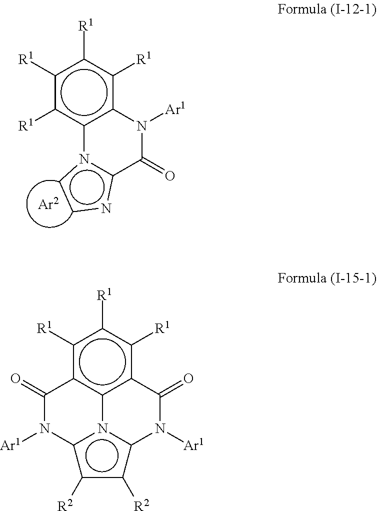

where the symbols that occur are as defined above, and preferably correspond to their above-specified preferred embodiments.

Preferably, in the formulae (I-1-1) to (I-4-1), (I-11-1), (I-12-1) and (I-15-1), all R¹ groups are H. Further preferably, all R² groups are H. Further preferably, all R³ groups are H. Further preferably, all R^{A} groups are H. More preferably, all R¹, R², R³ and R^{A} groups are H.

In an alternative preferred embodiment, in the formulae (I-1-1) bis (I-4-1), (I-11-1), (I-12-1) and (I-15-1), at least one group selected from R¹, R², R³ and R^{A} groups is selected from the abovementioned radicals A, B and C. More preferably, in this case, the other R¹, R², R³ and R^{A} groups are H.

Further preferably, Are is a fused-on benzene group substituted by R^{A} radicals. Accordingly, preferred embodiments of the formulae (I-3-1), (I-4-1), (I-11-1) and (I-12-1) are selected from the following formulae:

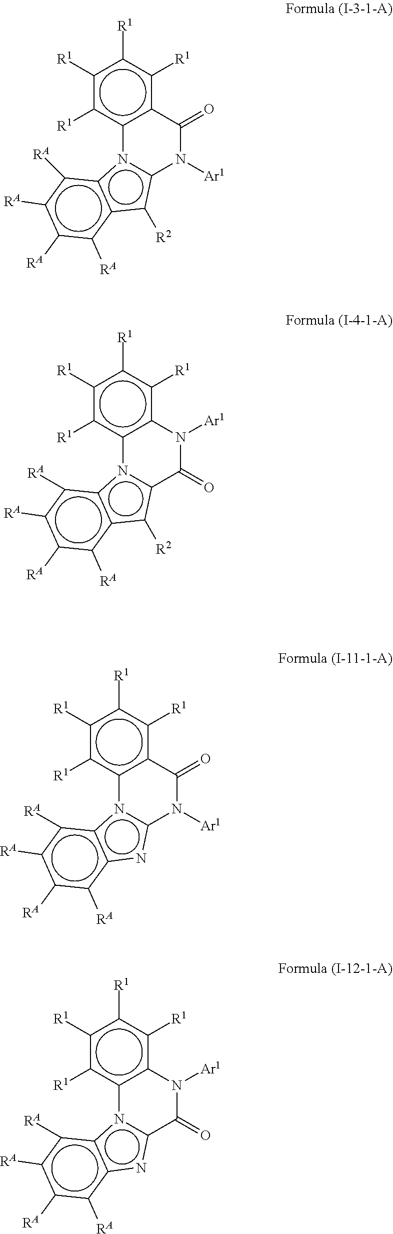

where the symbols that occur are as defined for formulae (I-1-1) to (I-4-1), (I-11-1), (I-12-1) and (I-15-1).

Preferably, in the formulae (I-3-1-A), (I-4-1-A), (I-11-1-A) and (I-12-1-A), all R¹ groups are H. Further preferably, all R² groups are H. Further preferably, all R³ groups are H. Further preferably, all R^{A} groups are H. More preferably, all R¹, R², R³ and R^{A} groups are H.

In an alternative preferred embodiment, in the formulae (I-3-1-A), (I-4-1-A), (I-11-1-A) and (I-12-1-A), at least one group selected from R¹, R², R³ and R^{A} groups is selected from the abovementioned radicals A, B and C. More preferably, in this case, the other R¹, R², R³ and R^{A} groups are H.

Particularly preferred embodiments of the compound conform to the following formulae:

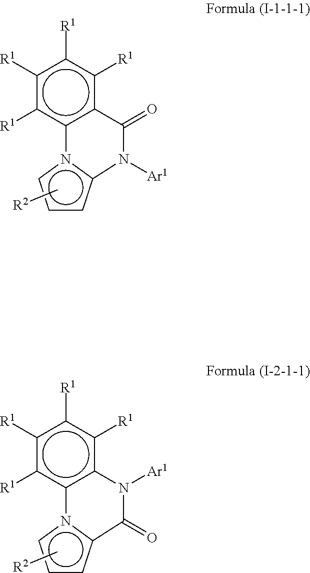

where the symbols that occur are as defined above, and where R² is preferably selected from radicals A, B and C. Preferably, R¹ in the formulae is H.

Particularly preferred embodiments of the compound conform to the following formulae:

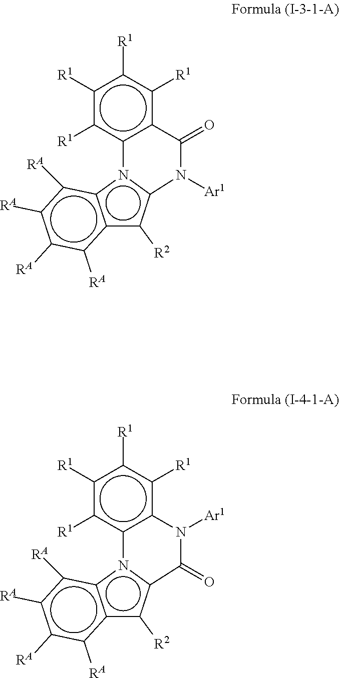

where the symbols that occur are as defined above, and where R² is preferably selected from radicals A, B and C. Preferably, in the formulae, R¹ is H and/or R^{A} is H; more preferably, R¹ and R^{A} are H.

Particularly preferred embodiments of the compound conform to the following formulae:

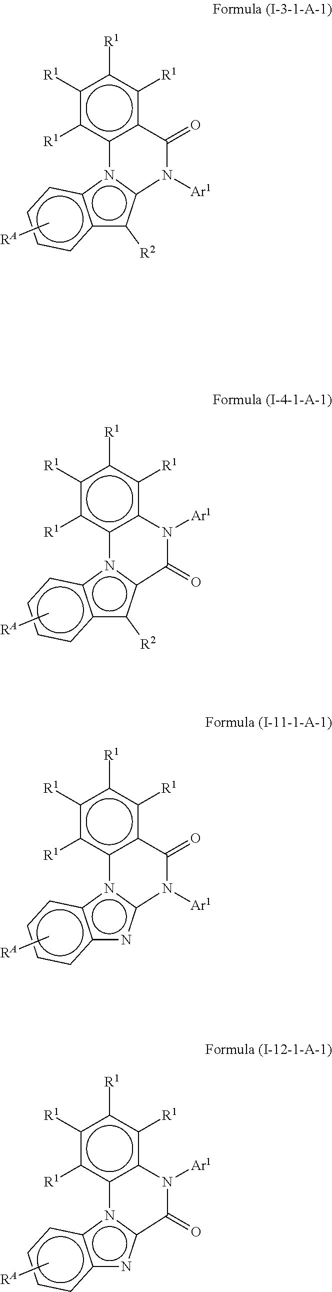

where the symbols that occur are as defined above, and where R^{A} is preferably selected from radicals A, B and C. Preferably, in the formulae, R¹ is H and/or R² is H; more preferably, R¹ and R² are H.

Particularly preferred embodiments of the compound conform to the following formulae:

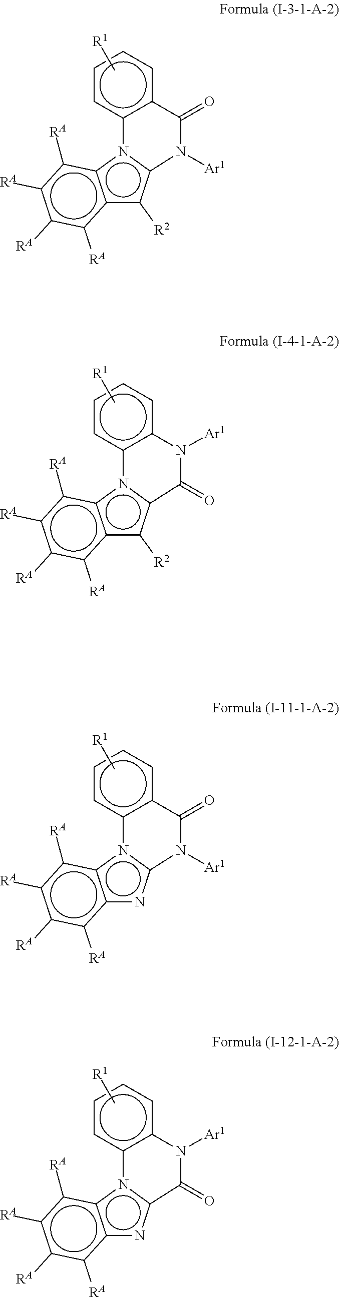

where the symbols that occur are as defined above, and where R¹ is preferably selected from radicals A, B and C. Preferably, in the formulae, R² is H and/or R^{A} is H; more preferably, R² and R^{A} are H.

Particularly preferred embodiments of the compound conform to the following formula:

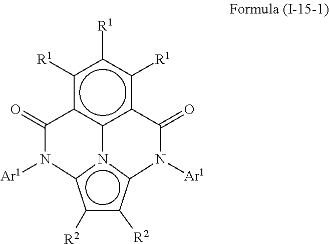

where the symbols that occur are as defined above, and where, preferably, R¹ is H and/or R² is H; more preferably, R¹ and R² are H.

Preferred compounds containing a structural unit of the formula (I) are depicted below:

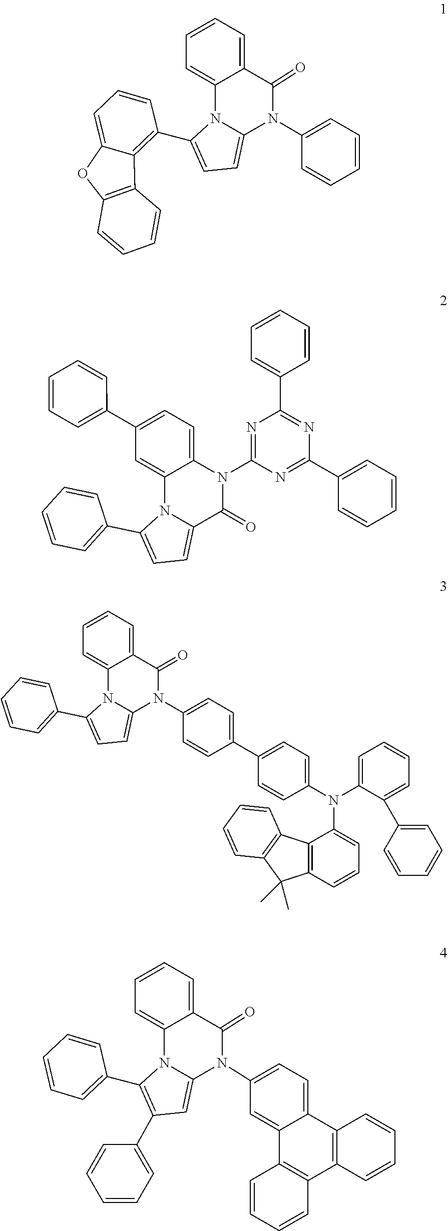

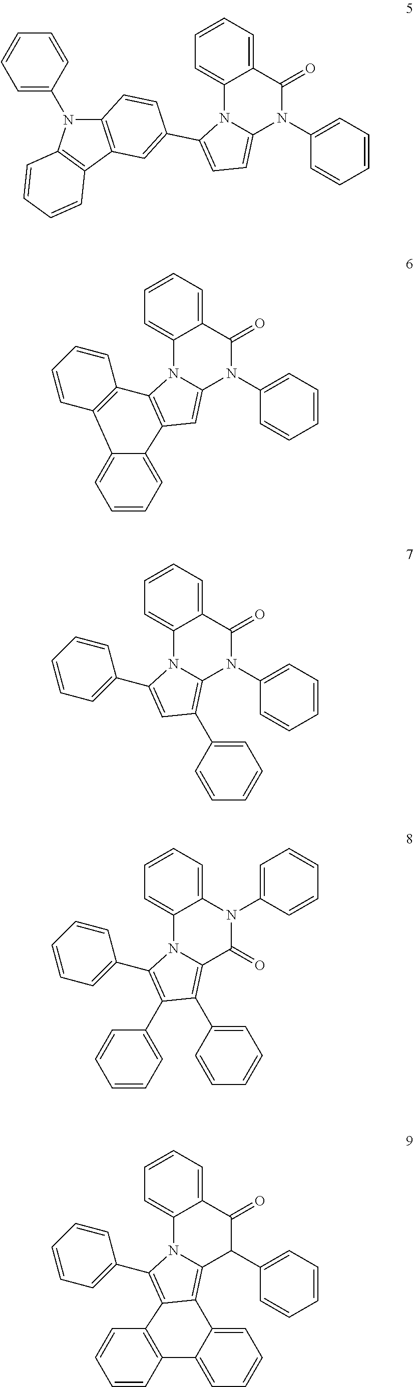

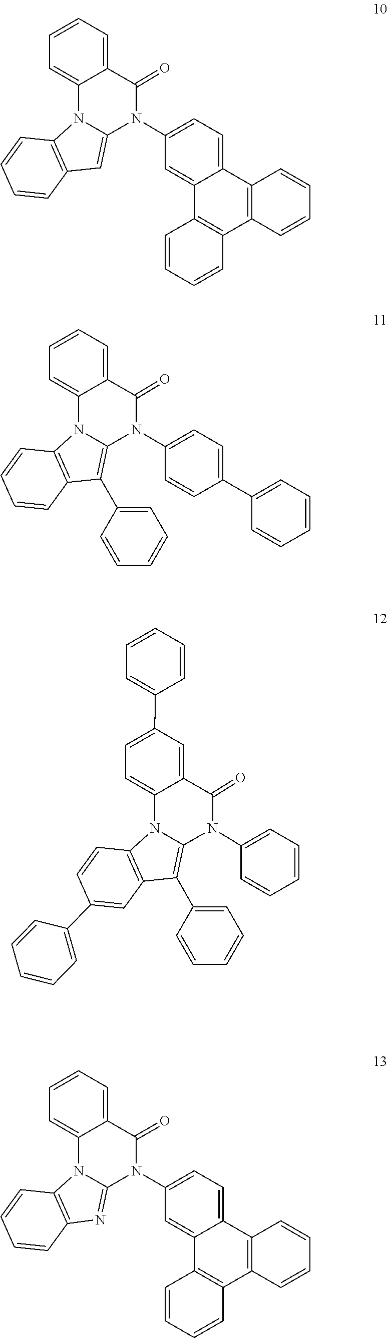

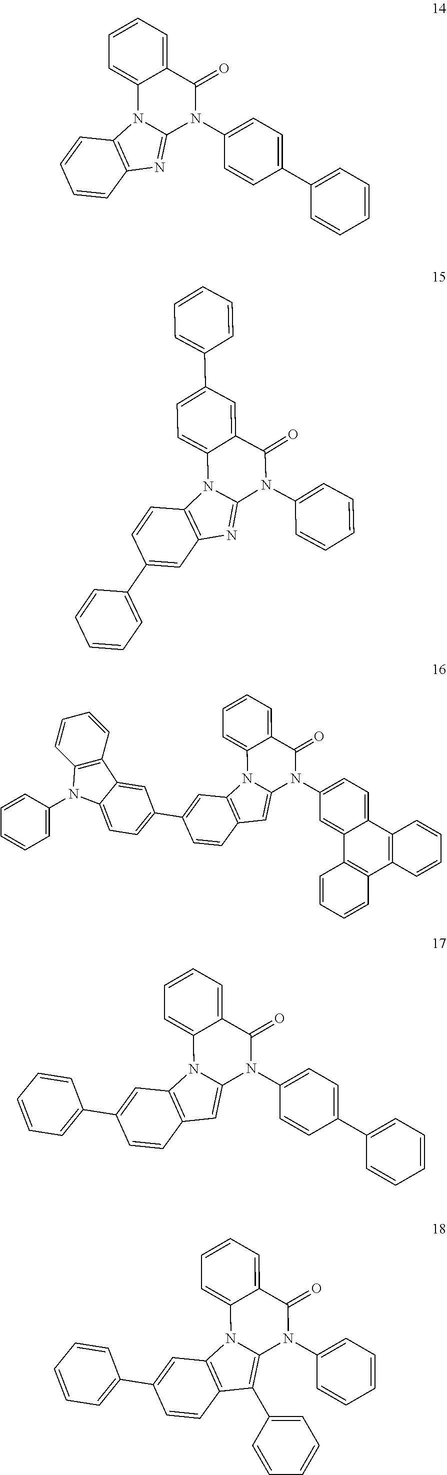

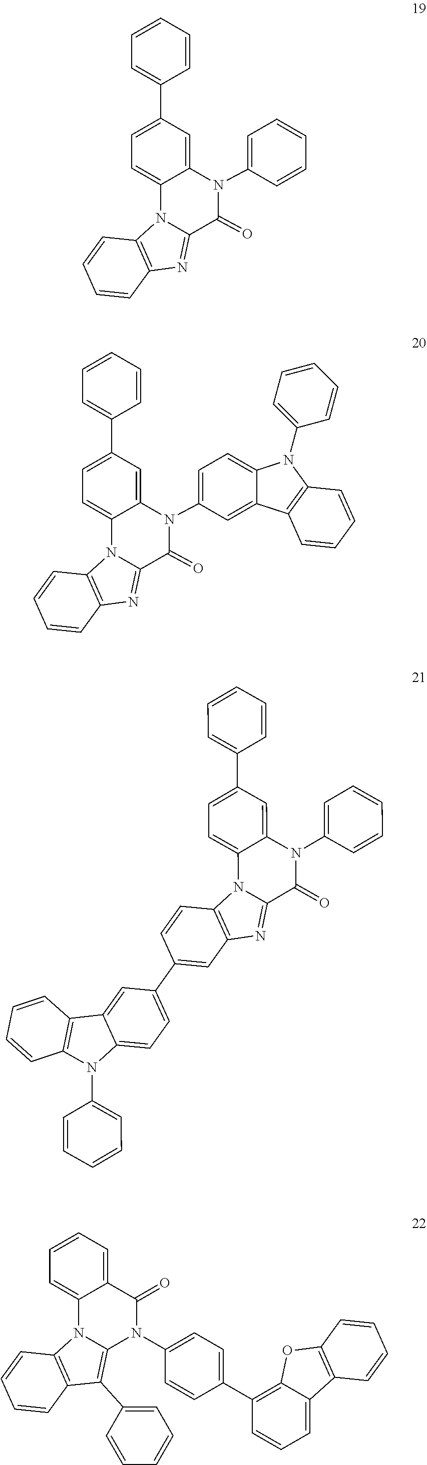

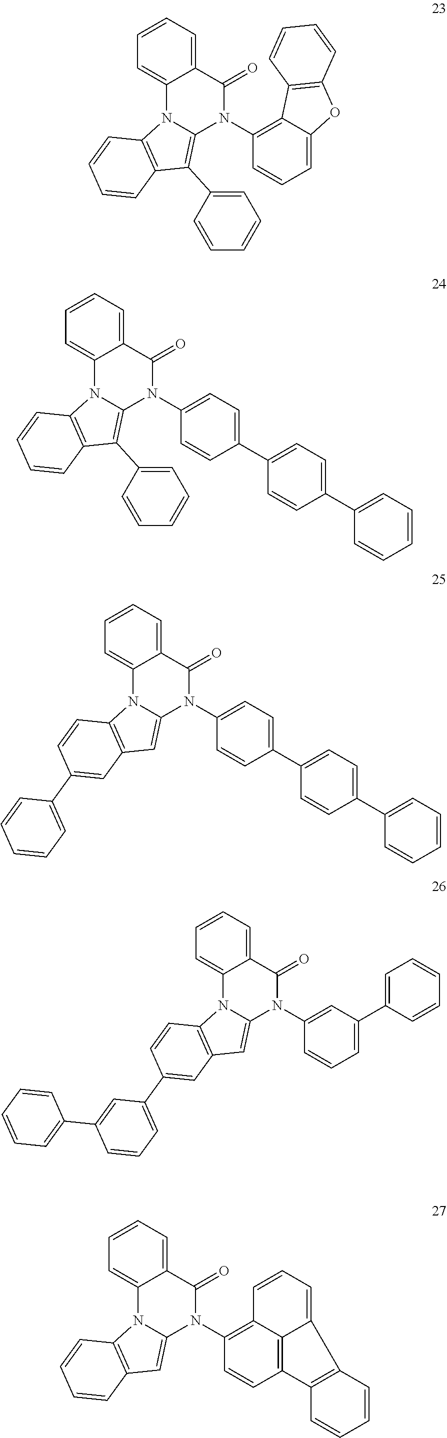

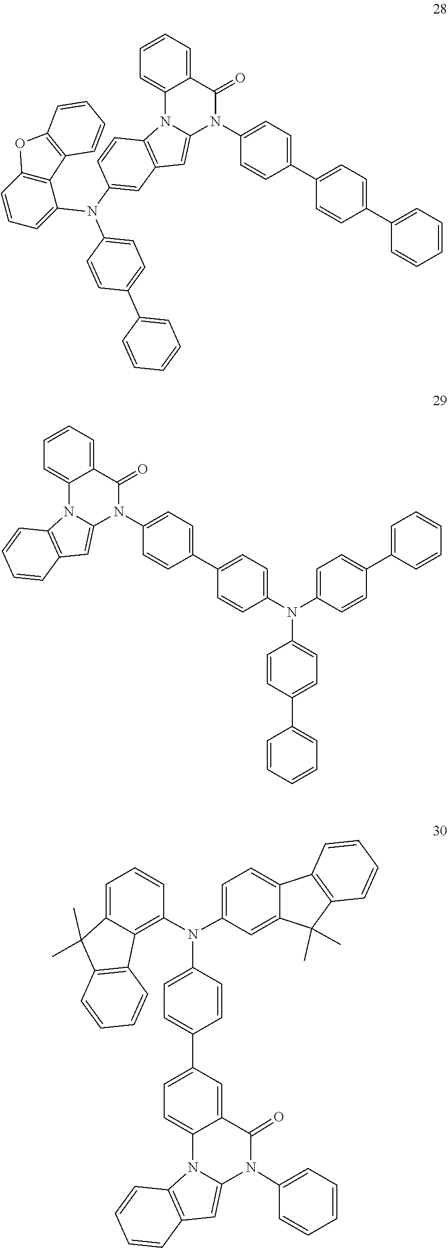

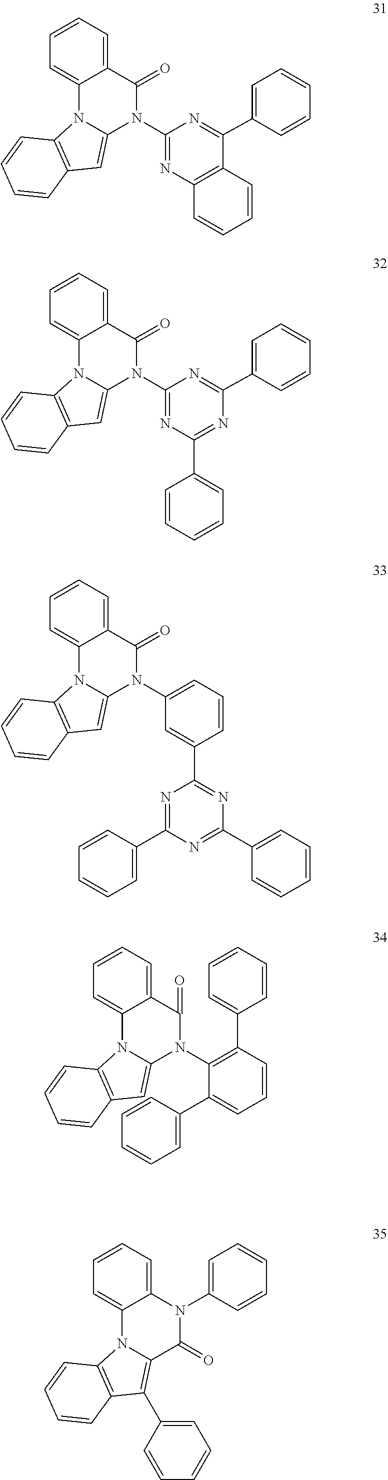

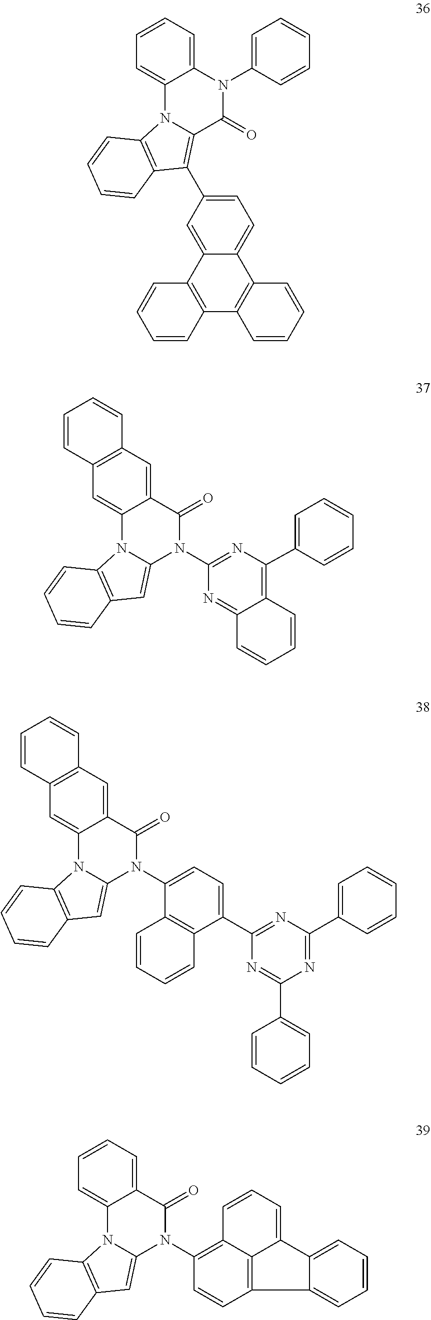

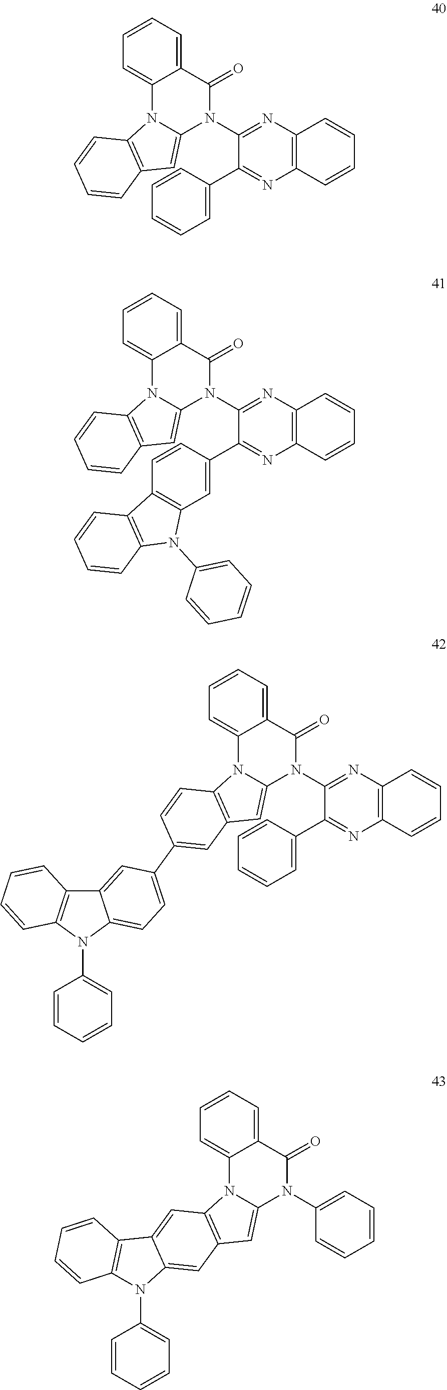

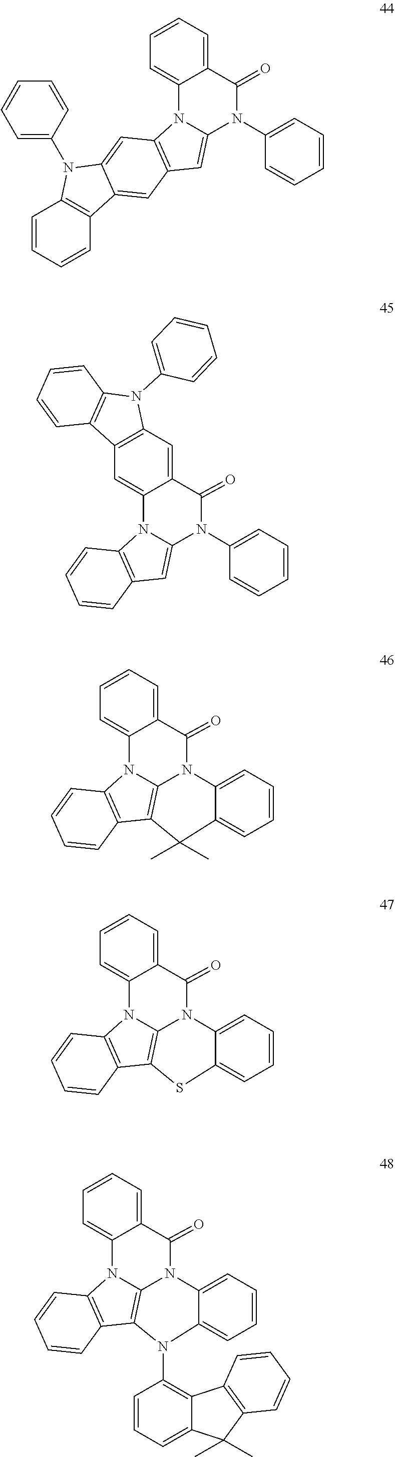

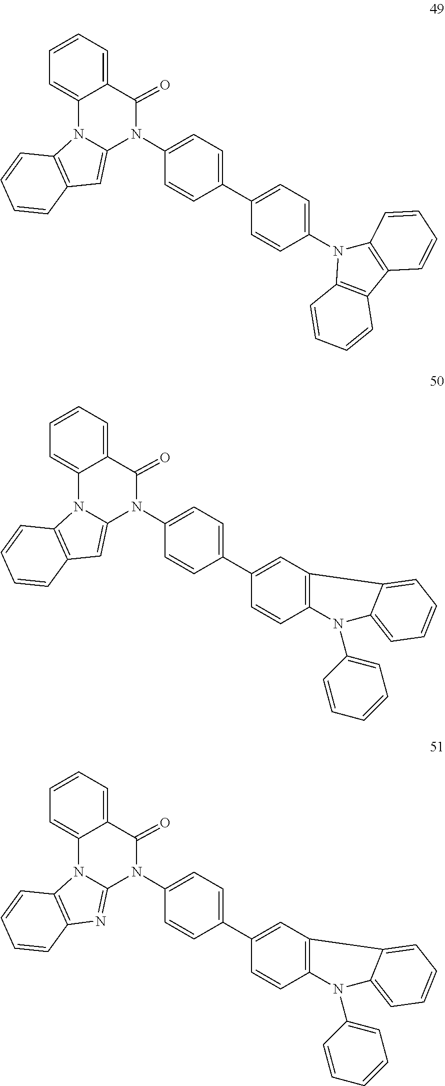

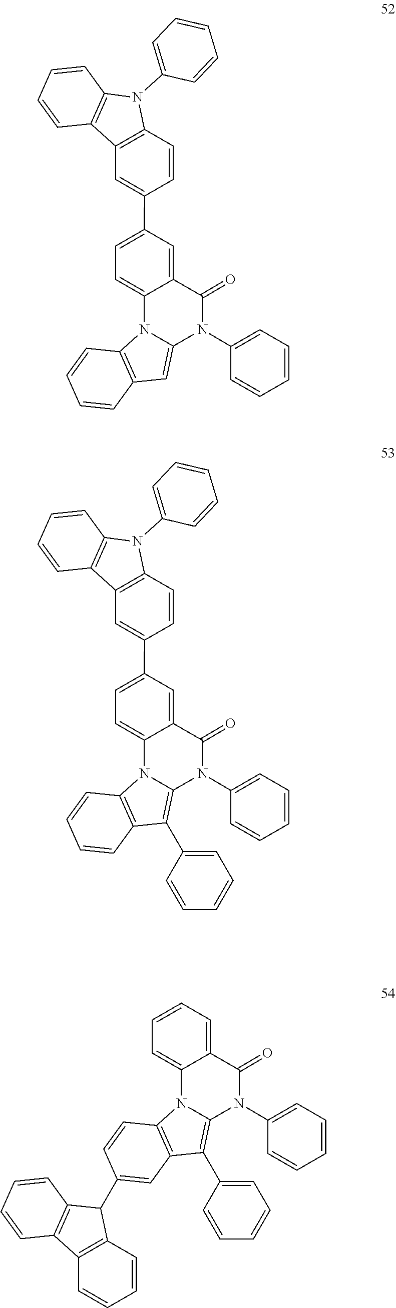

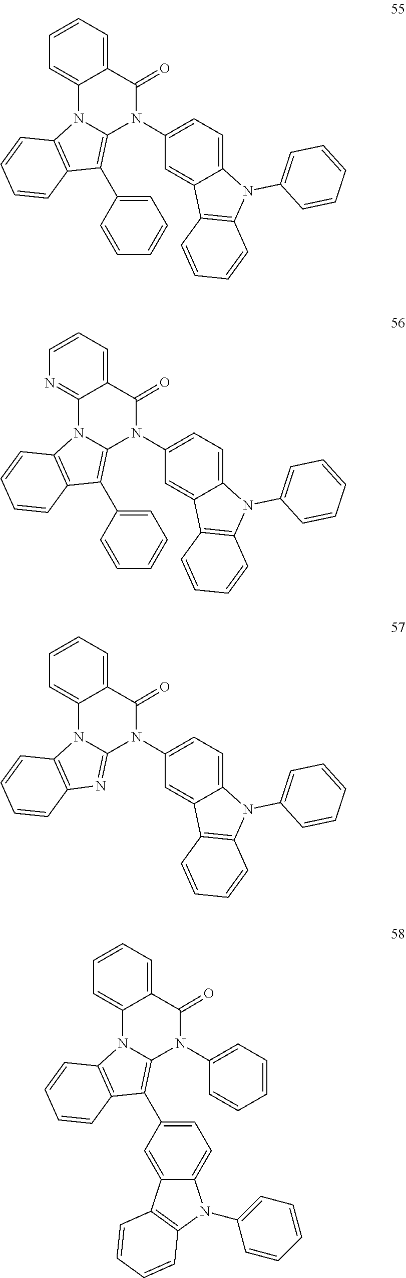

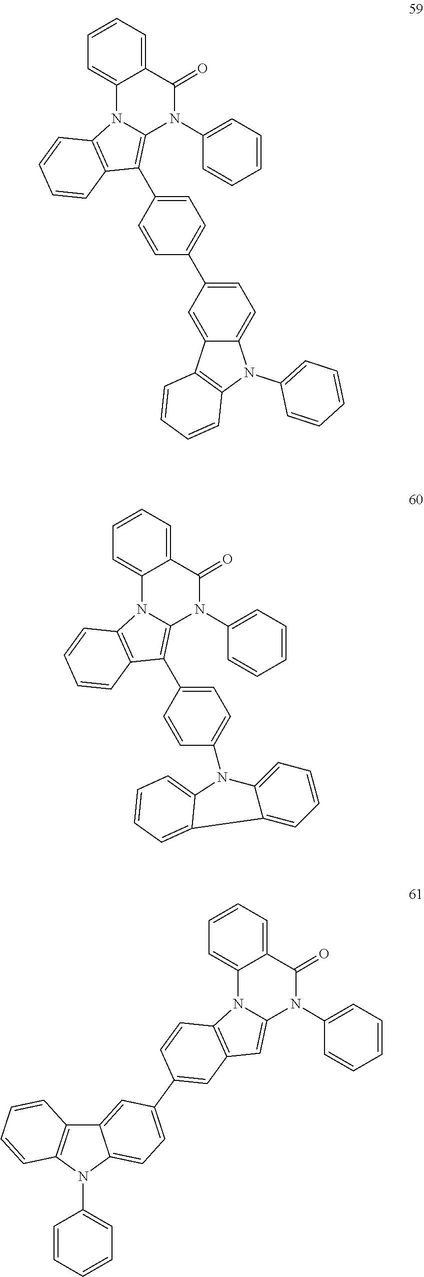

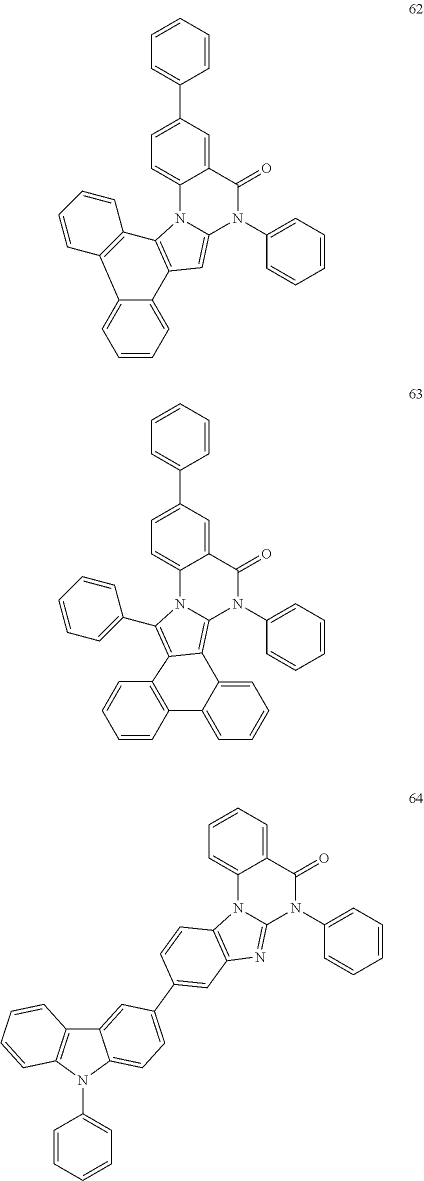

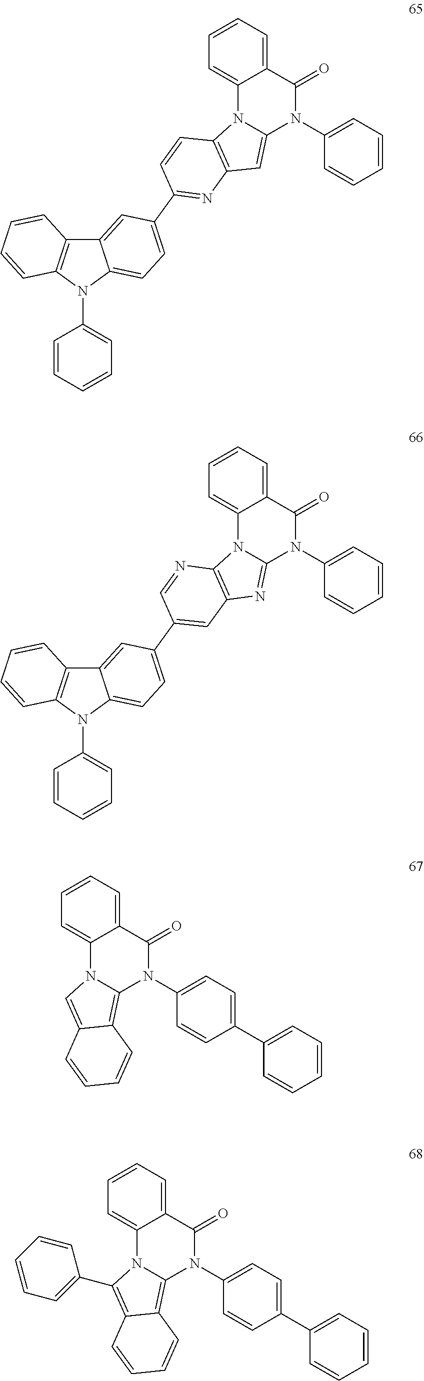

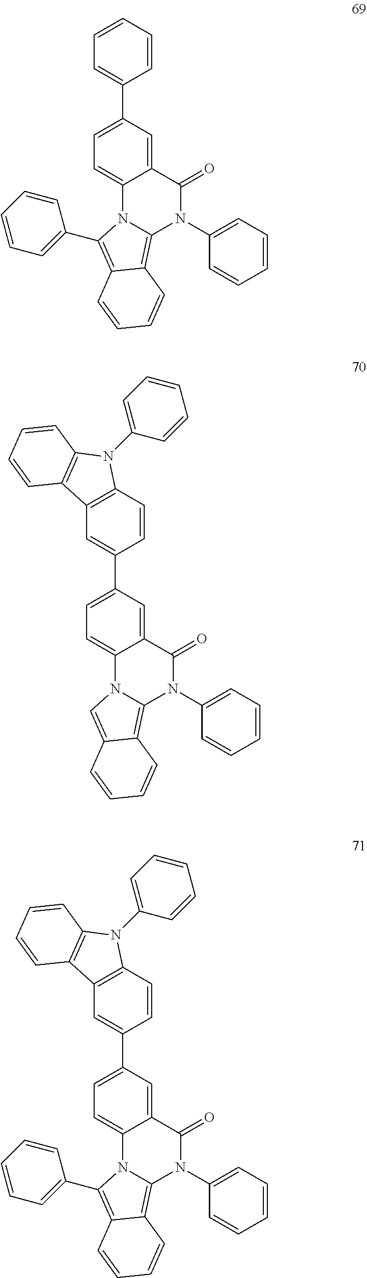

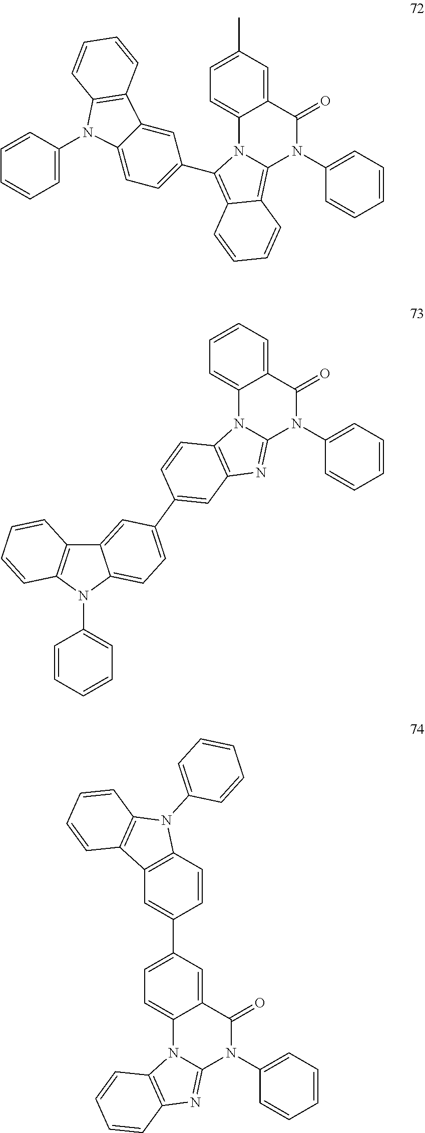

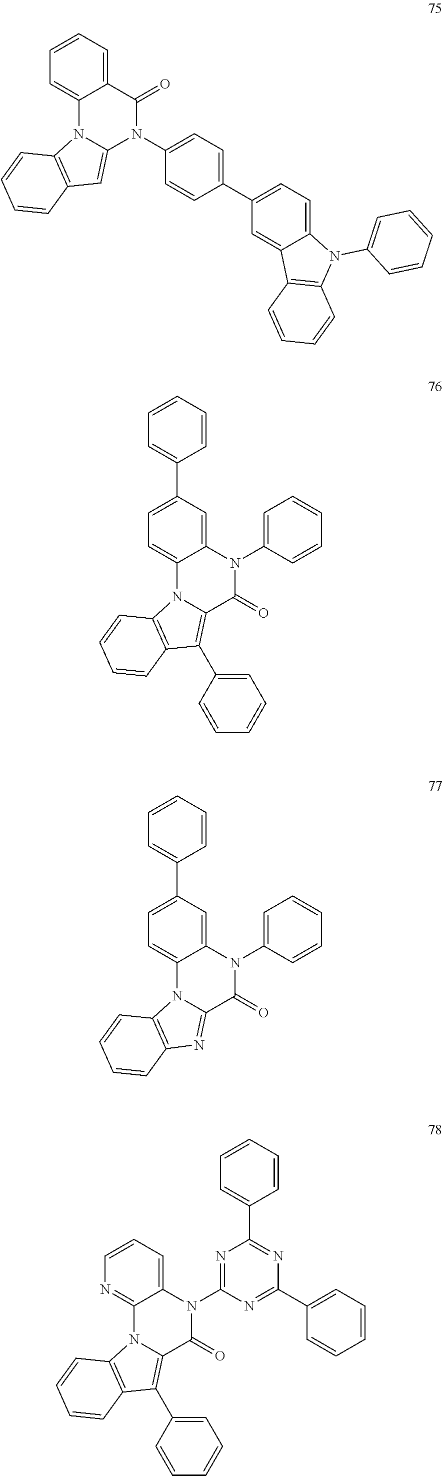

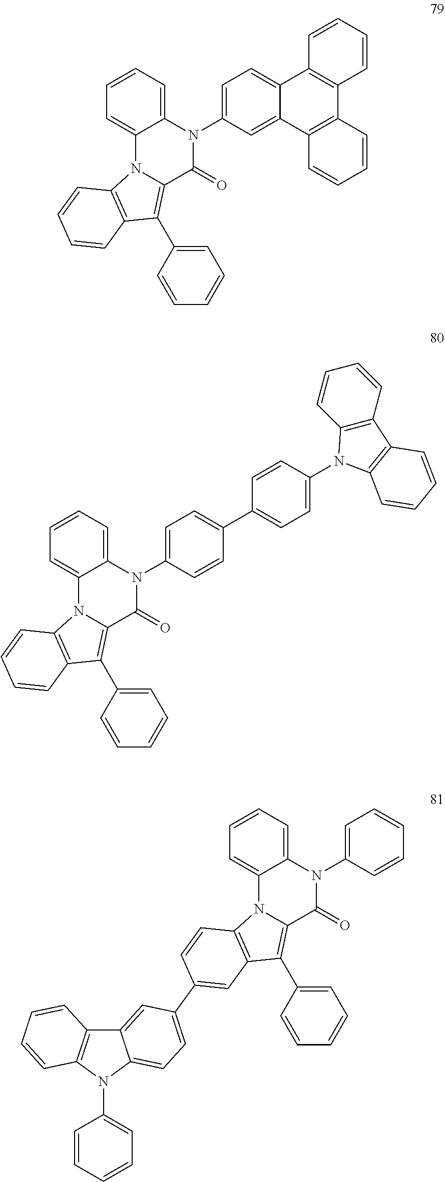

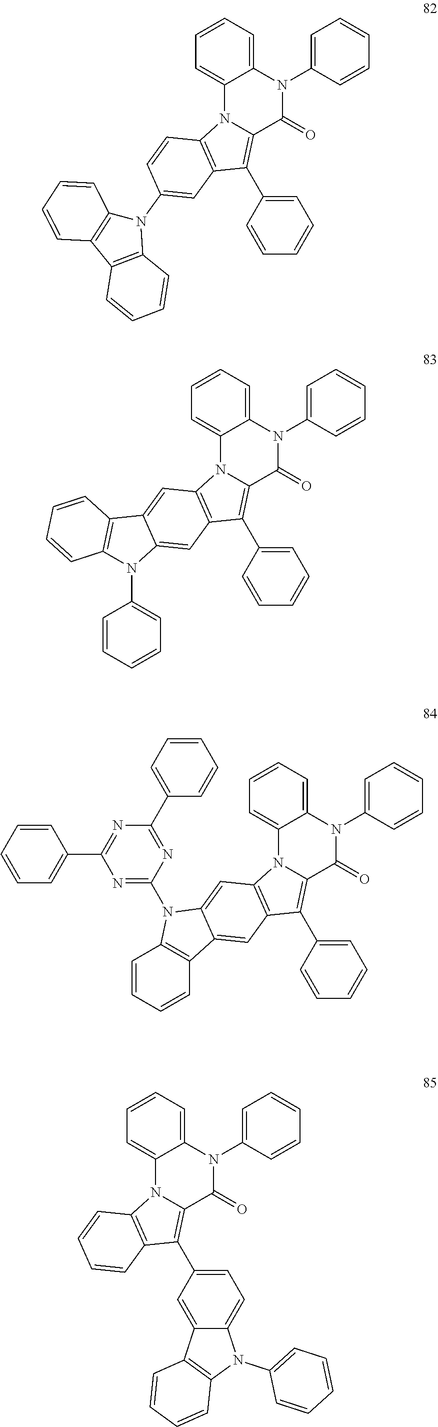

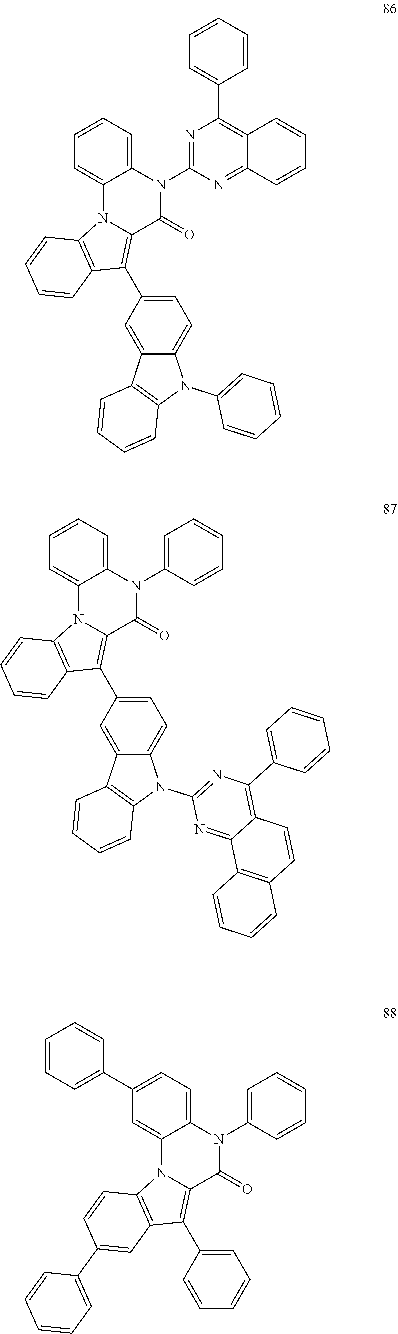

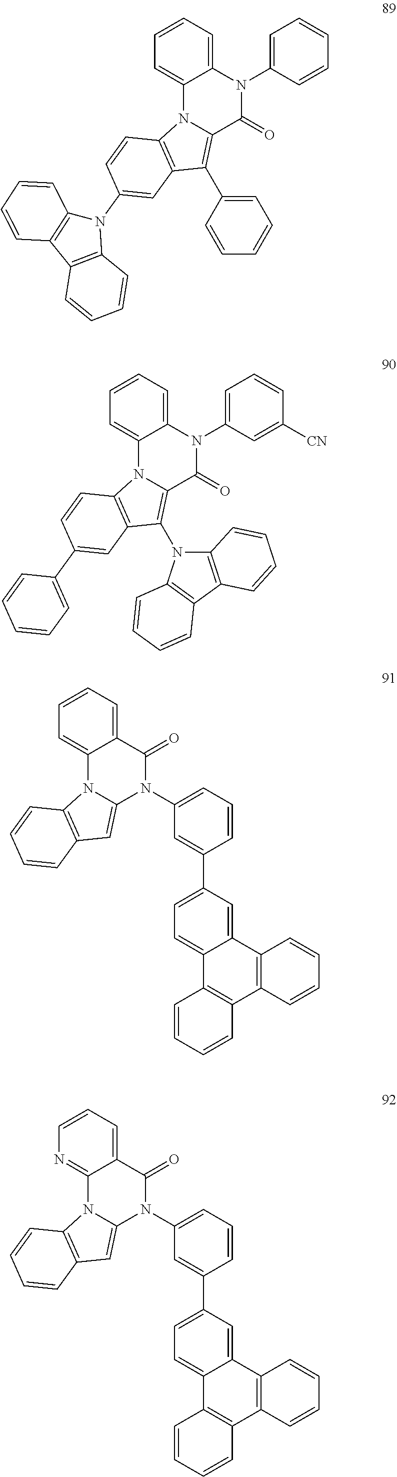

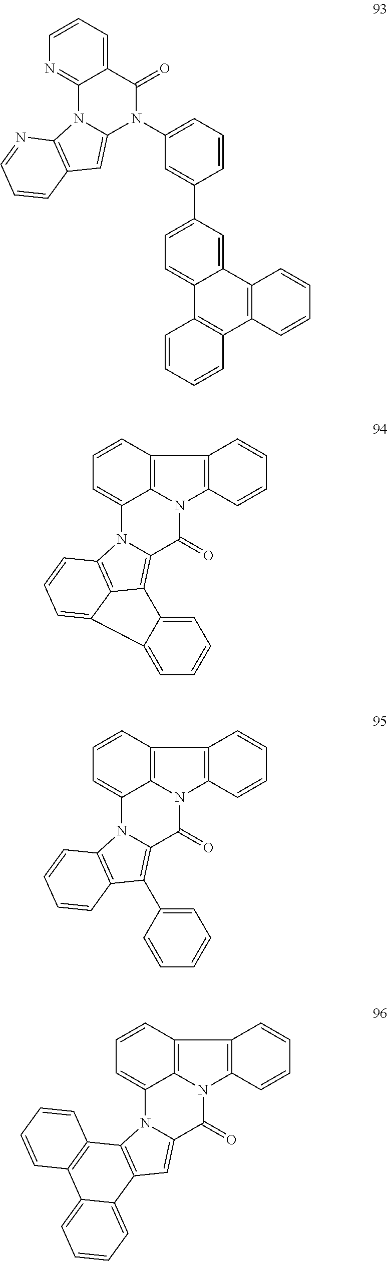

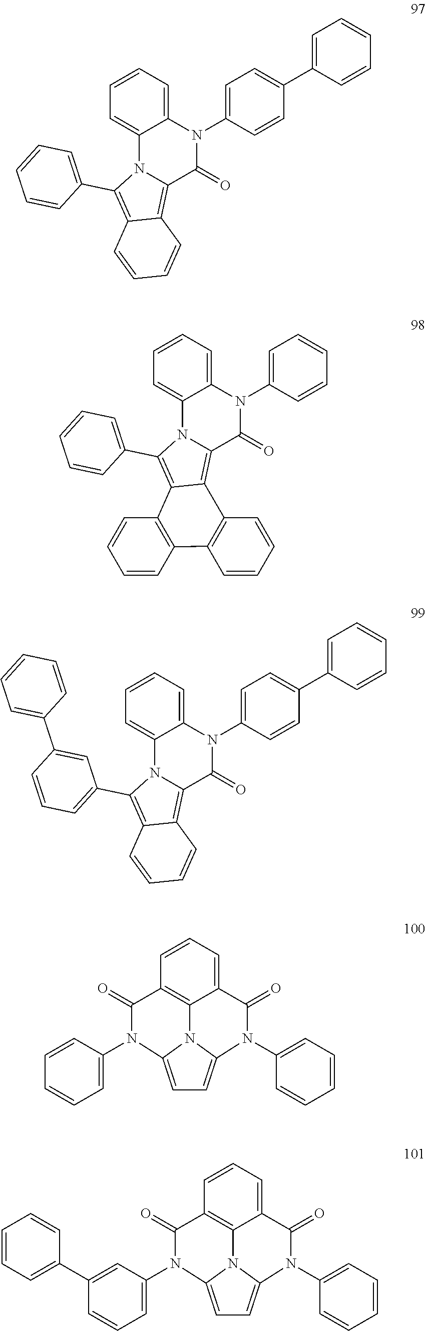

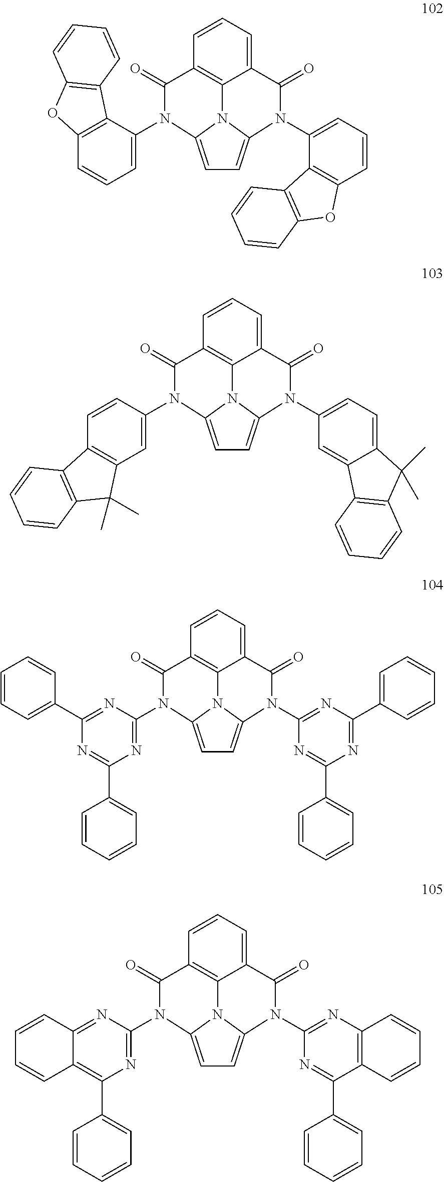

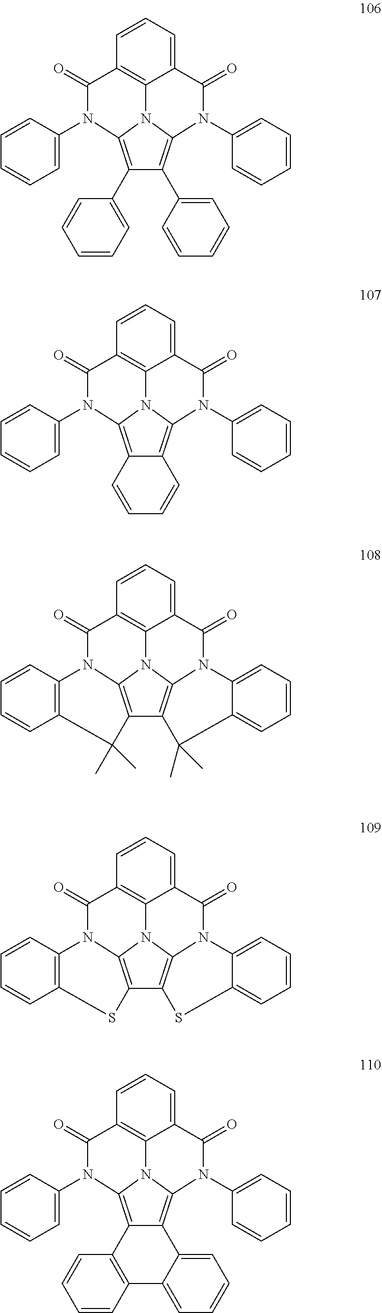

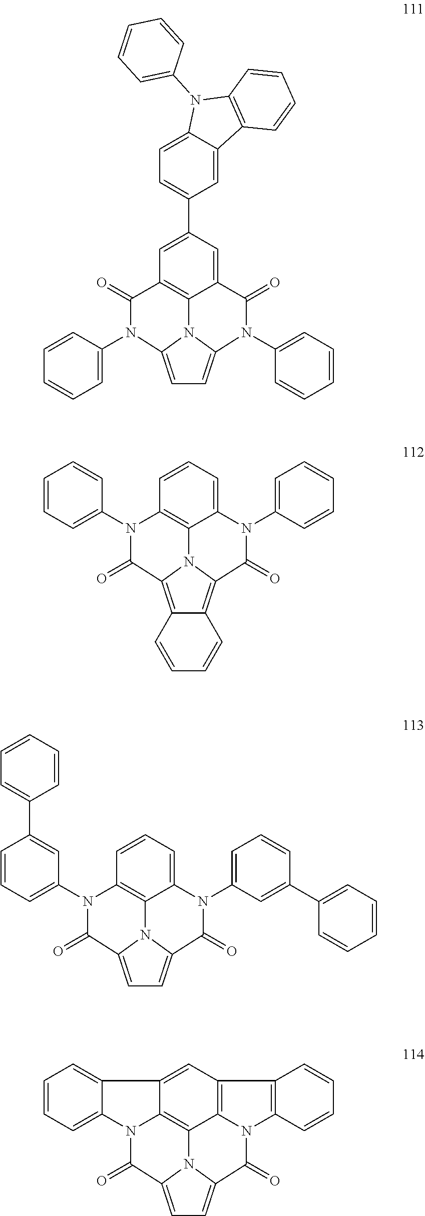

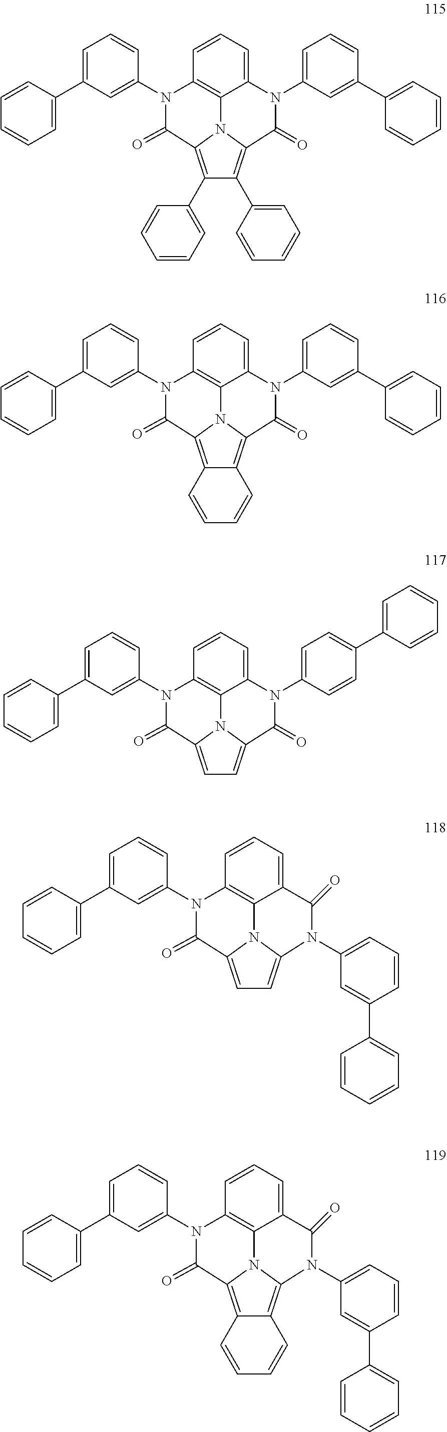

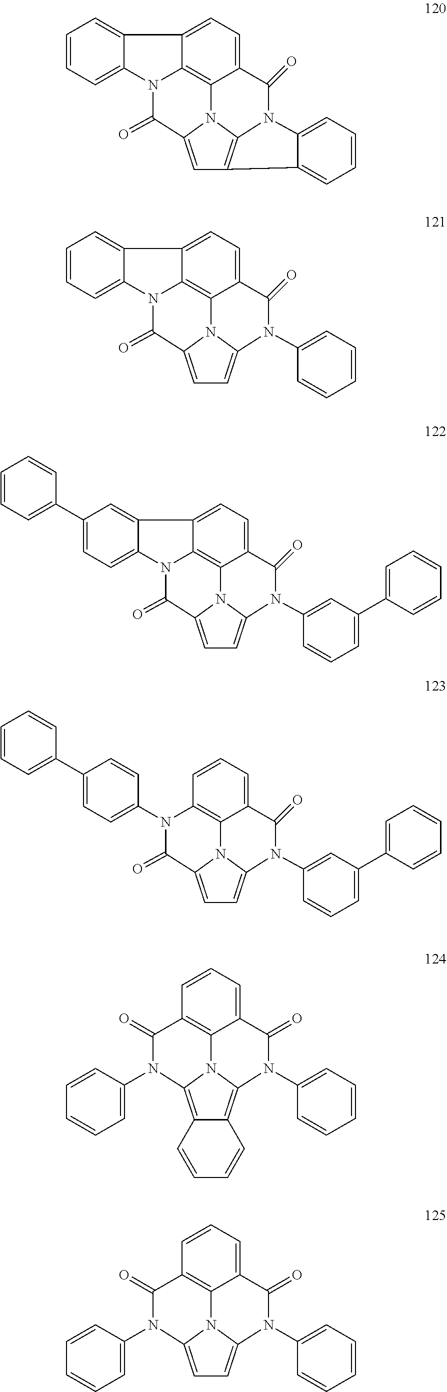

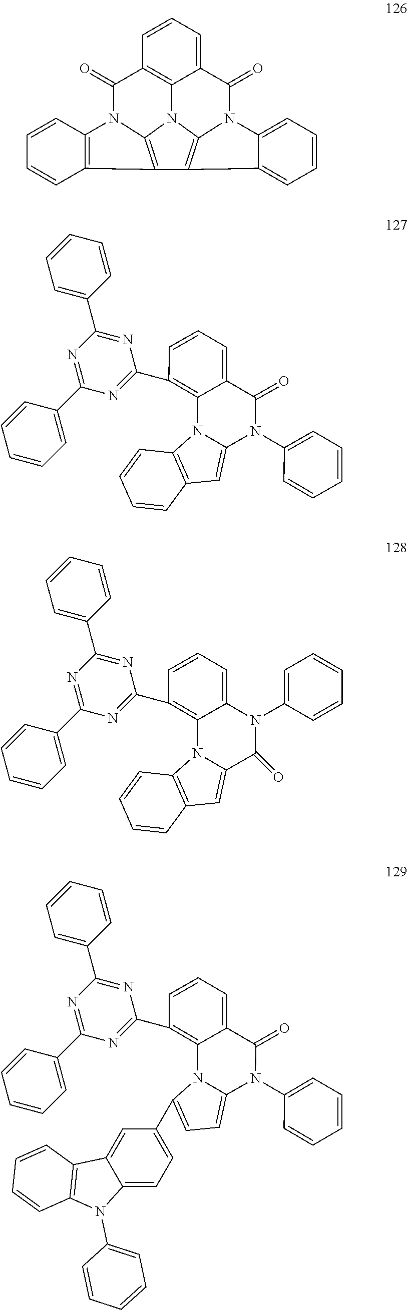

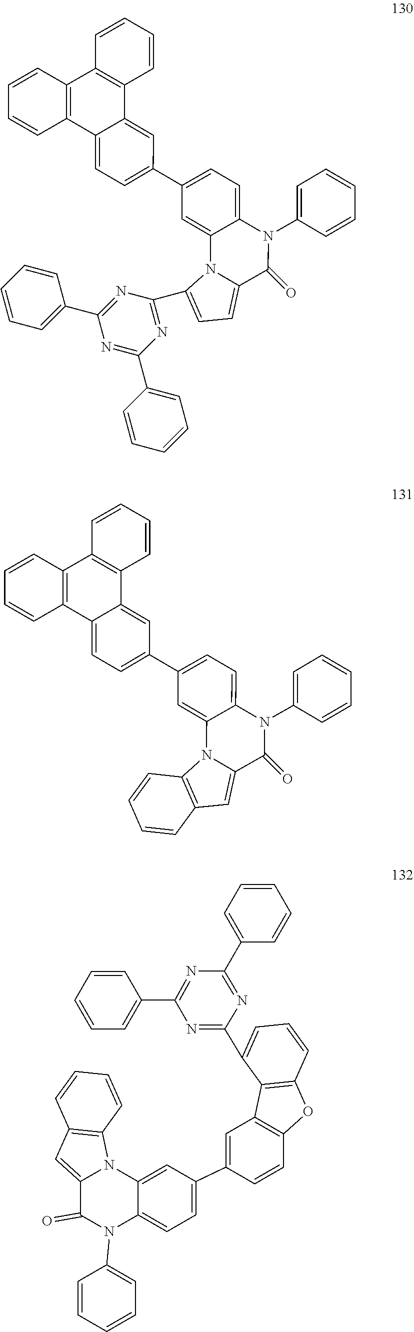

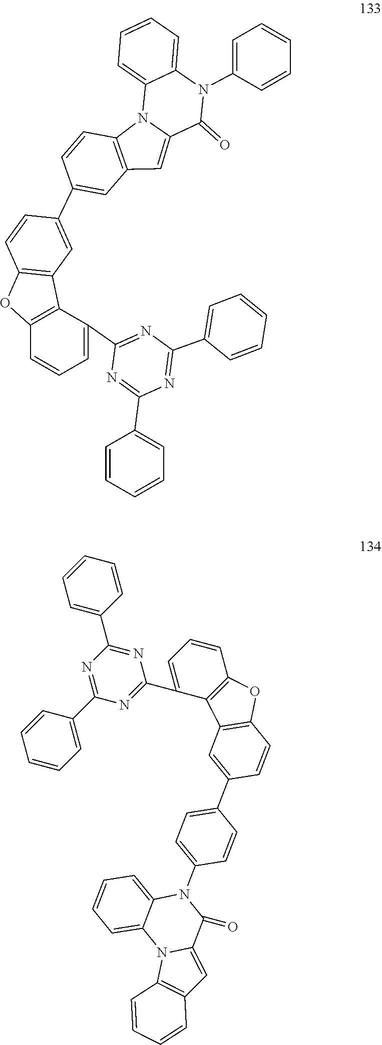

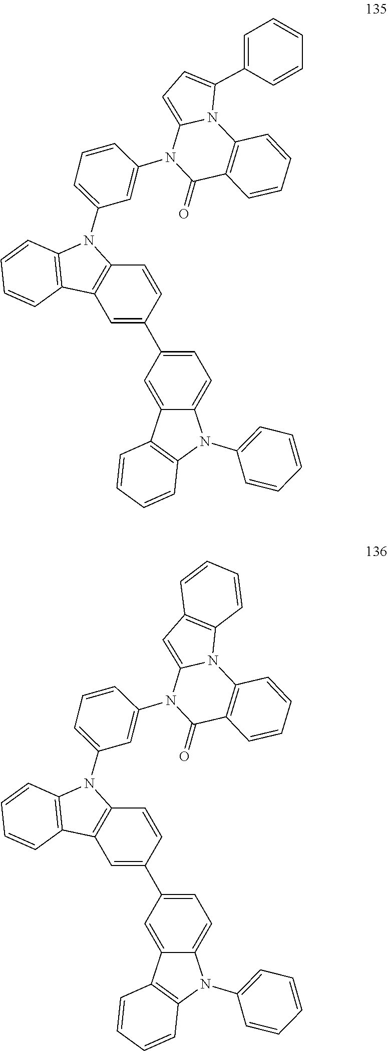

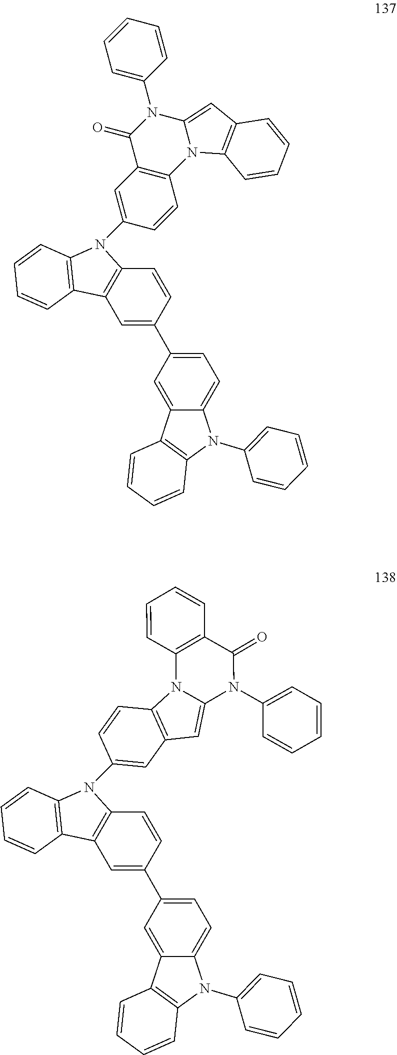

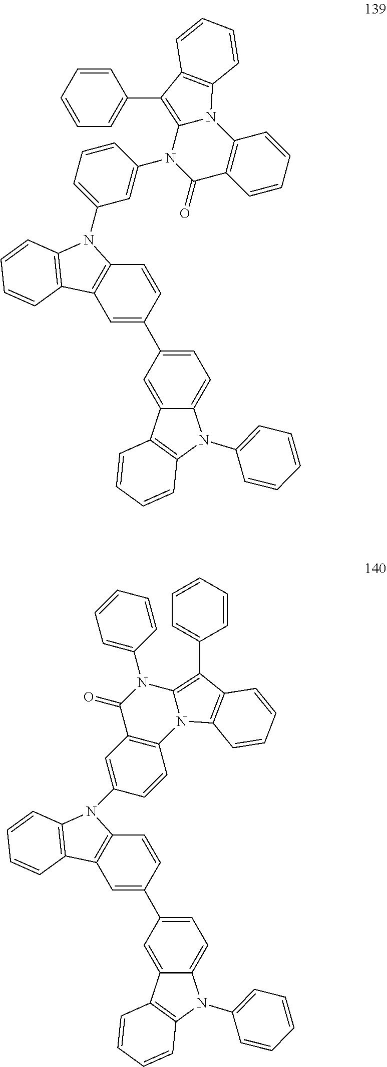

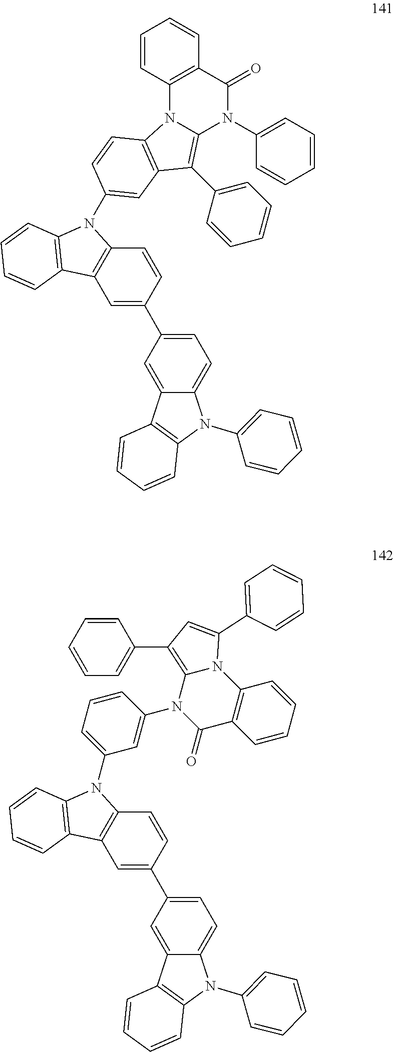

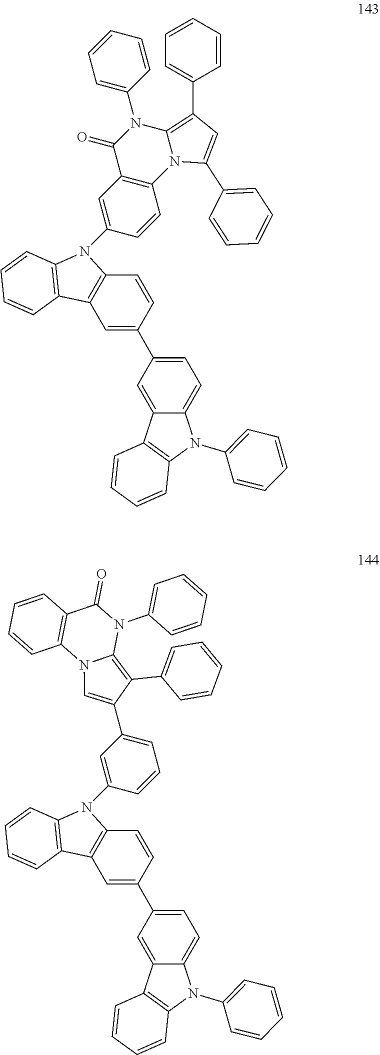

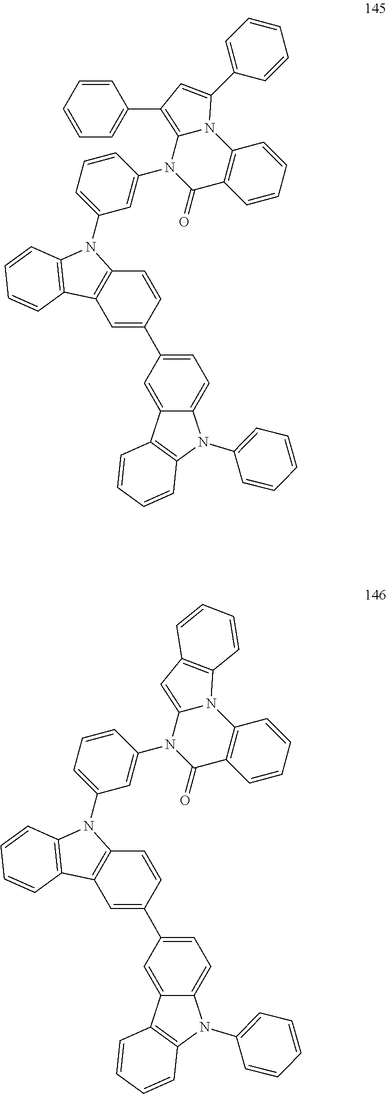

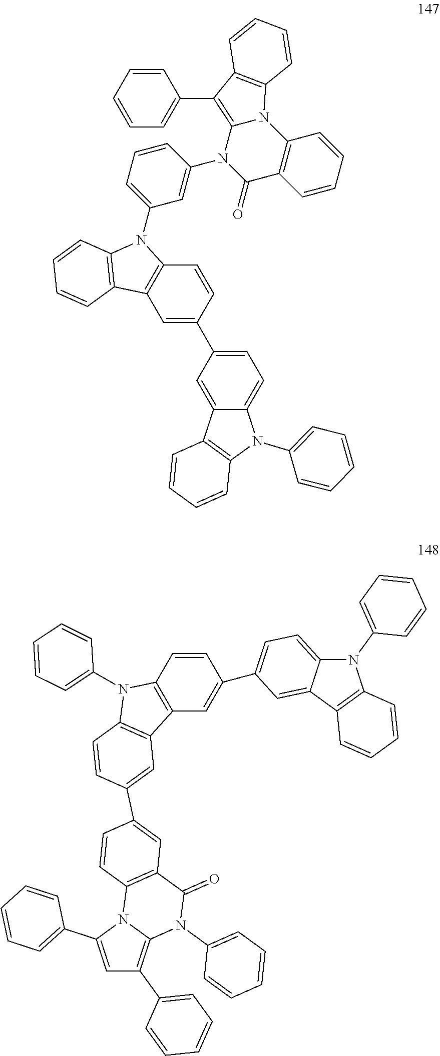

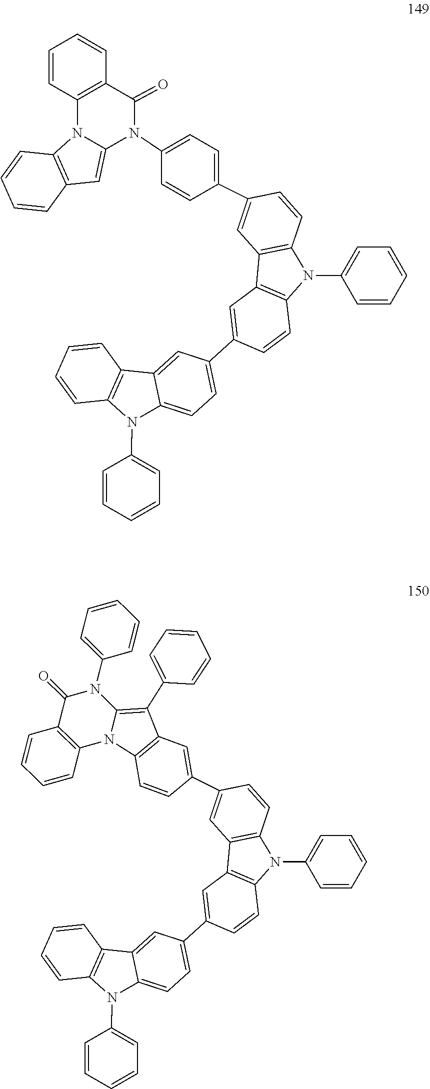

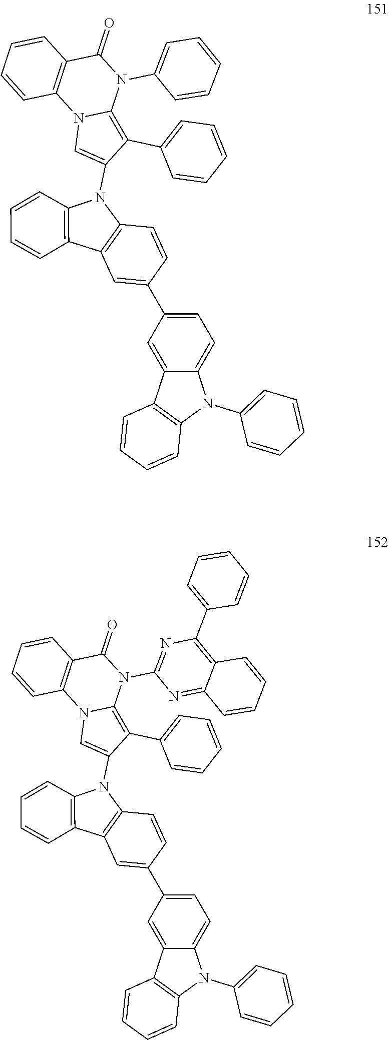

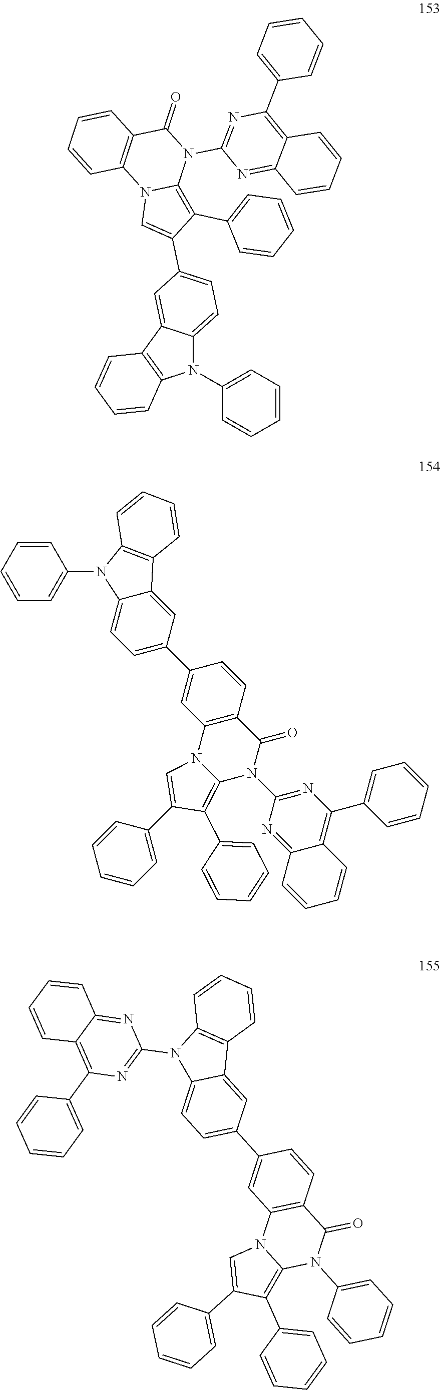

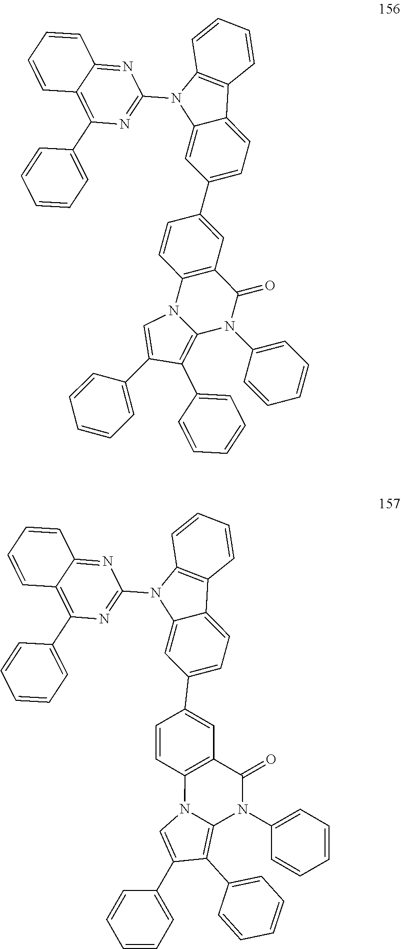

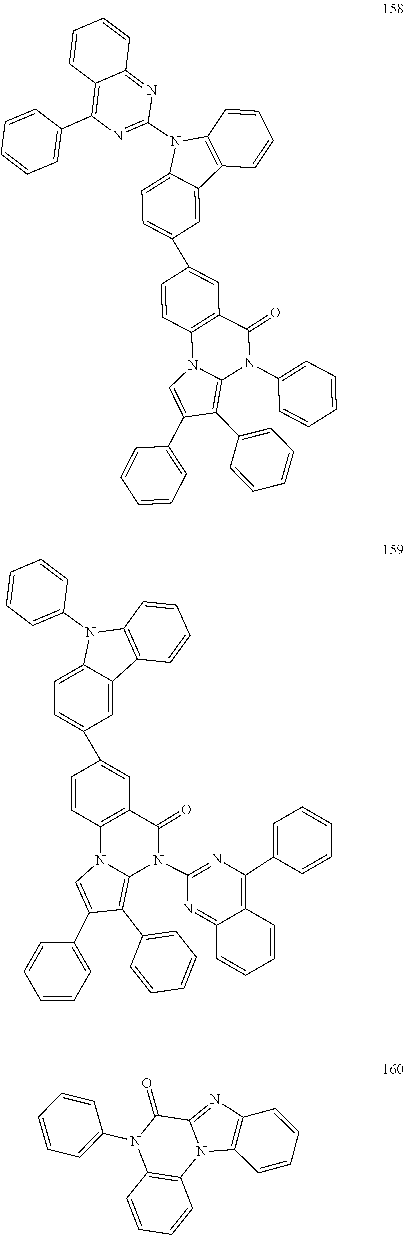

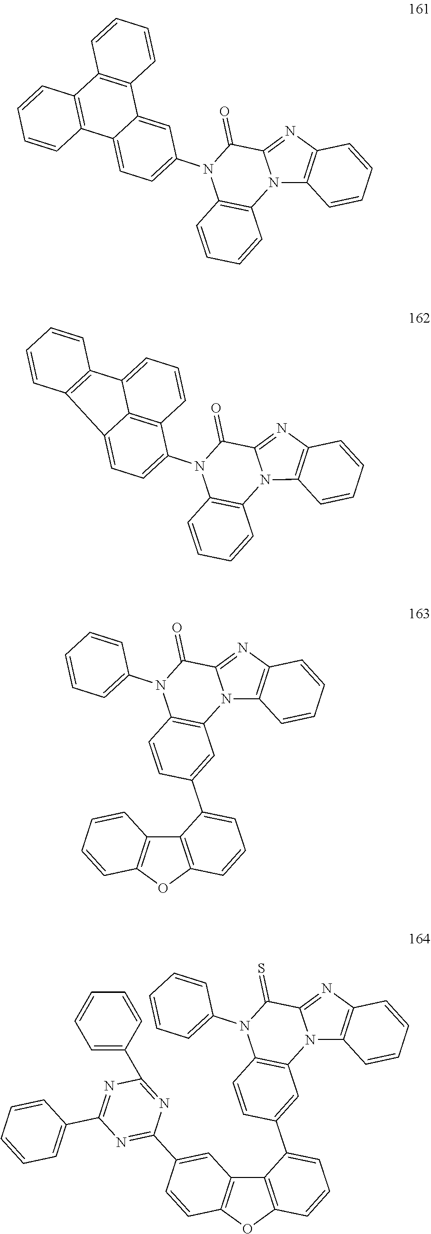

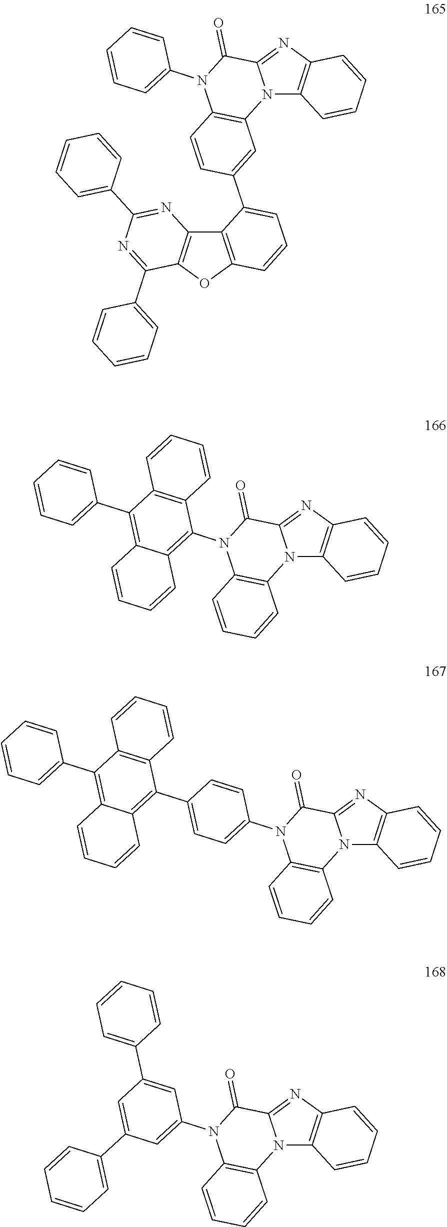

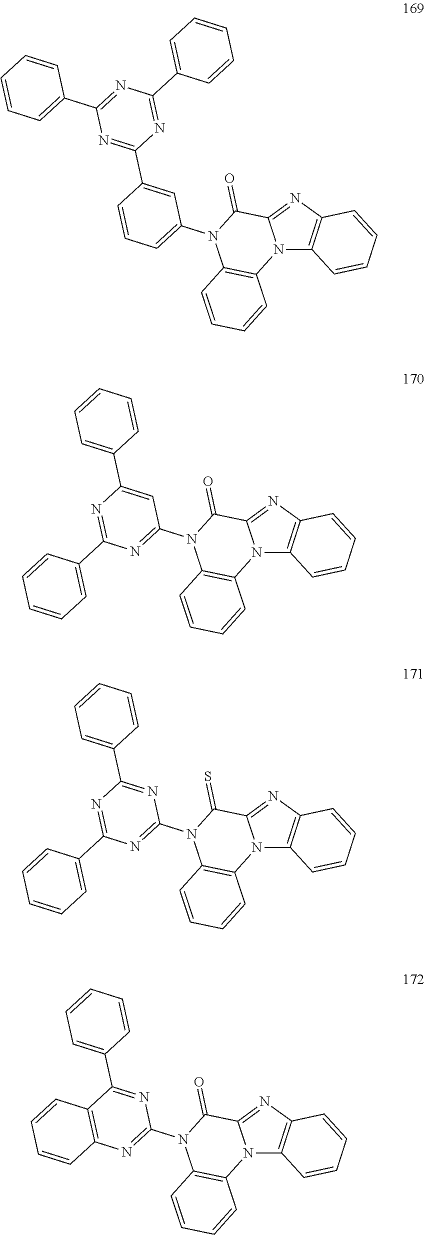

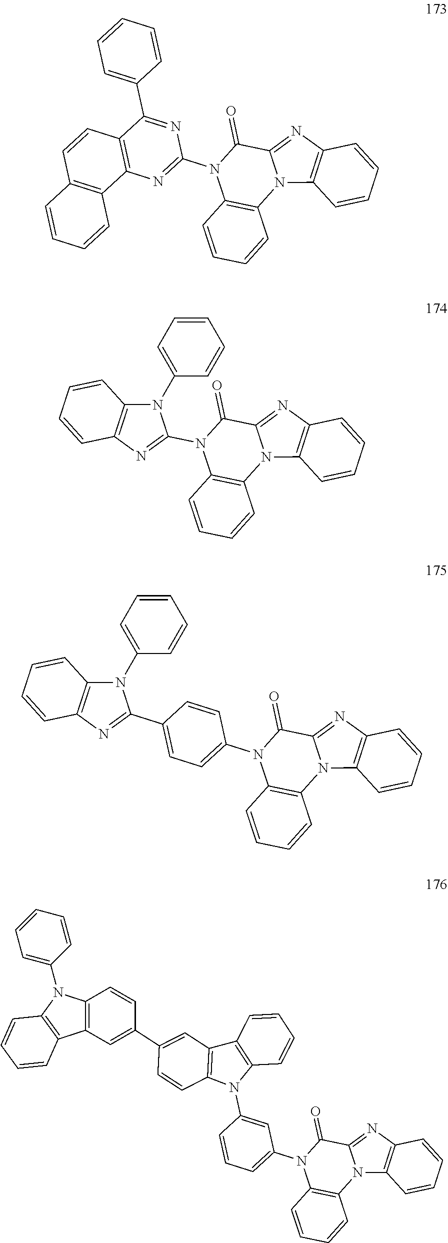

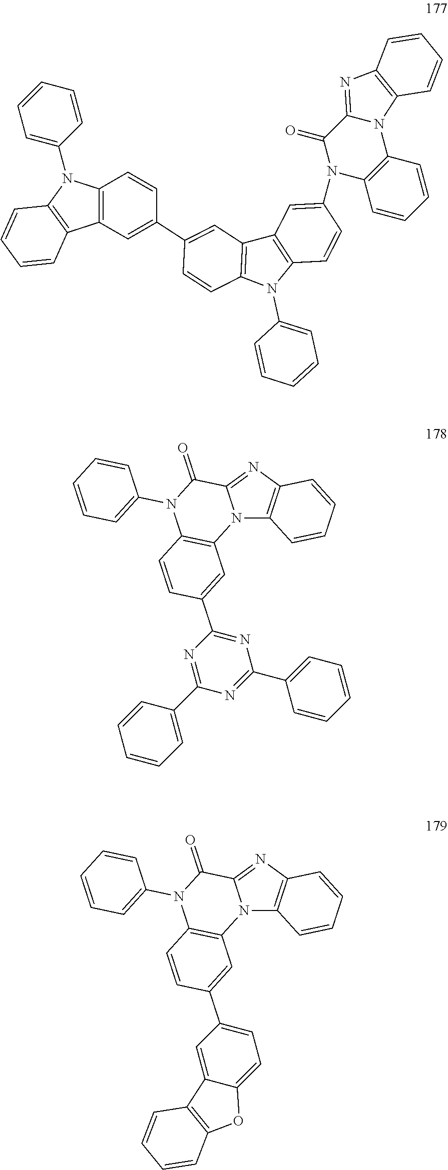

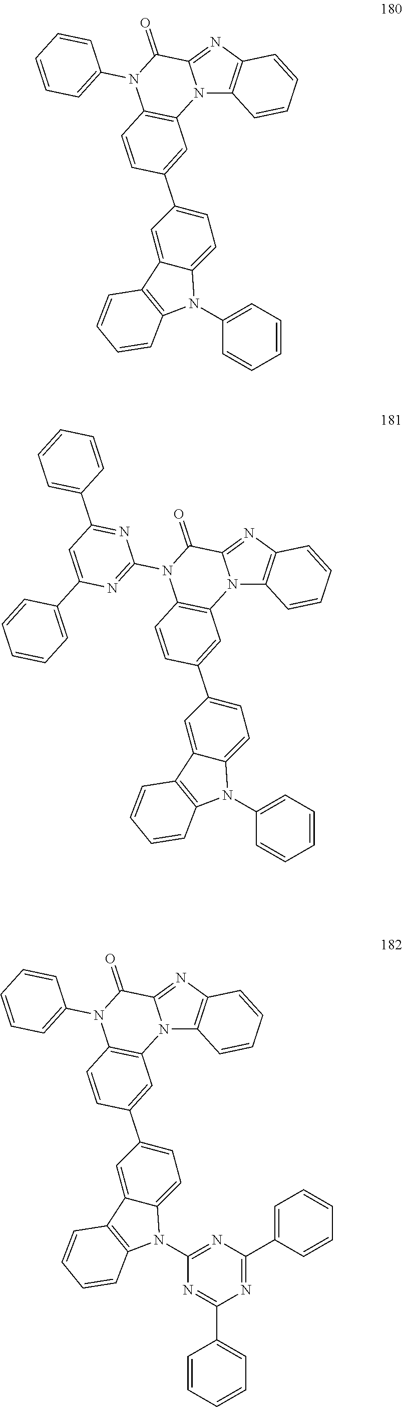

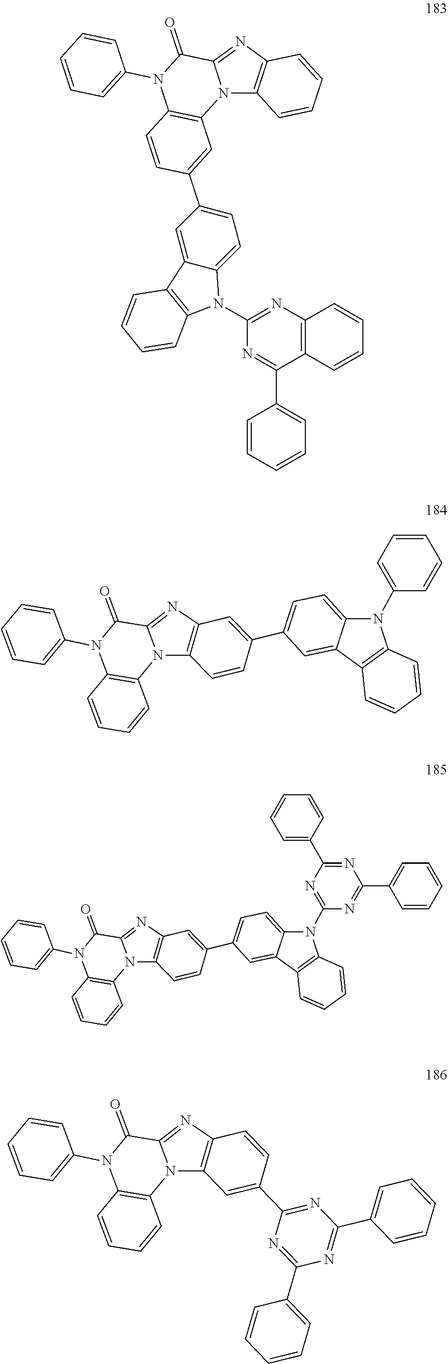

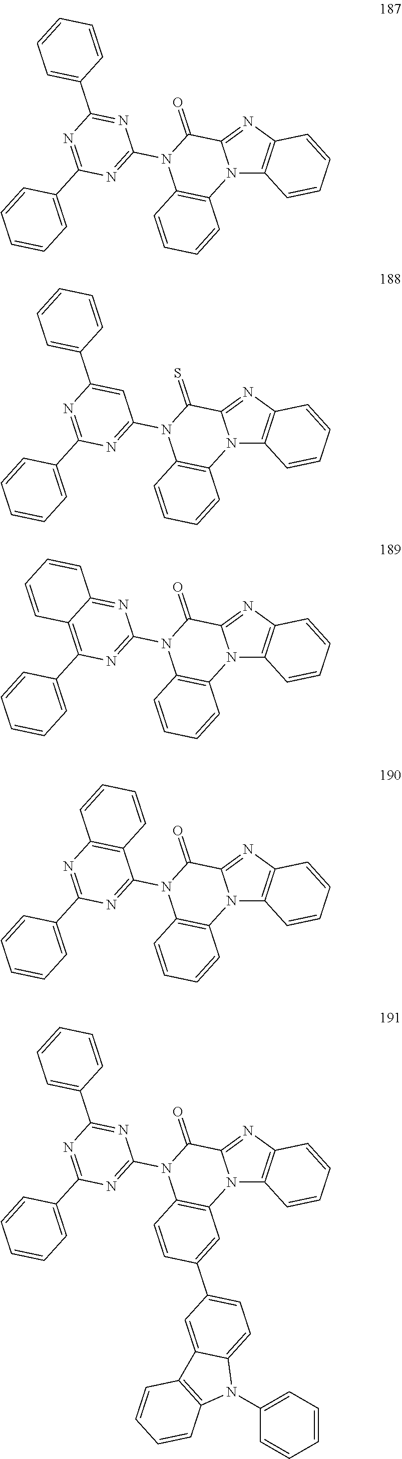

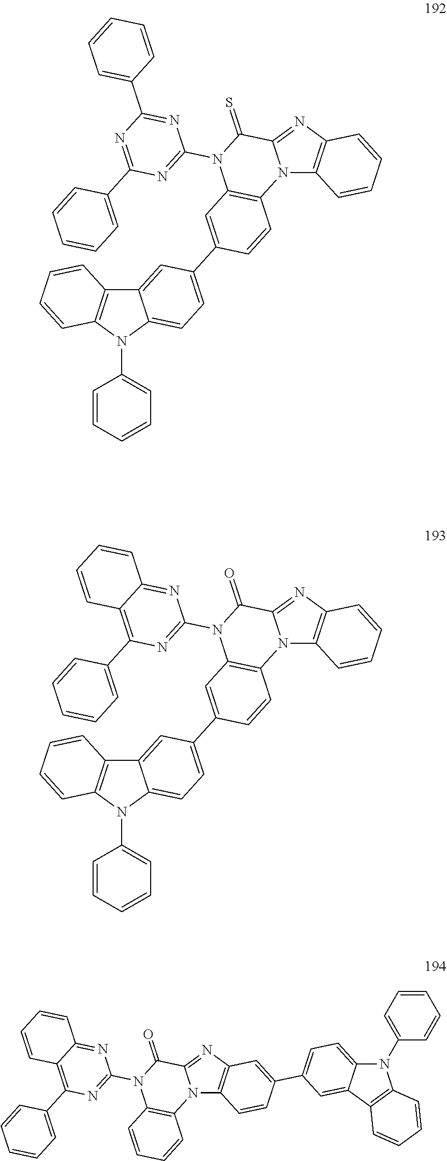

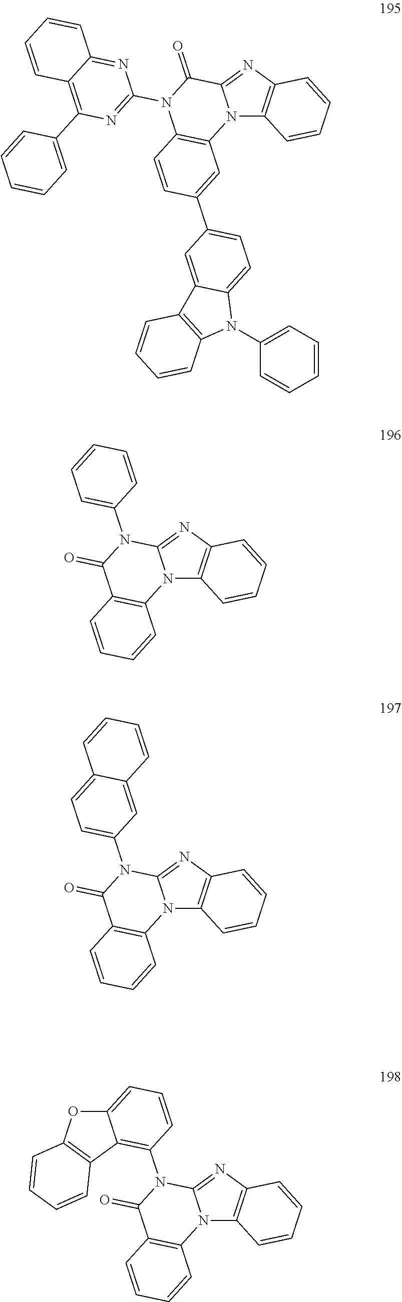

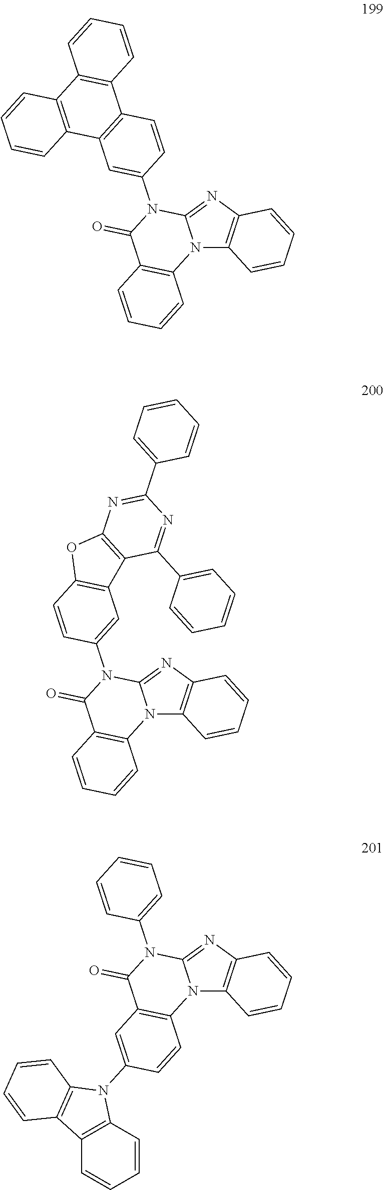

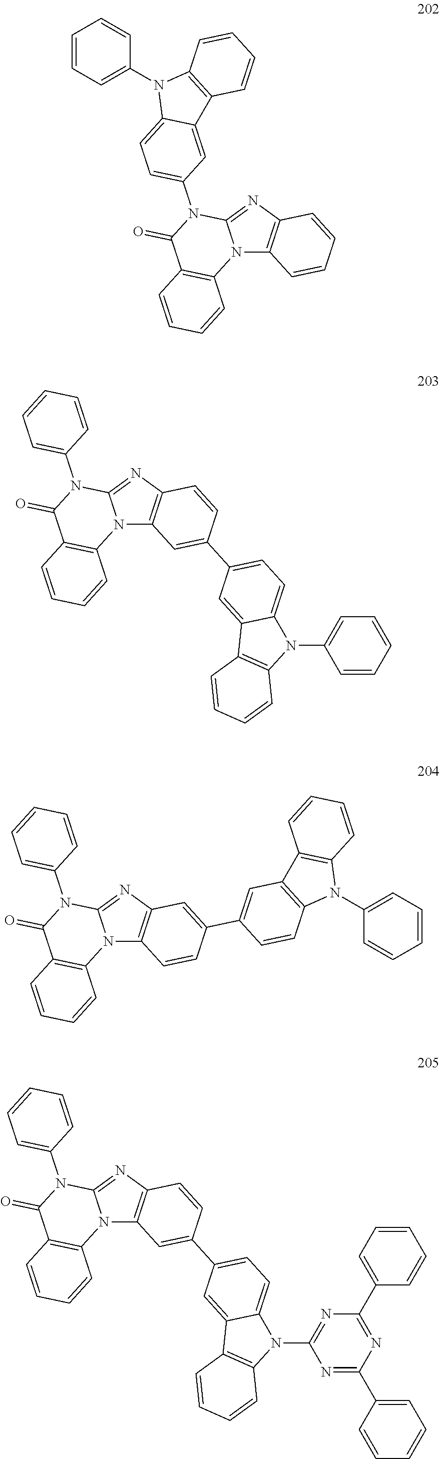

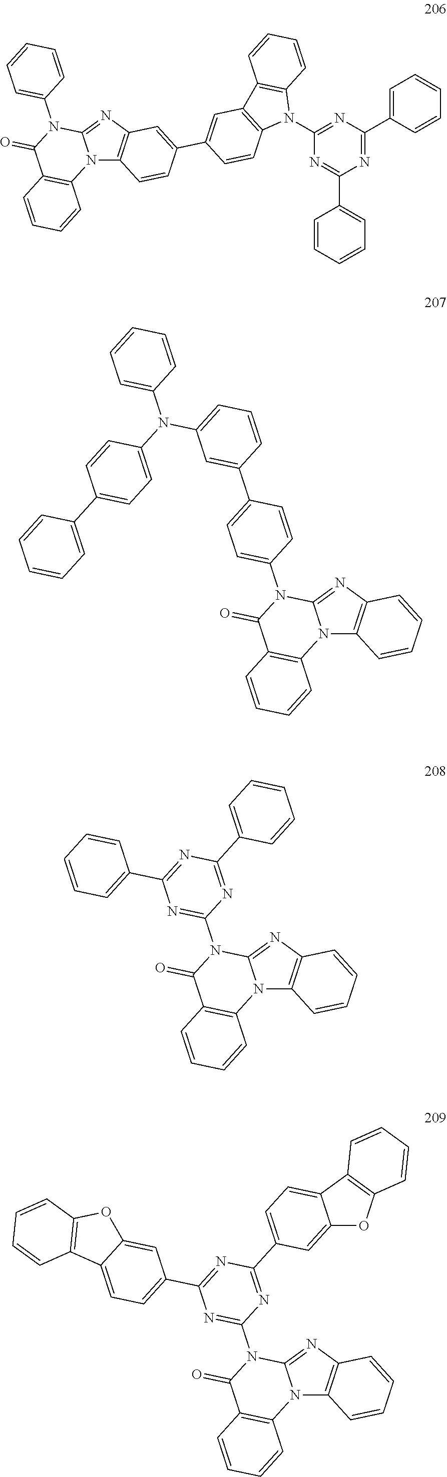

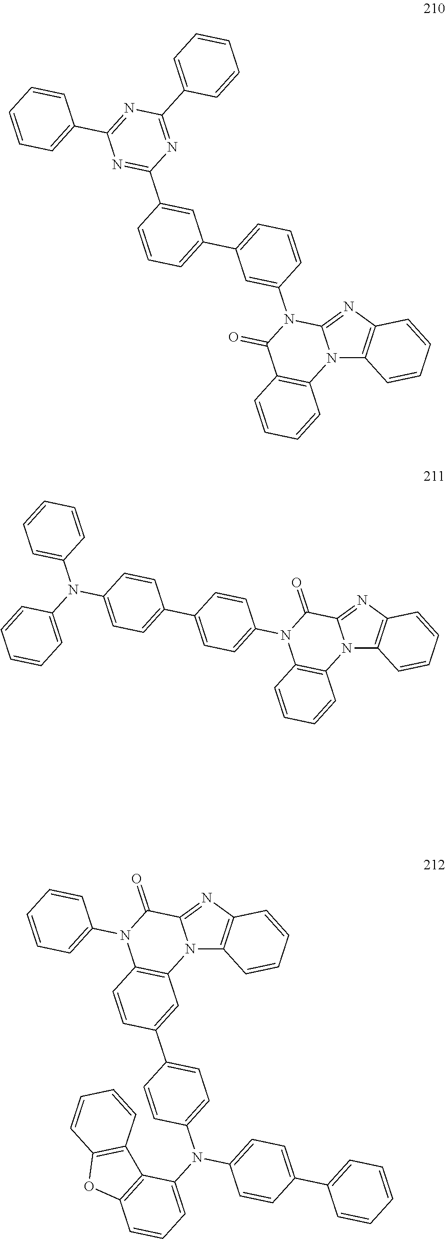

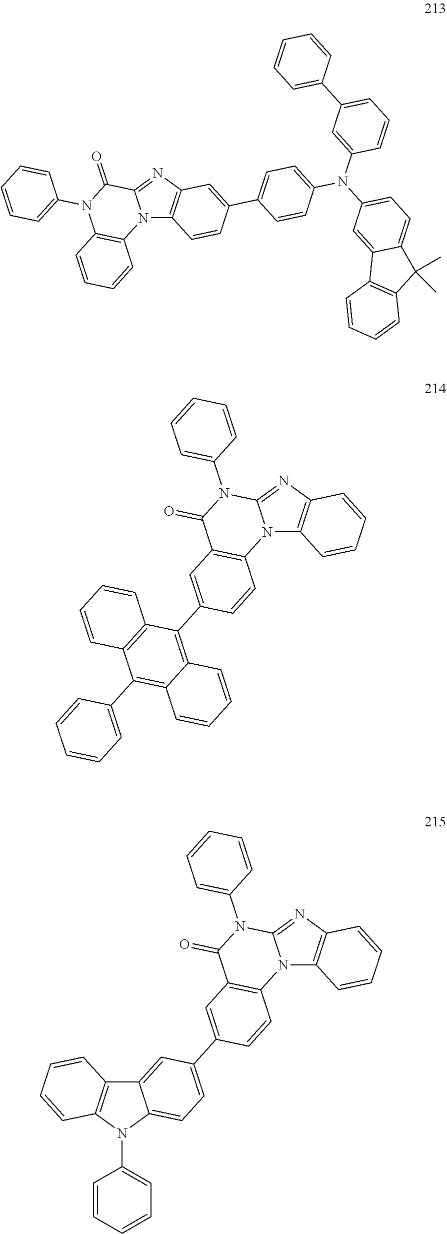

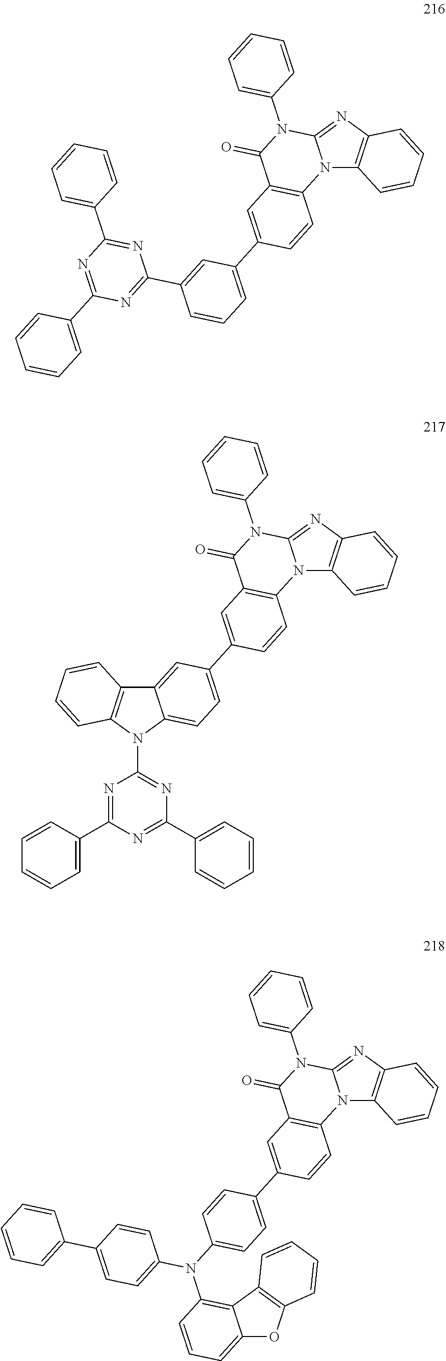

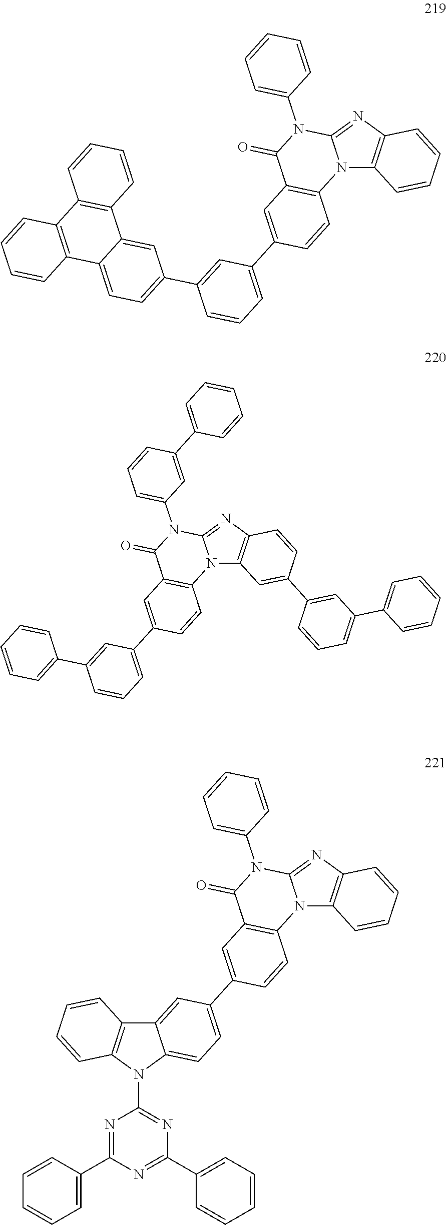

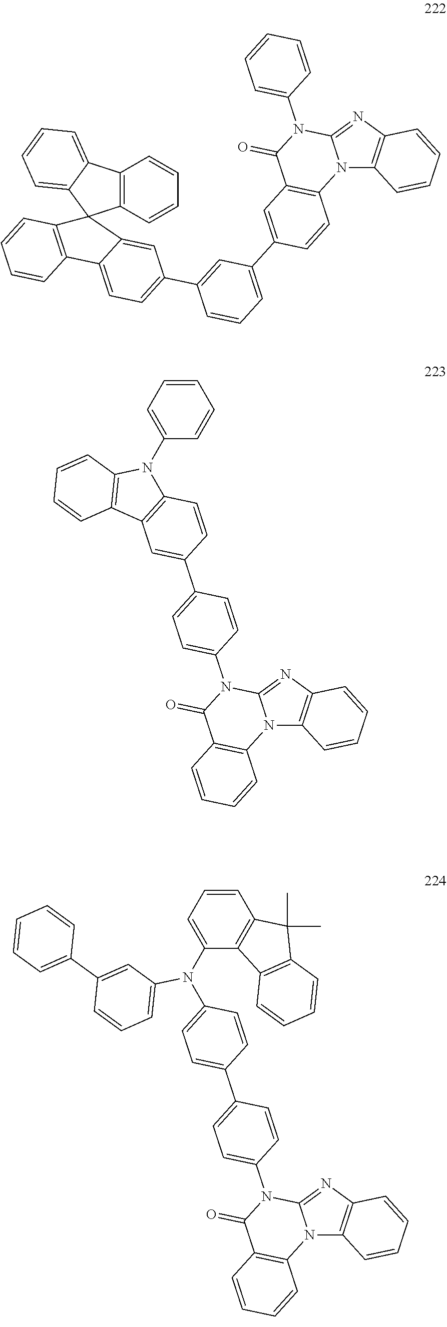

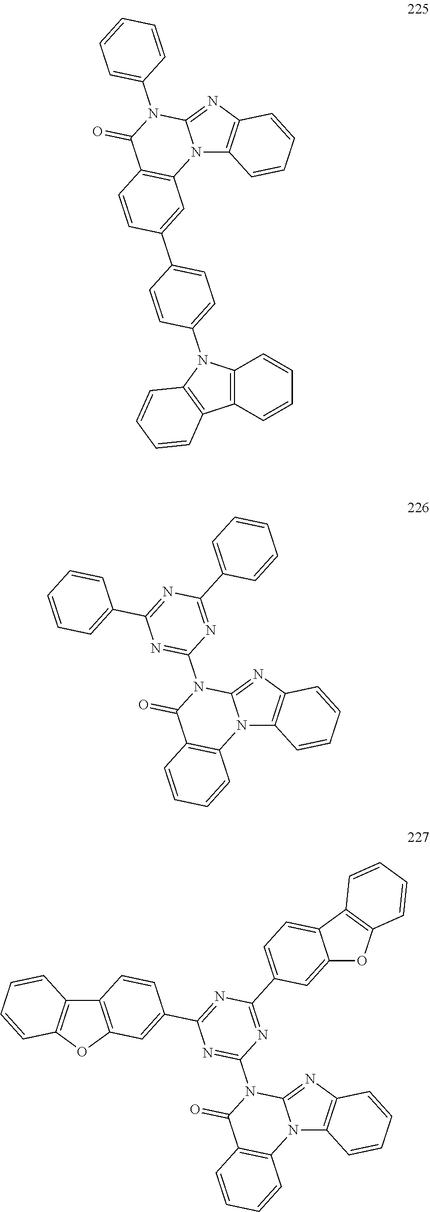

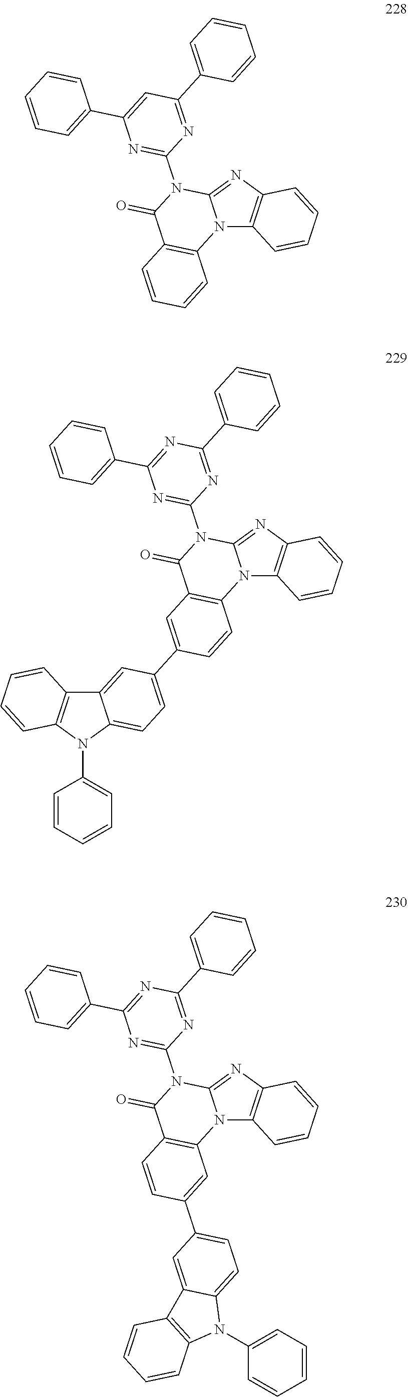

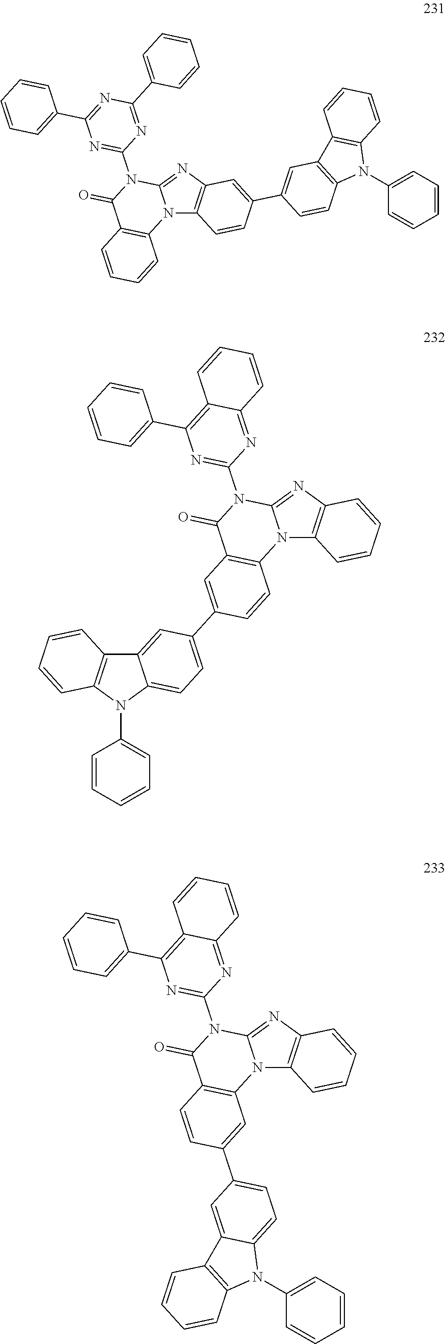

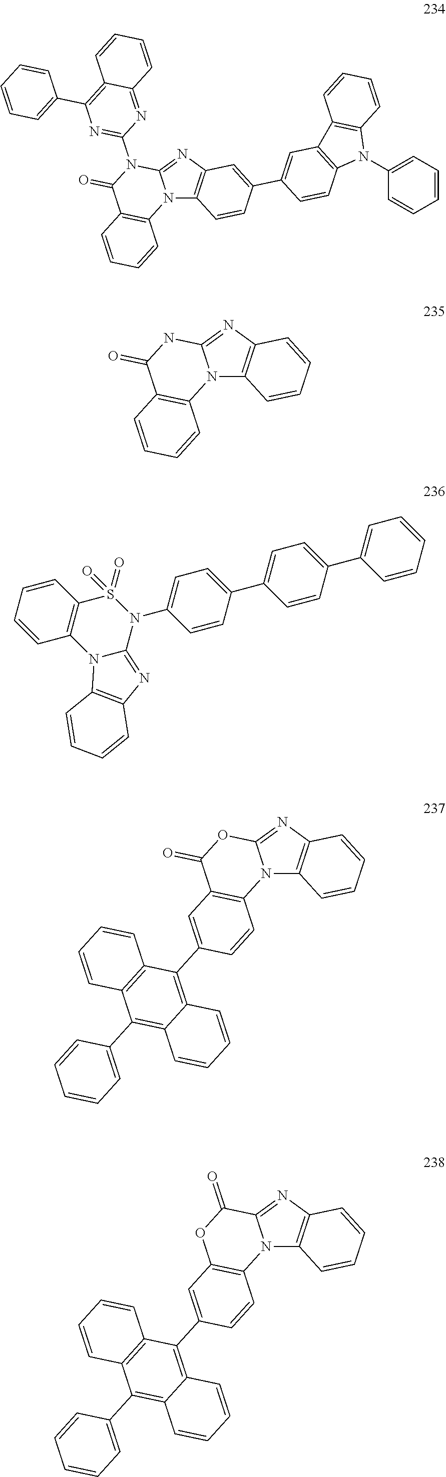

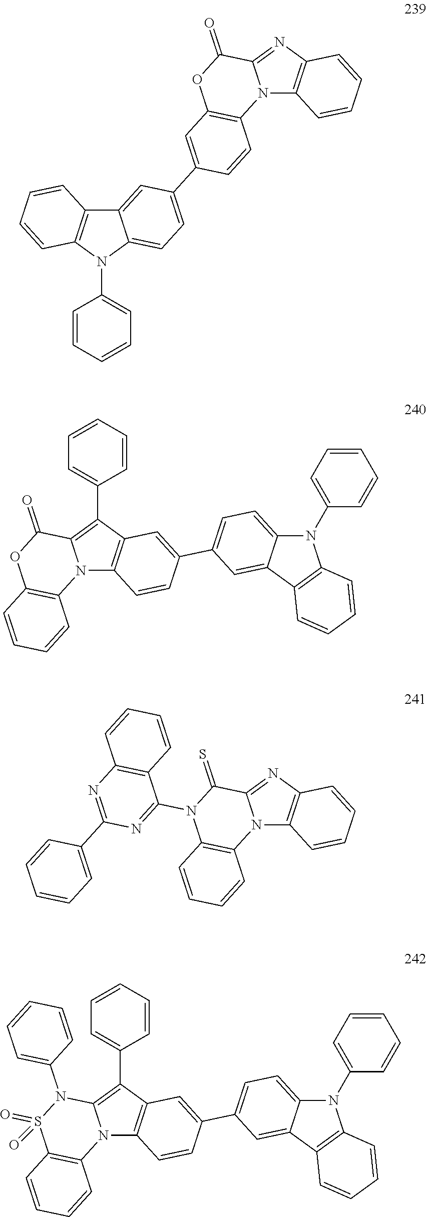

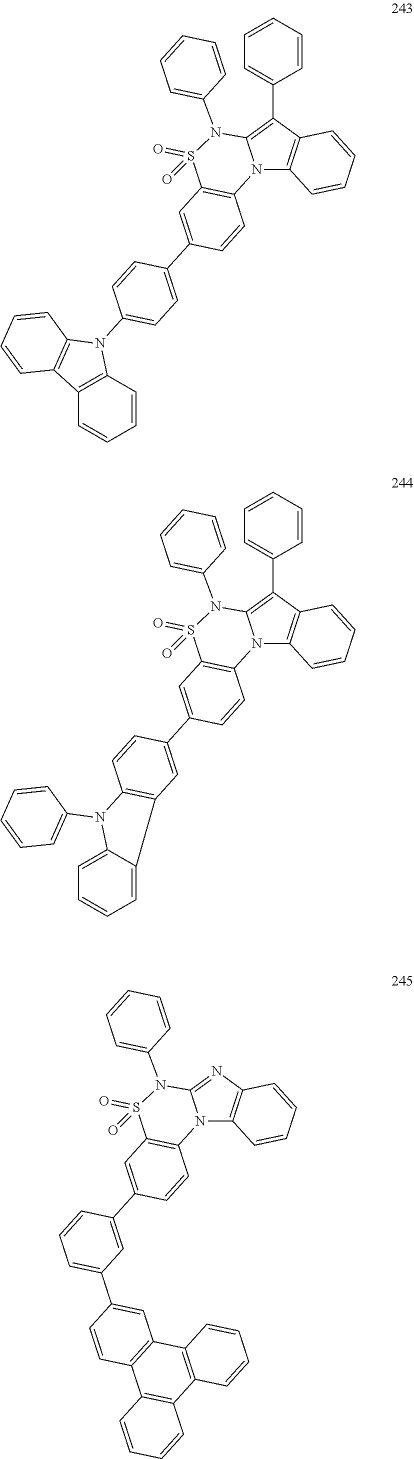

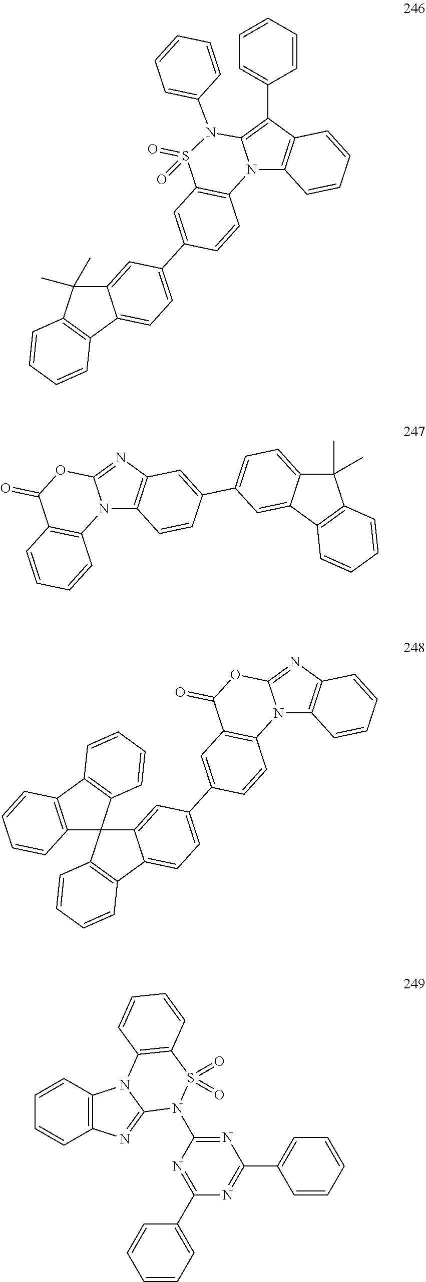

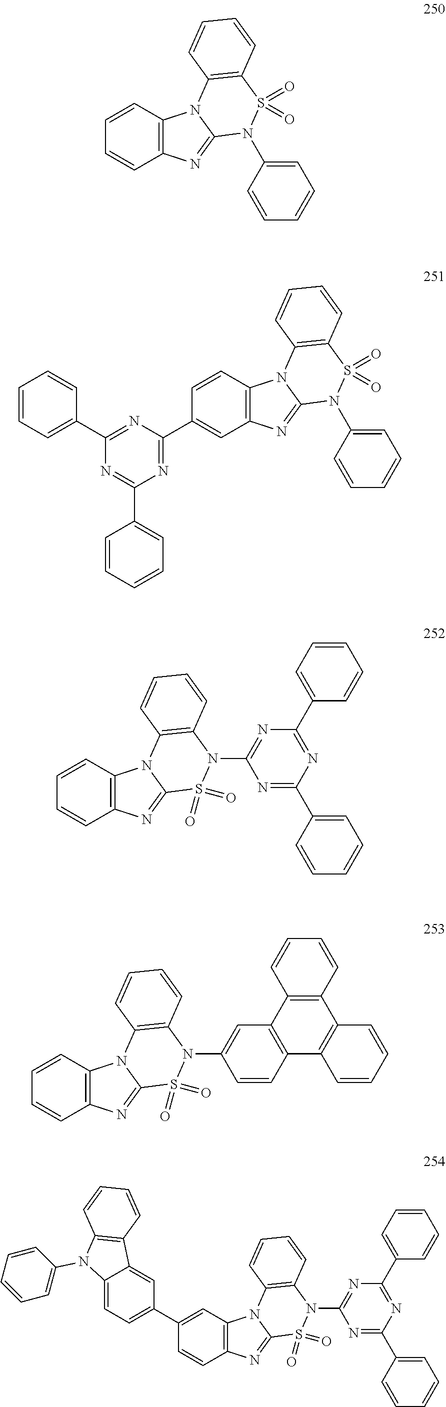

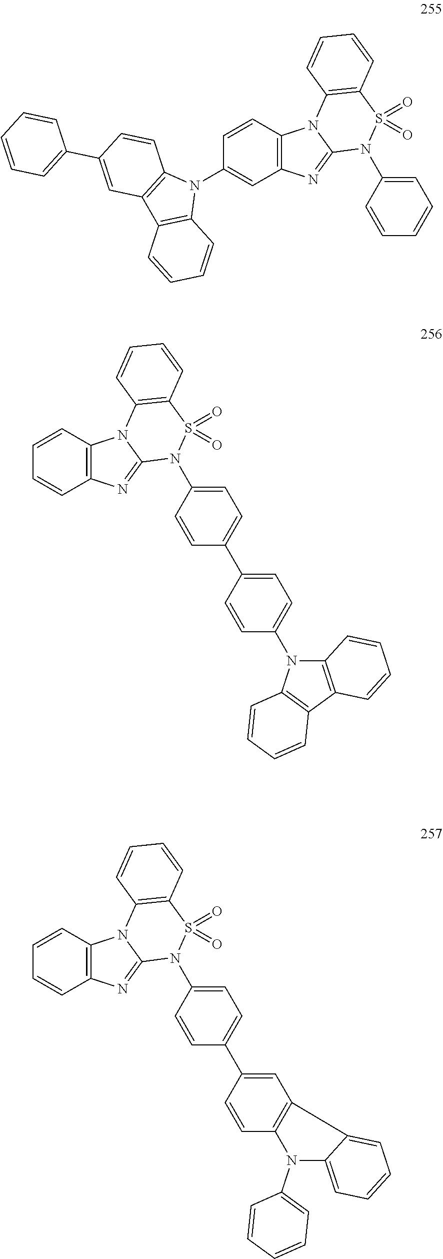

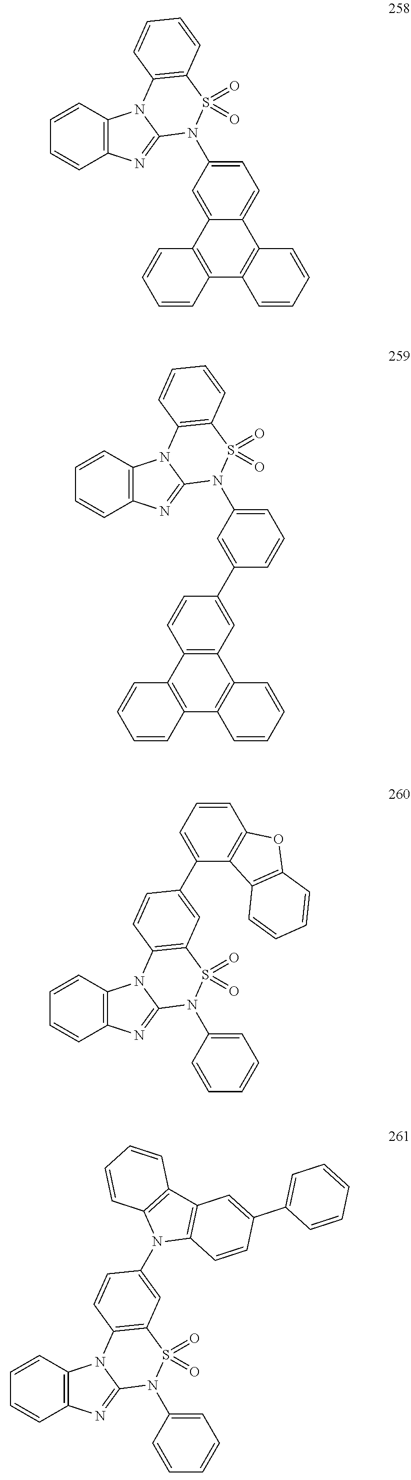

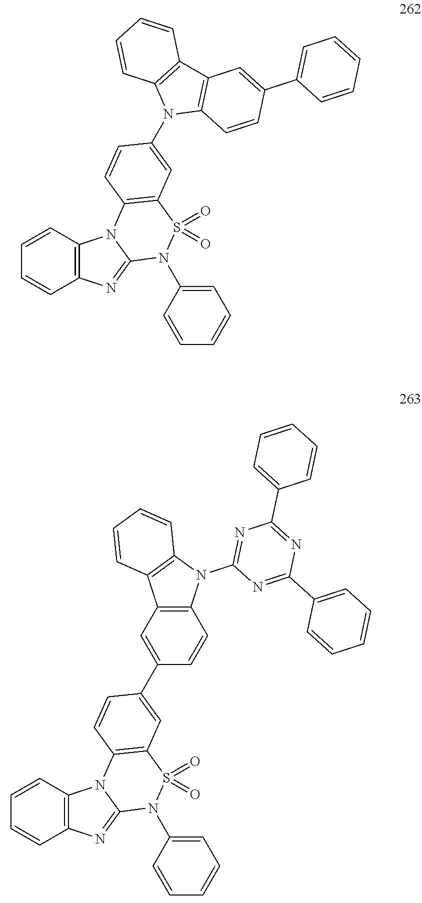

For synthesis of the compounds of the invention, it is possible to proceed from commercially available compounds already containing the pyrrolo-quinazolinone base skeleton or derivatives thereof of formula (I).

These starting compounds are then halogenated, and an Ullmann coupling is conducted in order to introduce an aryl or heteroaryl group on the nitrogen atom of the lactam group (scheme 1). In a subsequent step (scheme 2), an amino group is introduced in a Buchwald coupling, or an aryl or heteroaryl group is introduced in a Suzuki coupling.

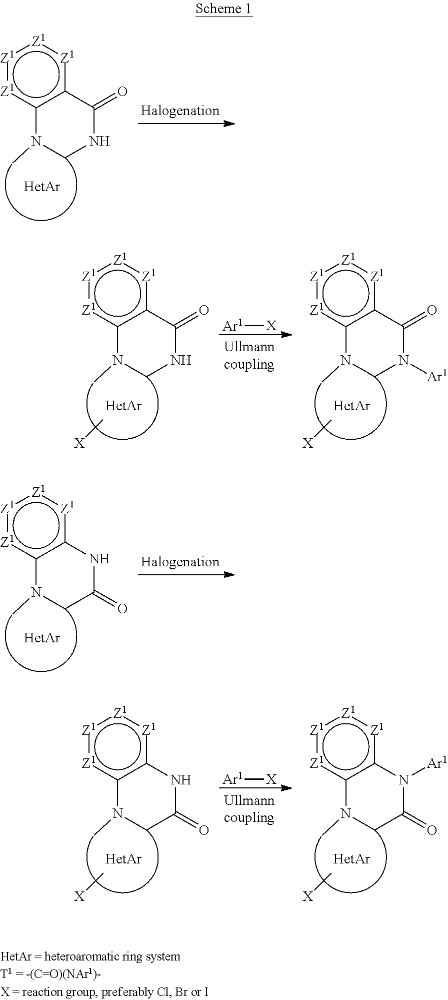

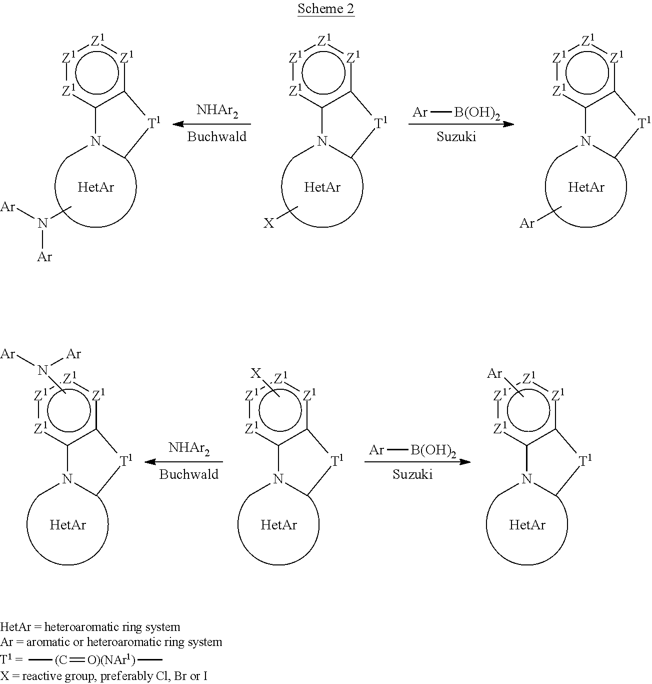

In an alternative synthesis method for compounds containing a structural element of the formula (I), it is possible to proceed as shown in scheme 3 below.

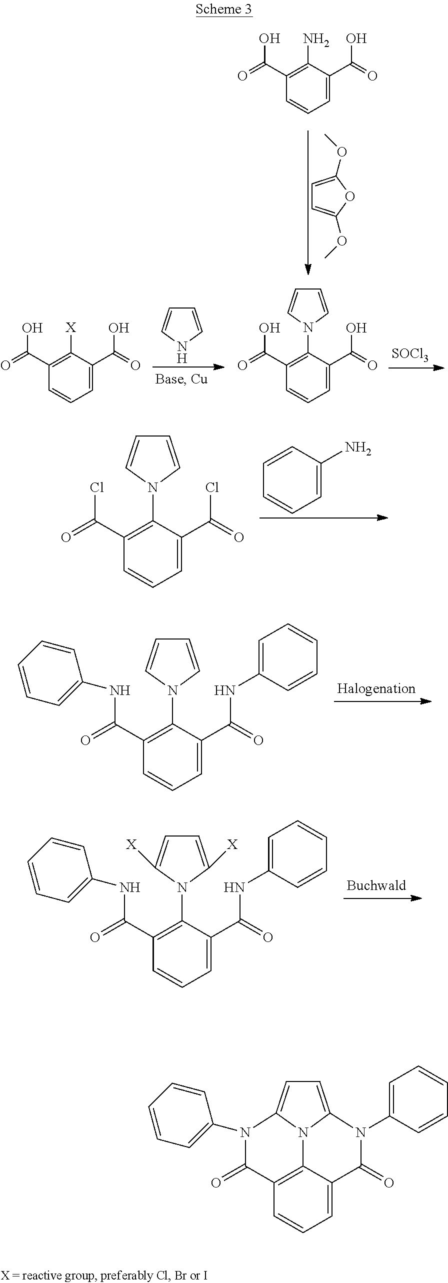

This proceeds from a benzene-1,3-dicarboxylic acid bearing an amino group in the 2 position, or bearing a halogen group in the 2 position, to prepare a benzene-1,3-dicarboxylic acid bearing an N-pyrrole group in the 2 position. The two carboxylic acid groups are converted to arylamide groups via the corresponding carbonyl chlorides. Subsequently, the compound is brominated on the pyrrole group in positions 2 and 5. Finally, a Buchwald reaction is conducted, in which the two nitrogen atoms of the two arylamide groups each form a ring with the pyrrole group.

In an alternative method for preparation of compounds containing a structural element of formula (I), it is possible to proceed as shown in the following scheme:

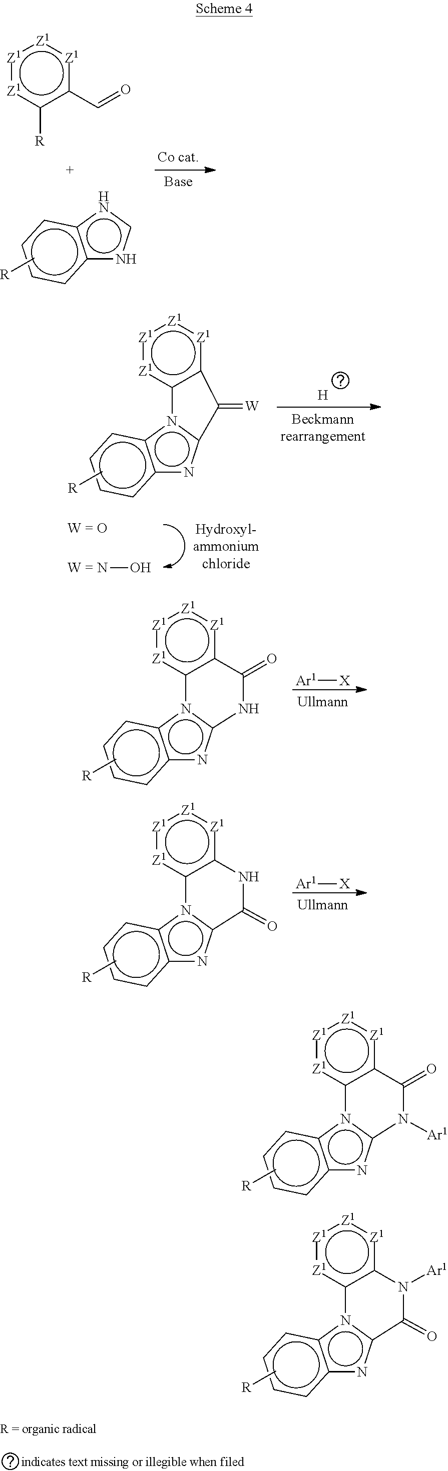

This proceeds from a benzimidazole derivative and a 2-fluorobenzaldehyde derivative, at first to form a coupling product having a ketone group. This is then converted to an oxime group. The oxime is converted in a Beckmann rearrangement to the two isomeric cyclic lactams. These can then be preparatively separated from one another, for example by chromatography. In a last step, the NH group of the lactam unit is converted further in an Ullmann coupling, such that an aromatic group is bound to the NH group of the lactam unit.

The application thus also provides a process for preparing a compound containing a structural element of the formula (I), characterized in that, proceeding from a compound of the formula (Int-I) or (Int-II)

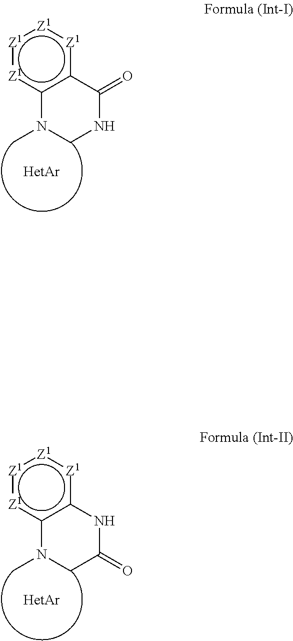

where HetAr is a heteroaromatic ring system which has 5 to 40 aromatic ring atoms and is substituted by R² radicals, and Z¹ is the same or different at each instance and is selected from CR¹ or N,

in a first step a halogen substituent is introduced, preferably Cl, Br or I, in a second step an aromatic or heteroaromatic ring system is introduced on the nitrogen atom of the lactam group in an Ullmann coupling reaction, and in a third step an amino group bearing the aromatic or heteroaromatic ring systems as substituents is introduced in the position of the halogen substituent via a Buchwald coupling, or an aromatic or heteroaromatic ring system is introduced via a Suzuki coupling.

An alternative process for preparing a compound containing a structural element of formula (I) is characterized in that, proceeding from a compound of a formula (Int-III)

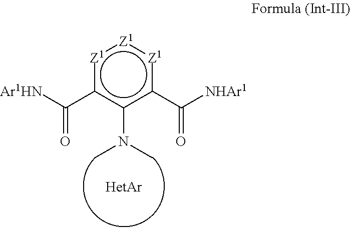

where Z¹ is the same or different at each instance and is CR¹ or N; and HetAr is a heteroaromatic ring system which has 5 to 40 aromatic ring atoms and is substituted by R² radicals; and Ar¹ is the same or different at each instance and is selected from aromatic ring systems which have 6 to 40 aromatic ring atoms and are substituted by one or more R³ radicals, and heteroaromatic ring systems which have 5 to 40 aromatic ring atoms and are substituted by one or more R³ radicals;

characterized in that in a first step halogen substituents, preferably Br, are introduced in the two positions vicinal to the nitrogen atom of HetAr, and in a second step the amide groups of the formula (Int-III) bind to the positions of the halogen substituents on HetAr in a metal-catalyzed coupling reaction, so as to form two lactam rings.

An alternative process for preparing a compound containing a structural element of formula (I) is characterized in that a starting compound of a formula (Int-IV)

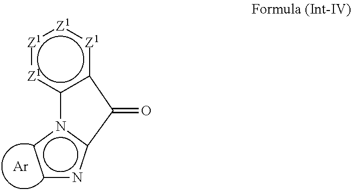

where Ar is an aromatic ring system which has 6 to 40 aromatic ring atoms and is substituted by R² radicals, and where Z¹ is the same or different at each instance and is selected from CR¹ or N, in a first step is converted at its ketone group to the corresponding hydroxylamine derivative, this hydroxylamine derivative is then converted further in a second step in a Beckmann rearrangement, and the resultant lactam derivative is then converted in a third step at the nitrogen of the lactam unit in an Ullmann coupling.

The Beckmann rearrangement preferably gives rise to two isomeric lactam derivatives that are separated by chromatography before further reaction.

Intermediates containing the pyrrolo-quinazolinone base skeleton or the pyrrolo-quinoxalinone base skeleton are commercially available in some cases. Synthesis methods known in the art for preparation of compounds containing the abovementioned base skeleton or variants thereof are shown below. They show that such starting compounds are available to the person skilled in the art within the scope of his common art knowledge.

The method below (scheme 5) can be used to prepare indolo-quinazolinones:

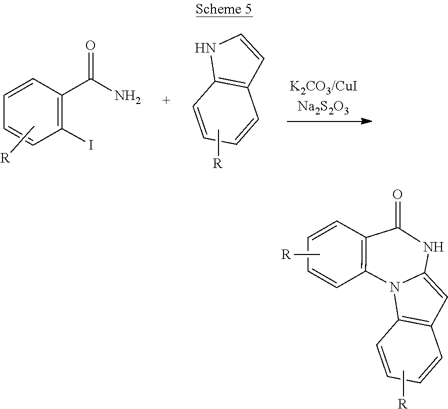

The method below (schemes 6 and 7) can be used to prepare indolo-quinoxalinones:

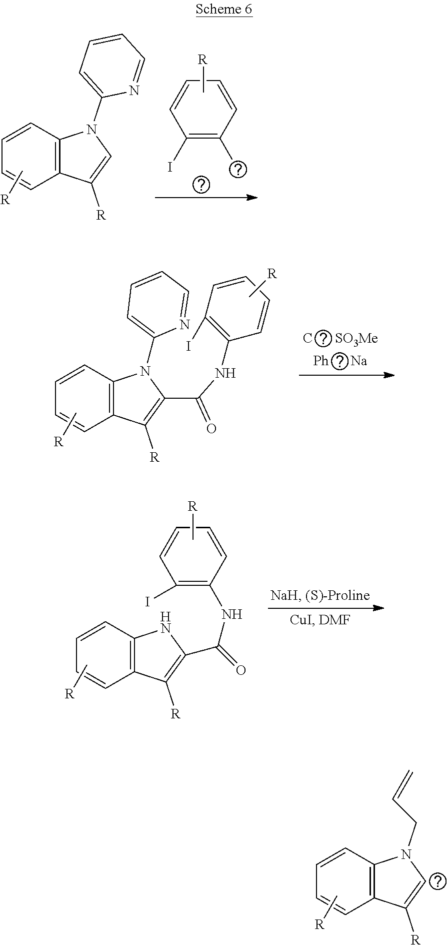

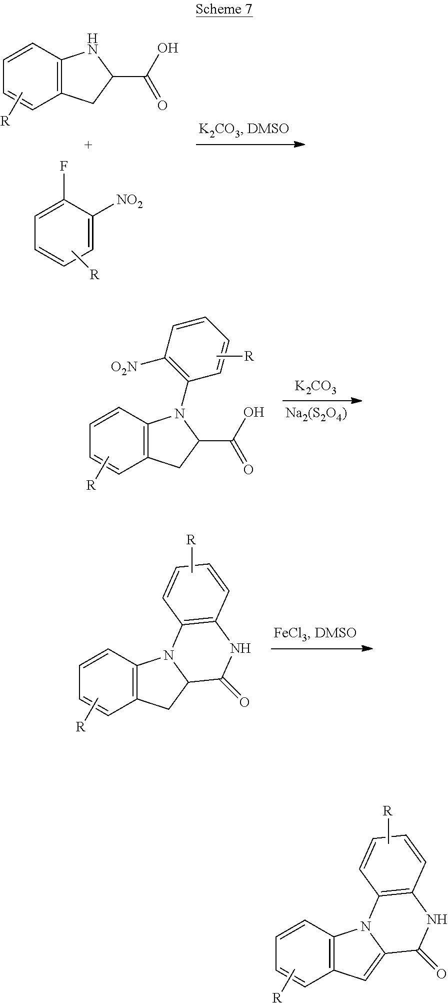

The method below (schemes 8 and 9) can be used to prepare indolo-quinoxalinones:

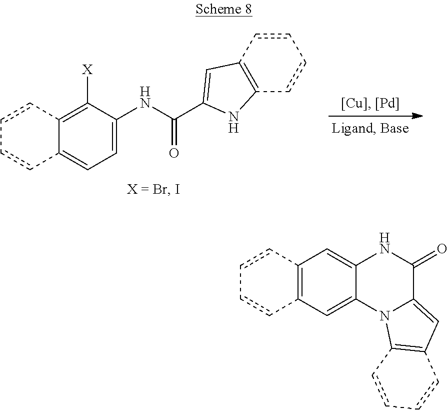

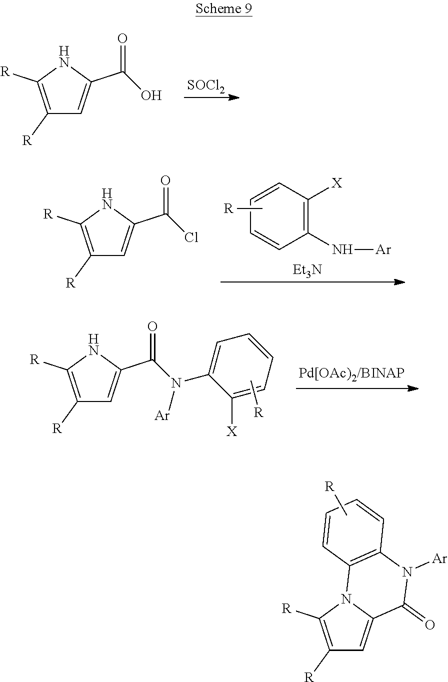

A process for preparing benzimidazolo-quinazolinones is shown in the following schemes:

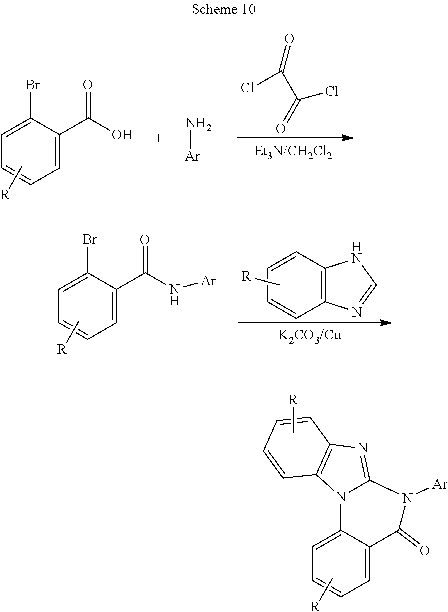

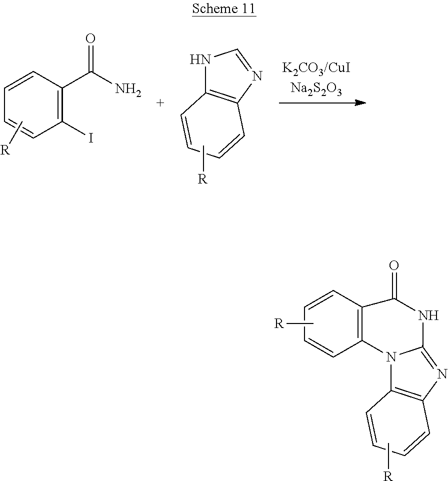

A process for preparing benzimidazolo-quinoxazolinones is shown in the following scheme:

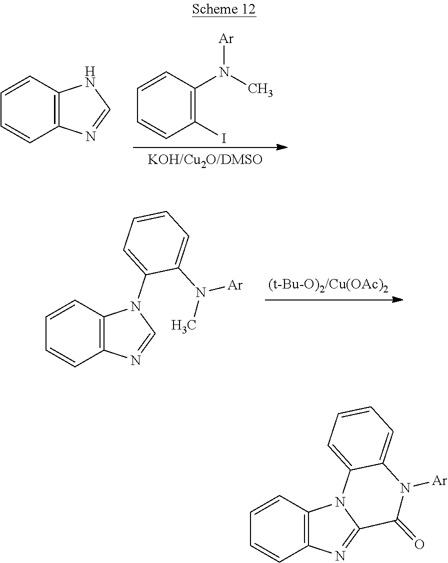

The compounds containing a structural element of formula (I), especially compounds substituted by reactive leaving groups, such as bromine, iodine, chlorine, boronic acid or boronic esters, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C—C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more compounds containing a structural element of formula (I), wherein the bond(s) to the polymer, oligomer or dendrimer may be localized at any desired positions substituted by R¹, R² or R³ in formula (I). According to the linkage of the compound, it is part of a side chain of the oligomer or polymer or part of the main chain. An oligomer in the context of this invention is understood to mean a compound formed from at least three monomer units. A polymer in the context of the invention is understood to mean a compound formed from at least ten monomer units. The polymers, oligomers or dendrimers of the invention may be conjugated, partly conjugated or nonconjugated. The oligomers or polymers of the invention may be linear, branched or dendritic. In the structures having linear linkage, the units of formula (I) may be joined directly to one another, or they may be joined to one another via a bivalent group, for example via a substituted or unsubstituted alkylene group, via a heteroatom or via a bivalent aromatic or heteroaromatic group. In branched and dendritic structures, it is possible, for example, for three or more units of formula (I) to be joined via a trivalent or higher-valency group, for example via a trivalent or higher-valency aromatic or heteroaromatic group, to give a branched or dendritic oligomer or polymer.

For the repeat units in oligomers, dendrimers and polymers, the same preferences apply as described above for compounds containing a structural element of formula (I).

For preparation of the oligomers or polymers, the monomers of the invention are homopolymerized or copolymerized with further monomers. Suitable and preferred comonomers are selected from fluorenes, spirobifluorenes, paraphenylenes, carbazoles, thiophenes, dihydrophenanthrenes, cis- and trans-indenofluorenes, ketones, phenanthrenes or else two or more of these units. The polymers, oligomers and dendrimers typically contain still further units, for example emitting (fluorescent or phosphorescent) units, for example vinyltriarylamines or phosphorescent metal complexes, and/or charge transport units, especially those based on triarylamines.

The polymers, oligomers and dendrimers of the invention have advantageous properties, especially high lifetimes, high efficiencies and good color coordinates.

The polymers and oligomers of the invention are generally prepared by polymerization of one or more monomer types, of which at least one monomer leads to repeat units of the formula (I) in the polymer. Suitable polymerization reactions are known to those skilled in the art and are described in the literature. Particularly suitable and preferred polymerization reactions which lead to C—C and C—N couplings are as follows:
(A) SUZUKI polymerization;
(B) YAMAMOTO polymerization;
(C) STILLE polymerization; and
(D) HARTWIG-BUCHWALD polymerization.

How the polymerization can be conducted by these methods and how the polymers can then be separated from the reaction medium and purified is known to those skilled in the art and is described in detail in the literature.

For the processing of the compounds containing a structural element of formula (I) from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (—)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane or mixtures of these solvents.

The invention therefore further provides a formulation, especially a solution, dispersion or emulsion, comprising at least one compound containing a structural element of formula (I) or at least one polymer, oligomer or dendrimer containing at least one unit of formula (I) and at least one solvent, preferably an organic solvent. The way in which such solutions can be prepared is known to those skilled in the art.

In a preferred embodiment of the invention, the formulation, apart from the compound of the application, also contains at least one further matrix material and at least one phosphorescent emitter. The at least one further matrix material and the at least one phosphorescent emitter are selected from the embodiments specified as preferred below. Application and evaporation of the solvent out of the formulation leaves the mixture of the materials as phosphorescent emitting layer with a mixed matrix.

The compounds of the application are suitable for use in electronic devices, especially in organic electroluminescent devices (OLEDs). Depending on the substitution, the compounds are used in different functions and layers.

The invention therefore further provides for the use of the compounds of the application in electronic devices. These electronic devices are preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and more preferably organic electroluminescent devices (OLEDs).

The invention further provides, as already set out above, an electronic device comprising at least one compound as defined above. This electronic device is preferably selected from the abovementioned devices.

It is more preferably an organic electroluminescent device (OLED) comprising anode, cathode and at least one emitting layer, characterized in that the at least one organic layer, which is preferably selected from emitting layers, electron transport layers and hole blocker layers, and which is more preferably selected from emitting layers, most preferably phosphorescent emitting layers, comprises at least one compound as defined above.

Apart from the cathode, anode and at least one emitting layer, the organic electroluminescent device may also comprise further layers. These are selected, for example, from in each case one or more hole injection layers, hole transport layers, hole blocker layers, electron transport layers, electron injection layers, electron blocker layers, exciton blocker layers, interlayers, charge generation layers and/or organic or inorganic p/n junctions.

The sequence of layers in the organic electroluminescent device is preferably:

anode/hole injection layer/hole transport layer/optionally further hole transport layer(s)/electron blocker layer/emitting layer/hole blocker layer/electron transport layer/optionally further electron transport layer(s)/electron injection layer/cathode. It is additionally possible for further layers to be present in the OLED.

It is preferable when at least one hole-transporting layer of the apparatus is p-doped, i.e. contains at least one p-dopant. p-Dopants are preferably selected from electron acceptor compounds.

Particularly preferred p-dopants are selected from quinodimethane compounds, azaindenofluorenediones, azaphenalenes, azatriphenylenes, I₂, metal halides, preferably transition metal halides, metal oxides, preferably metal oxides containing at least one transition metal or a metal of main group 3, and transition metal complexes, preferably complexes of Cu, Co, Ni, Pd and Pt with ligands containing at least one oxygen atom as binding site. Preference is further given to transition metal oxides as dopants, preferably oxides of rhenium, molybdenum and tungsten, more preferably Re₂O₇, MoO₃, WO₃ and ReO₃. Also preferred are bismuth complexes, especially Bi(III) complexes, especially bismuth complexes with benzoic acid derivatives as complex ligands.

The organic electroluminescent device of the invention may contain two or more emitting layers. More preferably, these emission layers have several emission maxima between 380 nm and 750 nm overall, such that the overall result is white emission; in other words, various emitting compounds which may fluoresce or phosphoresce and which emit blue, green yellow, orange or red light are used in the emitting layers. Especially preferred are three-layer systems, i.e. systems having three emitting layers, wherein one of the three layers in each case shows blue emission, one of the three layers in each case shows green emission, and one of the three layers in each case shows orange or red emission. The compounds of the invention are preferably present in the emitting layer. For the generation of white light, rather than multiple color-emitting emitter compounds, an emitter compound used individually that emits over a broad wavelength range is also suitable.

It is preferable in accordance with the invention when the compounds are used in an electronic device comprising one or more phosphorescent emitting compounds in an emitting layer. The compounds are preferably present in the emitting layer in combination with the phosphorescent emitting compound, more preferably in a mixture with at least one further matrix material. The latter is preferably selected from hole-conducting matrix materials, electron-conducting matrix materials and matrix materials having both hole-conducting and electron-conducting properties (bipolar matrix materials), more preferably from electron-conducting matrix materials and bipolar matrix materials, most preferably from electron-conducting matrix materials.

The term “phosphorescent emitting compounds” preferably encompasses those compounds where the emission of light is effected through a spin-forbidden transition, for example a transition from an excited triplet state or a state having a higher spin quantum number, for example a quintet state.

In a preferred embodiment of the present invention, the compound containing a structural element of the formula (I) is used in an emitting layer as matrix material in combination with one or more phosphorescent emitting compounds. The phosphorescent emitting compound is preferably a red- or green-phosphorescing emitter.

The total proportion of all matrix materials in the phosphorescent emitting layer in this case is between 50.0% and 99.9% by volume, preferably between 80.0% and 99.5% by volume, and more preferably between 85.0% and 97.0% by volume.

Correspondingly, the proportion of the phosphorescent emitting compound is between 0.1% and 50.0% by volume, preferably between 0.5% and 20.0% by volume, and more preferably between 3.0% and 15.0% by volume.

In an alternative embodiment, the phosphorescent emitting layer comprises just one matrix compound which is preferably a compound containing a structural element of the formula (I). In this case, the emitting layer is preferably a red-phosphorescing emitting layer.

The phosphorescent emitting layer of the organic electroluminescent device preferably comprises two or more matrix materials (mixed matrix systems). One of these is especially a compound containing a structural element of the formula (I). The mixed matrix systems preferably comprise two or three different matrix materials, more preferably two different matrix materials, one of which is preferably a compound containing a structural element of the formula (I).

In a preferred embodiment, one of the two matrix materials fulfills the function of a hole-transporting material, and the other of the two matrix materials fulfills the function of an electron-transporting material. More preferably, the compound containing a structural element of the formula (I) here is the electron-transporting material, and the further compound present in the emitting layer in a mixture with the compound containing a structural element of the formula (I) is the hole-transporting material. In this case, the further compound is preferably selected from carbazole compounds, biscarbazole compounds, indolocarbazole compounds and indenocarbazole compounds. These preferably do not have any electron-deficient heteroaromatic systems as substituents. In this case, the compound containing a structural element of the formula (I) preferably has one or more electron-deficient heteroaromatic systems, preferably triazine, as substituents.

In an alternative preferred embodiment, the compound containing a structural element of the formula (I) is the hole-transporting matrix material, and the further compound present in the emitting layer in a mixture with the compound containing a structural element of the formula (I) is the electron-transporting matrix material. In this case, the further compound is preferably selected from carbazole compounds and indenocarbazole compounds. These preferably have one or more electron-deficient heteroaromatic systems, preferably triazine, as substituents. In this case, the compound containing a structural element of the formula (I) preferably does not have any electron-deficient heteroaromatic systems as substituents.

In a further preferred embodiment of the invention, one of the two materials is a wide bandgap material, and one or two further matrix materials are present in the emitting layer, which fulfill an electron-transporting function and/or a hole-transporting function of the mixed matrix. In a preferred embodiment, this can be accomplished in that not only the wide bandgap material but also a further matrix material having electron-transporting properties is present in the emitting layer, and yet a further matrix material having hole-transporting properties is present in the emitting layer. Alternatively and more preferably, this can be accomplished in that not only the wide bandgap material but also a single further matrix material having both electron-transporting and hole-transporting properties is present in the emitting layer. Such matrix materials are also referred to as bipolar matrix materials.

In another alternative embodiment, as well as the wide bandgap matrix material, only a single further matrix material having either predominantly hole-transporting properties or predominantly electron-transporting properties may be present in the emitting layer.

In the preferred case that two different matrix materials are present in the emitting layer, these may be present in a volume ratio of 1:50 to 1:1, preferably 1:20 to 1:1, more preferably 1:10 to 1:1 and most preferably 1:4 to 1:1. Preferably, the compound containing a structural element of the formula (I) is present in the same proportion as the further matrix compound, or it is present in a higher proportion than the further matrix compound.

The absolute proportion of the compound containing a structural element of the formula (I) in the mixture of the emitting layer, in the case of use as matrix material in a phosphorescent emitting layer, is preferably 10% by volume to 85% by volume, more preferably 20% by volume to 85% by volume, even more preferably 30% by volume to 80% by volume, very especially preferably 20% by volume to 60% by volume and most preferably 30% by volume to 50% by volume. The absolute proportion of the second matrix compound in this case is preferably 15% by volume to 90% by volume, more preferably 15% by volume to 80% by volume, even more preferably 20% by volume to 70% by volume, very especially preferably 40% by volume to 80% by volume, and most preferably 50% by volume to 70% by volume.

For production of phosphorescent emitting layers of the mixed matrix type, in a preferred embodiment of the invention, a solution comprising the phosphorescent emitter and the two or more matrix materials may be produced. This can be applied by means of spin-coating, printing methods or other methods. Evaporation of the solvent in this case leaves the phosphorescent emitting layer of the mixed matrix type.

In an alternative, more preferred embodiment of the invention, the phosphorescent emitting layer of the mixed matrix type is produced by vapor phase deposition. For this purpose, there are two methods by which the layer can be applied. Firstly, each of the at least two different matrix materials may be initially charged in a material source, followed by simultaneous evaporation (“coevaporation”) from the two or more different material sources. Secondly, the at least two matrix materials may be premixed and the mixture obtained may be initially charged in a single material source from which it is ultimately evaporated. The latter method is referred to as the premix method.

The present application therefore also provides a mixture comprising a compound of the above-specified formulae and at least one further compound selected from matrix compounds. In this respect, the preferred embodiments with regard to proportions of the matrix compounds and their chemical structure that are specified in this application are likewise considered to be preferable.

In an alternative preferred embodiment of the invention, the compound is used as electron-transporting material. This is especially true when the compound contains at least one group selected from electron-deficient heteroaryl groups, preferably azine groups, especially triazine groups, pyrimidine groups and pyridine groups, and benzimidazole groups.

When the compound is used as electron-transporting material, it is preferably used in a hole blocker layer, an electron transport layer or in an electron injection layer. In a preferred embodiment, the layer comprising the compound containing a structural element of the formula (I) in that case is n-doped, or it is in a mixture with a further electron-transporting compound, preferably lithium quinolinate (LiQ). The compound containing a structural element of the formula (I) may alternatively be present as a pure material in the layer selected from hole blocker layer, electron transport layer and electron injection layer.

In the present context, an n-dopant is understood to mean an organic or inorganic compound capable of releasing electrons (electron donor), i.e. a compound that acts as a reducing agent. The compounds used for n-doping can be used in the form of a precursor, in which case these precursor compounds release n-dopants through activation. Preferably, n-dopants are selected from electron-rich metal complexes; P═N compounds; N-heterocycles, more preferably naphthylenecarbodiimides, pyridines, acridines and phenazines; fluorenes and free-radical compounds.

Preferred embodiments of the different functional materials in the electronic device are listed hereinafter.

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9, 10 position. Aromatic pyreneamines, pyrenediamines, chryseneamines and chrysenediamines are defined analogously, where the diarylamino groups are bonded to the pyrene preferably in the 1 position or 1, 6 position. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units.

Preferred matrix materials for fluorescent emitters are selected from the classes of the oligoarylenes (e.g. 2,2′,7,7′-tetraphenylspirobifluorene), especially the oligoarylenes containing fused aromatic groups, the oligoarylenevinylenes, the polypodal metal complexes, the hole-conducting compounds, the electron-conducting compounds, especially ketones, phosphine oxides and sulfoxides; the atropisomers, the boronic acid derivatives or the benzanthracenes. Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another.

Suitable phosphorescent emitting compounds (=triplet emitters) are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitting compounds, compounds containing copper, molybdenum, tungsten, rhenium, ruthenium, osmium, rhodium, iridium, palladium, platinum, silver, gold or europium, especially compounds containing iridium, platinum or copper. In the context of the present invention, all luminescent iridium, platinum or copper complexes are considered to be phosphorescent emitting compounds.

In general, all phosphorescent complexes as used for phosphorescent OLEDs according to the prior art and as known to those skilled in the art in the field of organic electroluminescent devices are suitable. Explicit examples of particularly suitable complexes are shown in the following table:

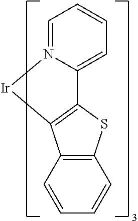

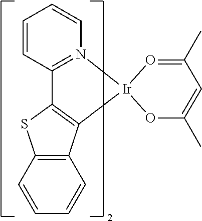

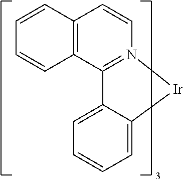

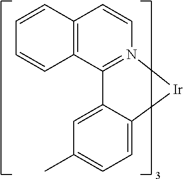

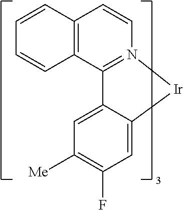

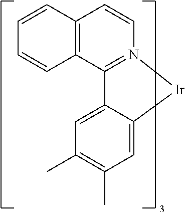

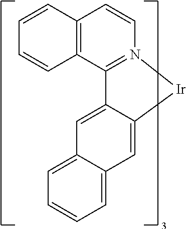

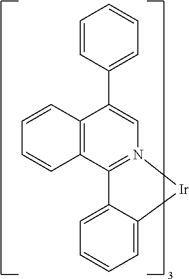

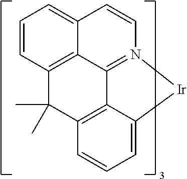

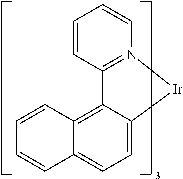

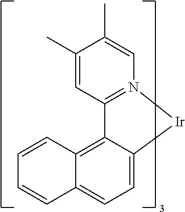

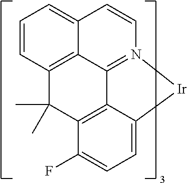

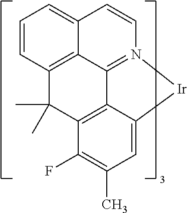

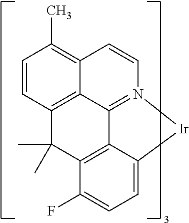

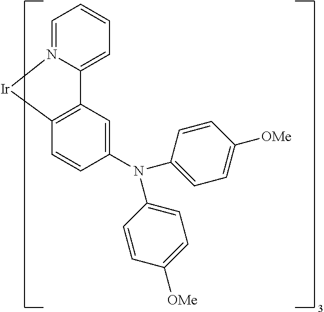

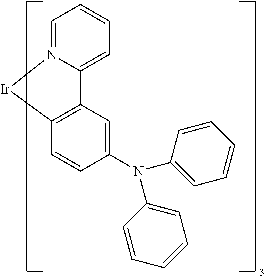

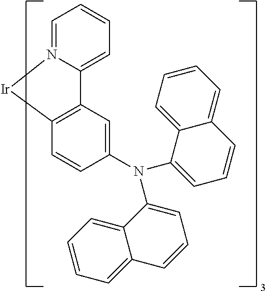

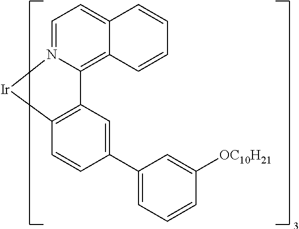

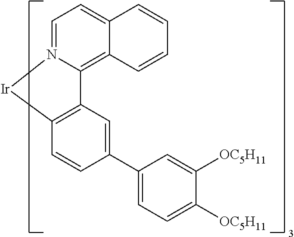

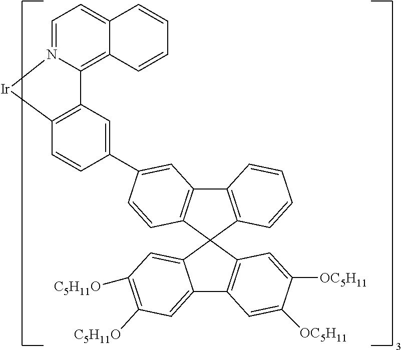

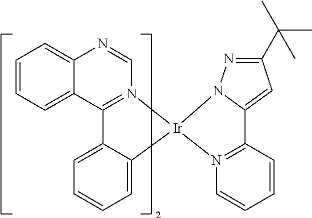

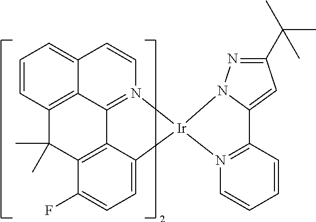

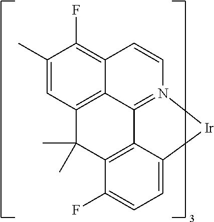

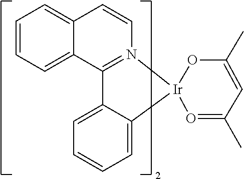

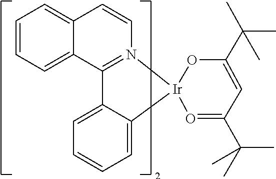

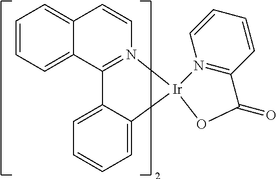

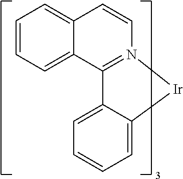

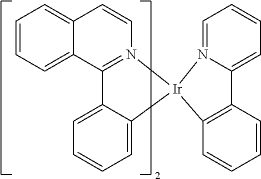

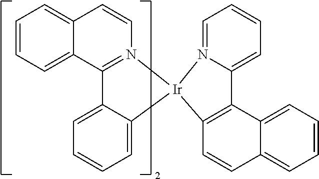

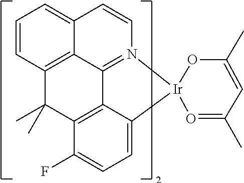

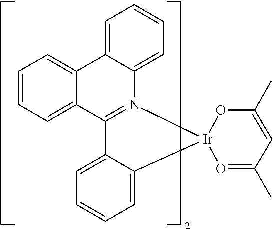

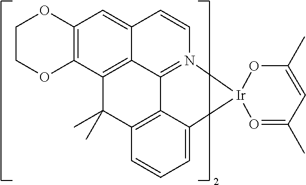

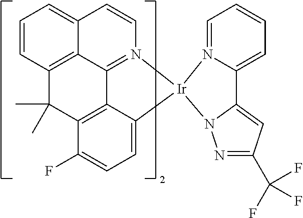

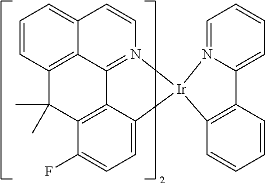

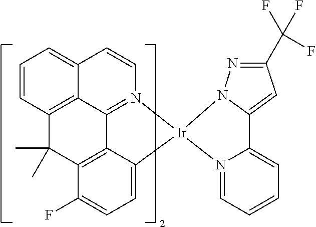

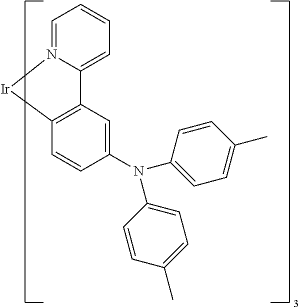

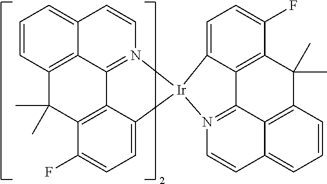

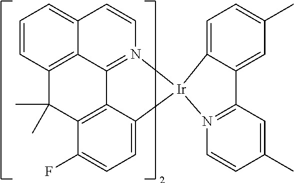

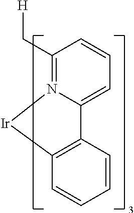

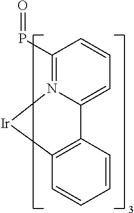

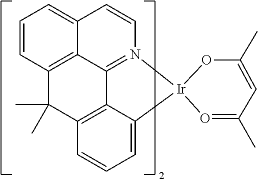

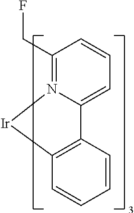

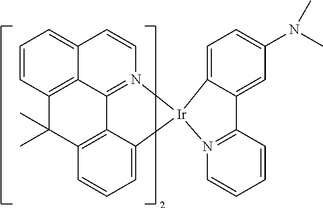

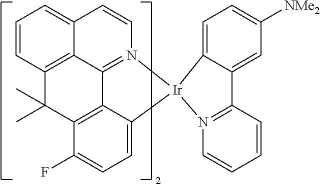

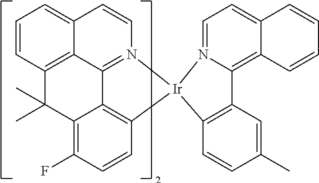

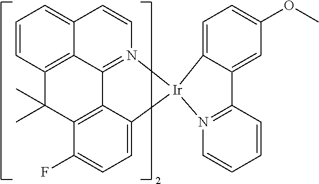

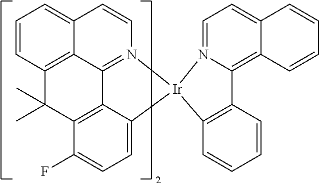

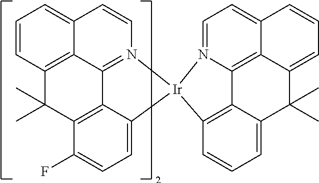

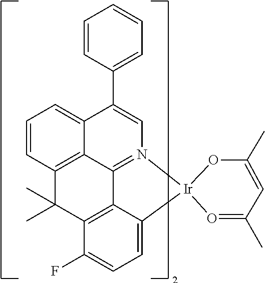

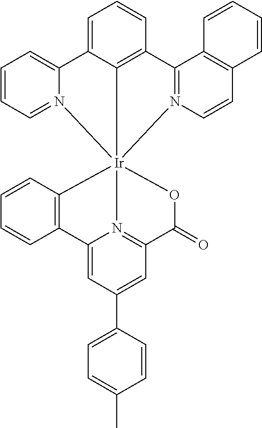

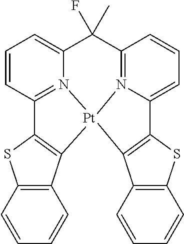

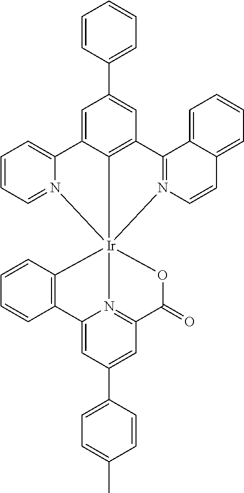

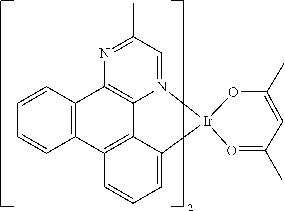

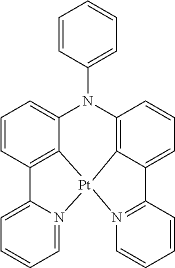

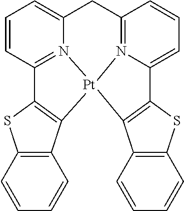

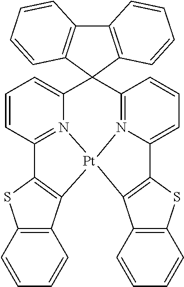

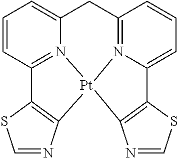

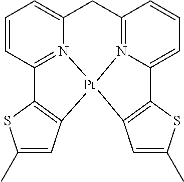

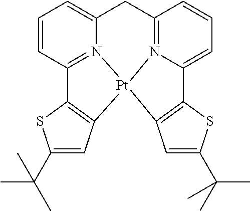

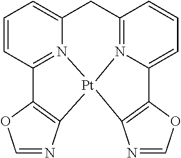

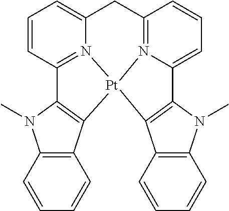

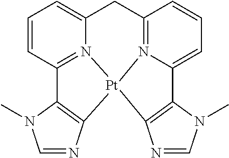

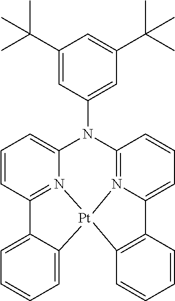

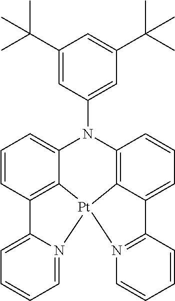

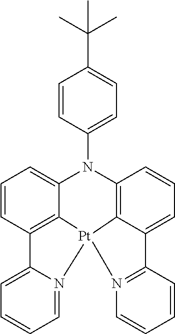

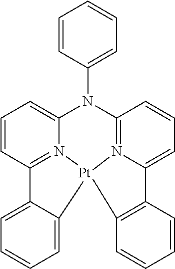

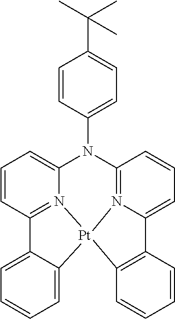

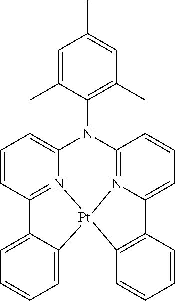

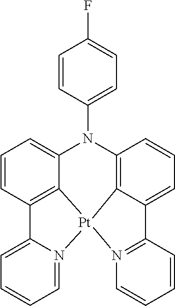

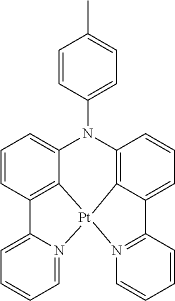

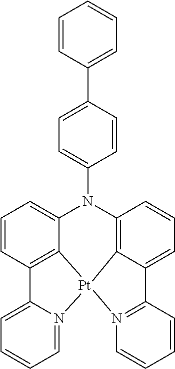

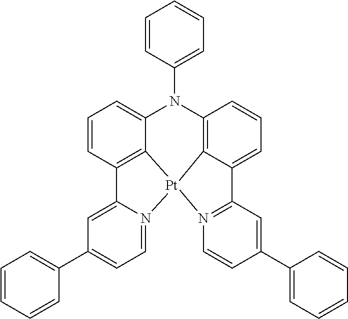

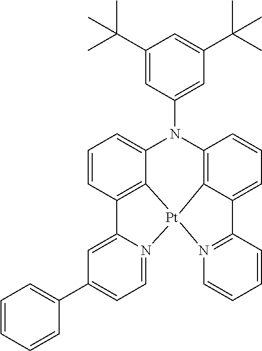

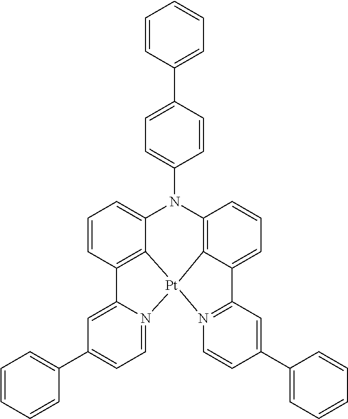

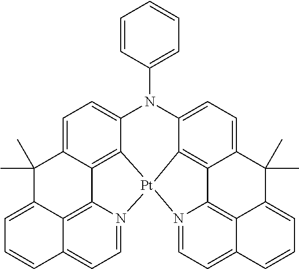

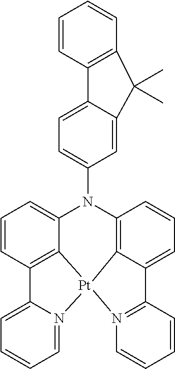

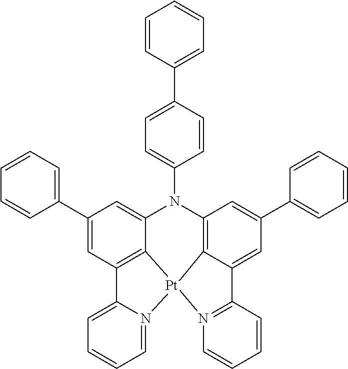

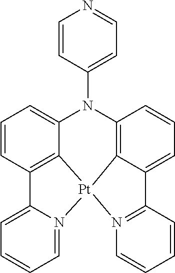

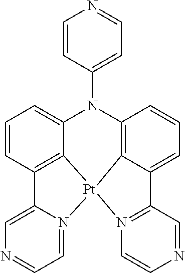

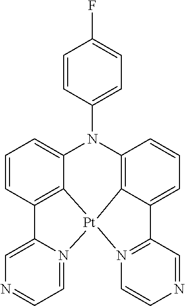

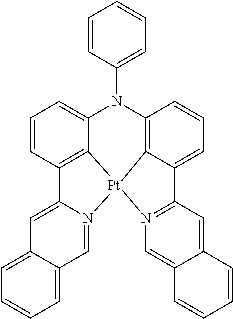

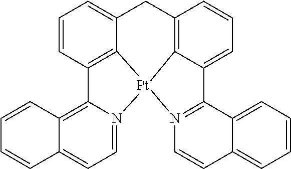

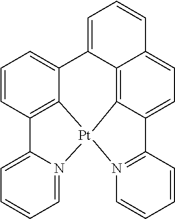

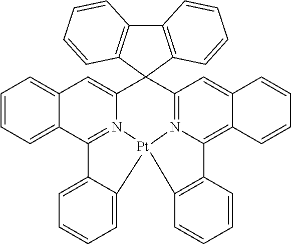

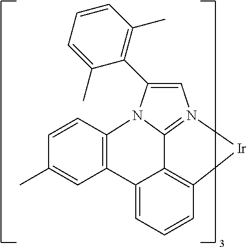

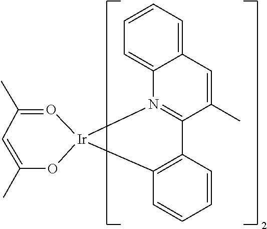

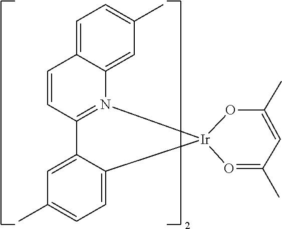

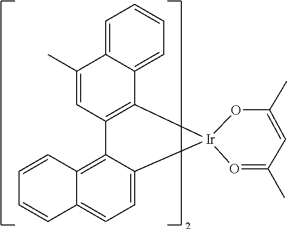

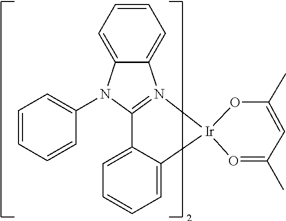

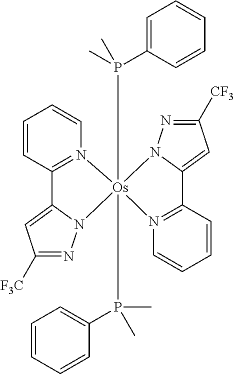

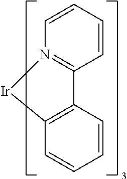

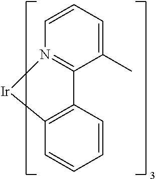

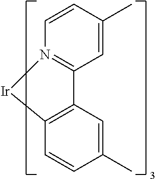

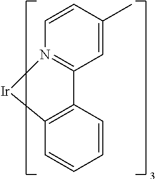

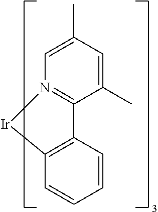

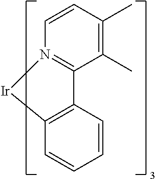

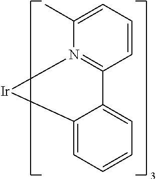

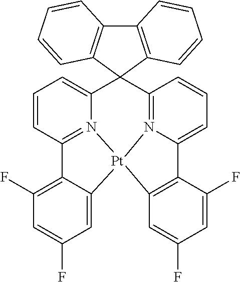

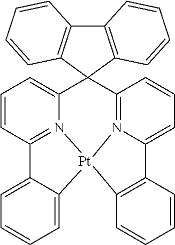

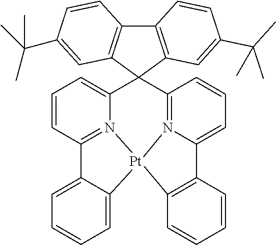

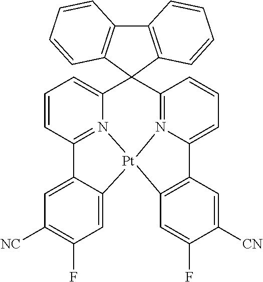

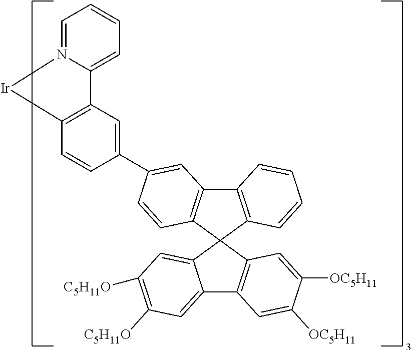

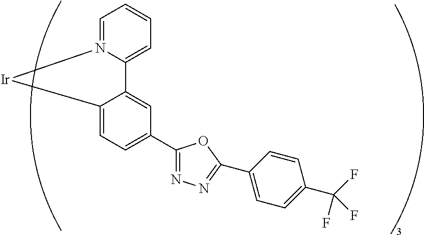

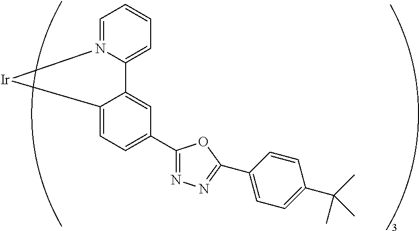

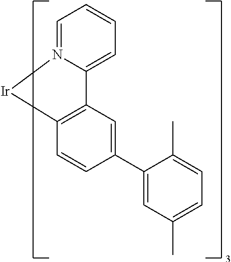

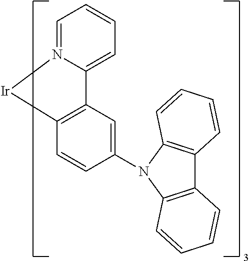

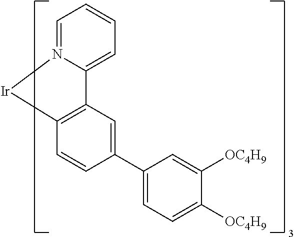

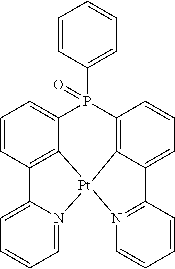

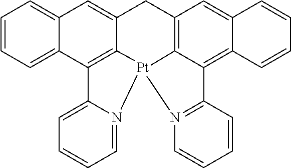

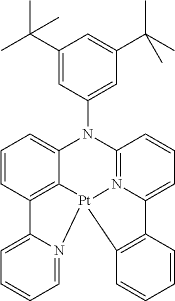

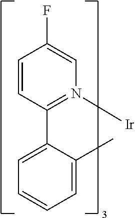

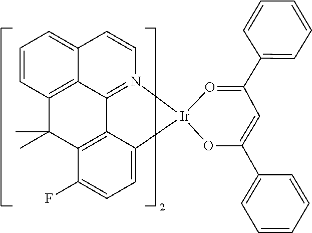

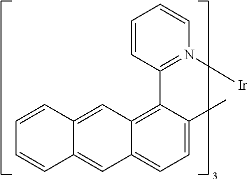

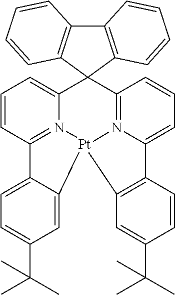

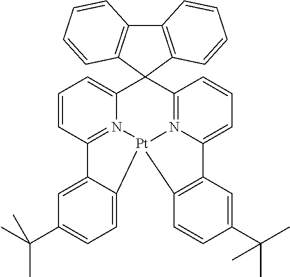

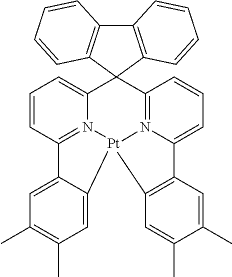

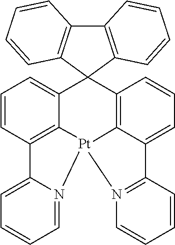

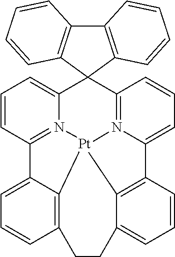

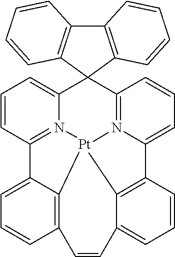

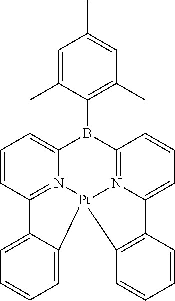

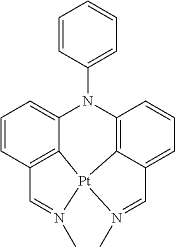

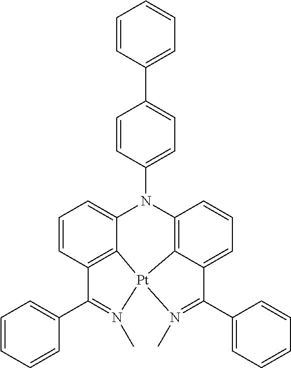

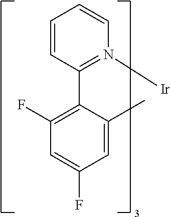

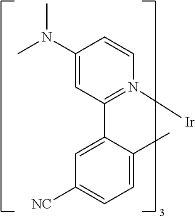

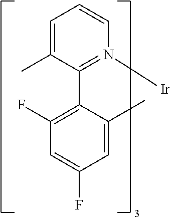

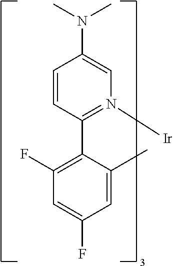

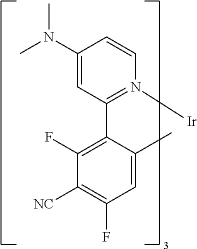

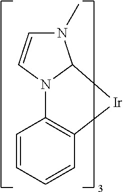

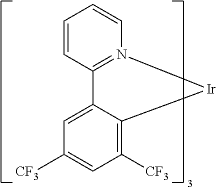

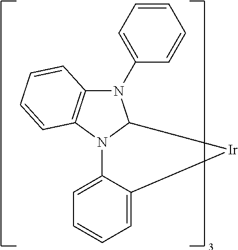

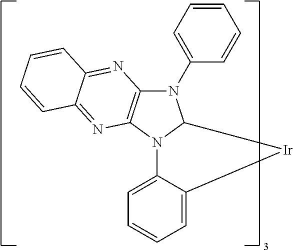

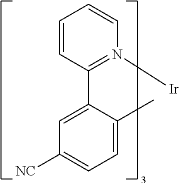

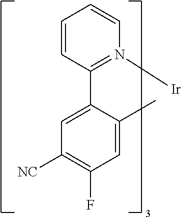

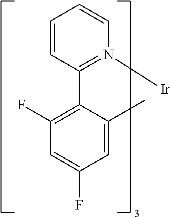

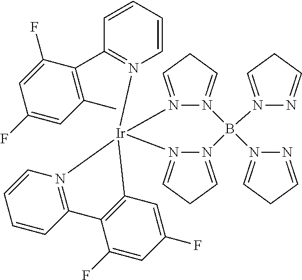

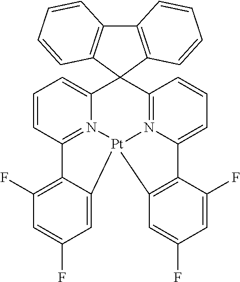

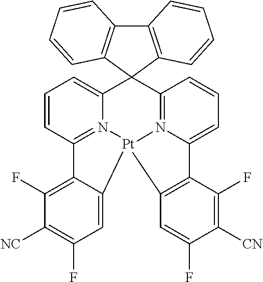

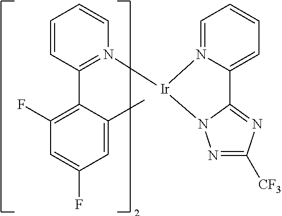

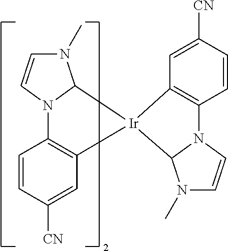

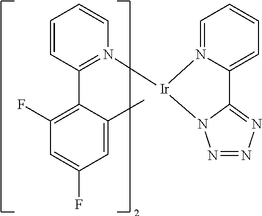

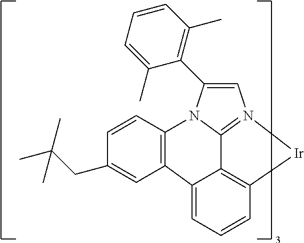

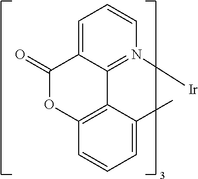

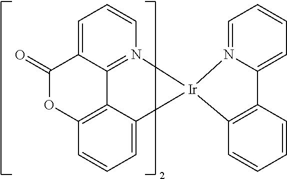

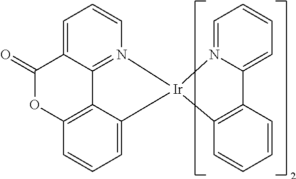

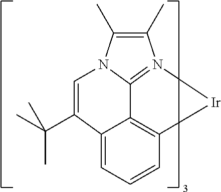

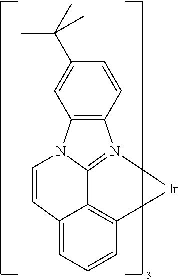

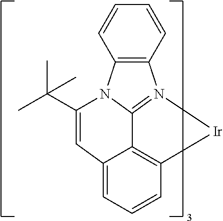

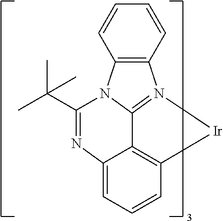

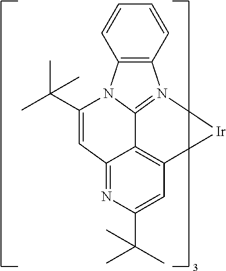

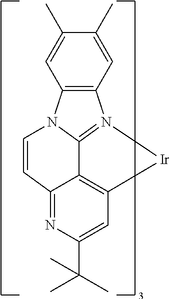

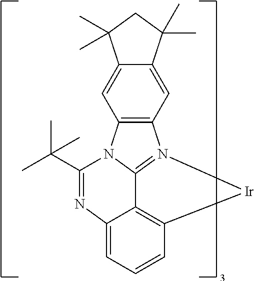

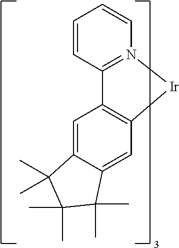

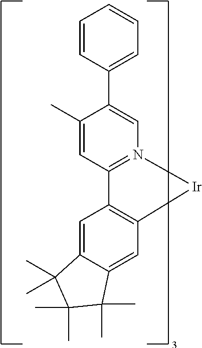

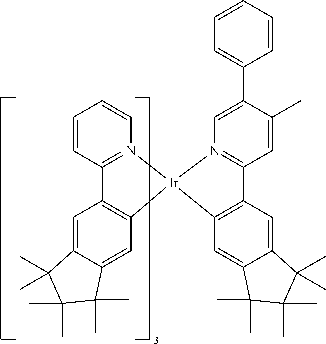

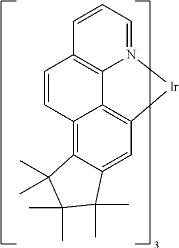

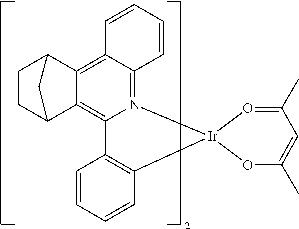

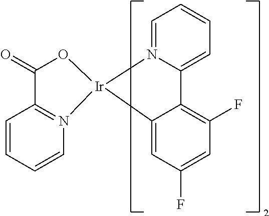

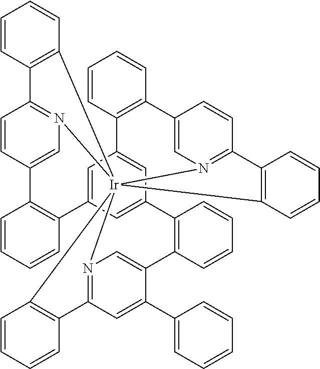

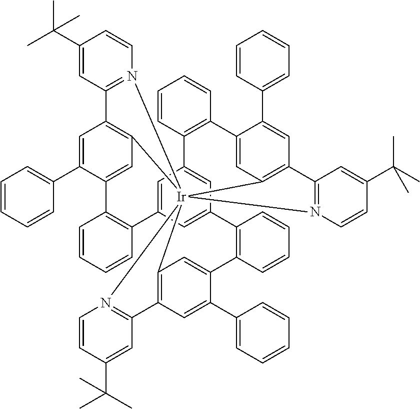

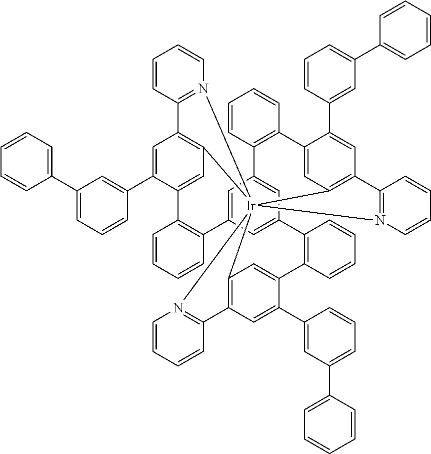

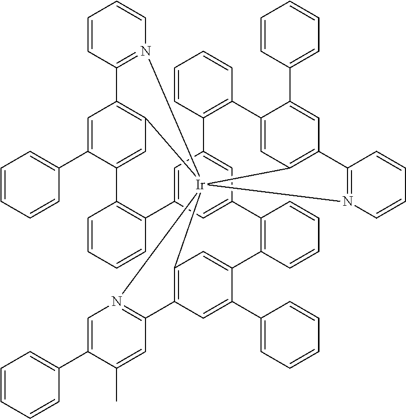

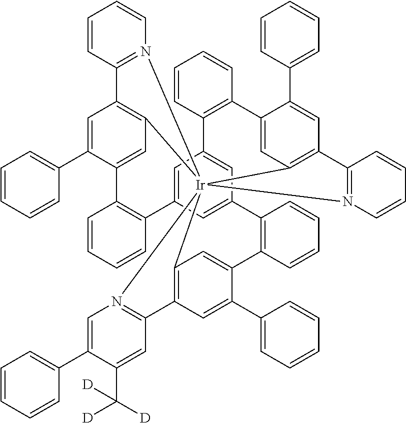

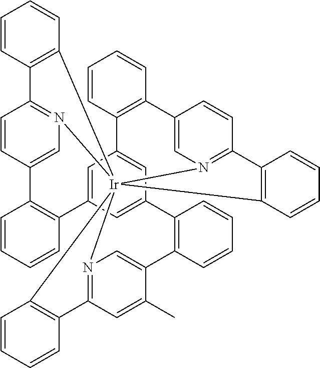

Preferred matrix materials for phosphorescent emitters, as well as the compounds of the application, are aromatic ketones, aromatic phosphine oxides or aromatic sulfoxides or sulfones, triarylamines, carbazole derivatives, e.g. CBP (N,N-biscarbazolylbiphenyl) or carbazole derivatives, indolocarbazole derivatives, indenocarbazole derivatives, azacarbazole derivatives, bipolar matrix materials, silanes, azaboroles or boronic esters, triazine derivatives, zinc complexes, diazasilole or tetraazasilole derivatives, diazaphosphole derivatives, bridged carbazole derivatives, triphenylene derivatives, or lactams. More preferably, the compound containing a structural element of the formula (I) is used in the emitting layer in combination with a phosphorescent emitter and a further matrix material which is preferably selected from the abovementioned preferred matrix materials and is more preferably selected from carbazole compounds, biscarbazole compounds, indolocarbazole compounds and indenocarbazole compounds.

Suitable charge transport materials as usable in the hole injection layer or hole transport layer or electron blocker layer or in the electron transport layer of the electronic device of the invention are, for example, the compounds disclosed in Y. Shirota et al., Chem. Rev. 2007, 107(4), 953-1010, or other materials as used in these layers according to the prior art.

Suitable materials for the electron-transporting layers of the device are especially aluminum complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives.

Particularly preferred compounds for use in electron-transporting layers are shown in the following table:

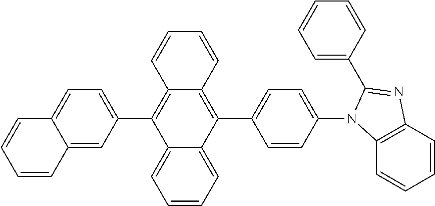 [SMILES: c1ccc(-c2nc3ccccc3n2-c2ccc(-c3c4ccccc4c(-c4ccc5ccccc5c4)c4ccccc34)cc2)cc1]

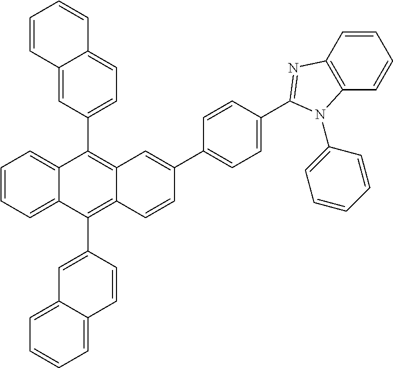 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

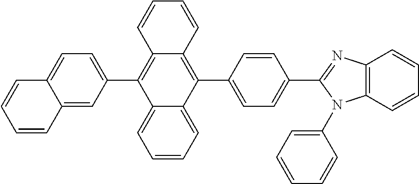 [SMILES: c1ccc(-n2c(-c3ccc(-c4c5ccccc5c(-c5ccc6ccccc6c5)c5ccccc45)cc3)nc3ccccc32)cc1]

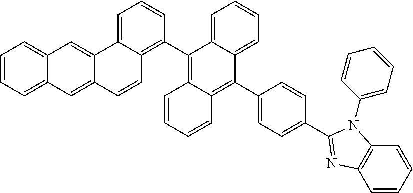 [SMILES: c1ccc(-n2c(-c3ccc(-c4c5ccccc5c(-c5cccc6c5ccc5cc7ccccc7cc56)c5ccccc45)cc3)nc3ccccc32)cc1]

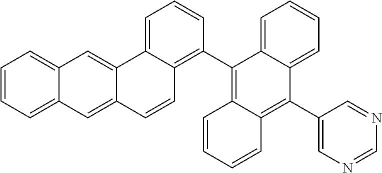 [SMILES: c1ccc2cc3c(ccc4c(-c5c6ccccc6c(-c6cncnc6)c6ccccc56)cccc43)cc2c1]

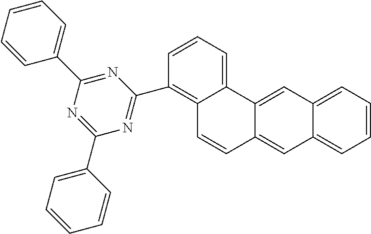 [SMILES: c1ccc(-c2nc(-c3ccccc3)nc(-c3cccc4c3ccc3cc5ccccc5cc34)n2)cc1]

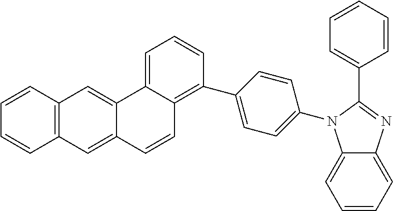 [SMILES: c1ccc(-c2nc3ccccc3n2-c2ccc(-c3cccc4c3ccc3cc5ccccc5cc34)cc2)cc1]

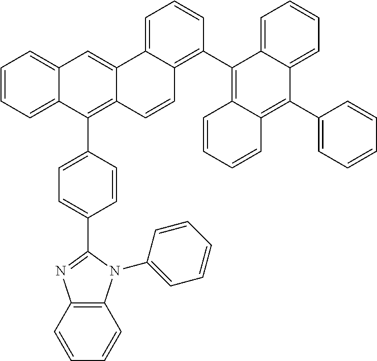 [SMILES: c1ccc(-c2c3ccccc3c(-c3cccc4c3ccc3c(-c5ccc(-c6nc7ccccc7n6-c6ccccc6)cc5)c5ccccc5cc34)c3ccccc23)cc1]

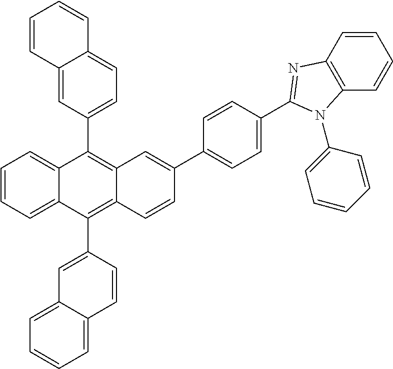 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

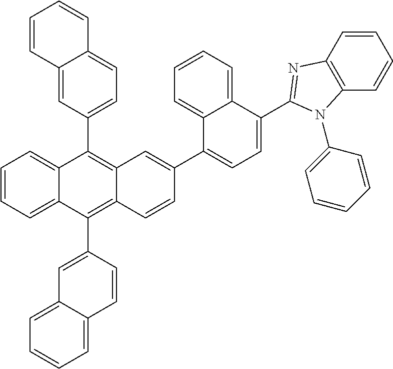 [SMILES: c1ccc(-n2c(-c3ccc(-c4ccc5c(-c6ccc7ccccc7c6)c6ccccc6c(-c6ccc7ccccc7c6)c5c4)c4ccccc34)nc3ccccc32)cc1]

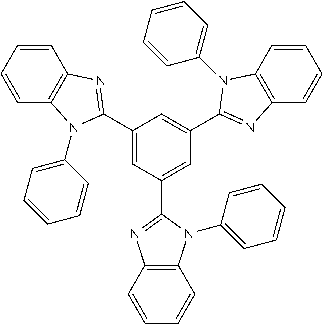 [SMILES: c1ccc(-n2c(-c3cc(-c4nc5ccccc5n4-c4ccccc4)cc(-c4nc5ccccc5n4-c4ccccc4)c3)nc3ccccc32)cc1]

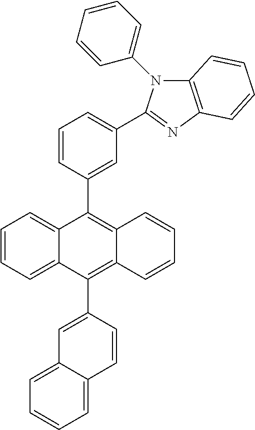 [SMILES: c1ccc(-n2c(-c3cccc(-c4c5ccccc5c(-c5ccc6ccccc6c5)c5ccccc45)c3)nc3ccccc32)cc1]

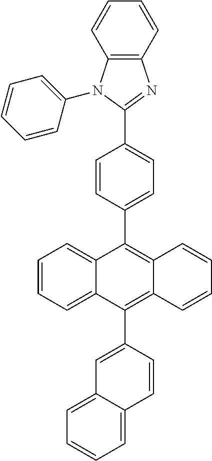 [SMILES: c1ccc(-n2c(-c3ccc(-c4c5ccccc5c(-c5ccc6ccccc6c5)c5ccccc45)cc3)nc3ccccc32)cc1]

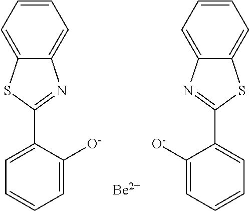

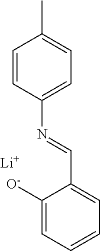 [SMILES: Cc1ccc(/N=C/c2ccccc2[O-])cc1 ; [Li+]]

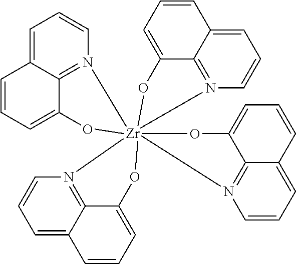

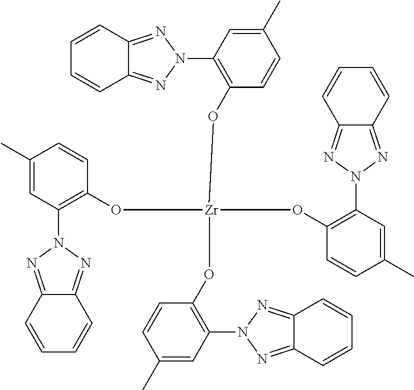

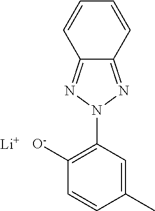 [SMILES: [Li+] ; Cc1ccc([O-])c(-n2nc3ccccc3n2)c1]

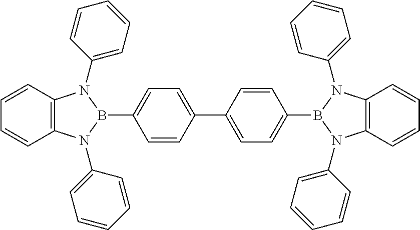 [SMILES: c1ccc(N2B(c3ccc(-c4ccc(B5N(c6ccccc6)c6ccccc6N5c5ccccc5)cc4)cc3)N(c3ccccc3)c3ccccc32)cc1]

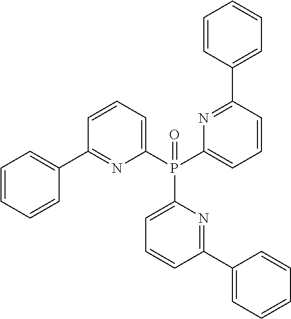 [SMILES: O=P(c1cccc(-c2ccccc2)n1)(c1cccc(-c2ccccc2)n1)c1cccc(-c2ccccc2)n1]

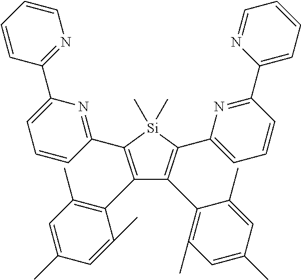 [SMILES: Cc1cc(C)c(C2=C(c3cccc(-c4ccccn4)n3)[Si](C)(C)C(c3cccc(-c4ccccn4)n3)=C2c2c(C)cc(C)cc2C)c(C)c1]

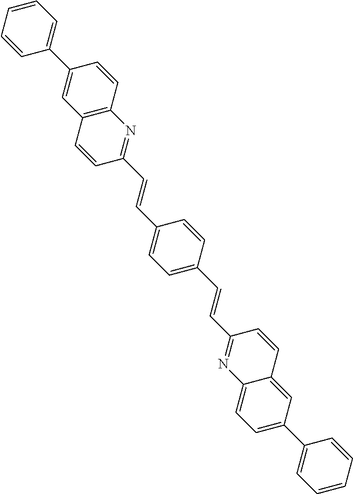 [SMILES: C(=C\c1ccc2cc(-c3ccccc3)ccc2n1)/c1ccc(/C=C/c2ccc3cc(-c4ccccc4)ccc3n2)cc1]

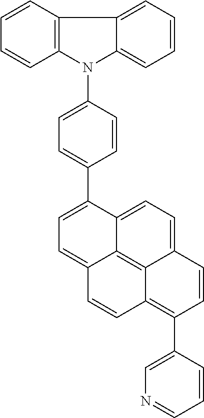 [SMILES: c1cncc(-c2ccc3ccc4c(-c5ccc(-n6c7ccccc7c7ccccc76)cc5)ccc5ccc2c3c54)c1]

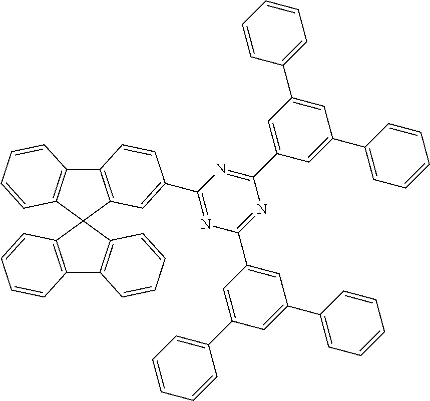 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

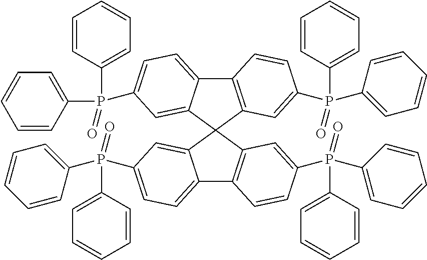 [SMILES: O=P(c1ccccc1)(c1ccccc1)c1ccc2c(c1)C1(c3cc(P(=O)(c4ccccc4)c4ccccc4)ccc3-2)c2cc(P(=O)(c3ccccc3)c3ccccc3)ccc2-c2ccc(P(=O)(c3ccccc3)c3ccccc3)cc21]

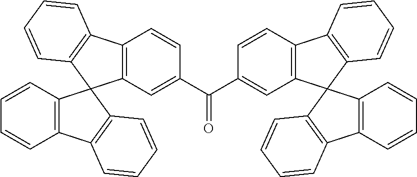 [SMILES: O=C(c1ccc2c(c1)C1(c3ccccc3-c3ccccc31)c1ccccc1-2)c1ccc2c(c1)C1(c3ccccc3-c3ccccc31)c1ccccc1-2]

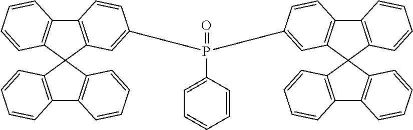 [SMILES: O=P(c1ccccc1)(c1ccc2c(c1)C1(c3ccccc3-c3ccccc31)c1ccccc1-2)c1ccc2c(c1)C1(c3ccccc3-c3ccccc31)c1ccccc1-2]

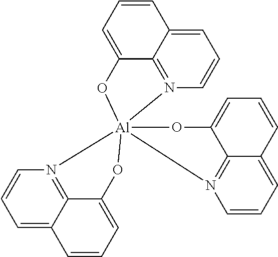

Materials used for hole-transporting layers of OLEDs may preferably be indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups.

In addition, the following compounds HT-1 to HT-72 are suitable for use in a layer having a hole-transporting function, especially in a hole injection layer, a hole transport layer and/or an electron blocker layer, or for use in an emitting layer as matrix material, especially as matrix material in an emitting layer comprising one or more phosphorescent emitters:

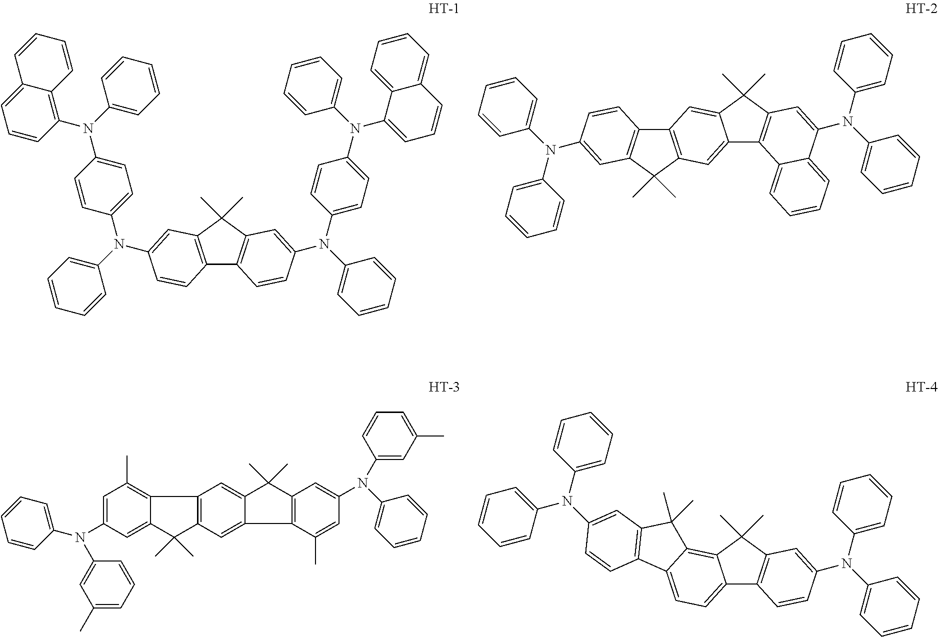

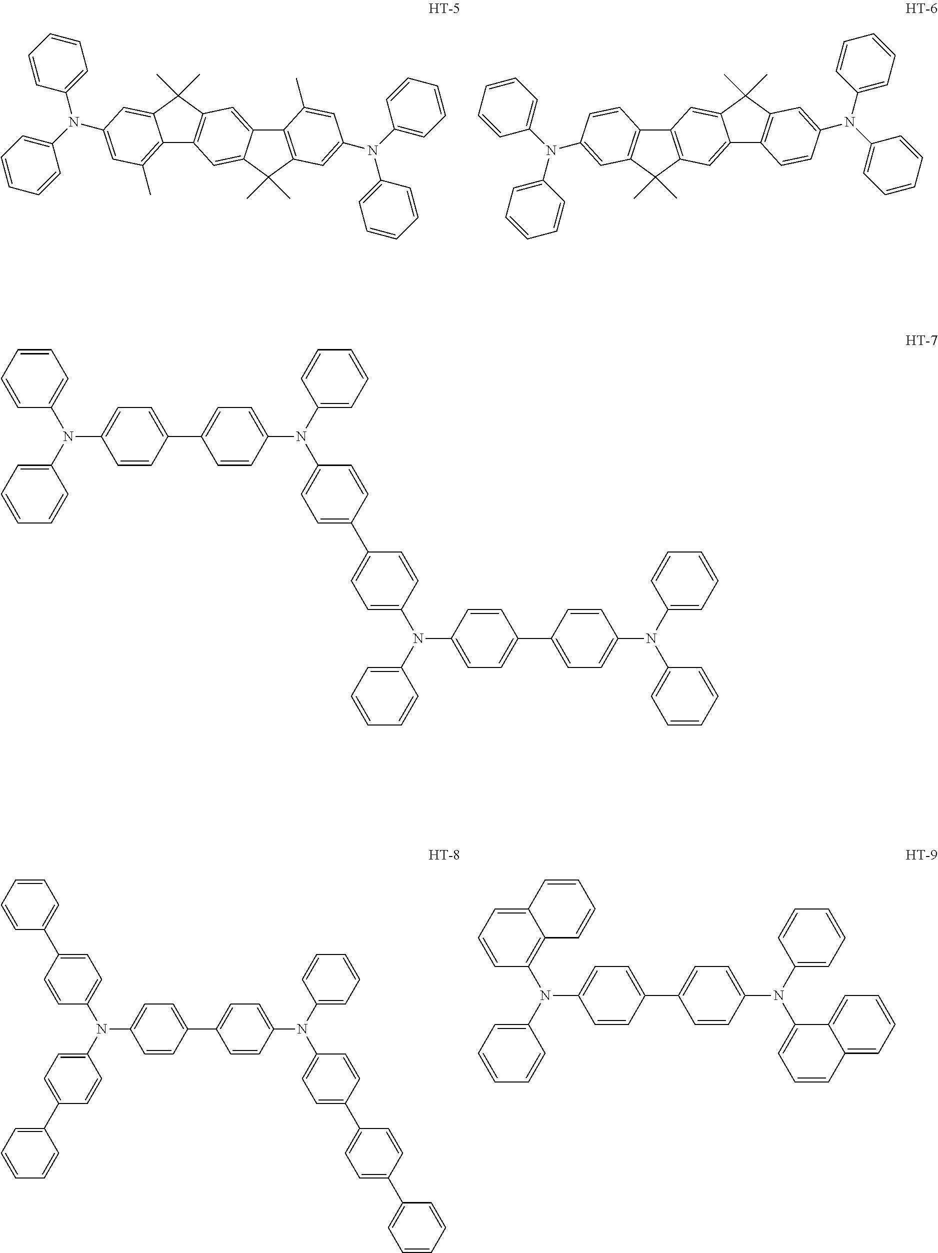

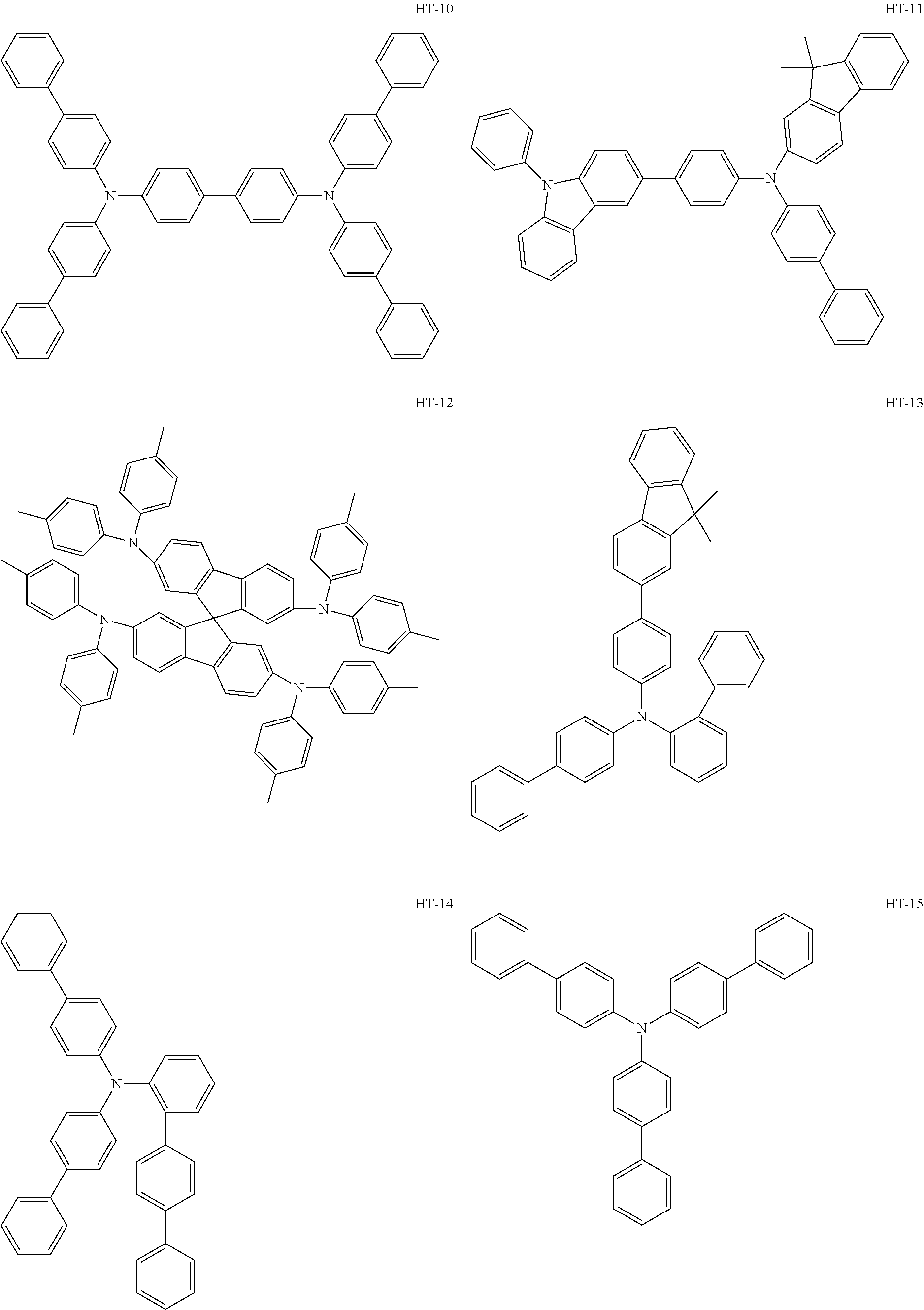

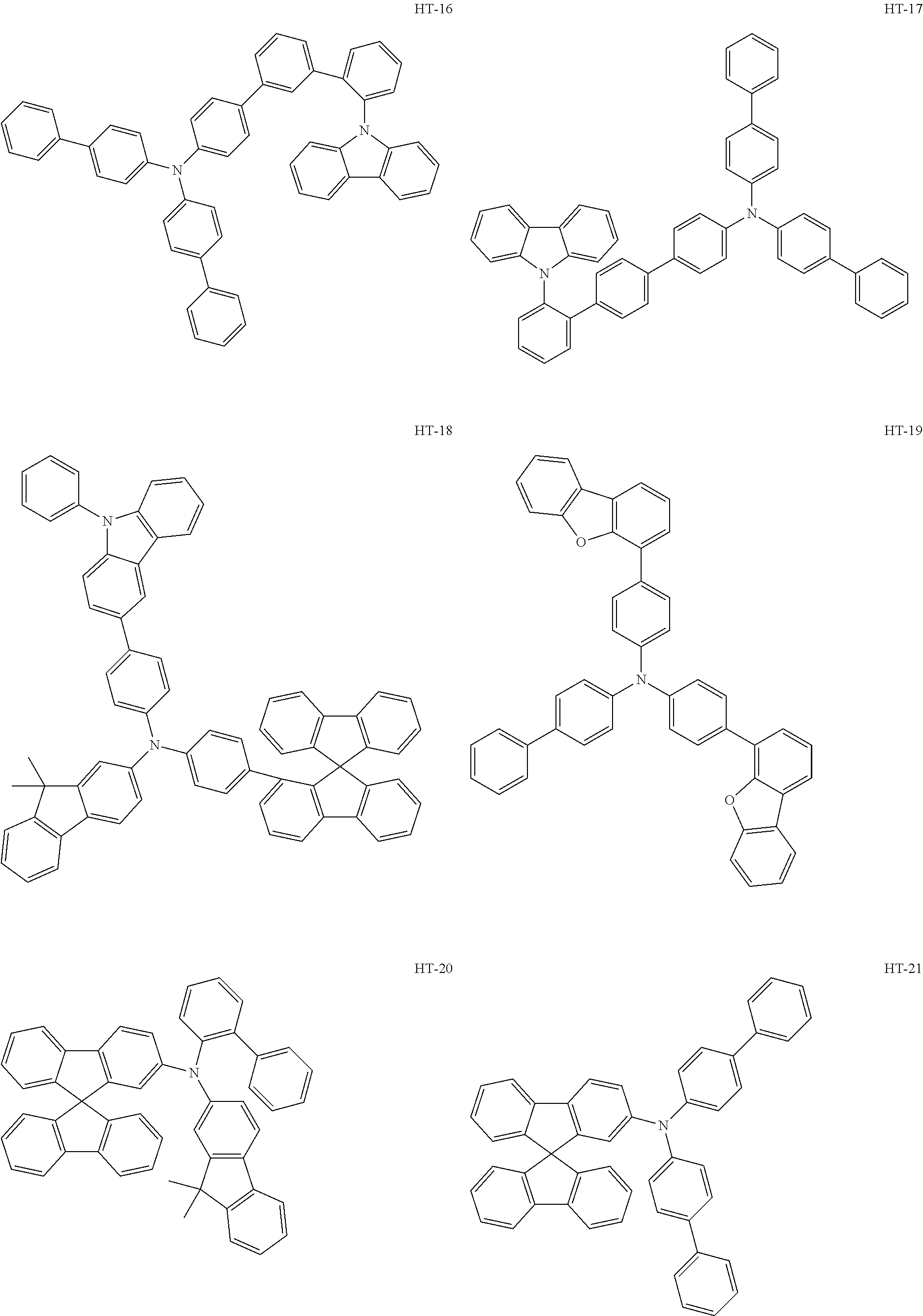

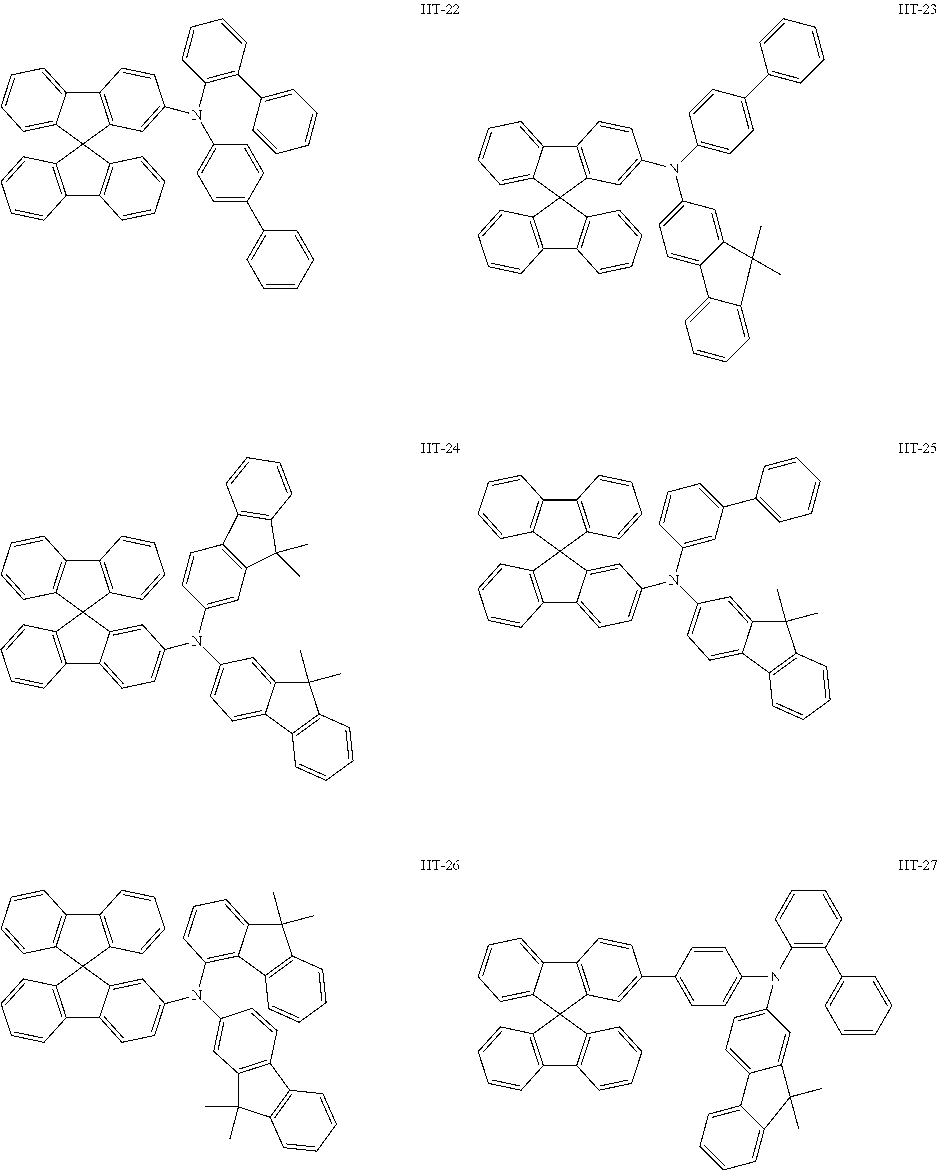

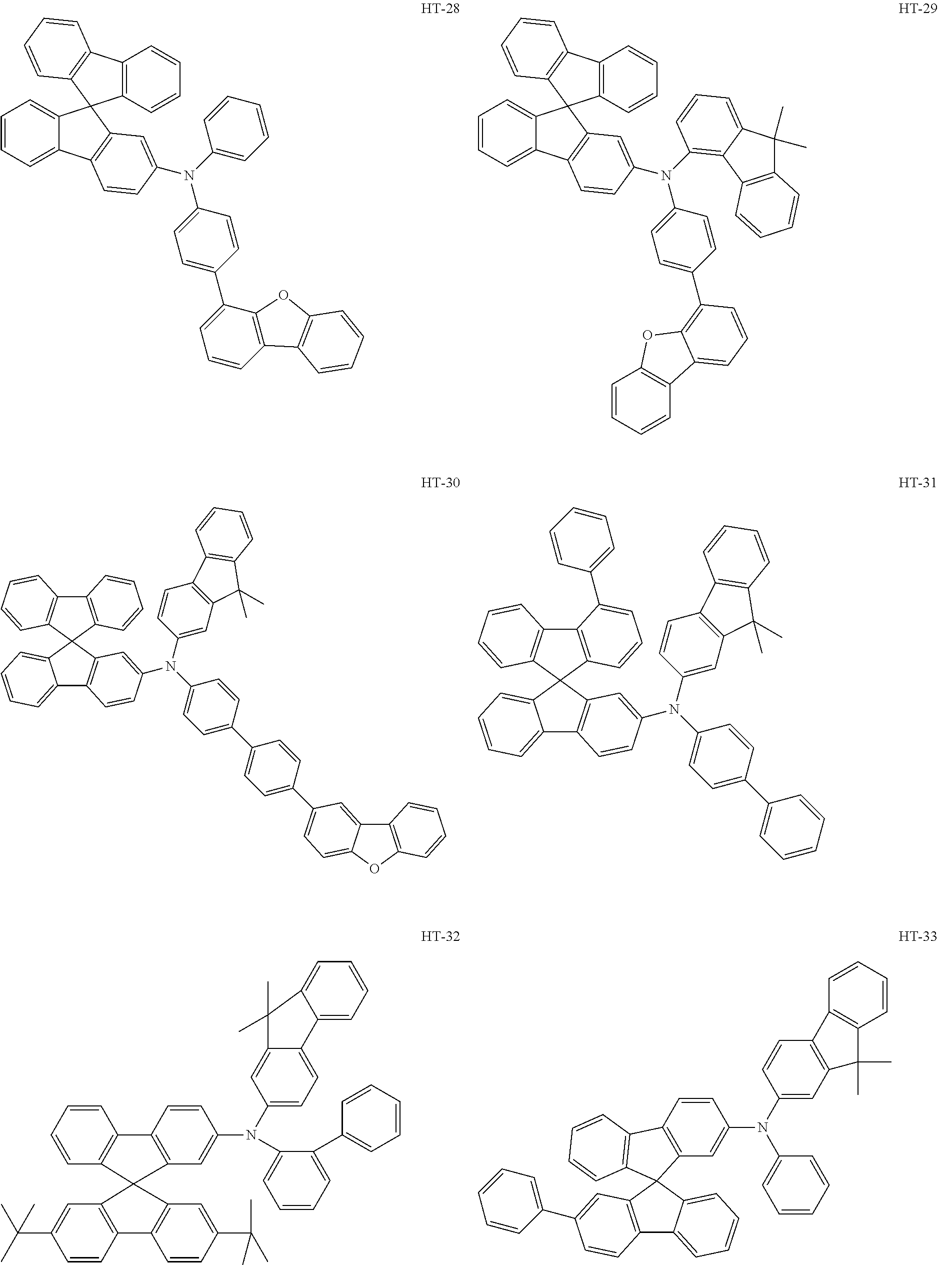

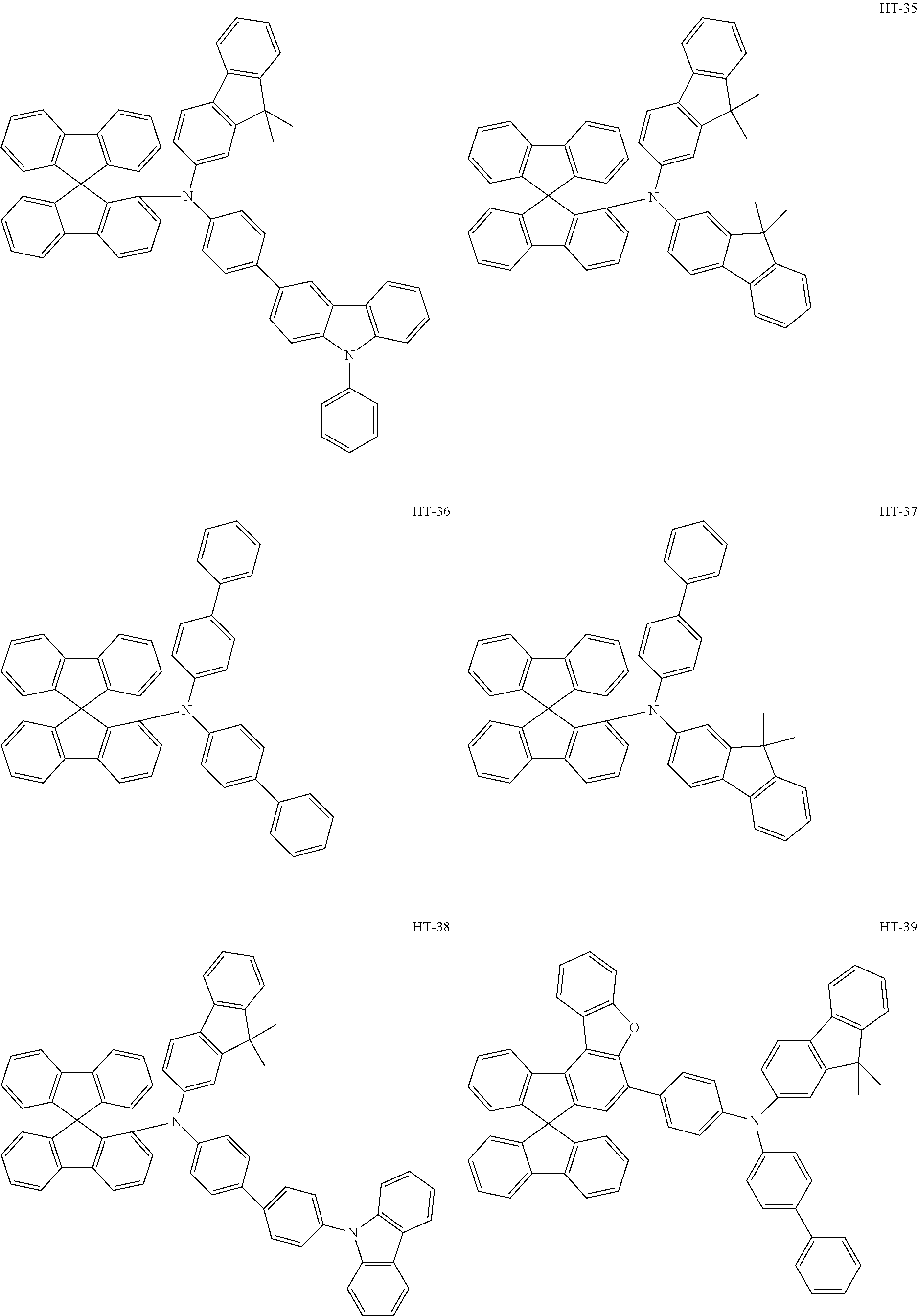

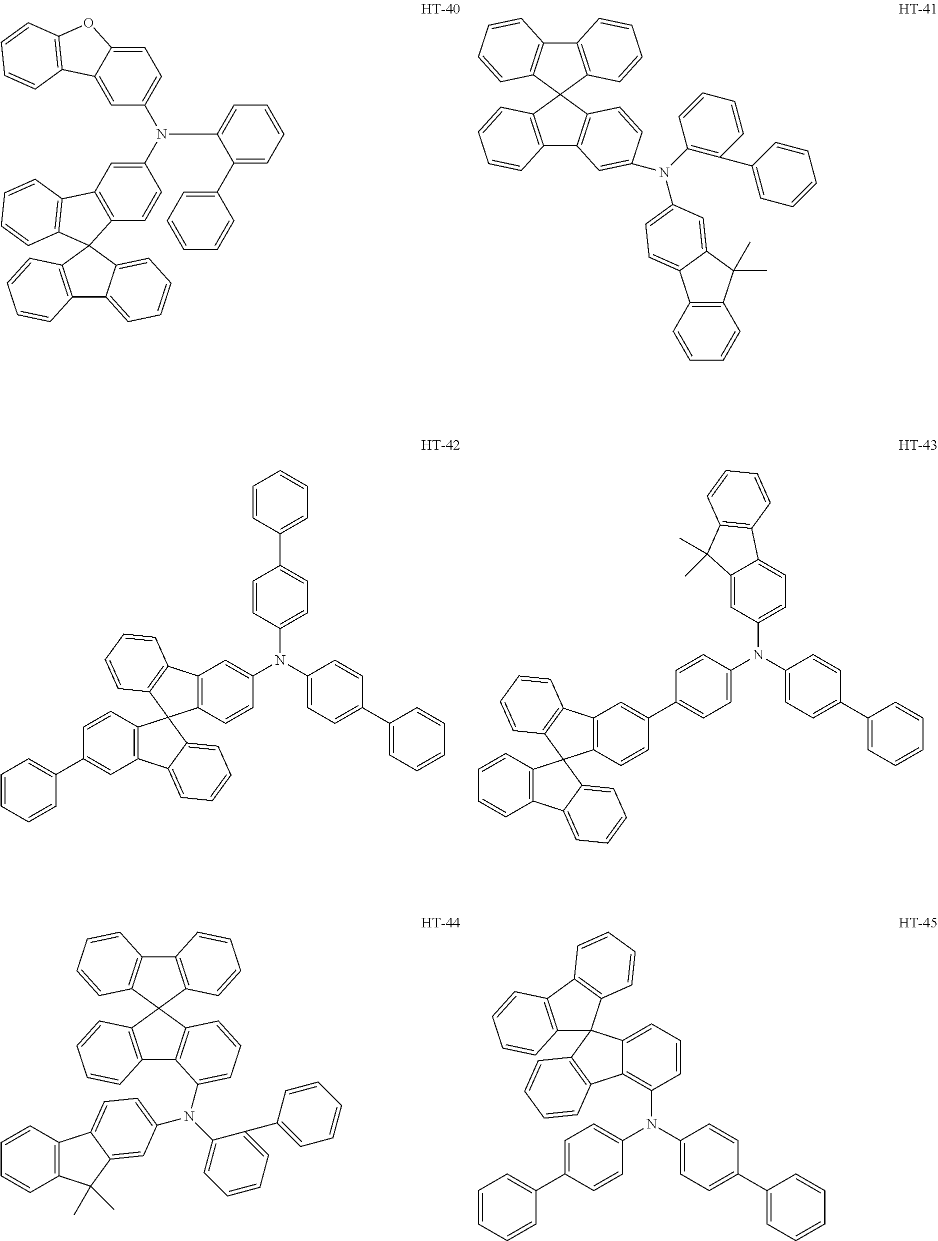

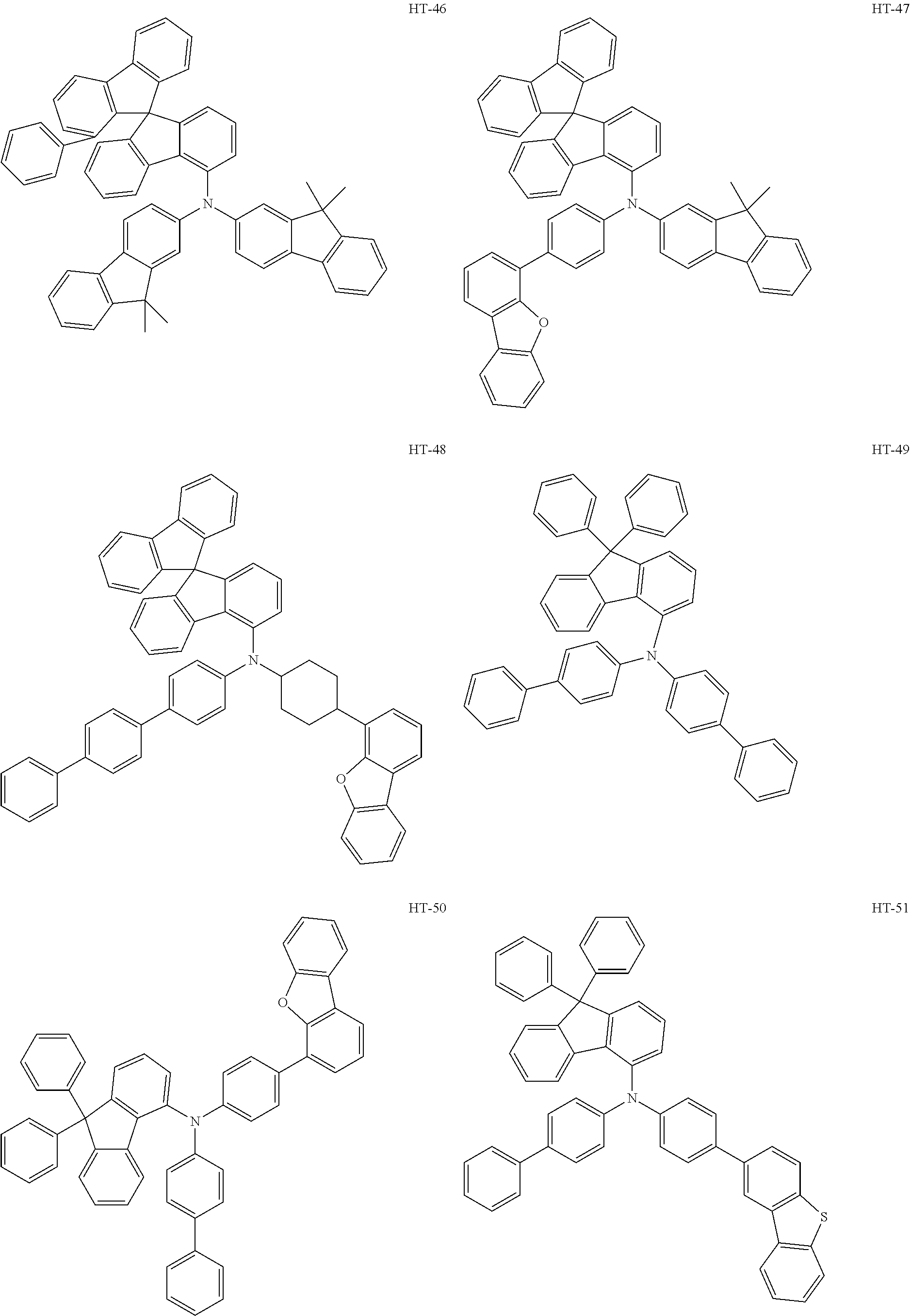

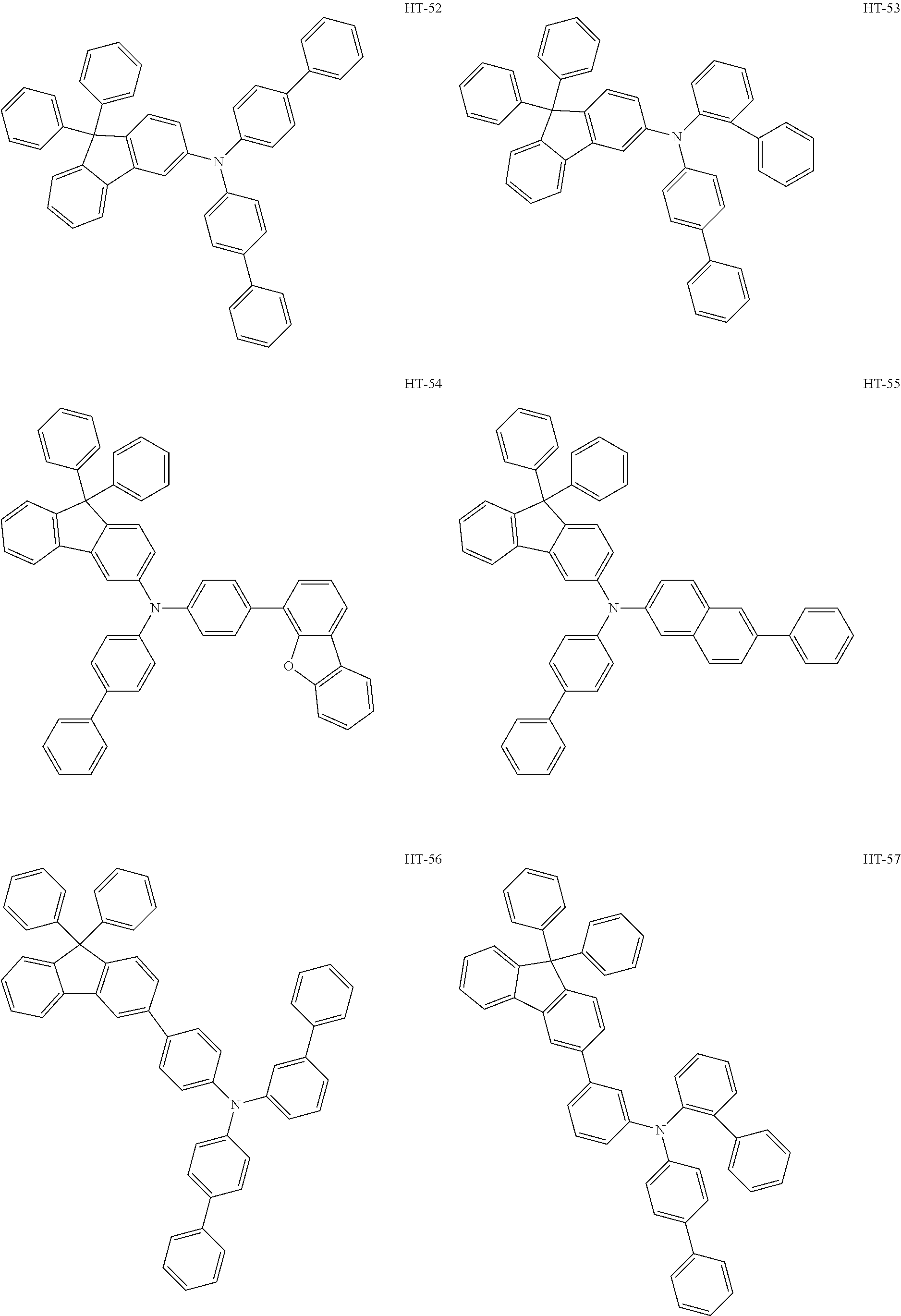

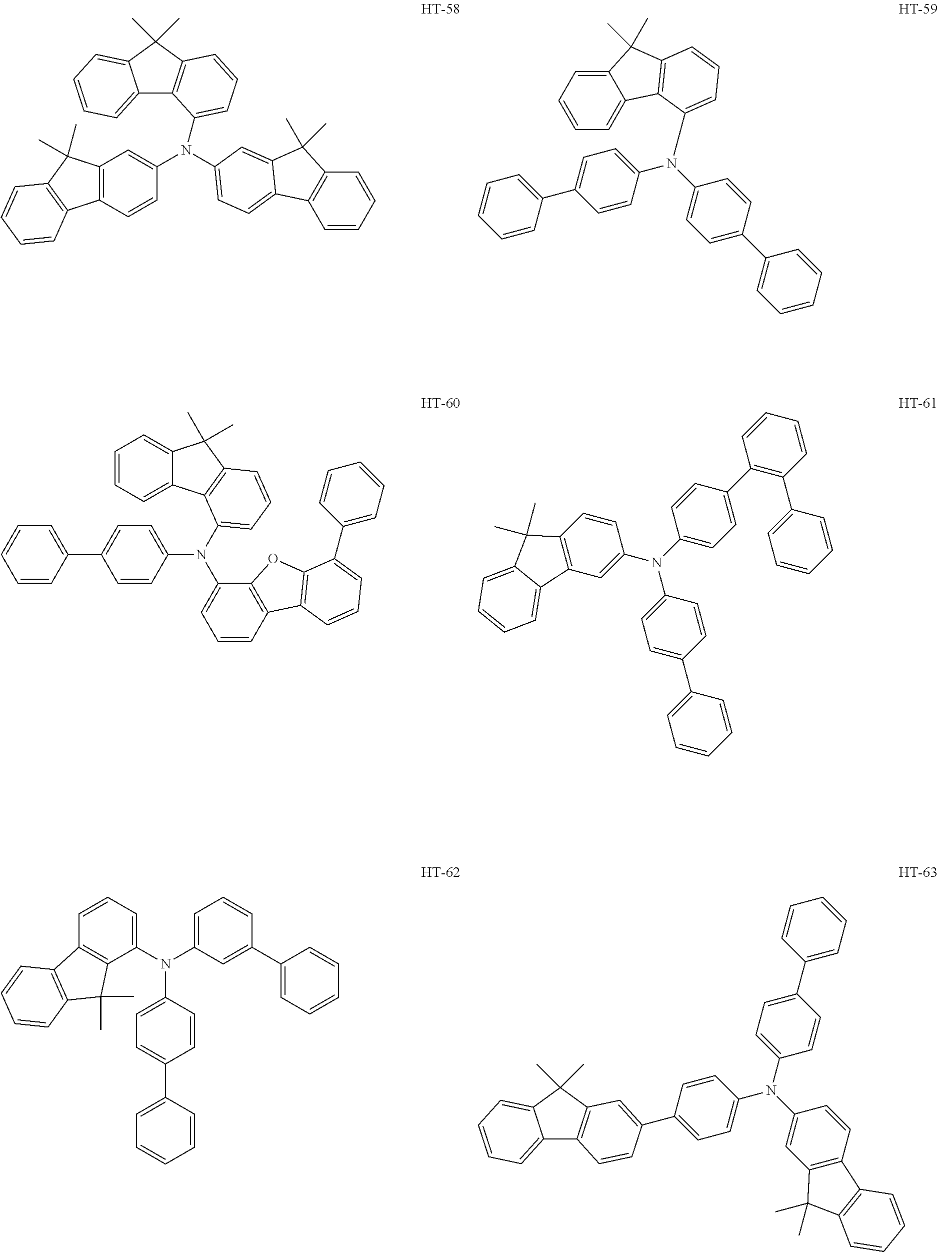 [SMILES: CC1(C)c2ccccc2-c2ccc(N(c3ccc4c(c3)C(C)(C)c3ccccc3-4)c3cccc4c3-c3ccccc3C4(C)C)cc21 ; CC1(C)c2ccccc2-c2c(N(c3ccc(-c4ccccc4)cc3)c3ccc(-c4ccccc4)cc3)cccc21 ; CC1(C)c2ccccc2-c2c(N(c3ccc(-c4ccccc4)cc3)c3cccc4c3oc3c(-c5ccccc5)cccc34)cccc21 ; CC1(C)c2ccccc2-c2cc(N(c3ccc(-c4ccccc4)cc3)c3ccc(-c4ccccc4-c4ccccc4)cc3)ccc21 ; CC1(C)c2ccccc2-c2cccc(N(c3ccc(-c4ccccc4)cc3)c3cccc(-c4ccccc4)c3)c21 ; CC1(C)c2ccccc2-c2ccc(-c3ccc(N(c4ccc(-c5ccccc5)cc4)c4ccc5c(c4)C(C)(C)c4ccccc4-5)cc3)cc21]

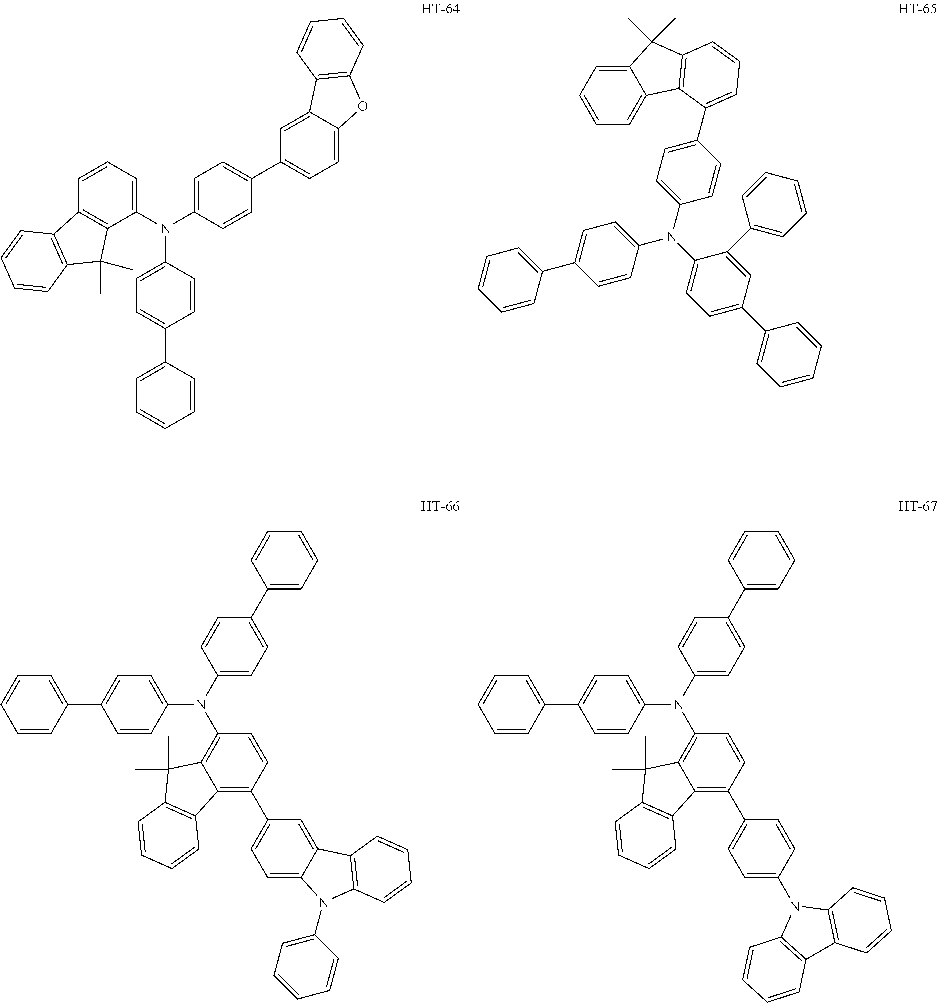 [SMILES: CC1(C)c2ccccc2-c2cccc(N(c3ccc(-c4ccccc4)cc3)c3ccc(-c4ccc5oc6ccccc6c5c4)cc3)c21 ; CC1(C)c2ccccc2-c2c(-c3ccc(N(c4ccc(-c5ccccc5)cc4)c4ccc(-c5ccccc5)cc4-c4ccccc4)cc3)cccc21 ; CC1(C)c2ccccc2-c2c(-c3ccc4c(c3)c3ccccc3n4-c3ccccc3)ccc(N(c3ccc(-c4ccccc4)cc3)c3ccc(-c4ccccc4)cc3)c21 ; CC1(C)c2ccccc2-c2c(-c3ccc(-n4c5ccccc5c5ccccc54)cc3)ccc(N(c3ccc(-c4ccccc4)cc3)c3ccc(-c4ccccc4)cc3)c21]

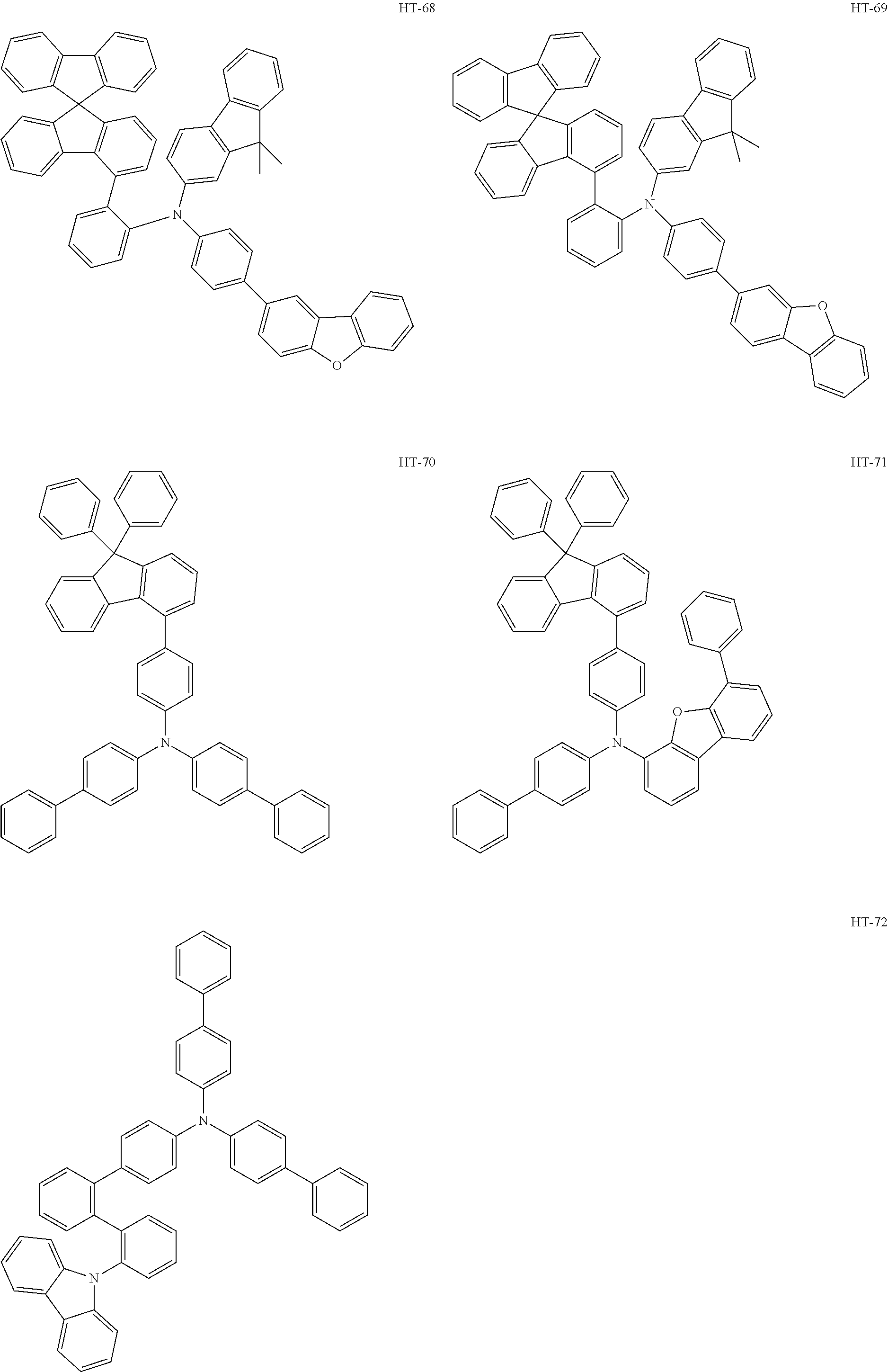

The compounds HT-1 to HT-72 are generally of good suitability for the abovementioned uses in OLEDs of any design and composition, not just in OLEDs according to the present application. The compounds show good performance data in OLEDs, especially good lifetime and good efficiency.

Processes for preparing the compounds HT-1 to HT-72 are known in the prior art. For example, processes for preparing the compounds HT-16, HT-17 and HT-72 are disclosed in WO2014/079,527, on pages 32-33 and in the working examples therein. Processes for preparing the compound HT-18 are disclosed in WO 2013/120,577 and WO2017/144,150, in the description and the working examples. Processes for preparing the compounds HT-20 to HT-32 are disclosed in WO2012/034,627, on pages 39-40 and in the working examples therein.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the emission of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

The device is structured appropriately (according to the application), contact-connected and finally sealed, in order to rule out damaging effects of water and air.

In a preferred embodiment, the electronic device is characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

Preference is likewise given to an electronic device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds are needed. High solubility can be achieved by suitable substitution of the compounds.

It is further preferable that an electronic device of the invention is produced by applying one or more layers from solution and one or more layers by a sublimation method.

Electronic devices comprising one or more compounds as defined above can preferably be used in displays, as light sources in lighting applications and as light sources in medical and/or cosmetic applications (e.g. light therapy).

### EXAMPLES

### A) Synthesis Examples

### Example a: 7-Bromo-6H-indolo[1,2-a]quinazolin-5-one

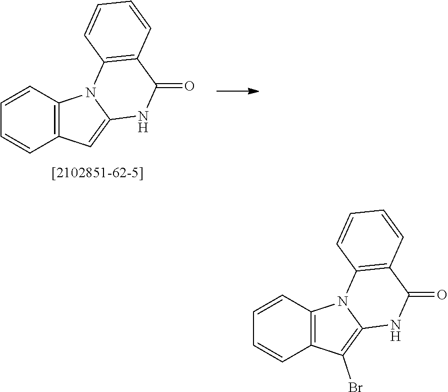 [SMILES: O=c1[nH]c2cc3ccccc3n2c2ccccc12 ; O=c1[nH]c2c(Br)c3ccccc3n2c2ccccc12]

5.5 g (23.5 mmol) of 6H-indolo[1,2-a]quinazolin-5-one are initially charged in 150 ml of CH₂Cl₂. Subsequently, a solution of 4 g (22.5 mmol) of NBS in 100 ml of acetonitrile is added dropwise in the dark at 0° C., the mixture is allowed to come to room temperature and stirring is continued at this temperature for 4 h. Subsequently, 150 ml of water are added to the mixture and extraction is effected with CH₂Cl₂. The organic phase is dried over MgSO₄ and the solvents are removed under reduced pressure. The product is subjected to extractive stirring with hot hexane and filtered off with suction. Yield: 5.5 g (17.6 mmol), 75% of theory, purity by ¹H NMR about 98%.

The following compounds are obtained in an analogous manner:

| Ex. | Reactant | Product | Yield |
|---|---|---|---|
| 1a | 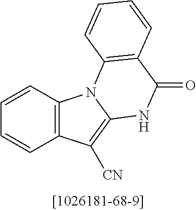 [SMILES: N#Cc1c2ccccc2n2c1[nH]c(=O)c1ccccc12] | 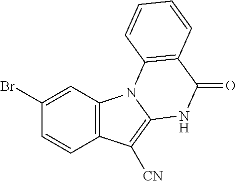 [SMILES: N#Cc1c2ccc(Br)cc2n2c1[nH]c(=O)c1ccccc12] | 69% |
| 2a | 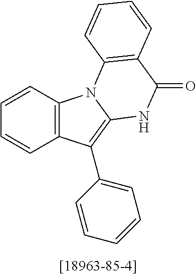 [SMILES: O=c1[nH]c2c(-c3ccccc3)c3ccccc3n2c2ccccc12] | 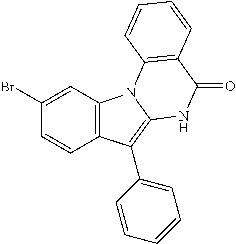 [SMILES: O=c1[nH]c2c(-c3ccccc3)c3ccc(Br)cc3n2c2ccccc12] | 58% |
| 2a | 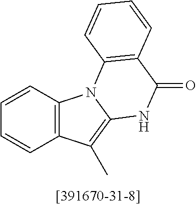 [SMILES: Cc1c2ccccc2n2c1[nH]c(=O)c1ccccc12] | 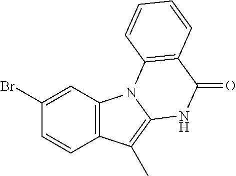 [SMILES: Cc1c2ccc(Br)cc2n2c1[nH]c(=O)c1ccccc12] | 52% |
| 4a | 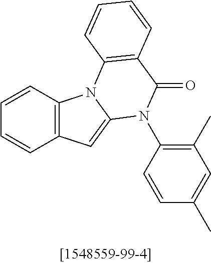 [SMILES: Cc1ccc(-n2c(=O)c3ccccc3n3c4ccccc4cc23)c(C)c1] | 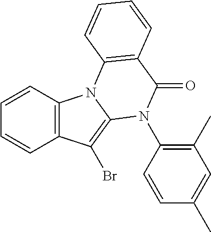 [SMILES: Cc1ccc(-n2c(=O)c3ccccc3n3c4ccccc4c(Br)c23)c(C)c1] | 63% |
| 5a | 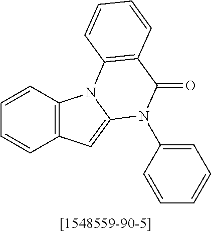 [SMILES: O=c1c2ccccc2n2c3ccccc3cc2n1-c1ccccc1] | 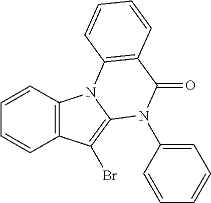 [SMILES: O=c1c2ccccc2n2c3ccccc3c(Br)c2n1-c1ccccc1] | 66% |
| 6a | 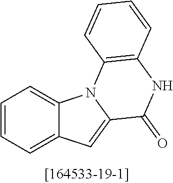 [SMILES: O=c1[nH]c2ccccc2n2c1cc1ccccc12] | 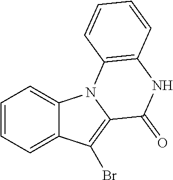 [SMILES: O=c1[nH]c2ccccc2n2c1c(Br)c1ccccc12] | 69% |
| 7a | 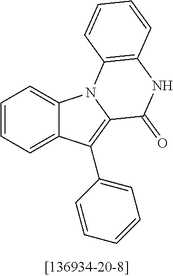 [SMILES: O=c1[nH]c2ccccc2n2c1c(-c1ccccc1)c1ccccc12] | 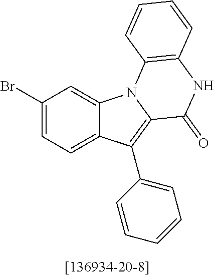 [SMILES: O=c1[nH]c2ccccc2n2c1c(-c1ccccc1)c1ccc(Br)cc12] | 57% |
| 8a | 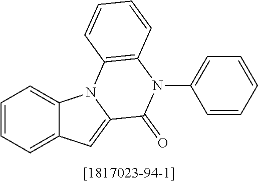 [SMILES: O=c1c2cc3ccccc3n2c2ccccc2n1-c1ccccc1] | 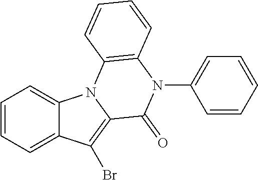 [SMILES: O=c1c2c(Br)c3ccccc3n2c2ccccc2n1-c1ccccc1] | 71% |
| 9a | 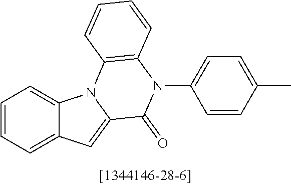 [SMILES: Cc1ccc(-n2c(=O)c3cc4ccccc4n3c3ccccc32)cc1] | 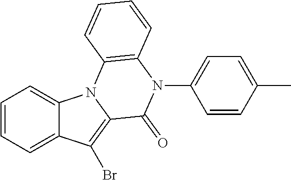 [SMILES: Cc1ccc(-n2c(=O)c3c(Br)c4ccccc4n3c3ccccc32)cc1] | 70% |
| 11a | 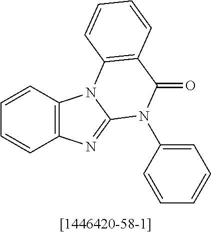 [SMILES: O=c1c2ccccc2n2c3ccccc3nc2n1-c1ccccc1] | 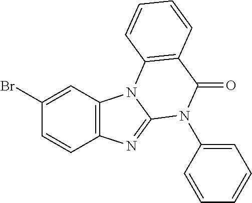 [SMILES: O=c1c2ccccc2n2c3cc(Br)ccc3nc2n1-c1ccccc1] | 88% |
| 12a | 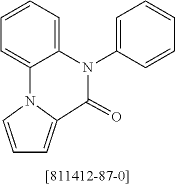 [SMILES: O=c1c2cccn2c2ccccc2n1-c1ccccc1] | 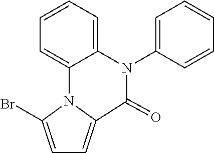 [SMILES: O=c1c2ccc(Br)n2c2ccccc2n1-c1ccccc1] | 89% |
| 13a | 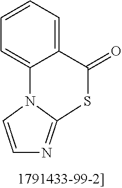 [SMILES: O=c1sc2nccn2c2ccccc12] | 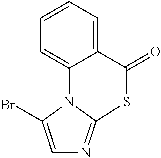 [SMILES: O=c1sc2ncc(Br)n2c2ccccc12] | 78% |
| 14a | 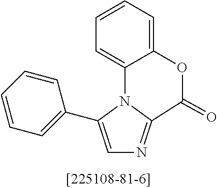 [SMILES: O=c1oc2ccccc2n2c(-c3ccccc3)cnc12] | 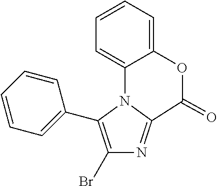 [SMILES: O=c1oc2ccccc2n2c(-c3ccccc3)c(Br)nc12] | 71% |
| 15a | 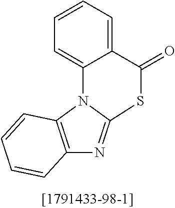 [SMILES: O=c1sc2nc3ccccc3n2c2ccccc12] | 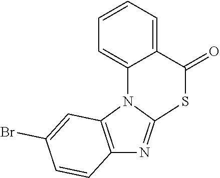 [SMILES: O=c1sc2nc3ccc(Br)cc3n2c2ccccc12] | 57% |
| 16a | 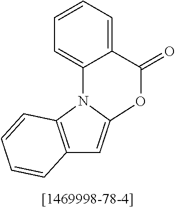 [SMILES: O=c1oc2cc3ccccc3n2c2ccccc12] | 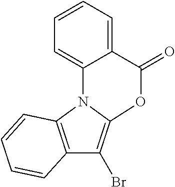 [SMILES: O=c1oc2c(Br)c3ccccc3n2c2ccccc12] | 69% |
| 17a | 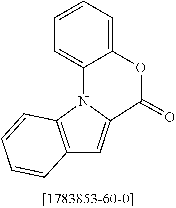 [SMILES: O=c1oc2ccccc2n2c1cc1ccccc12] | 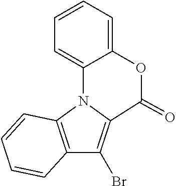 [SMILES: O=c1oc2ccccc2n2c1c(Br)c1ccccc12] | 73% |
| 18a | 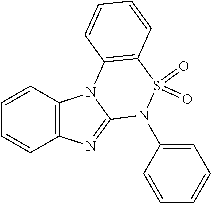 [SMILES: O=S1(=O)c2ccccc2-n2c(nc3ccccc32)N1c1ccccc1] | 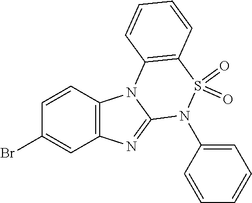 [SMILES: O=S1(=O)c2ccccc2-n2c(nc3cc(Br)ccc32)N1c1ccccc1] | 57% |

### Example b: 7-Bromo-6-(3,5-diphenylphenyl)indolo[1,2-a]quinazolin-5-one

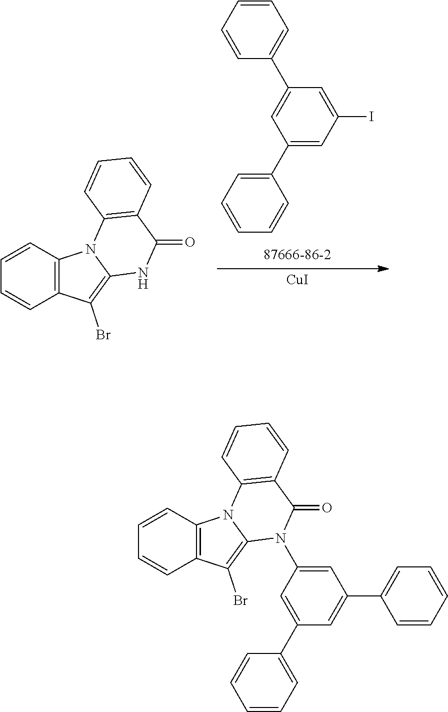 [SMILES: O=c1[nH]c2c(Br)c3ccccc3n2c2ccccc12 ; Ic1cc(-c2ccccc2)cc(-c2ccccc2)c1 ; "CuI" ; O=c1c2ccccc2n2c3ccccc3c(Br)c2n1-c1cc(-c2ccccc2)cc(-c2ccccc2)c1]

31 g (100 mmol) of 7-bromo-6H-indolo[1,2-a]quinazolin-5-one, 106 g (300 mmol) of 5′-iodo-[1,1′;3′,1″]terphenyl, 2.3 g (20 mmol) of L-proline and 5.2 g (27 mmol, 0.11 eq) of copper(I) iodide are stirred in 100 ml at 150° C. for 30 h. The solution is diluted with water and extracted twice with ethyl acetate, and the combined organic phases are dried over Na₂SO₄ and concentrated by rotary evaporation. The residue is purified by chromatography (EtOAc/hexane: 2/3). The yield is 30 g (57 mmol), 57% of theory.

The following compounds are obtained in an analogous manner:

| Ex. | Reactant 1 | Reactant 2 | Product | Yield |
|---|---|---|---|---|
| 1b | 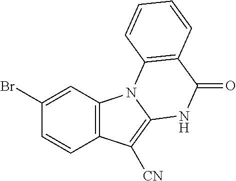 [SMILES: N#Cc1c2ccc(Br)cc2n2c1[nH]c(=O)c1ccccc12] | 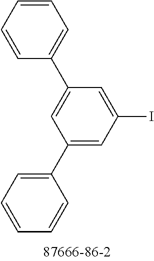 [SMILES: Ic1cc(-c2ccccc2)cc(-c2ccccc2)c1] | 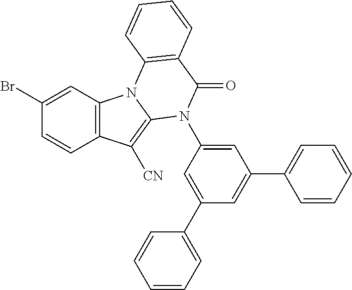 [SMILES: N#Cc1c2ccc(Br)cc2n2c3ccccc3c(=O)n(-c3cc(-c4ccccc4)cc(-c4ccccc4)c3)c12] | 58% |
| 2b | 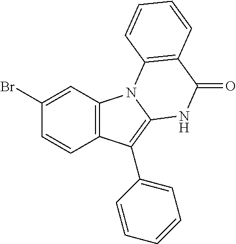 [SMILES: O=c1[nH]c2c(-c3ccccc3)c3ccc(Br)cc3n2c2ccccc12] | 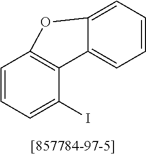 [SMILES: Ic1cccc2oc3ccccc3c12] | 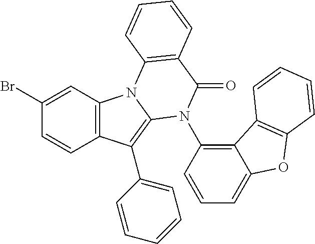 [SMILES: O=c1c2ccccc2n2c3cc(Br)ccc3c(-c3ccccc3)c2n1-c1cccc2oc3ccccc3c12] | 54% |
| 3b | 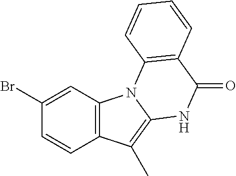 [SMILES: Cc1c2ccc(Br)cc2n2c1[nH]c(=O)c1ccccc12] | 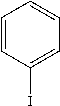 | 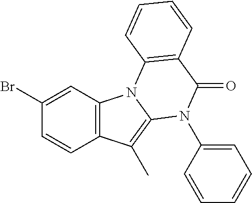 [SMILES: Cc1c2ccc(Br)cc2n2c3ccccc3c(=O)n(-c3ccccc3)c12] | 58% |
| 4b | 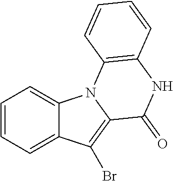 [SMILES: O=c1[nH]c2ccccc2n2c1c(Br)c1ccccc12] | 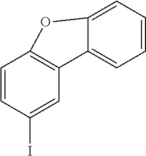 [SMILES: Ic1ccc2oc3ccccc3c2c1] | 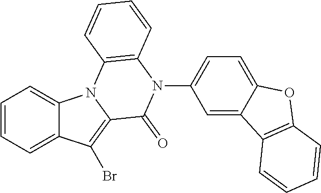 [SMILES: O=c1c2c(Br)c3ccccc3n2c2ccccc2n1-c1ccc2oc3ccccc3c2c1] | 57% |
| 5b | 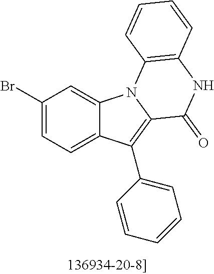 [SMILES: O=c1[nH]c2ccccc2n2c1c(-c1ccccc1)c1ccc(Br)cc12] | 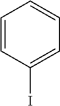 | 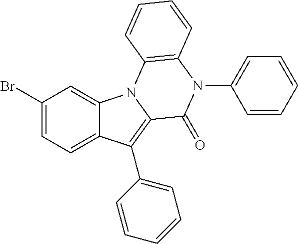 [SMILES: O=c1c2c(-c3ccccc3)c3ccc(Br)cc3n2c2ccccc2n1-c1ccccc1] | 59% |
| 6b | 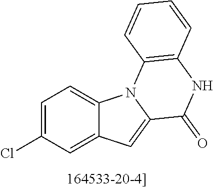 [SMILES: O=c1[nH]c2ccccc2n2c1cc1cc(Cl)ccc12] | 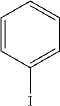 | 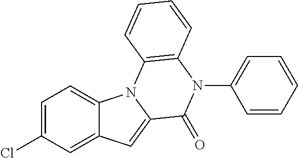 [SMILES: O=c1c2cc3cc(Cl)ccc3n2c2ccccc2n1-c1ccccc1] | 61% |
| 7b | 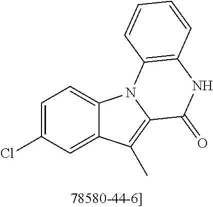 [SMILES: Cc1c2cc(Cl)ccc2n2c1c(=O)[nH]c1ccccc12] | 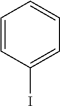 | 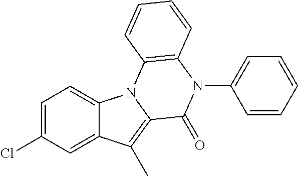 [SMILES: Cc1c2cc(Cl)ccc2n2c1c(=O)n(-c1ccccc1)c1ccccc12] | 64% |
| 8b | 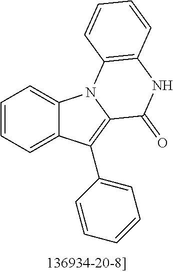 [SMILES: O=c1[nH]c2ccccc2n2c1c(-c1ccccc1)c1ccccc12] | 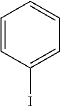 | 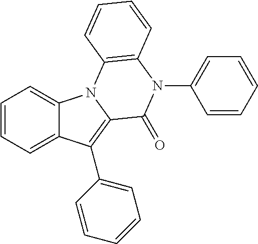 [SMILES: O=c1c2c(-c3ccccc3)c3ccccc3n2c2ccccc2n1-c1ccccc1] | 79% |
| 9b | 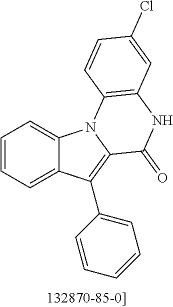 [SMILES: O=c1[nH]c2cc(Cl)ccc2n2c1c(-c1ccccc1)c1ccccc12] | 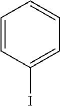 | 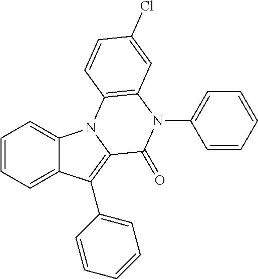 [SMILES: O=c1c2c(-c3ccccc3)c3ccccc3n2c2ccc(Cl)cc2n1-c1ccccc1] | 64% |
| 10b | 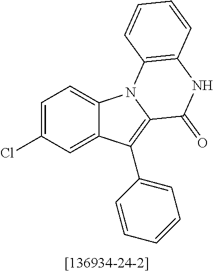 [SMILES: O=c1[nH]c2ccccc2n2c1c(-c1ccccc1)c1cc(Cl)ccc12] | 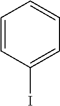 | 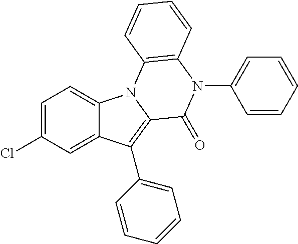 [SMILES: O=c1c2c(-c3ccccc3)c3cc(Cl)ccc3n2c2ccccc2n1-c1ccccc1] | 68% |
| 11b | 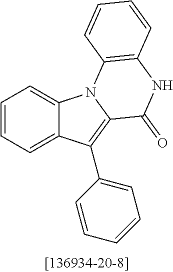 [SMILES: O=c1[nH]c2ccccc2n2c1c(-c1ccccc1)c1ccccc12] | 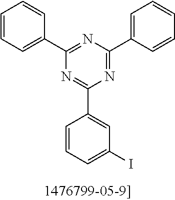 [SMILES: Ic1cccc(-c2nc(-c3ccccc3)nc(-c3ccccc3)n2)c1] | 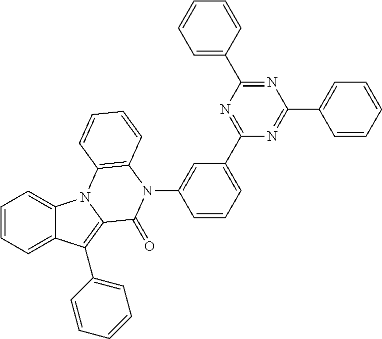 [SMILES: O=c1c2c(-c3ccccc3)c3ccccc3n2c2ccccc2n1-c1cccc(-c2nc(-c3ccccc3)nc(-c3ccccc3)n2)c1] | 64% |
| 12b | 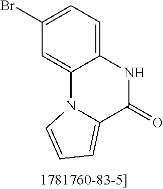 [SMILES: O=c1[nH]c2ccc(Br)cc2n2cccc12] | 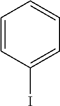 | 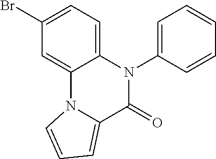 [SMILES: O=c1c2cccn2c2cc(Br)ccc2n1-c1ccccc1] | 72% |
| 13b | 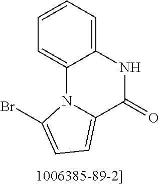 [SMILES: O=c1[nH]c2ccccc2n2c(Br)ccc12] | 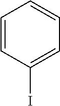 | 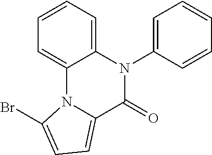 [SMILES: O=c1c2ccc(Br)n2c2ccccc2n1-c1ccccc1] | 76% |
| 14b | 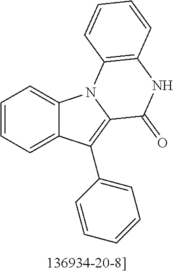 [SMILES: O=c1[nH]c2ccccc2n2c1c(-c1ccccc1)c1ccccc12] | 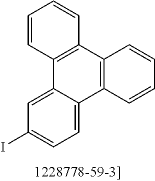 [SMILES: Ic1ccc2c3ccccc3c3ccccc3c2c1] | 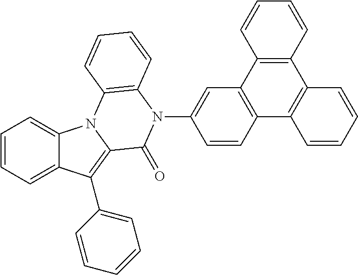 [SMILES: O=c1c2c(-c3ccccc3)c3ccccc3n2c2ccccc2n1-c1ccc2c3ccccc3c3ccccc3c2c1] | 71% |
| 15b | 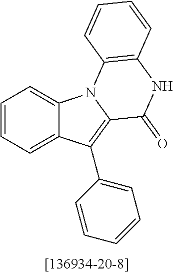 [SMILES: O=c1[nH]c2ccccc2n2c1c(-c1ccccc1)c1ccccc12] | 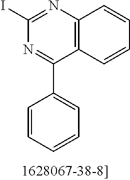 [SMILES: Ic1nc(-c2ccccc2)c2ccccc2n1] | 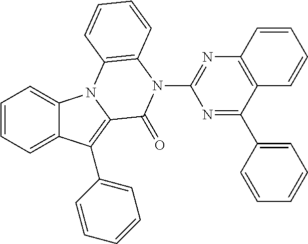 [SMILES: O=c1c2c(-c3ccccc3)c3ccccc3n2c2ccccc2n1-c1nc(-c2ccccc2)c2ccccc2n1] | 61% |

### Example c: 6-(3,5-Diphenylphenyl)-7-phenylindolo[1,2-a]quinazolin-5-one

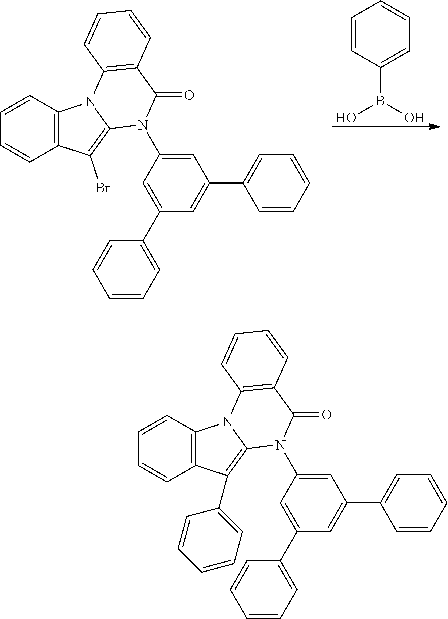 [SMILES: O=c1c2ccccc2n2c3ccccc3c(Br)c2n1-c1cc(-c2ccccc2)cc(-c2ccccc2)c1 ; OB(O)c1ccccc1 ; O=c1c2ccccc2n2c3ccccc3c(-c3ccccc3)c2n1-c1cc(-c2ccccc2)cc(-c2ccccc2)c1]

13.3 g (110.0 mmol) of phenylboronic acid, 59 g (110.0 mmol) of 7-bromo-6-(3,5-diphenylphenyl)indolo[1,2-a]quinazolin-5-one and 44.6 g (210.0 mmol) of tripotassium phosphate are suspended in 500 ml of toluene, 500 ml of dioxane and 500 ml of water. To this suspension are added 913 mg (3.0 mmol) of tri-o-tolylphosphine and then 112 mg (0.5 mmol) of palladium(II) acetate, and the reaction mixture is heated under reflux for 16 h. After cooling, the organic phase is removed, filtered through silica gel, washed three times with 200 ml of water and then concentrated to dryness. The residue is recrystallized from toluene and from dichloromethane/iso-propanol and finally sublimed under high vacuum. The purity is 99.9%. The yield is 77 g (88 mmol), corresponding to 80% of theory.

The following compounds are obtained in an analogous manner:

| Ex. | Reactant 1 | Reactant 2 | Product | Yield |
|---|---|---|---|---|
| 1c | 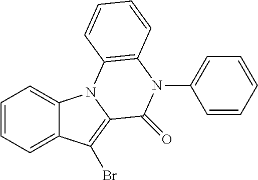 [SMILES: O=c1c2c(Br)c3ccccc3n2c2ccccc2n1-c1ccccc1] | 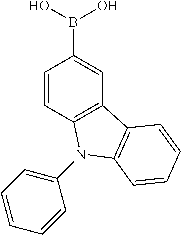 [SMILES: OB(O)c1ccc2c(c1)c1ccccc1n2-c1ccccc1] | 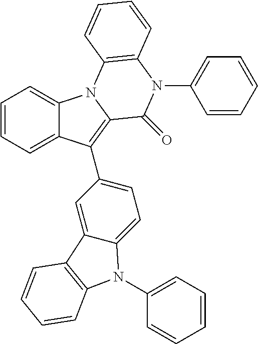 [SMILES: O=c1c2c(-c3ccc4c(c3)c3ccccc3n4-c3ccccc3)c3ccccc3n2c2ccccc2n1-c1ccccc1] | 75% |
| 2c | 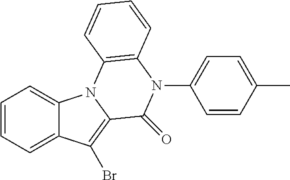 [SMILES: Cc1ccc(-n2c(=O)c3c(Br)c4ccccc4n3c3ccccc32)cc1] | 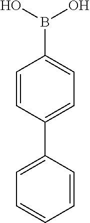 [SMILES: OB(O)c1ccc(-c2ccccc2)cc1] | 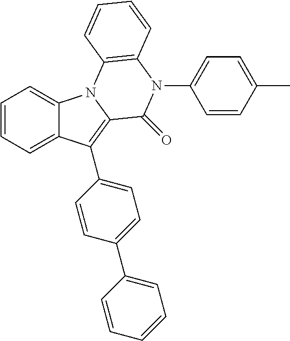 [SMILES: Cc1ccc(-n2c(=O)c3c(-c4ccc(-c5ccccc5)cc4)c4ccccc4n3c3ccccc32)cc1] | 70% |
| 3c | 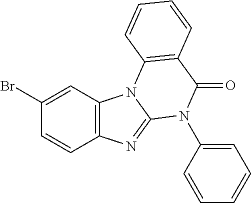 [SMILES: O=c1c2ccccc2n2c3cc(Br)ccc3nc2n1-c1ccccc1] | 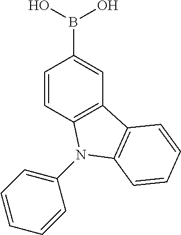 [SMILES: OB(O)c1ccc2c(c1)c1ccccc1n2-c1ccccc1] | 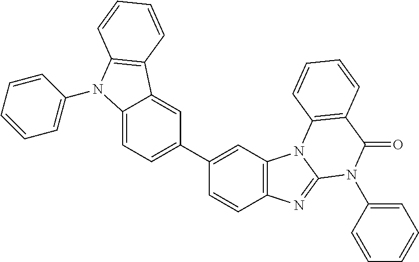 [SMILES: O=c1c2ccccc2n2c3cc(-c4ccc5c(c4)c4ccccc4n5-c4ccccc4)ccc3nc2n1-c1ccccc1] | 79% |
| 4c | 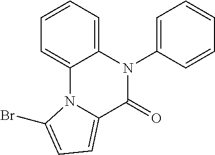 [SMILES: O=c1c2ccc(Br)n2c2ccccc2n1-c1ccccc1] | 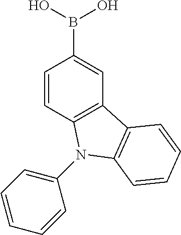 [SMILES: OB(O)c1ccc2c(c1)c1ccccc1n2-c1ccccc1] | 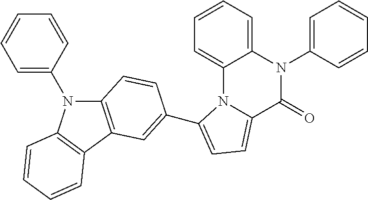 [SMILES: O=c1c2ccc(-c3ccc4c(c3)c3ccccc3n4-c3ccccc3)n2c2ccccc2n1-c1ccccc1] | 69% |
| 5c | 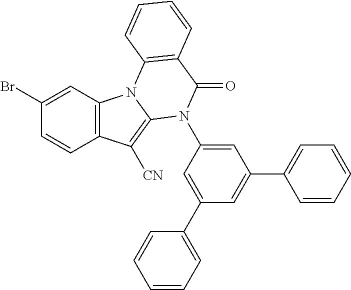 [SMILES: N#Cc1c2ccc(Br)cc2n2c3ccccc3c(=O)n(-c3cc(-c4ccccc4)cc(-c4ccccc4)c3)c12] | 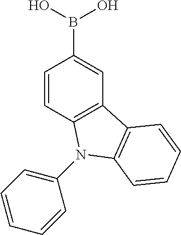 [SMILES: OB(O)c1ccc2c(c1)c1ccccc1n2-c1ccccc1] | 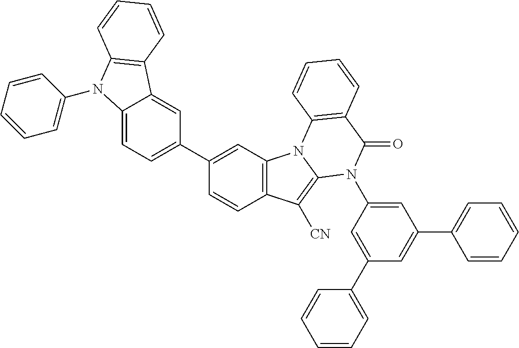 [SMILES: N#Cc1c2ccc(-c3ccc4c(c3)c3ccccc3n4-c3ccccc3)cc2n2c3ccccc3c(=O)n(-c3cc(-c4ccccc4)cc(-c4ccccc4)c3)c12] | 63% |
| 6c | 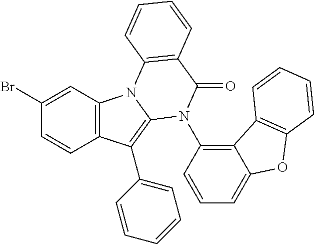 [SMILES: O=c1c2ccccc2n2c3cc(Br)ccc3c(-c3ccccc3)c2n1-c1cccc2oc3ccccc3c12] | 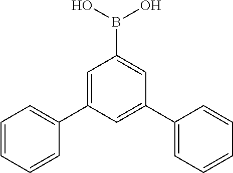 [SMILES: OB(O)c1cc(-c2ccccc2)cc(-c2ccccc2)c1] | 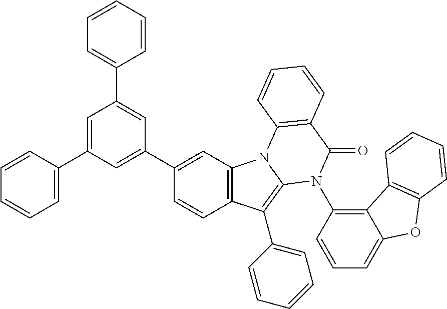 [SMILES: O=c1c2ccccc2n2c3cc(-c4cc(-c5ccccc5)cc(-c5ccccc5)c4)ccc3c(-c3ccccc3)c2n1-c1cccc2oc3ccccc3c12] | 82% |
| 7c | 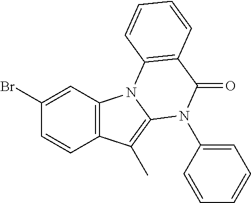 [SMILES: Cc1c2ccc(Br)cc2n2c3ccccc3c(=O)n(-c3ccccc3)c12] | 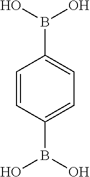 [SMILES: OB(O)c1ccc(B(O)O)cc1] | 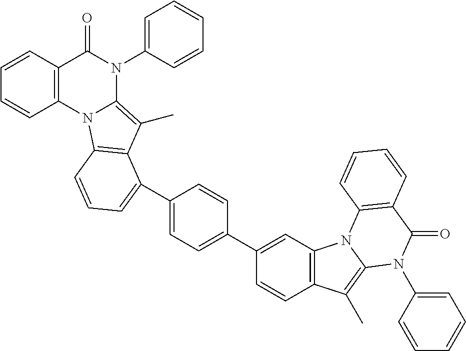 [SMILES: Cc1c2ccc(-c3ccc(-c4cccc5c4c(C)c4n(-c6ccccc6)c(=O)c6ccccc6n54)cc3)cc2n2c3ccccc3c(=O)n(-c3ccccc3)c12] | 75% |
| 8c | 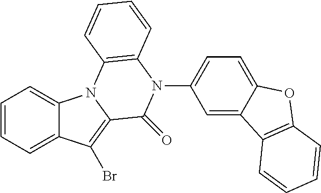 [SMILES: O=c1c2c(Br)c3ccccc3n2c2ccccc2n1-c1ccc2oc3ccccc3c2c1] | 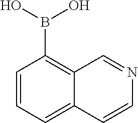 [SMILES: OB(O)c1cccc2ccncc12] | 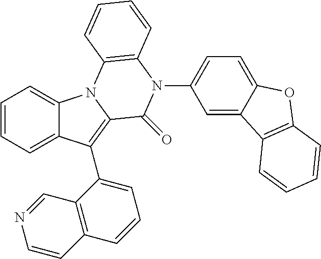 [SMILES: O=c1c2c(-c3cccc4ccncc34)c3ccccc3n2c2ccccc2n1-c1ccc2oc3ccccc3c2c1] | 88% |
| 9c | 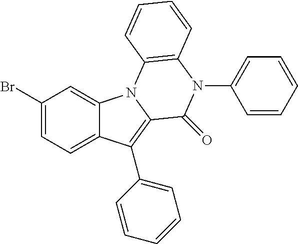 [SMILES: O=c1c2c(-c3ccccc3)c3ccc(Br)cc3n2c2ccccc2n1-c1ccccc1] | 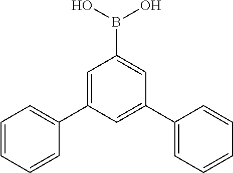 [SMILES: OB(O)c1cc(-c2ccccc2)cc(-c2ccccc2)c1] | 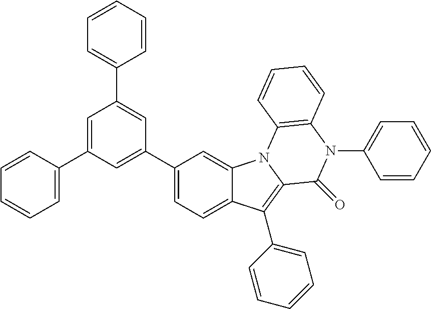 [SMILES: O=c1c2c(-c3ccccc3)c3ccc(-c4cc(-c5ccccc5)cc(-c5ccccc5)c4)cc3n2c2ccccc2n1-c1ccccc1] | 84% |
| 10c | 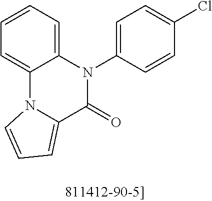 [SMILES: O=c1c2cccn2c2ccccc2n1-c1ccc(Cl)cc1] | 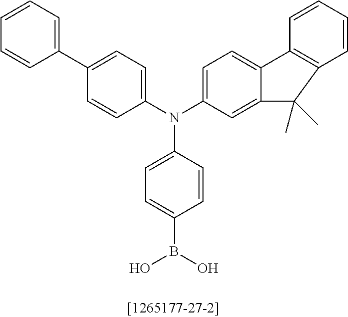 [SMILES: CC1(C)c2ccccc2-c2ccc(N(c3ccc(B(O)O)cc3)c3ccc(-c4ccccc4)cc3)cc21] | 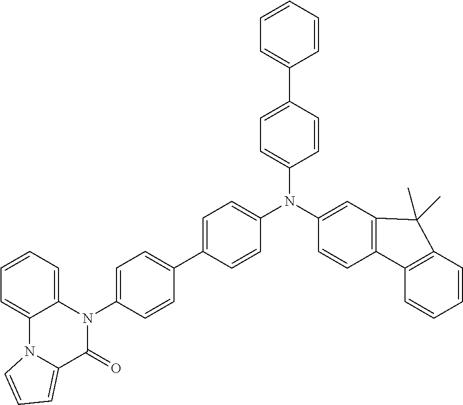 [SMILES: CC1(C)c2ccccc2-c2ccc(N(c3ccc(-c4ccccc4)cc3)c3ccc(-c4ccc(-n5c(=O)c6cccn6c6ccccc65)cc4)cc3)cc21] | 63% |
| 11c | 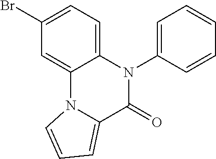 [SMILES: O=c1c2cccn2c2cc(Br)ccc2n1-c1ccccc1] | 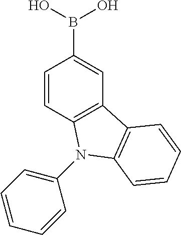 [SMILES: OB(O)c1ccc2c(c1)c1ccccc1n2-c1ccccc1] | 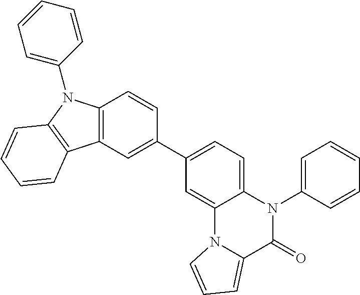 [SMILES: O=c1c2cccn2c2cc(-c3ccc4c(c3)c3ccccc3n4-c3ccccc3)ccc2n1-c1ccccc1] | 77% |
| 12c | 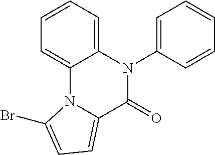 [SMILES: O=c1c2ccc(Br)n2c2ccccc2n1-c1ccccc1] | 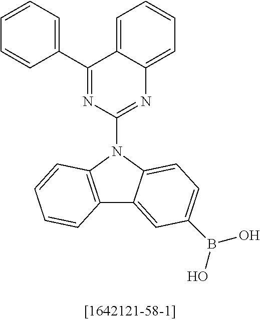 [SMILES: OB(O)c1ccc2c(c1)c1ccccc1n2-c1nc(-c2ccccc2)c2ccccc2n1] | 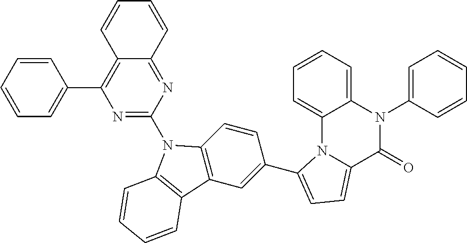 [SMILES: O=c1c2ccc(-c3ccc4c(c3)c3ccccc3n4-c3nc(-c4ccccc4)c4ccccc4n3)n2c2ccccc2n1-c1ccccc1] | 74% |
| 13c | 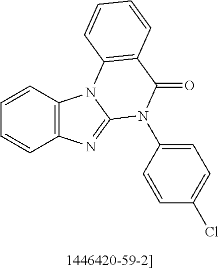 [SMILES: O=c1c2ccccc2n2c3ccccc3nc2n1-c1ccc(Cl)cc1] | 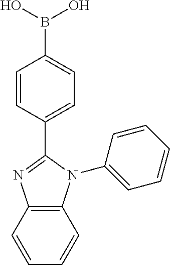 [SMILES: OB(O)c1ccc(-c2nc3ccccc3n2-c2ccccc2)cc1] | 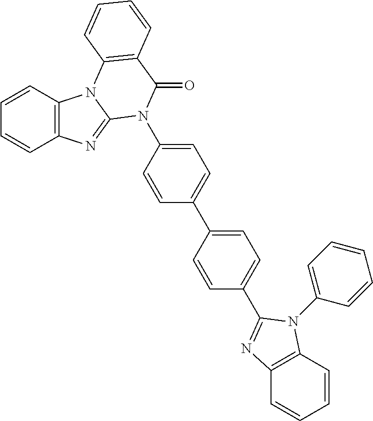 [SMILES: O=c1c2ccccc2n2c3ccccc3nc2n1-c1ccc(-c2ccc(-c3nc4ccccc4n3-c3ccccc3)cc2)cc1] | 89% |
| 14c | 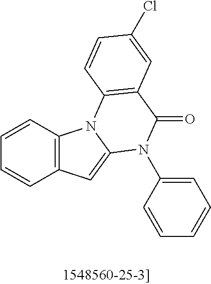 [SMILES: O=c1c2cc(Cl)ccc2n2c3ccccc3cc2n1-c1ccccc1] | 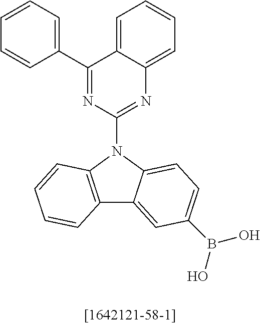 [SMILES: OB(O)c1ccc2c(c1)c1ccccc1n2-c1nc(-c2ccccc2)c2ccccc2n1] | 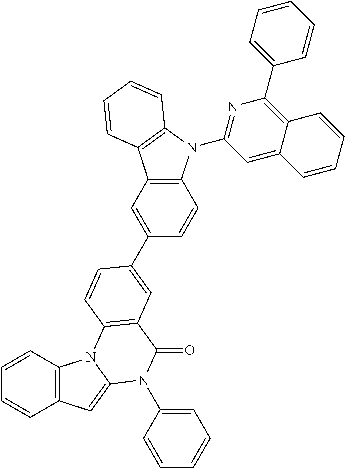 [SMILES: O=c1c2cc(-c3ccc4c(c3)c3ccccc3n4-c3cc4ccccc4c(-c4ccccc4)n3)ccc2n2c3ccccc3cc2n1-c1ccccc1] | 75% |
| 15c | 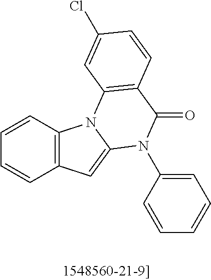 [SMILES: O=c1c2ccc(Cl)cc2n2c3ccccc3cc2n1-c1ccccc1] | 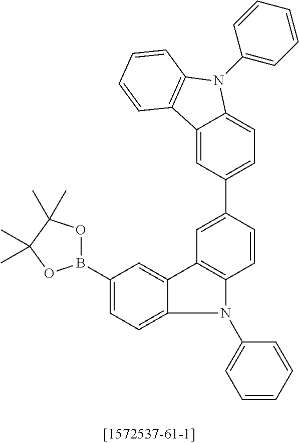 [SMILES: CC1(C)OB(c2ccc3c(c2)c2cc(-c4ccc5c(c4)c4ccccc4n5-c4ccccc4)ccc2n3-c2ccccc2)OC1(C)C] | 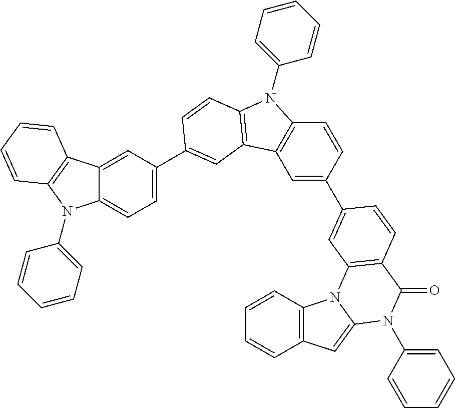 [SMILES: O=c1c2ccc(-c3ccc4c(c3)c3cc(-c5ccc6c(c5)c5ccccc5n6-c5ccccc5)ccc3n4-c3ccccc3)cc2n2c3ccccc3cc2n1-c1ccccc1] | 59% |
| 16c | 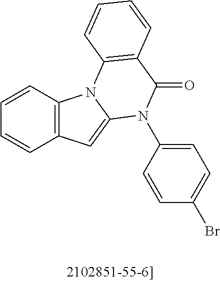 [SMILES: O=c1c2ccccc2n2c3ccccc3cc2n1-c1ccc(Br)cc1] | 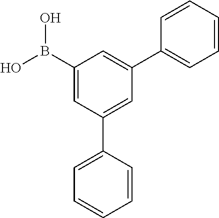 [SMILES: OB(O)c1cc(-c2ccccc2)cc(-c2ccccc2)c1] | 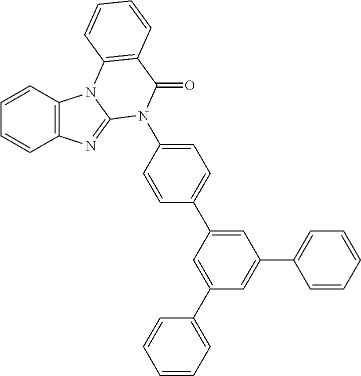 [SMILES: O=c1c2ccccc2n2c3ccccc3nc2n1-c1ccc(-c2cc(-c3ccccc3)cc(-c3ccccc3)c2)cc1] | 67% |
| 17c | 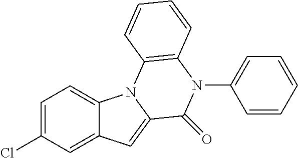 [SMILES: O=c1c2cc3cc(Cl)ccc3n2c2ccccc2n1-c1ccccc1] | 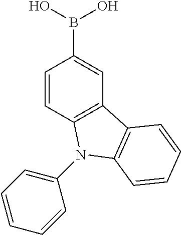 [SMILES: OB(O)c1ccc2c(c1)c1ccccc1n2-c1ccccc1] | 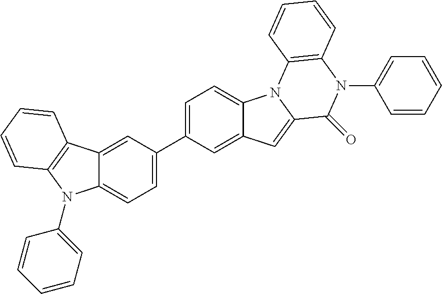 [SMILES: O=c1c2cc3cc(-c4ccc5c(c4)c4ccccc4n5-c4ccccc4)ccc3n2c2ccccc2n1-c1ccccc1] | 66% |
| 18c | 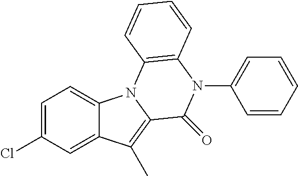 [SMILES: Cc1c2cc(Cl)ccc2n2c1c(=O)n(-c1ccccc1)c1ccccc12] | 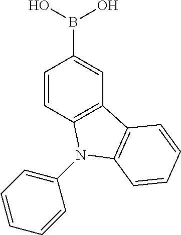 [SMILES: OB(O)c1ccc2c(c1)c1ccccc1n2-c1ccccc1] | 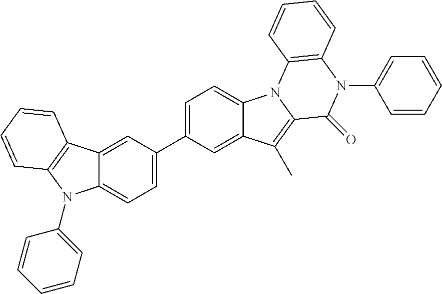 [SMILES: Cc1c2cc(-c3ccc4c(c3)c3ccccc3n4-c3ccccc3)ccc2n2c1c(=O)n(-c1ccccc1)c1ccccc12] | 62% |
| 19c | 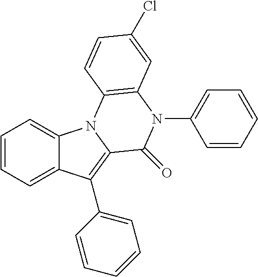 [SMILES: O=c1c2c(-c3ccccc3)c3ccccc3n2c2ccc(Cl)cc2n1-c1ccccc1] | 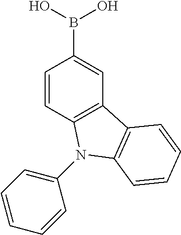 [SMILES: OB(O)c1ccc2c(c1)c1ccccc1n2-c1ccccc1] | 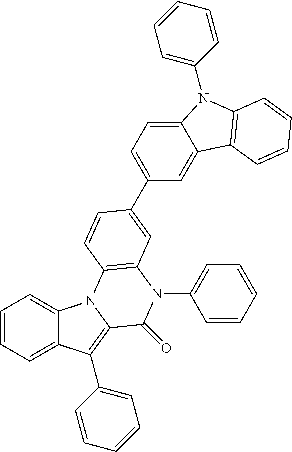 [SMILES: O=c1c2c(-c3ccccc3)c3ccccc3n2c2ccc(-c3ccc4c(c3)c3ccccc3n4-c3ccccc3)cc2n1-c1ccccc1] | 71% |
| 20c | 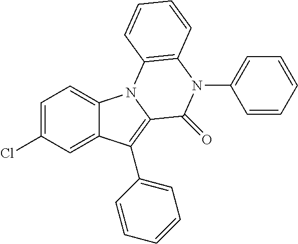 [SMILES: O=c1c2c(-c3ccccc3)c3cc(Cl)ccc3n2c2ccccc2n1-c1ccccc1] | 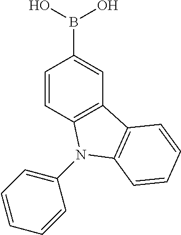 [SMILES: OB(O)c1ccc2c(c1)c1ccccc1n2-c1ccccc1] | 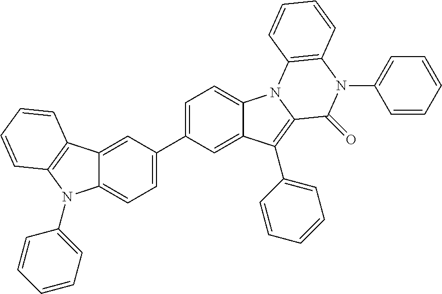 [SMILES: O=c1c2c(-c3ccccc3)c3cc(-c4ccc5c(c4)c4ccccc4n5-c4ccccc4)ccc3n2c2ccccc2n1-c1ccccc1] | 70% |
| 21c | 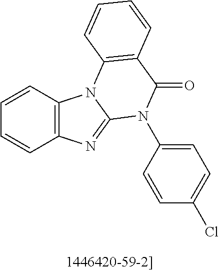 [SMILES: O=c1c2ccccc2n2c3ccccc3nc2n1-c1ccc(Cl)cc1] | 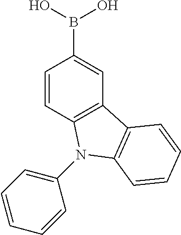 [SMILES: OB(O)c1ccc2c(c1)c1ccccc1n2-c1ccccc1] | 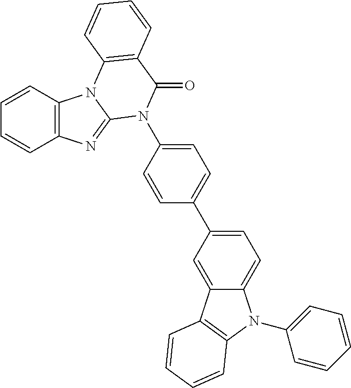 [SMILES: O=c1c2ccccc2n2c3ccccc3nc2n1-c1ccc(-c2ccc3c(c2)c2ccccc2n3-c2ccccc2)cc1] | 73% |
| 22c | 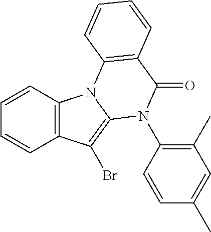 [SMILES: Cc1ccc(-n2c(=O)c3ccccc3n3c4ccccc4c(Br)c23)c(C)c1] | 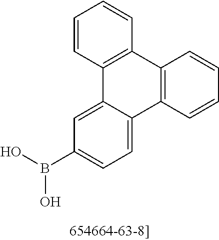 [SMILES: OB(O)c1ccc2c3ccccc3c3ccccc3c2c1] | 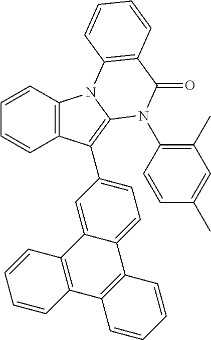 [SMILES: Cc1ccc(-n2c(=O)c3ccccc3n3c4ccccc4c(-c4ccc5c6ccccc6c6ccccc6c5c4)c23)c(C)c1] | 84% |
| 23c | 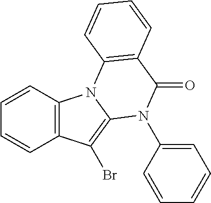 [SMILES: O=c1c2ccccc2n2c3ccccc3c(Br)c2n1-c1ccccc1] | 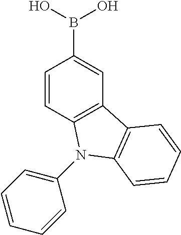 [SMILES: OB(O)c1ccc2c(c1)c1ccccc1n2-c1ccccc1] | 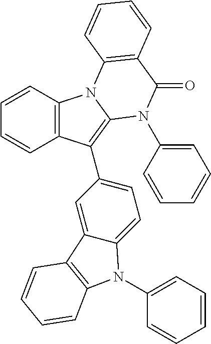 [SMILES: O=c1c2ccccc2n2c3ccccc3c(-c3ccc4c(c3)c3ccccc3n4-c3ccccc3)c2n1-c1ccccc1] | 69% |
| 24c | 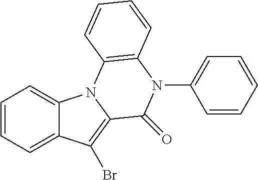 [SMILES: O=c1c2c(Br)c3ccccc3n2c2ccccc2n1-c1ccccc1] | 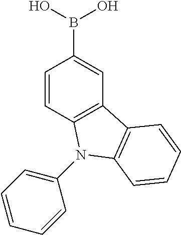 [SMILES: OB(O)c1ccc2c(c1)c1ccccc1n2-c1ccccc1] | 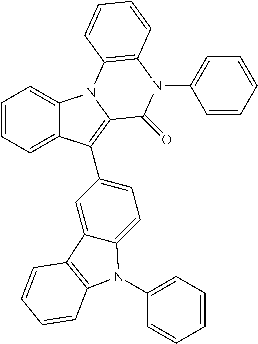 [SMILES: O=c1c2c(-c3ccc4c(c3)c3ccccc3n4-c3ccccc3)c3ccccc3n2c2ccccc2n1-c1ccccc1] | 77% |
| 25c | 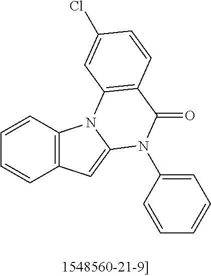 [SMILES: O=c1c2ccc(Cl)cc2n2c3ccccc3cc2n1-c1ccccc1] | 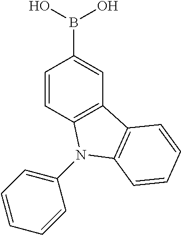 [SMILES: OB(O)c1ccc2c(c1)c1ccccc1n2-c1ccccc1] | 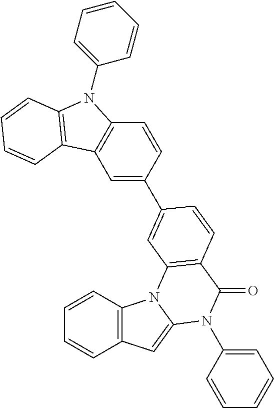 [SMILES: O=c1c2ccc(-c3ccc4c(c3)c3ccccc3n4-c3ccccc3)cc2n2c3ccccc3cc2n1-c1ccccc1] | 81% |
| 26c | 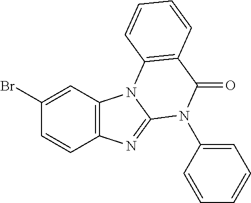 [SMILES: O=c1c2ccccc2n2c3cc(Br)ccc3nc2n1-c1ccccc1] | 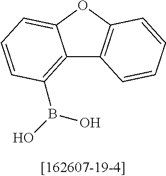 [SMILES: OB(O)c1cccc2oc3ccccc3c12] | 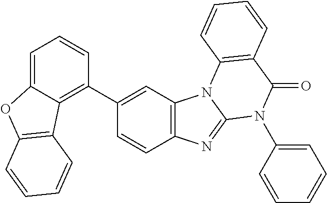 [SMILES: O=c1c2ccccc2n2c3cc(-c4cccc5oc6ccccc6c45)ccc3nc2n1-c1ccccc1] | 86% |
| 27c | 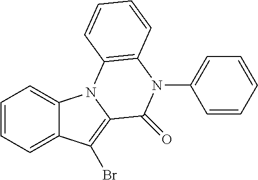 [SMILES: O=c1c2c(Br)c3ccccc3n2c2ccccc2n1-c1ccccc1] | 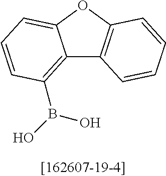 [SMILES: OB(O)c1cccc2oc3ccccc3c12] | 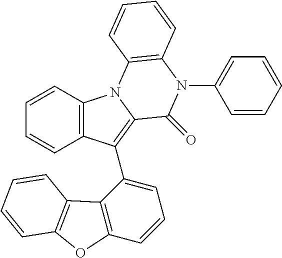 [SMILES: O=c1c2c(-c3cccc4oc5ccccc5c34)c3ccccc3n2c2ccccc2n1-c1ccccc1] | 80% |
| 28c | 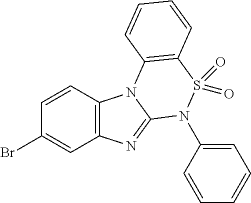 [SMILES: O=S1(=O)c2ccccc2-n2c(nc3cc(Br)ccc32)N1c1ccccc1] | 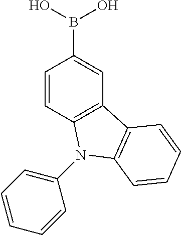 [SMILES: OB(O)c1ccc2c(c1)c1ccccc1n2-c1ccccc1] | 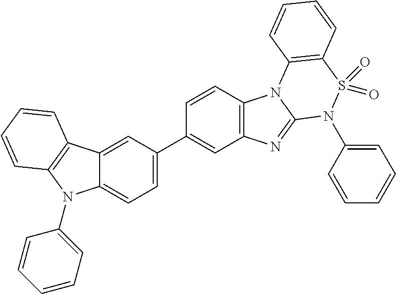 [SMILES: O=S1(=O)c2ccccc2-n2c(nc3cc(-c4ccc5c(c4)c4ccccc4n5-c4ccccc4)ccc32)N1c1ccccc1] | 76% |
| 29c | 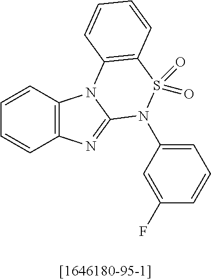 [SMILES: O=S1(=O)c2ccccc2-n2c(nc3ccccc32)N1c1cccc(F)c1] | 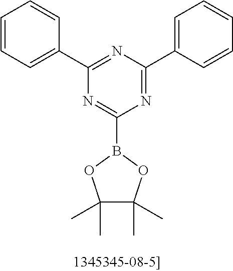 [SMILES: CC1(C)OB(c2nc(-c3ccccc3)nc(-c3ccccc3)n2)OC1(C)C] | 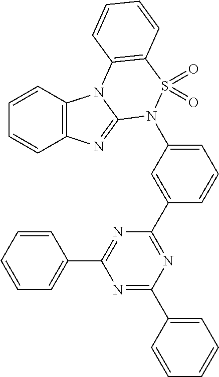 [SMILES: O=S1(=O)c2ccccc2-n2c(nc3ccccc32)N1c1cccc(-c2nc(-c3ccccc3)nc(-c3ccccc3)n2)c1] | 71% |
| 30c | 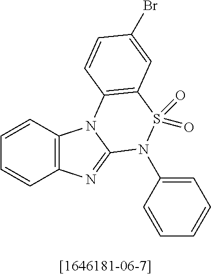 [SMILES: O=S1(=O)c2cc(Br)ccc2-n2c(nc3ccccc32)N1c1ccccc1] | 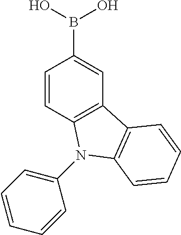 [SMILES: OB(O)c1ccc2c(c1)c1ccccc1n2-c1ccccc1] | 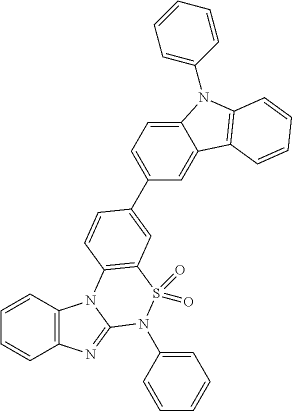 [SMILES: O=S1(=O)c2cc(-c3ccc4c(c3)c3ccccc3n4-c3ccccc3)ccc2-n2c(nc3ccccc32)N1c1ccccc1] | 65% |

### Example d: Indolo[1,2-a]benzimidazol-11-one Oxime

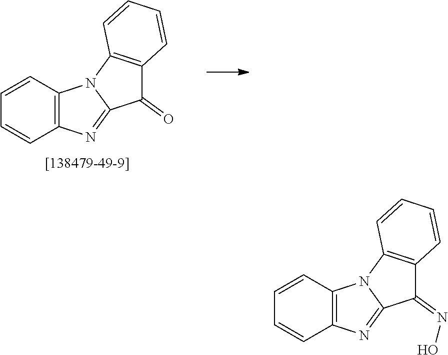 [SMILES: O=C1c2ccccc2-n2c1nc1ccccc12 ; O/N=C1/c2ccccc2-n2c1nc1ccccc12]

To an initial charge of 33 g (147 mmol) of indolo[1,2-a]benzimidazol-11-one in 300 ml of pyridine/200 of methanol is then added 20.5 g of hydroxylammonium chloride in portions. This is followed by heating at 60° C. for 3.5 hours. After the reaction has ended, the precipitated solids are filtered off with suction and washed with water and 1M HCl, and then with methanol. The yield is 32.4 g (138 mmol), corresponding to 92% of theory. The following compounds can be prepared in an analogous manner:

| Ex. | Reactant 1 | Product | Yield |
|---|---|---|---|
| 1d | 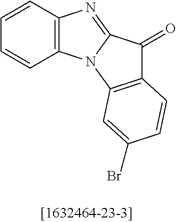 [SMILES: O=C1c2ccc(Br)cc2-n2c1nc1ccccc12] | 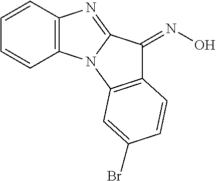 [SMILES: O/N=C1\c2ccc(Br)cc2-n2c1nc1ccccc12] | 89% |
| 2d | 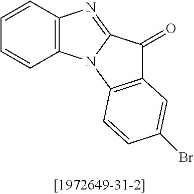 [SMILES: O=C1c2cc(Br)ccc2-n2c1nc1ccccc12] | 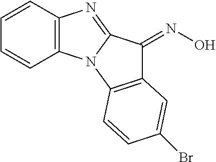 [SMILES: O/N=C1\c2cc(Br)ccc2-n2c1nc1ccccc12] | 91% |
| 3d | 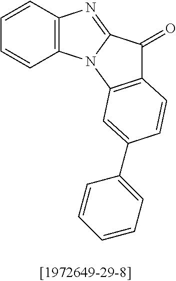 [SMILES: O=C1c2ccc(-c3ccccc3)cc2-n2c1nc1ccccc12] | 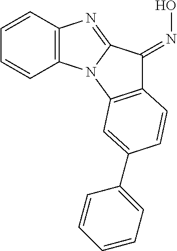 [SMILES: O/N=C1/c2ccc(-c3ccccc3)cc2-n2c1nc1ccccc12] | 90% |
| 4d | 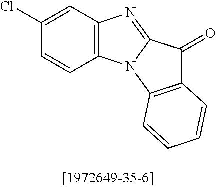 [SMILES: O=C1c2ccccc2-n2c1nc1cc(Cl)ccc12] | 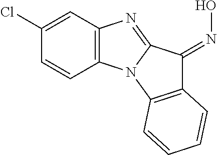 [SMILES: O/N=C1/c2ccccc2-n2c1nc1cc(Cl)ccc12] | 93% |

### Example e: Lactam Synthesis

### A) 5H-Benzimidazolo[1,2-a]quinoxalin-6-one

### B) 6H-Benzimidazolo[1,2-a]quinazolin-5-one

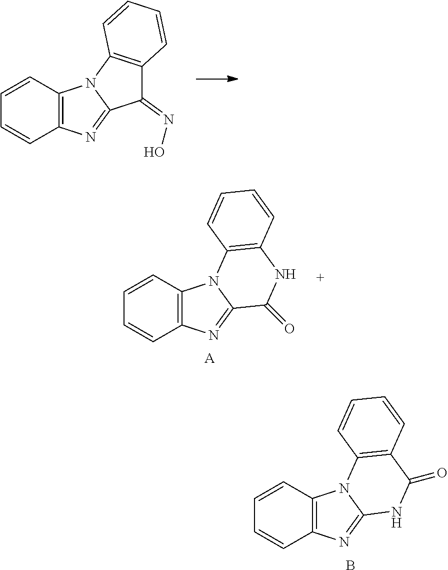

An initial charge of 33 g (140 mmol) of indolo[1,2-a]benzimidazol-11-one oxime in 300 ml of polyphosphoric acid is finally heated to 170° C. for 12 hours. After the reaction has ended, the mixture is added to ice, extracted with ethyl acetate, separated and concentrated. The precipitated solids are filtered off with suction and washed with ethanol. The isomers are separated by chromatography.

Yield: 30 g (127 mmol) of the A+B mixture, 94% of theory, purity: 98.0% by HPLC. Recrystallization from ethyl acetate/toluene (1:3) affords 14 g (42%) of (A) and 16 g (48%) of (B).

The following compounds are prepared in an analogous manner:

| | | | | Yield |
|---|---|---|---|---|
| Ex. | Reactant 1 | Product (A) | Product (B) | (A)/(B) |
| 1e | 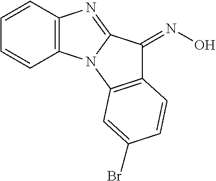 [SMILES: O/N=C1\c2ccc(Br)cc2-n2c1nc1ccccc12] | 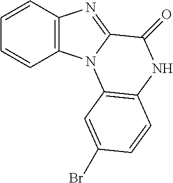 [SMILES: O=c1[nH]c2ccc(Br)cc2n2c1nc1ccccc12] | 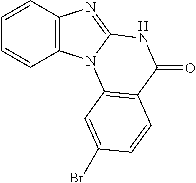 [SMILES: O=c1[nH]c2nc3ccccc3n2c2cc(Br)ccc12] | 44%/45% |
| 2e | 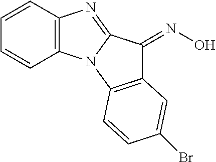 [SMILES: O/N=C1\c2cc(Br)ccc2-n2c1nc1ccccc12] | 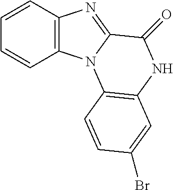 [SMILES: O=c1[nH]c2cc(Br)ccc2n2c1nc1ccccc12] | 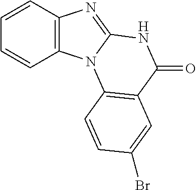 [SMILES: O=c1[nH]c2nc3ccccc3n2c2ccc(Br)cc12] | 40%/43% |
| 3e | 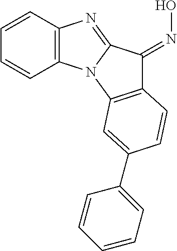 [SMILES: O/N=C1/c2ccc(-c3ccccc3)cc2-n2c1nc1ccccc12] | 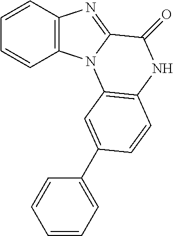 [SMILES: O=c1[nH]c2ccc(-c3ccccc3)cc2n2c1nc1ccccc12] | 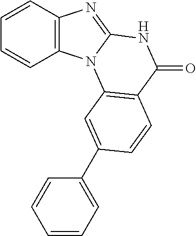 [SMILES: O=c1[nH]c2nc3ccccc3n2c2cc(-c3ccccc3)ccc12] | 42%/40% |
| 4e | 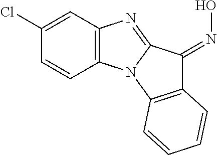 [SMILES: O/N=C1/c2ccccc2-n2c1nc1cc(Cl)ccc12] | 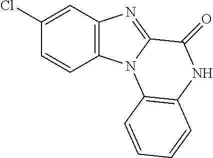 [SMILES: O=c1[nH]c2ccccc2n2c1nc1cc(Cl)ccc12] | 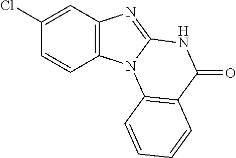 [SMILES: O=c1[nH]c2nc3cc(Cl)ccc3n2c2ccccc12] | 38%/41% |

### Example 5-(3-Phenylphenyl)benzimidazolo[1,2-a]quinoxalin-6-one

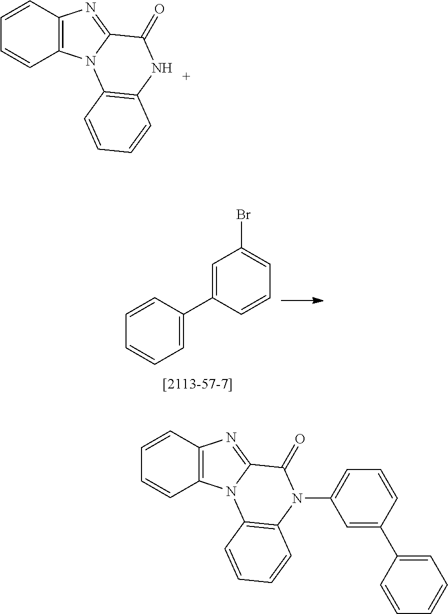 [SMILES: O=c1[nH]c2ccccc2n2c1nc1ccccc12 ; Brc1cccc(-c2ccccc2)c1 ; O=c1c2nc3ccccc3n2c2ccccc2n1-c1cccc(-c2ccccc2)c1]

An initial charge of 13.5 g (25 mmol, 1.00 eq.) of 5H-benzimidazolo[1,2-a]quinoxalin-6-one, 21.3 ml (128 mmol, 5.2 eq.) of 3-bromobiphenyl and 7.20 g of potassium carbonate (52.1 mmol, 2.10 eq.) in 220 ml of dry DMF is inertized under argon. Subsequently, 0.62 g (2.7 mmol, 0.11 eq) of 1,3-di(2-pyridyl)propane-1,3-dione and 0.52 g (2.7 mmol, 0.11 eq) of copper(I) iodide are added and the mixture is heated at 140° C. for three days. After the reaction has ended, the mixture is concentrated cautiously on a rotary evaporator, and the precipitated solids are filtered off with suction and washed with water and ethanol. The crude product is purified twice by means of a hot extractor (toluene/heptane 1:1), and the solids obtained are recrystallized from toluene. After sublimation, 8.2 g (12 mmol, 48%) of the desired target compound is obtained.

The following compounds can be prepared in an analogous manner:

| Ex. | Reactant 1 | Reactant 1 | Product | Yield |
|---|---|---|---|---|
| 2f | 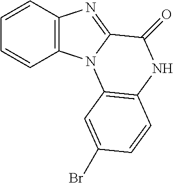 [SMILES: O=c1[nH]c2ccc(Br)cc2n2c1nc1ccccc12] | 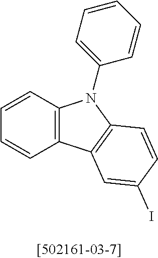 [SMILES: Ic1ccc2c(c1)c1ccccc1n2-c1ccccc1] | 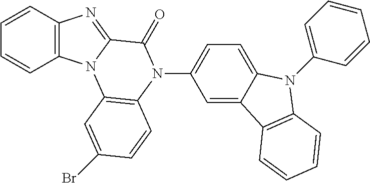 [SMILES: O=c1c2nc3ccccc3n2c2cc(Br)ccc2n1-c1ccc2c(c1)c1ccccc1n2-c1ccccc1] | 68% |
| 3f | 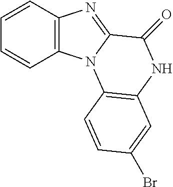 [SMILES: O=c1[nH]c2cc(Br)ccc2n2c1nc1ccccc12] | 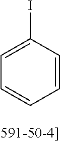 | 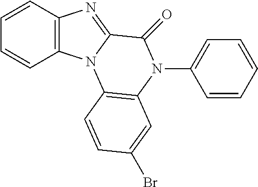 [SMILES: O=c1c2nc3ccccc3n2c2ccc(Br)cc2n1-c1ccccc1] | 71% |
| 4f | 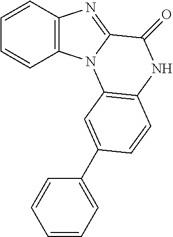 [SMILES: O=c1[nH]c2ccc(-c3ccccc3)cc2n2c1nc1ccccc12] | 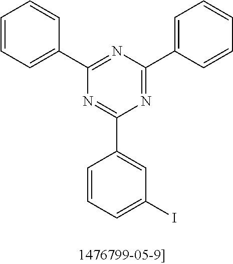 [SMILES: Ic1cccc(-c2nc(-c3ccccc3)nc(-c3ccccc3)n2)c1] | 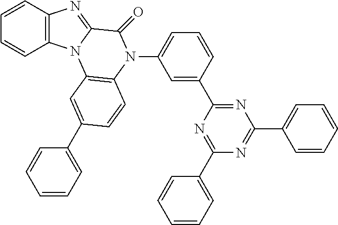 [SMILES: O=c1c2nc3ccccc3n2c2cc(-c3ccccc3)ccc2n1-c1cccc(-c2nc(-c3ccccc3)nc(-c3ccccc3)n2)c1] | 63% |
| 5f | 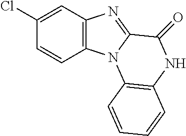 [SMILES: O=c1[nH]c2ccccc2n2c1nc1cc(Cl)ccc12] | 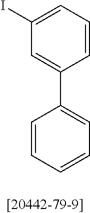 [SMILES: Ic1cccc(-c2ccccc2)c1] | 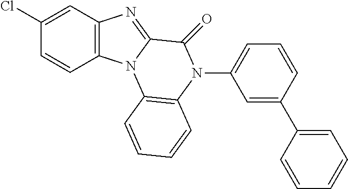 [SMILES: O=c1c2nc3cc(Cl)ccc3n2c2ccccc2n1-c1cccc(-c2ccccc2)c1] | 77% |
| 6f | 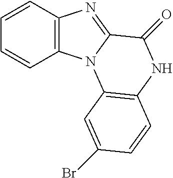 [SMILES: O=c1[nH]c2ccc(Br)cc2n2c1nc1ccccc12] | 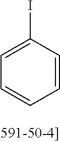 | 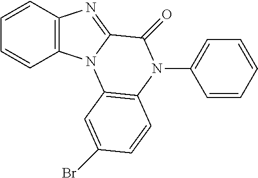 [SMILES: O=c1c2nc3ccccc3n2c2cc(Br)ccc2n1-c1ccccc1] | 65% |
| 7f | 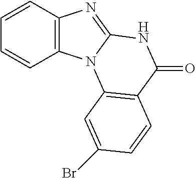 [SMILES: O=c1[nH]c2nc3ccccc3n2c2cc(Br)ccc12] | 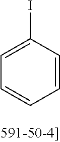 | 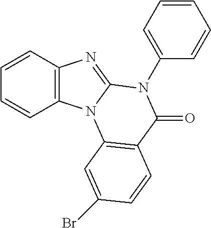 [SMILES: O=c1c2ccc(Br)cc2n2c3ccccc3nc2n1-c1ccccc1] | 64% |
| 8f | 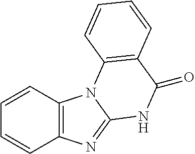 [SMILES: O=c1[nH]c2nc3ccccc3n2c2ccccc12] | 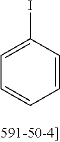 | 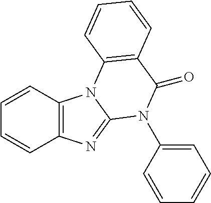 [SMILES: O=c1c2ccccc2n2c3ccccc3nc2n1-c1ccccc1] | 64% |
| 9f | 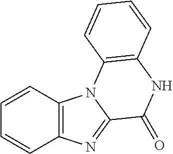 [SMILES: O=c1[nH]c2ccccc2n2c1nc1ccccc12] | 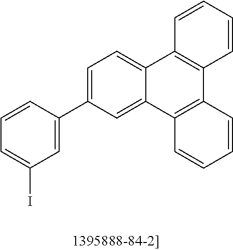 [SMILES: Ic1cccc(-c2ccc3c4ccccc4c4ccccc4c3c2)c1] | 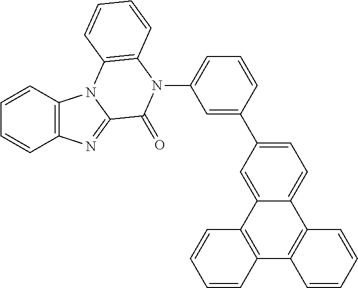 [SMILES: O=c1c2nc3ccccc3n2c2ccccc2n1-c1cccc(-c2ccc3c4ccccc4c4ccccc4c3c2)c1] | 67% |
| 10f | 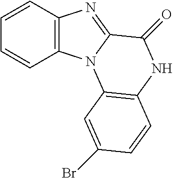 [SMILES: O=c1[nH]c2ccc(Br)cc2n2c1nc1ccccc12] | 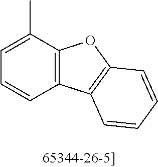 | 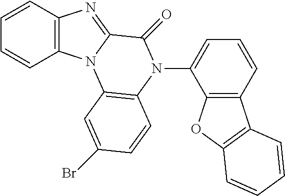 [SMILES: O=c1c2nc3ccccc3n2c2cc(Br)ccc2n1-c1cccc2c1oc1ccccc12] | 71% |
| 11f | 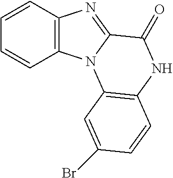 [SMILES: O=c1[nH]c2ccc(Br)cc2n2c1nc1ccccc12] | 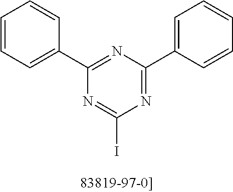 [SMILES: Ic1nc(-c2ccccc2)nc(-c2ccccc2)n1] | 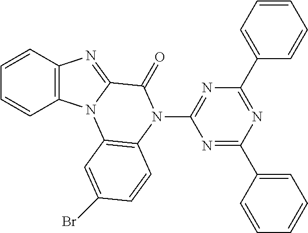 [SMILES: O=c1c2nc3ccccc3n2c2cc(Br)ccc2n1-c1nc(-c2ccccc2)nc(-c2ccccc2)n1] | 52% |
| 12f | 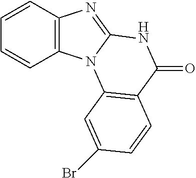 [SMILES: O=c1[nH]c2nc3ccccc3n2c2cc(Br)ccc12] | 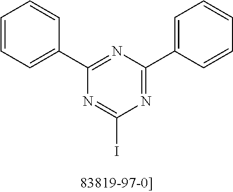 [SMILES: Ic1nc(-c2ccccc2)nc(-c2ccccc2)n1] | 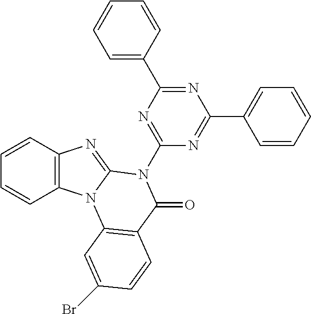 [SMILES: O=c1c2ccc(Br)cc2n2c3ccccc3nc2n1-c1nc(-c2ccccc2)nc(-c2ccccc2)n1] | 50% |
| 13f | 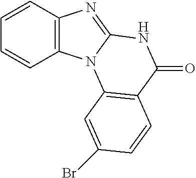 [SMILES: O=c1[nH]c2nc3ccccc3n2c2cc(Br)ccc12] | 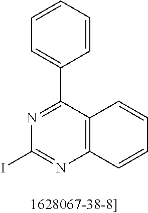 [SMILES: Ic1nc(-c2ccccc2)c2ccccc2n1] | 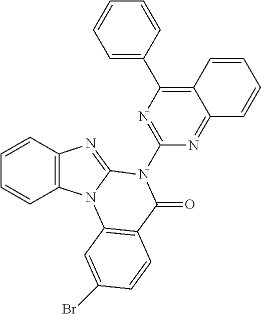 [SMILES: O=c1c2ccc(Br)cc2n2c3ccccc3nc2n1-c1nc(-c2ccccc2)c2ccccc2n1] | 52% |
| 14f | 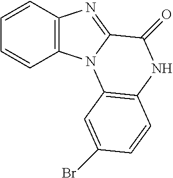 [SMILES: O=c1[nH]c2ccc(Br)cc2n2c1nc1ccccc12] | 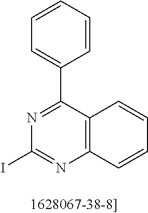 [SMILES: Ic1nc(-c2ccccc2)c2ccccc2n1] | 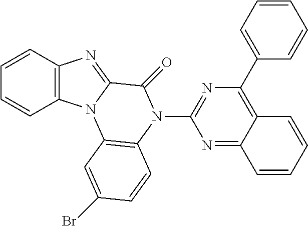 [SMILES: O=c1c2nc3ccccc3n2c2cc(Br)ccc2n1-c1nc(-c2ccccc2)c2ccccc2n1] | 61% |
| 15f | 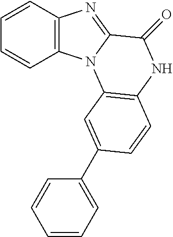 [SMILES: O=c1[nH]c2ccc(-c3ccccc3)cc2n2c1nc1ccccc12] | 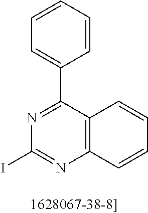 [SMILES: Ic1nc(-c2ccccc2)c2ccccc2n1] | 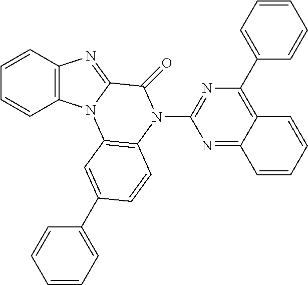 [SMILES: O=c1c2nc3ccccc3n2c2cc(-c3ccccc3)ccc2n1-c1nc(-c2ccccc2)c2ccccc2n1] | 69% |
| 16f | 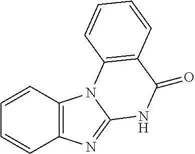 [SMILES: O=c1[nH]c2nc3ccccc3n2c2ccccc12] | 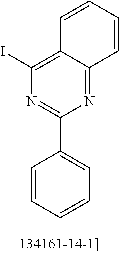 [SMILES: Ic1nc(-c2ccccc2)nc2ccccc12] | 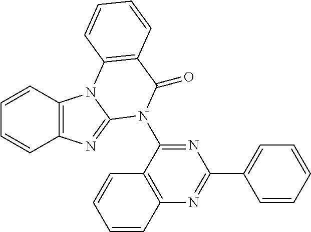 [SMILES: O=c1c2ccccc2n2c3ccccc3nc2n1-c1nc(-c2ccccc2)nc2ccccc12] | 54% |

### Example g) 5-Phenyl-3-(9-phenylcarbazol-3-yl)benzimidazolo[1,2-a]quinoxalin-6-one

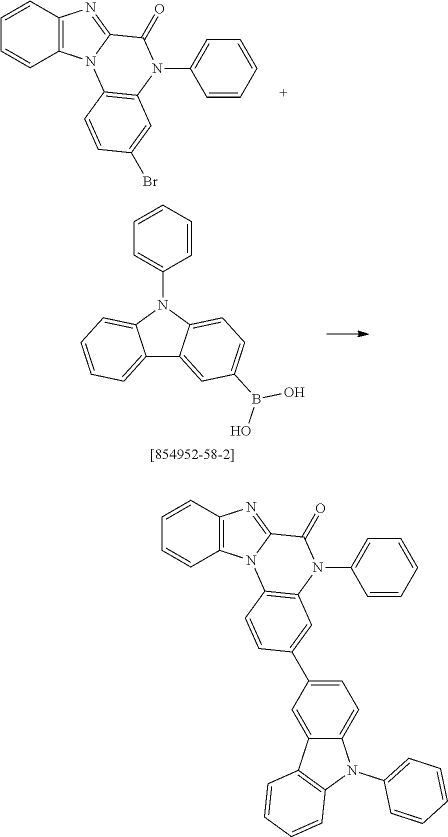

27.3 (70 mmol) of 3-bromo-5-phenylbenzimidazolo[1,2-a]quinoxalin-6-one, 20.8 g (75 mmol) of phenylcarbazole-3-boronic acid and 14.7 g (139 mmol) of sodium carbonate are suspended in 200 ml of toluene, 52 ml of ethanol and 100 ml of water. 80 mg (0.69 mmol) of tetrakisphenylphosphinepalladium(0) are added to this suspension, and the reaction mixture is heated under reflux for 16 h. After cooling, the organic phase is removed, filtered through silica gel, washed three times with 200 ml of water and then concentrated to dryness. The residue is recrystallized from heptane/dichloromethane. The yield is 29 g (54 mmol), corresponding to 77% of theory.

The following compound is obtained in an analogous manner:

| Ex. | Reactant 1 | Reactant 2 | Product | Yield |
|---|---|---|---|---|
| 2g | 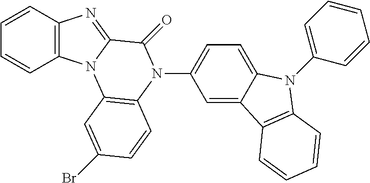 [SMILES: O=c1c2nc3ccccc3n2c2cc(Br)ccc2n1-c1ccc2c(c1)c1ccccc1n2-c1ccccc1] | 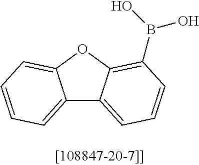 [SMILES: OB(O)c1cccc2c1oc1ccccc12] | 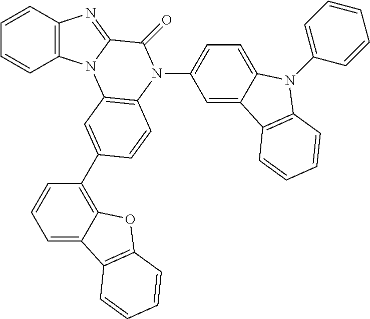 [SMILES: O=c1c2nc3ccccc3n2c2cc(-c3cccc4c3oc3ccccc34)ccc2n1-c1ccc2c(c1)c1ccccc1n2-c1ccccc1] | 71% |
| 3g | 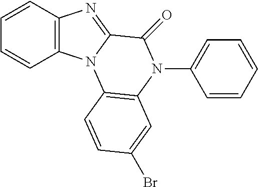 [SMILES: O=c1c2nc3ccccc3n2c2ccc(Br)cc2n1-c1ccccc1] | 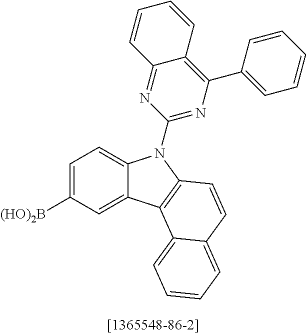 [SMILES: OB(O)c1ccc2c(c1)c1c3ccccc3ccc1n2-c1nc(-c2ccccc2)c2ccccc2n1] | 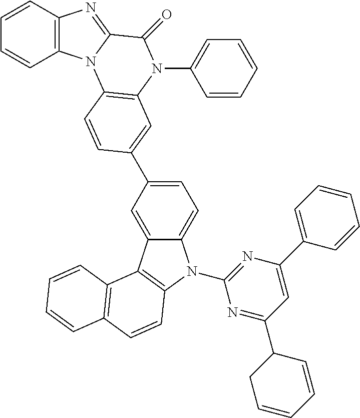 [SMILES: O=c1c2nc3ccccc3n2c2ccc(-c3ccc4c(c3)c3c5ccccc5ccc3n4-c3nc(-c4ccccc4)cc(C4C=CC=CC4)n3)cc2n1-c1ccccc1] | 82% |
| 4g | 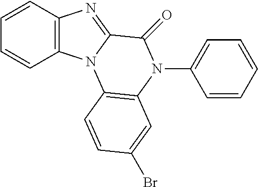 [SMILES: O=c1c2nc3ccccc3n2c2ccc(Br)cc2n1-c1ccccc1] | 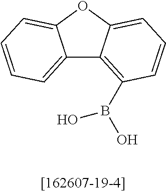 [SMILES: OB(O)c1cccc2oc3ccccc3c12] | 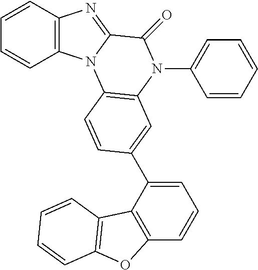 [SMILES: O=c1c2nc3ccccc3n2c2ccc(-c3cccc4oc5ccccc5c34)cc2n1-c1ccccc1] | 76% |
| 5g | 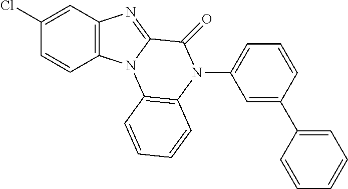 [SMILES: O=c1c2nc3cc(Cl)ccc3n2c2ccccc2n1-c1cccc(-c2ccccc2)c1] | 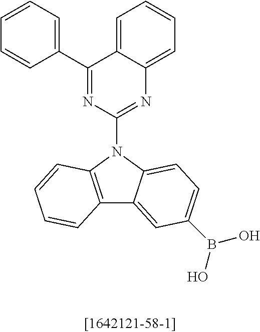 [SMILES: OB(O)c1ccc2c(c1)c1ccccc1n2-c1nc(-c2ccccc2)c2ccccc2n1] | 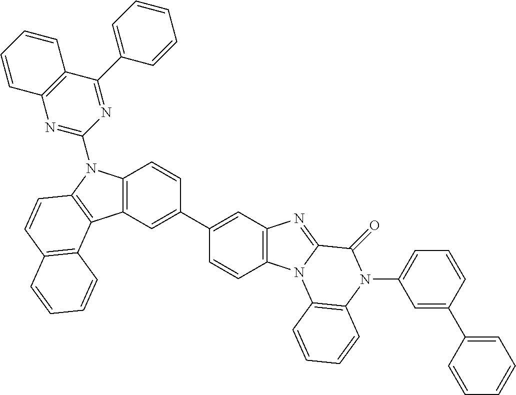 [SMILES: O=c1c2nc3cc(-c4ccc5c(c4)c4c6ccccc6ccc4n5-c4nc(-c5ccccc5)c5ccccc5n4)ccc3n2c2ccccc2n1-c1cccc(-c2ccccc2)c1] | 77% |
| 6g | 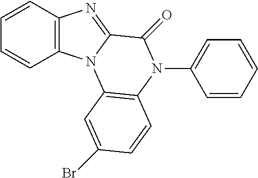 [SMILES: O=c1c2nc3ccccc3n2c2cc(Br)ccc2n1-c1ccccc1] | 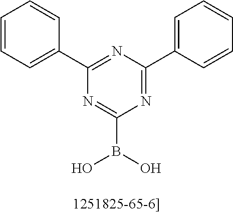 [SMILES: OB(O)c1nc(-c2ccccc2)nc(-c2ccccc2)n1] | 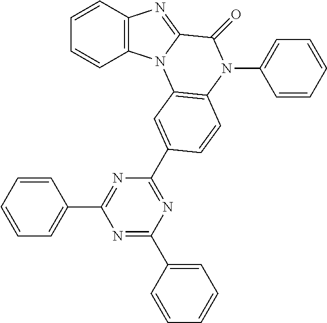 [SMILES: O=c1c2nc3ccccc3n2c2cc(-c3nc(-c4ccccc4)nc(-c4ccccc4)n3)ccc2n1-c1ccccc1] | 54% |
| 7g | 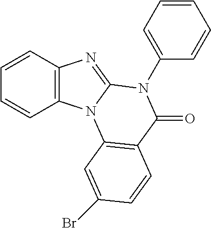 [SMILES: O=c1c2ccc(Br)cc2n2c3ccccc3nc2n1-c1ccccc1] | 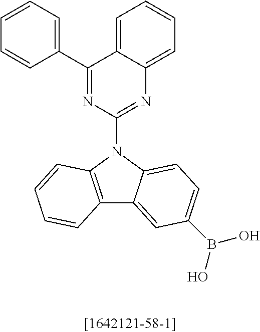 [SMILES: OB(O)c1ccc2c(c1)c1ccccc1n2-c1nc(-c2ccccc2)c2ccccc2n1] | 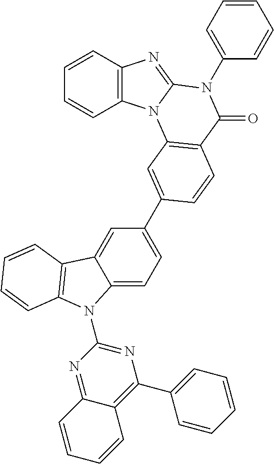 [SMILES: O=c1c2ccc(-c3ccc4c(c3)c3ccccc3n4-c3nc(-c4ccccc4)c4ccccc4n3)cc2n2c3ccccc3nc2n1-c1ccccc1] | 77% |
| 8g | 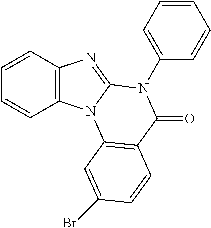 [SMILES: O=c1c2ccc(Br)cc2n2c3ccccc3nc2n1-c1ccccc1] | 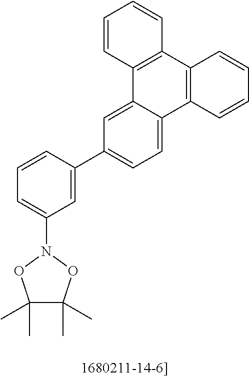 [SMILES: CC1(C)ON(c2cccc(-c3ccc4c5ccccc5c5ccccc5c4c3)c2)OC1(C)C] | 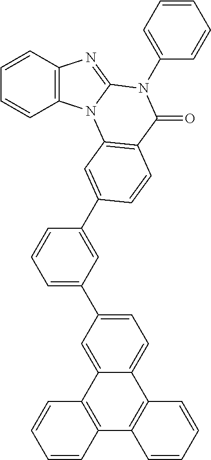 [SMILES: O=c1c2ccc(-c3cccc(-c4ccc5c6ccccc6c6ccccc6c5c4)c3)cc2n2c3ccccc3nc2n1-c1ccccc1] | 70% |
| 9g | 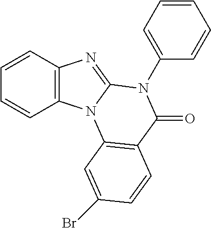 [SMILES: O=c1c2ccc(Br)cc2n2c3ccccc3nc2n1-c1ccccc1] | 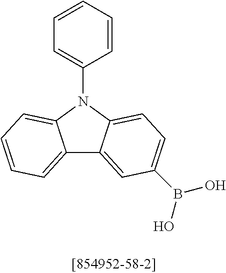 [SMILES: OB(O)c1ccc2c(c1)c1ccccc1n2-c1ccccc1] | 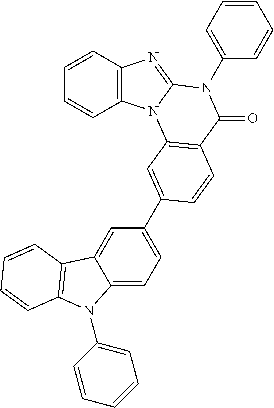 [SMILES: O=c1c2ccc(-c3ccc4c(c3)c3ccccc3n4-c3ccccc3)cc2n2c3ccccc3nc2n1-c1ccccc1] | 62% |
| 10g | 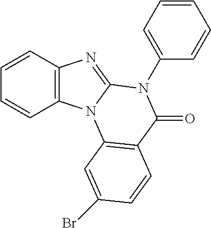 [SMILES: O=c1c2ccc(Br)cc2n2c3ccccc3nc2n1-c1ccccc1] | 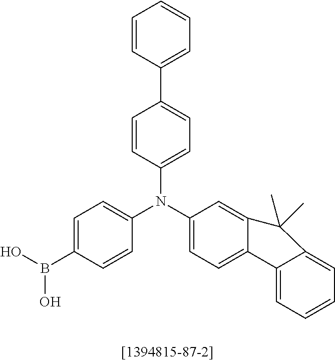 [SMILES: CC1(C)c2ccccc2-c2ccc(N(c3ccc(B(O)O)cc3)c3ccc(-c4ccccc4)cc3)cc21] | 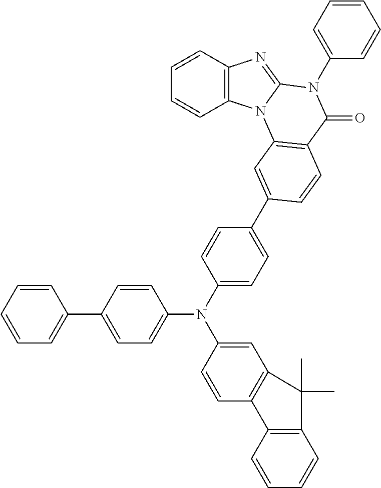 [SMILES: CC1(C)c2ccccc2-c2ccc(N(c3ccc(-c4ccccc4)cc3)c3ccc(-c4ccc5c(=O)n(-c6ccccc6)c6nc7ccccc7n6c5c4)cc3)cc21] | 62% |
| 11g | 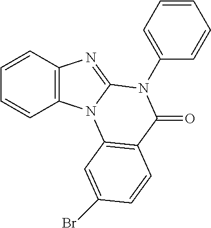 [SMILES: O=c1c2ccc(Br)cc2n2c3ccccc3nc2n1-c1ccccc1] | 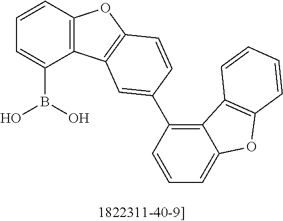 [SMILES: OB(O)c1cccc2oc3ccc(-c4cccc5oc6ccccc6c45)cc3c12] | 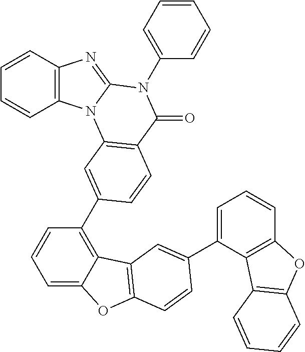 [SMILES: O=c1c2ccc(-c3cccc4oc5ccc(-c6cccc7oc8ccccc8c67)cc5c34)cc2n2c3ccccc3nc2n1-c1ccccc1] | 60% |
| 12g | 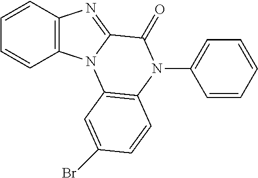 [SMILES: O=c1c2nc3ccccc3n2c2cc(Br)ccc2n1-c1ccccc1] | 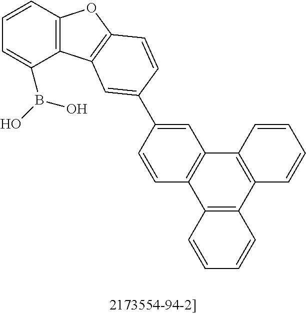 [SMILES: OB(O)c1cccc2oc3ccc(-c4ccc5c6ccccc6c6ccccc6c5c4)cc3c12] | 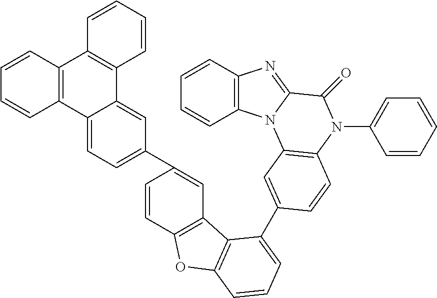 [SMILES: O=c1c2nc3ccccc3n2c2cc(-c3cccc4oc5ccc(-c6ccc7c8ccccc8c8ccccc8c7c6)cc5c34)ccc2n1-c1ccccc1] | 74% |
| 13g | 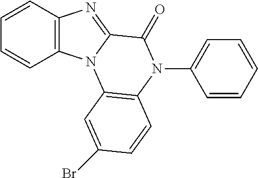 [SMILES: O=c1c2nc3ccccc3n2c2cc(Br)ccc2n1-c1ccccc1] | 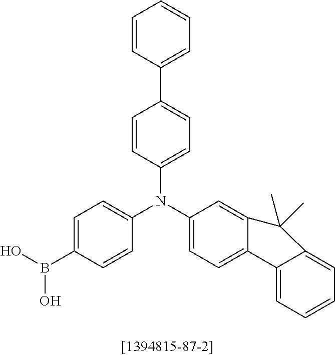 [SMILES: CC1(C)c2ccccc2-c2ccc(N(c3ccc(B(O)O)cc3)c3ccc(-c4ccccc4)cc3)cc21] | 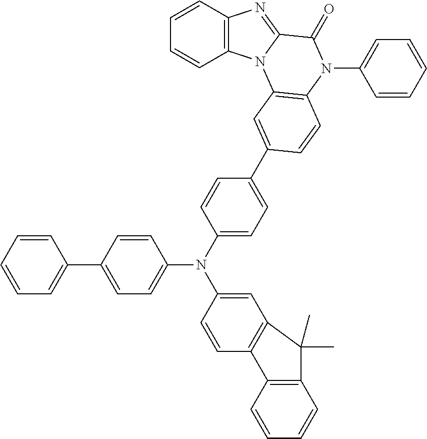 [SMILES: CC1(C)c2ccccc2-c2ccc(N(c3ccc(-c4ccccc4)cc3)c3ccc(-c4ccc5c(c4)n4c(nc6ccccc64)c(=O)n5-c4ccccc4)cc3)cc21] | 70% |
| 14g | 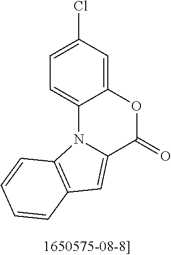 [SMILES: O=c1oc2cc(Cl)ccc2n2c1cc1ccccc12] | 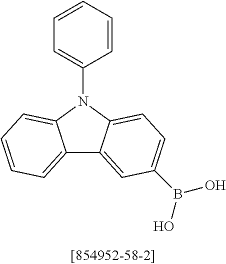 [SMILES: OB(O)c1ccc2c(c1)c1ccccc1n2-c1ccccc1] | 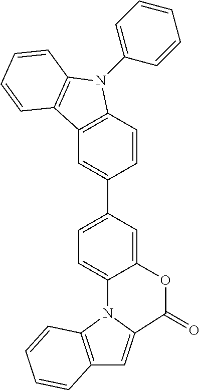 [SMILES: O=c1oc2cc(-c3ccc4c(c3)c3ccccc3n4-c3ccccc3)ccc2n2c1cc1ccccc12] | 68% |
| 15g | 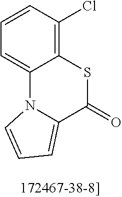 [SMILES: O=c1sc2c(Cl)cccc2n2cccc12] | 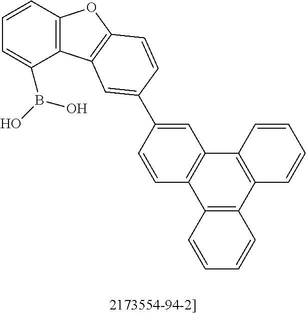 [SMILES: OB(O)c1cccc2oc3ccc(-c4ccc5c6ccccc6c6ccccc6c5c4)cc3c12] | 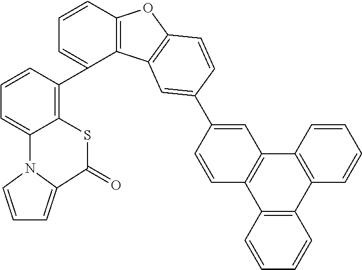 [SMILES: O=c1sc2c(-c3cccc4oc5ccc(-c6ccc7c8ccccc8c8ccccc8c7c6)cc5c34)cccc2n2cccc12] | 71% |
| 16g | 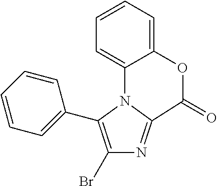 [SMILES: O=c1oc2ccccc2n2c(-c3ccccc3)c(Br)nc12] | 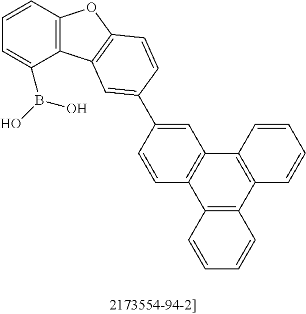 [SMILES: OB(O)c1cccc2oc3ccc(-c4ccc5c6ccccc6c6ccccc6c5c4)cc3c12] | 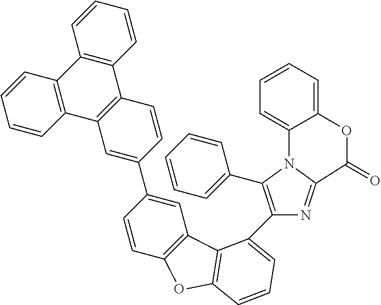 [SMILES: O=c1oc2ccccc2n2c(-c3ccccc3)c(-c3cccc4oc5ccc(-c6ccc7c8ccccc8c8ccccc8c7c6)cc5c34)nc12] | 64% |
| 17g | 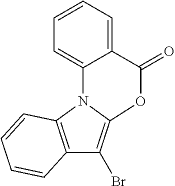 [SMILES: O=c1oc2c(Br)c3ccccc3n2c2ccccc12] | 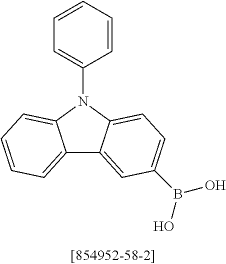 [SMILES: OB(O)c1ccc2c(c1)c1ccccc1n2-c1ccccc1] | 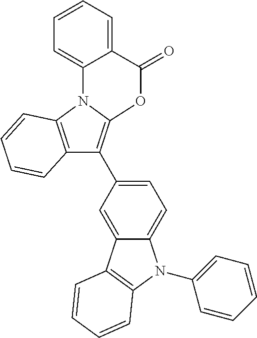 [SMILES: O=c1oc2c(-c3ccc4c(c3)c3ccccc3n4-c3ccccc3)c3ccccc3n2c2ccccc12] | 86% |
| 18g | 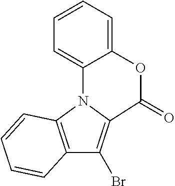 [SMILES: O=c1oc2ccccc2n2c1c(Br)c1ccccc12] | 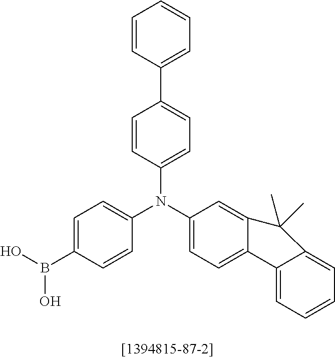 [SMILES: CC1(C)c2ccccc2-c2ccc(N(c3ccc(B(O)O)cc3)c3ccc(-c4ccccc4)cc3)cc21] | 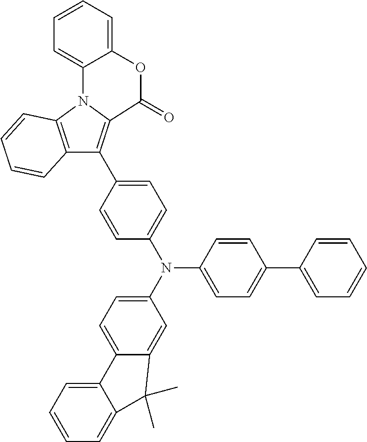 [SMILES: CC1(C)c2ccccc2-c2ccc(N(c3ccc(-c4ccccc4)cc3)c3ccc(-c4c5ccccc5n5c4c(=O)oc4ccccc45)cc3)cc21] | 80% |
| 19g | 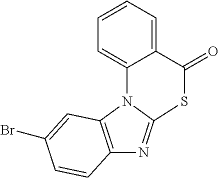 [SMILES: O=c1sc2nc3ccc(Br)cc3n2c2ccccc12] | 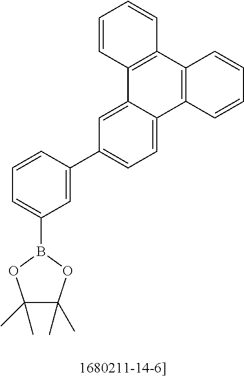 [SMILES: CC1(C)OB(c2cccc(-c3ccc4c5ccccc5c5ccccc5c4c3)c2)OC1(C)C] | 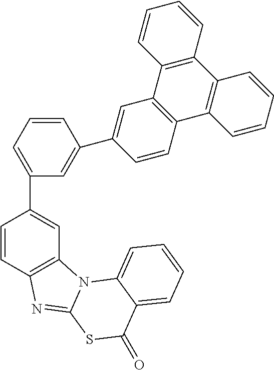 [SMILES: O=c1sc2nc3ccc(-c4cccc(-c5ccc6c7ccccc7c7ccccc7c6c5)c4)cc3n2c2ccccc12] | 84% |
| 20g | 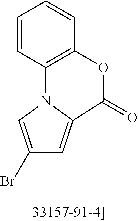 [SMILES: O=c1oc2ccccc2n2cc(Br)cc12] | 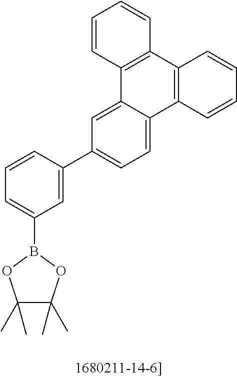 [SMILES: CC1(C)OB(c2cccc(-c3ccc4c5ccccc5c5ccccc5c4c3)c2)OC1(C)C] | 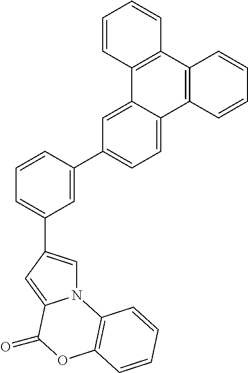 [SMILES: O=c1oc2ccccc2n2cc(-c3cccc(-c4ccc5c6ccccc6c6ccccc6c5c4)c3)cc12] | 76% |
| 21g | 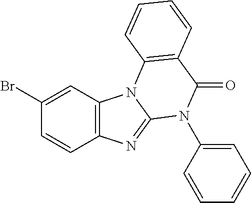 [SMILES: O=c1c2ccccc2n2c3cc(Br)ccc3nc2n1-c1ccccc1] | 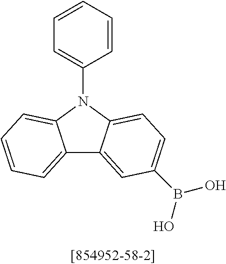 [SMILES: OB(O)c1ccc2c(c1)c1ccccc1n2-c1ccccc1] | 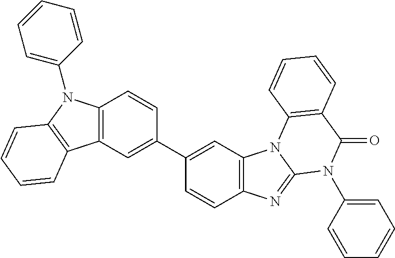 [SMILES: O=c1c2ccccc2n2c3cc(-c4ccc5c(c4)c4ccccc4n5-c4ccccc4)ccc3nc2n1-c1ccccc1] | 81% |
| 22g | 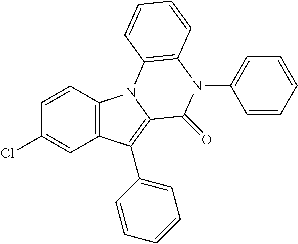 [SMILES: O=c1c2c(-c3ccccc3)c3cc(Cl)ccc3n2c2ccccc2n1-c1ccccc1] | 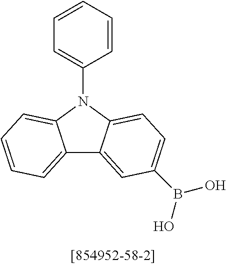 [SMILES: OB(O)c1ccc2c(c1)c1ccccc1n2-c1ccccc1] | 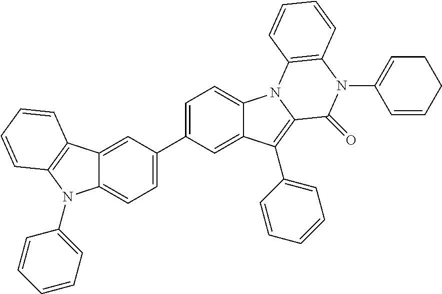 [SMILES: O=c1c2c(-c3ccccc3)c3cc(-c4ccc5c(c4)c4ccccc4n5-c4ccccc4)ccc3n2c2ccccc2n1C1=CCCC=C1] | 80% |
| 23g | 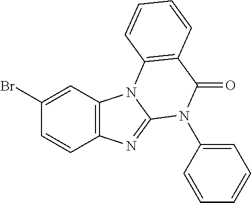 [SMILES: O=c1c2ccccc2n2c3cc(Br)ccc3nc2n1-c1ccccc1] | 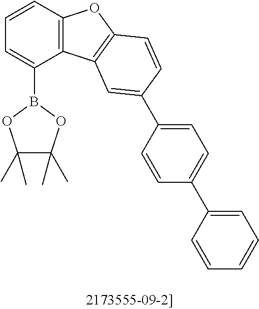 [SMILES: CC1(C)OB(c2cccc3oc4ccc(-c5ccc(-c6ccccc6)cc5)cc4c23)OC1(C)C] | 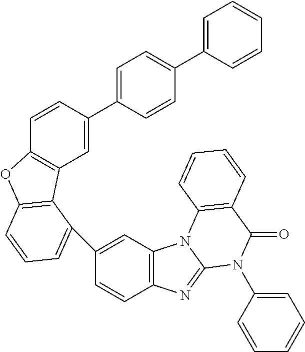 [SMILES: O=c1c2ccccc2n2c3cc(-c4cccc5oc6ccc(-c7ccc(-c8ccccc8)cc7)cc6c45)ccc3nc2n1-c1ccccc1] | 83% |
| 24g | 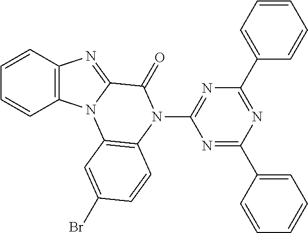 [SMILES: O=c1c2nc3ccccc3n2c2cc(Br)ccc2n1-c1nc(-c2ccccc2)nc(-c2ccccc2)n1] | 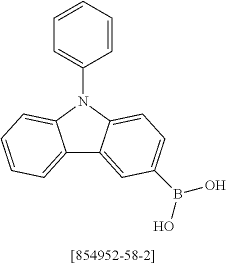 [SMILES: OB(O)c1ccc2c(c1)c1ccccc1n2-c1ccccc1] | 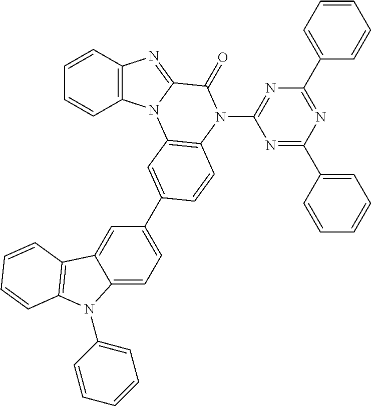 [SMILES: O=c1c2nc3ccccc3n2c2cc(-c3ccc4c(c3)c3ccccc3n4-c3ccccc3)ccc2n1-c1nc(-c2ccccc2)nc(-c2ccccc2)n1] | 80% |
| 25g | 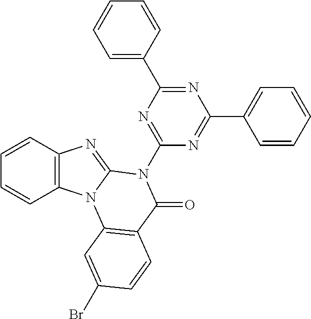 [SMILES: O=c1c2ccc(Br)cc2n2c3ccccc3nc2n1-c1nc(-c2ccccc2)nc(-c2ccccc2)n1] | 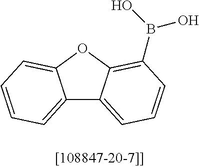 [SMILES: OB(O)c1cccc2c1oc1ccccc12] | 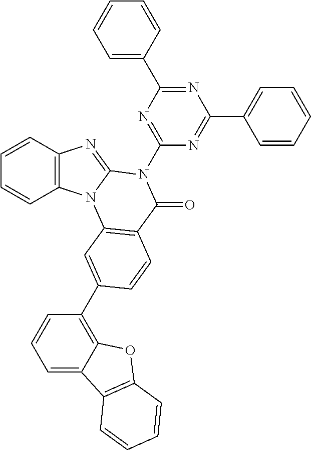 [SMILES: O=c1c2ccc(-c3cccc4c3oc3ccccc34)cc2n2c3ccccc3nc2n1-c1nc(-c2ccccc2)nc(-c2ccccc2)n1] | 79% |
| 26g | 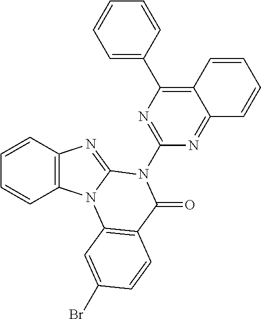 [SMILES: O=c1c2ccc(Br)cc2n2c3ccccc3nc2n1-c1nc(-c2ccccc2)c2ccccc2n1] | 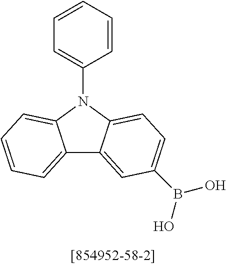 [SMILES: OB(O)c1ccc2c(c1)c1ccccc1n2-c1ccccc1] | 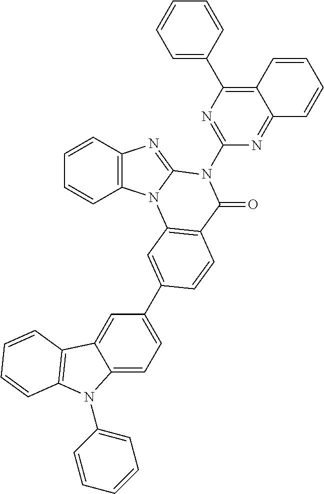 [SMILES: O=c1c2ccc(-c3ccc4c(c3)c3ccccc3n4-c3ccccc3)cc2n2c3ccccc3nc2n1-c1nc(-c2ccccc2)c2ccccc2n1] | 77% |
| 27g | 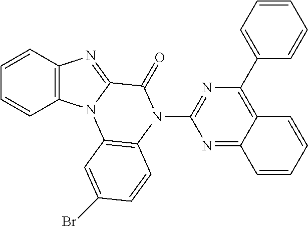 [SMILES: O=c1c2nc3ccccc3n2c2cc(Br)ccc2n1-c1nc(-c2ccccc2)c2ccccc2n1] | 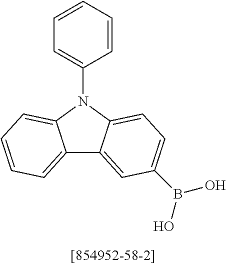 [SMILES: OB(O)c1ccc2c(c1)c1ccccc1n2-c1ccccc1] | 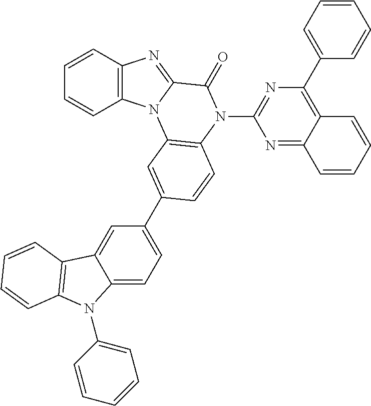 [SMILES: O=c1c2nc3ccccc3n2c2cc(-c3ccc4c(c3)c3ccccc3n4-c3ccccc3)ccc2n1-c1nc(-c2ccccc2)c2ccccc2n1] | 81% |
| 28g | 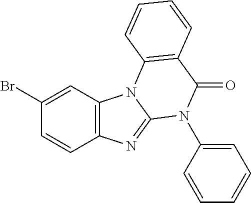 [SMILES: O=c1c2ccccc2n2c3cc(Br)ccc3nc2n1-c1ccccc1] | 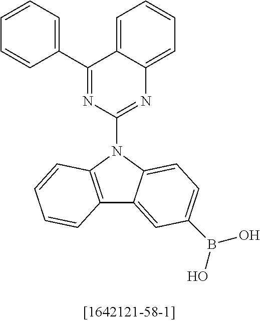 [SMILES: OB(O)c1ccc2c(c1)c1ccccc1n2-c1nc(-c2ccccc2)c2ccccc2n1] | 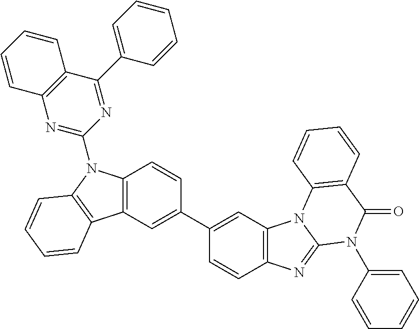 [SMILES: O=c1c2ccccc2n2c3cc(-c4ccc5c(c4)c4ccccc4n5-c4nc(-c5ccccc5)c5ccccc5n4)ccc3nc2n1-c1ccccc1] | 80% |
| 29g | 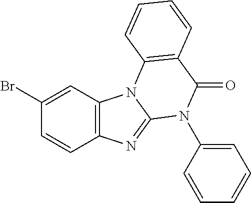 [SMILES: O=c1c2ccccc2n2c3cc(Br)ccc3nc2n1-c1ccccc1] | 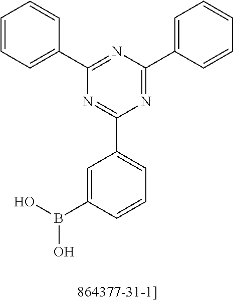 [SMILES: OB(O)c1cccc(-c2nc(-c3ccccc3)nc(-c3ccccc3)n2)c1] | 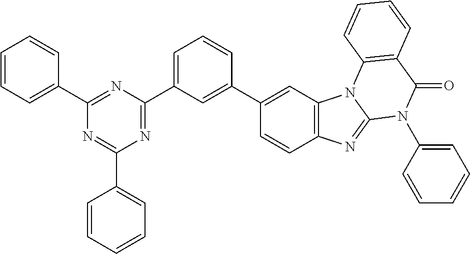 [SMILES: O=c1c2ccccc2n2c3cc(-c4cccc(-c5nc(-c6ccccc6)nc(-c6ccccc6)n5)c4)ccc3nc2n1-c1ccccc1] | 76% |
| 30g | 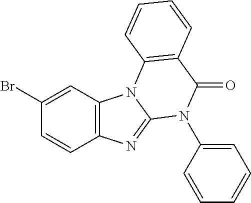 [SMILES: O=c1c2ccccc2n2c3cc(Br)ccc3nc2n1-c1ccccc1] | 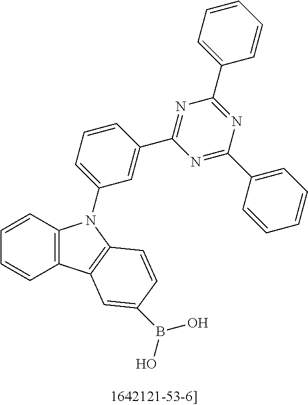 [SMILES: OB(O)c1ccc2c(c1)c1ccccc1n2-c1cccc(-c2nc(-c3ccccc3)nc(-c3ccccc3)n2)c1] | 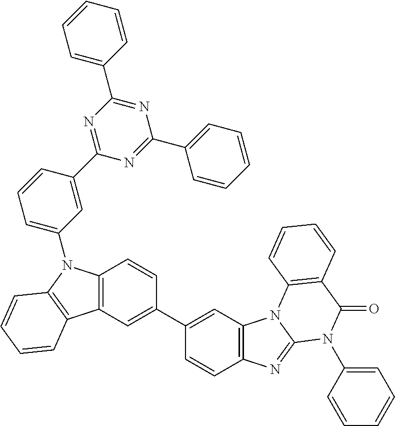 [SMILES: O=c1c2ccccc2n2c3cc(-c4ccc5c(c4)c4ccccc4n5-c4cccc(-c5nc(-c6ccccc6)nc(-c6ccccc6)n5)c4)ccc3nc2n1-c1ccccc1] | 69% |

### Example h: 2-(2,5-Dibromopyrrol-1-yl)-N1,N3-diphenylbenzene-1,3-dicarboxamide

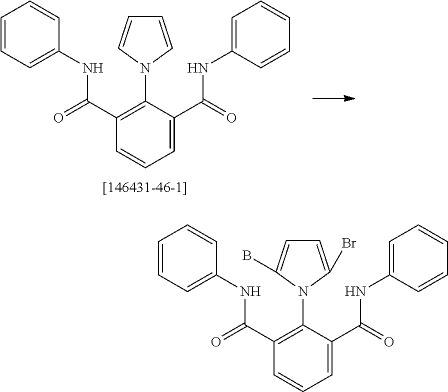

4.5 g (12 mmol) of N1,N3-diphenyl-2-pyrrol-1-ylbenzene-1,3-dicarboxamide are initially charged in 150 ml of CH₂Cl₂. Subsequently, a solution of 4 g (22.5 mmol) of NBS in 100 ml of acetonitrile is added dropwise in the dark at −5° C., the mixture is allowed to come to room temperature and stirring is continued at this temperature for 4 h. Subsequently, 150 ml of water are added to the mixture and extraction is effected with CH₂Cl₂. The organic phase is dried over MgSO₄ and the solvents are removed under reduced pressure. The product is subjected to extractive stirring with hot hexane and filtered off with suction. Yield: 3.9 g (17.6 mmol), 70% of theory, purity by ¹H NMR about 98%.

### Example i: Cyclization

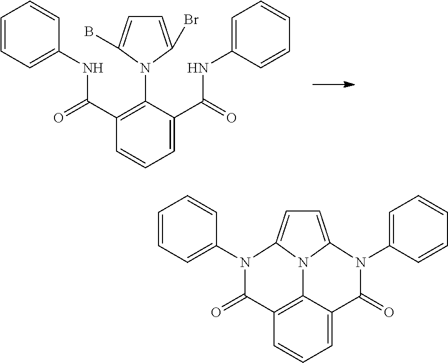

11.8 g (25 mmol) of 2-(2,5-dibromopyrrol-1-yl)-N1,N3-diphenylbenzene-1,3-dicarboxamide are dissolved in 600 ml of toluene and degassed with argon for 30 minutes. Subsequently, 8.1 g (84.8 mmol) of sodium t-butoxide, 476 mg (2.12 mmol) of palladium(II) acetate and 4.2 ml (4.24 mmol) of tri-t-butylphosphine (1.0M in toluene) are added and the mixture is stirred under reflux overnight. After the reaction has ended, 200 ml of water are added to the mixture, and the organic phase is removed and extracted twice with water. The organic phase is dried over sodium sulfate and concentrated to about 80 ml on a rotary evaporator. The precipitated solids are filtered off with suction and purified by means of hot extraction in toluene. The product is recrystallized three times with toluene/heptane and then sublimed. 6.6 g (17.0 mmol, 71%) of the desired target compound having HPLC purity >99.9% are obtained.

### B) Device Examples

The examples which follow present the use of the materials of the invention in OLEDs.

Glass plates coated with structured ITO (indium tin oxide) of thickness 50 nm are treated prior to coating, first with an oxygen plasma, followed by an argon plasma. These plasma-treated glass plates form the substrates to which the OLEDs are applied.

The OLEDs basically have the following layer structure: substrate/hole injection layer (HIL)/hole transport layer (HTL)/electron blocker layer (EBL)/emission layer (EML)/hole blocker layer (HBL)/electron transport layer (ETL)/electron injection layer (EIL) and finally a cathode. The cathode is formed by an aluminum layer of thickness 100 nm. The exact structure of the OLEDs can be found in tables 1a to 1c. The data of the OLEDs are listed in tables 2a to 2c. The materials required for production of the OLEDs are shown in table 3.

All materials are applied by thermal vapor deposition in a vacuum chamber. In this case, the emission layer always consists of at least one matrix material (host material) and an emitting dopant (emitter) which is added to the matrix material(s) in a particular proportion by volume by coevaporation. What is meant here by data in such a form as A:B:C (45%:45%:10%) is that material A is present in the layer in a proportion by volume of 45%, material B in a proportion by volume of 45%, and material C in a proportion by volume of 10%. In an analogous manner, the electron transport layer or one of the other layers may also consist of a mixture of two materials.

The OLEDs are characterized in a standard manner. For this purpose, the electroluminescence spectra and the external quantum efficiency (EQE, measured in %) as a function of the luminance, calculated from current-voltage-luminance characteristics assuming Lambertian emission characteristics, are determined. The electroluminescence spectra are determined at a luminance of 1000 cd/m², and the CIE 1931 x and y color coordinates are calculated therefrom. EQE1000 denotes the external quantum efficiency which is attained at 1000 cd/cm².

The materials of the invention are used in examples E1 to E6 as matrix material in the emission layer of green-phosphorescing OLEDs.

TABLE 1a
Structure of the OLEDs

| | HIL | HTL | EBL | EML | HBL | ETL | EIL |
|---|---|---|---|---|---|---|---|
| Ex. | thickness | thickness | thickness | thickness | thickness | thickness | thickness |
| E1 | HATCN | SpMA1 | SpMA2 | IC1:21c:TEG1 | ST2 | ST2:LiQ | LiQ |
| | 5 nm | 215 nm | 20 nm | (59%:29%:12%) 30 nm | 10 nm | (50%:50%) 30 nm | 1 nm |
| E2 | HATCN | SpMA1 | SpMA2 | 25g:IC2:TEG1 | ST2 | ST2:LiQ | LiQ |
| | 5 nm | 215 nm | 20 nm | (44%:44%:12%) 30 nm | 10 nm | (50%:50%) 30 nm | 1 nm |
| E3 | HATCN | SpMA1 | SpMA2 | 16g:IC2:TEG1 | ST2 | ST2:LiQ | LiQ |
| | 5 nm | 215 nm | 20 nm | (29%:59%:12%) 30 nm | 10 nm | (50%:50%) 30 nm | 1 nm |
| E4 | HATCN | SpMA1 | SpMA2 | IC1:19g:TEG1 | ST2 | ST2:LiQ | LiQ |
| | 5 nm | 215 nm | 20 nm | (54%:29%:17%) 30 nm | 10 nm | (50%:50%) 30 nm | 1 nm |
| E5 | HATCN | SpMA1 | SpMA2 | 29g:IC3:TEG1 | ST2 | ST2:LiQ | LiQ |
| | 5 nm | 215 nm | 20 nm | (29%:59%:12%) 30 nm | 10 nm | (50%:50%) 30 nm | 1 nm |
| E6 | HATCN | SpMA1 | SpMA2 | IC3:30g:TEG1 | ST2 | ST2:LiQ | LiQ |
| | 5 nm | 215 nm | 20 nm | (54%:29%:17%) 30 nm | 10 nm | (50%:50%) 30 nm | 1 nm |

All compounds of the invention give very good results for external quantum efficiency.

TABLE 2a
Data of the OLEDs

| | U1000 | EQE 1000 | CIE x/y at |
|---|---|---|---|
| Ex. | (V) | (%) | 1000 cd/m² |
| E1 | 3.3 | 18 | 0.36/0.61 |
| E2 | 3.5 | 18.5 | 0.35/0.61 |
| E3 | 3.6 | 16 | 0.34/0.62 |
| E4 | 3.9 | 19 | 0.35/0.61 |
| E5 | 3.1 | 20 | 0.35/0.64 |
| E6 | 3.2 | 19.5 | 0.36/0.61 |

Further materials of the invention are used in examples E7 to E9 as matrix material in the emission layer of red-phosphorescing OLEDs.

TABLE 1b
Structure of the OLEDs

| | HIL | HTL | EBL | EML | HBL | ETL | EIL |
|---|---|---|---|---|---|---|---|
| Ex. | thickness | thickness | thickness | thickness | thickness | thickness | thickness |
| E7 | HATCN | SpMA1 | SpMA2 | 11b:TER5 | ST2 | ST2:LiQ | LiQ |
| | 5 nm | 125 nm | 10 nm | (97%:3%) 35 nm | 10 nm | (50%:50%) 30 nm | 1 nm |
| E8 | HATCN | SpMA1 | SpMA2 | 15b:TER5 | ST2 | ST2:LiQ | LiQ |
| | 5 nm | 125 nm | 10 nm | (97%:3%) 35 nm | 10 nm | (50%:50%) 30 nm | 1 nm |
| E9 | HATCN | SpMA1 | SpMA2 | 28g:TER5 | ST2 | ST2:LiQ | LiQ |
| | 5 nm | 125 nm | 10 nm | (97%:3%) 35 nm | 10 nm | (50%:50%) 30 nm | 1 nm |

Both compounds of the invention give very good results for external quantum efficiency.

TABLE 2b
Data of the OLEDs

| | U1000 | CIE x/y at | EQE 1000 |
|---|---|---|---|
| Ex. | (V) | 1000 cd/m² | % |
| E7 | 3.5 | 0.67/0.34 | 17.1 |
| E8 | 3.1 | 0.67/0.33 | 19.6 |
| E9 | 3.2 | 0.67/0.34 | 18.9 |

A further material of the invention is used in examples E10 and E11 respectively as ETL and HBL of blue-fluorescing OLEDs. Use as ETL and HBL in phosphorescent OLEDs is likewise possible.

TABLE 1c
Structure of the OLEDs

| | HIL | HTL | EBL | EML | HBL | ETL | EIL |
|---|---|---|---|---|---|---|---|
| Ex. | thickness | thickness | thickness | thickness | thickness | thickness | thickness |
| E10 | HATCN | SpMA1 | SpMA2 | M2:SEB | — | 25g:LiQ | LiQ |
| | 5 nm | 195 nm | 10 nm | (95%:5%) 20 nm | | (50%:50%) 30 nm | 1 nm |
| E11 | HATCN | SpMA1 | SpMA2 | M2:SEB | 25g | ST2 | LiQ |
| | 5 nm | 195 nm | 10 nm | (95%:5%) 20 nm | 10 nm | 20 nm | 3 nm |

The compound of the invention gives very good results for external quantum efficiency, at operating voltages U1000 in the range of 4-5 V.

TABLE 2c
Data of the OLEDs

| | EQE 1000 | CIE x/y at |
|---|---|---|
| Ex. | (%) | 1000 cd/m² |
| E10 | 7 | 0.14/0.15 |
| E11 | 8 | 0.14/0.15 |

TABLE 3
Structural formulae of the materials for the OLEDs

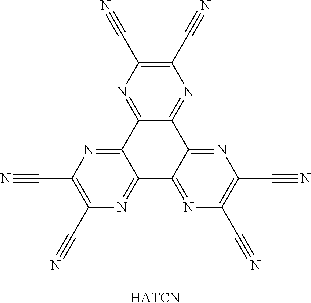

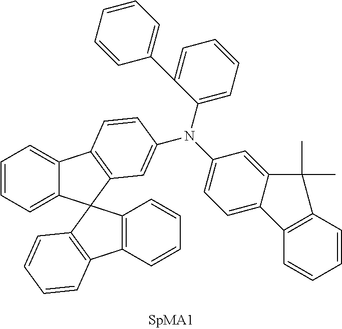 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

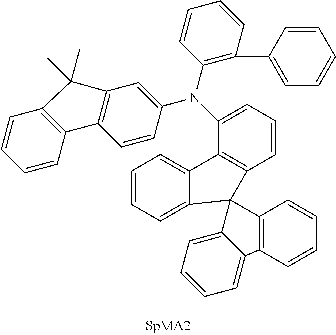 [SMILES: CC1(C)c2ccccc2-c2ccc(N(c3ccccc3-c3ccccc3)c3cccc4c3-c3ccccc3C43c4ccccc4-c4ccccc43)cc21]

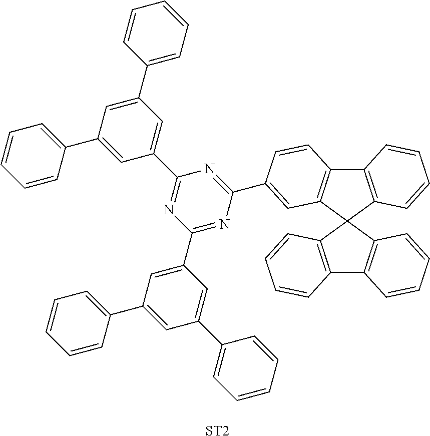 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1 ; "ST2"]

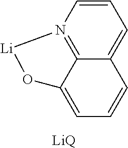

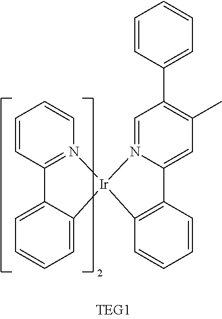

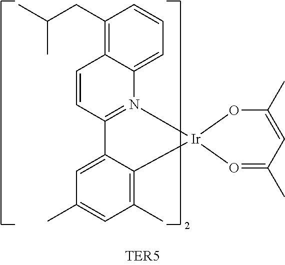

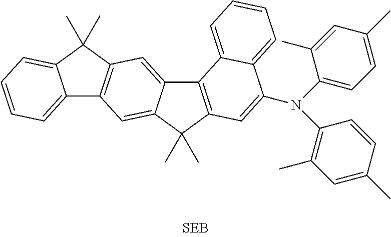 [SMILES: Cc1ccc(N(c2ccc(C)cc2C)c2cc3c(c4ccccc24)-c2cc4c(cc2C3(C)C)-c2ccccc2C4(C)C)c(C)c1]

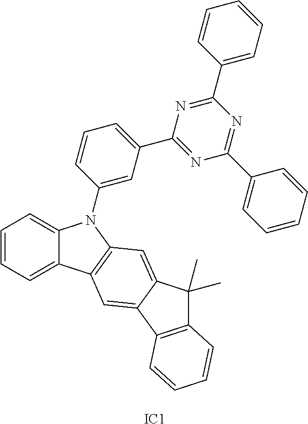 [SMILES: CC1(C)c2ccccc2-c2cc3c4ccccc4n(-c4cccc(-c5nc(-c6ccccc6)nc(-c6ccccc6)n5)c4)c3cc21 ; "IC1"]

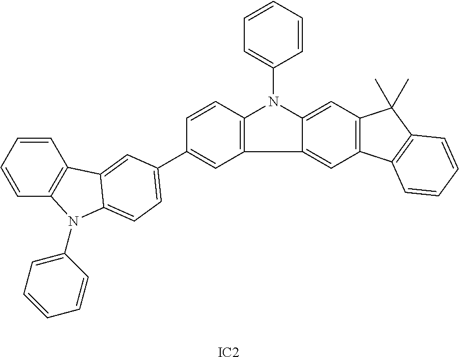 [SMILES: CC1(C)c2ccccc2-c2cc3c4cc(-c5ccc6c(c5)c5ccccc5n6-c5ccccc5)ccc4n(-c4ccccc4)c3cc21 ; "IC2"]

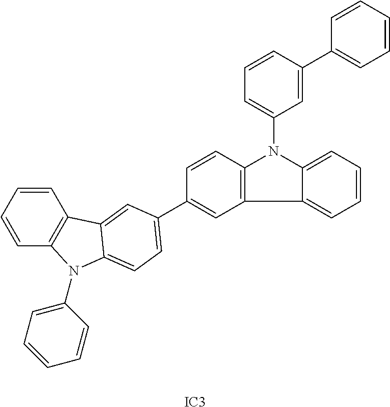 [SMILES: c1ccc(-c2cccc(-n3c4ccccc4c4cc(-c5ccc6c(c5)c5ccccc5n6-c5ccccc5)ccc43)c2)cc1 ; "IC3"]

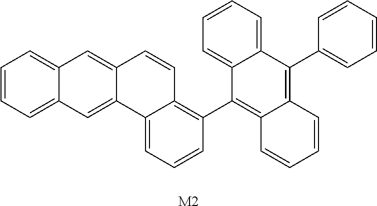

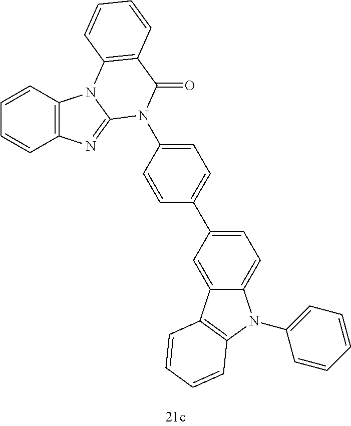 [SMILES: O=c1c2ccccc2n2c3ccccc3nc2n1-c1ccc(-c2ccc3c(c2)c2ccccc2n3-c2ccccc2)cc1]

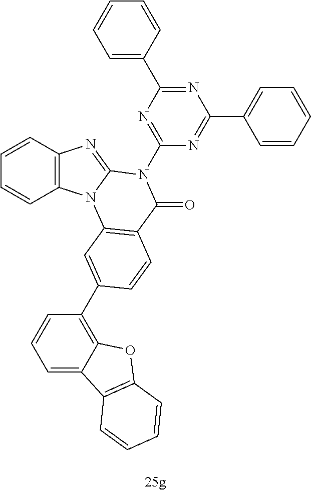 [SMILES: O=c1c2ccc(-c3cccc4c3oc3ccccc34)cc2n2c3ccccc3nc2n1-c1nc(-c2ccccc2)nc(-c2ccccc2)n1]

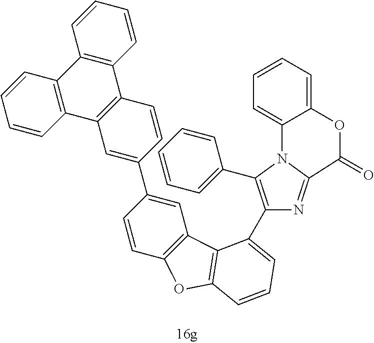 [SMILES: O=c1oc2ccccc2n2c(-c3ccccc3)c(-c3cccc4oc5ccc(-c6ccc7c8ccccc8c8ccccc8c7c6)cc5c34)nc12]

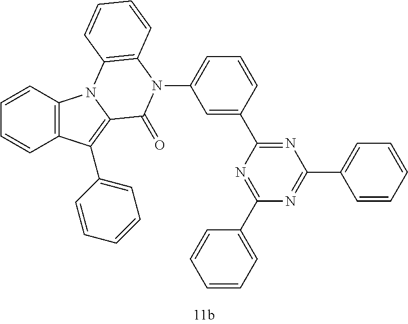 [SMILES: O=c1c2c(-c3ccccc3)c3ccccc3n2c2ccccc2n1-c1cccc(-c2nc(-c3ccccc3)nc(-c3ccccc3)n2)c1]

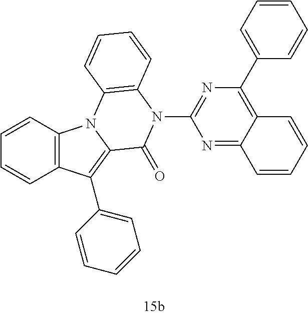 [SMILES: O=c1c2c(-c3ccccc3)c3ccccc3n2c2ccccc2n1-c1nc(-c2ccccc2)c2ccccc2n1]

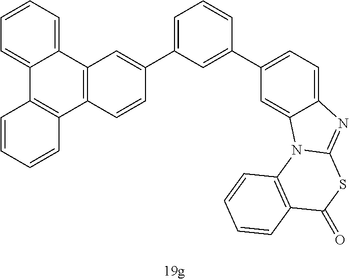 [SMILES: O=c1sc2nc3ccc(-c4cccc(-c5ccc6c7ccccc7c7ccccc7c6c5)c4)cc3n2c2ccccc12]

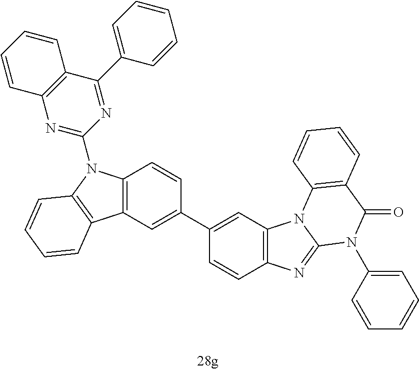 [SMILES: O=c1c2ccccc2n2c3cc(-c4ccc5c(c4)c4ccccc4n5-c4nc(-c5ccccc5)c5ccccc5n4)ccc3nc2n1-c1ccccc1]

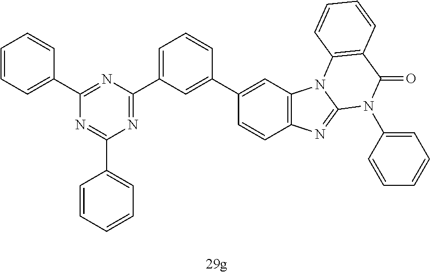 [SMILES: O=c1c2ccccc2n2c3cc(-c4cccc(-c5nc(-c6ccccc6)nc(-c6ccccc6)n5)c4)ccc3nc2n1-c1ccccc1]

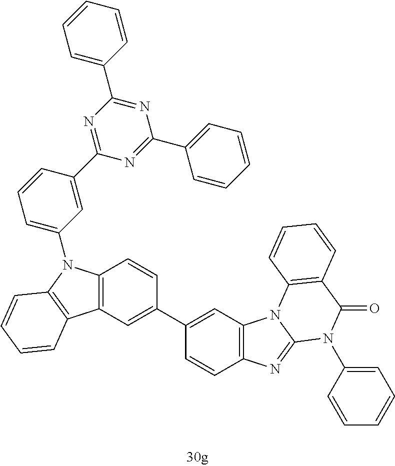 [SMILES: O=c1c2ccccc2n2c3cc(-c4ccc5c(c4)c4ccccc4n5-c4cccc(-c5nc(-c6ccccc6)nc(-c6ccccc6)n5)c4)ccc3nc2n1-c1ccccc1]

1. An electronic device comprising a compound containing a structural element of the formula (I) 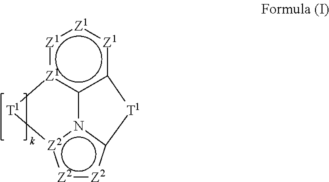
where:
Z¹ is the same or different at each instance and is C, CR¹ or N;
Z² is the same or different at each instance and is C, CR² or N;
T¹ is the same or different at each instance and is selected from (C═O)(NAr¹)—, —(C═S)(NAr¹)—, —(SO₂)(NAr¹)—, and —(C═O)O—;
Ar¹ is selected from aromatic ring systems which have 6 to 40 aromatic ring atoms and are substituted by one or more R³ radicals, and heteroaromatic ring systems which have 5 to 40 aromatic ring atoms and are substituted by one or more R³ radicals;
R¹ is the same or different at each instance and is selected from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form a ring; where the alkyl, alkoxy, alkenyl and alkynyl groups and the aromatic ring systems and heteroaromatic ring systems are each substituted by R⁴ radicals; and where one or more CH₂ groups in the alkyl, alkoxy, alkenyl and alkynyl groups may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂;
R² is the same or different at each instance and is selected from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where the alkyl, alkoxy, alkenyl and alkynyl groups and the aromatic ring systems and heteroaromatic ring systems are each substituted by R⁴ radicals; and where one or more CH₂ groups in the alkyl, alkoxy, alkenyl and alkynyl groups may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂;
R³ is the same or different at each instance and is selected from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups and the aromatic ring systems and heteroaromatic ring systems are each substituted by R⁴ radicals; and where one or more CH₂ groups in the alkyl, alkoxy, alkenyl and alkynyl groups may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂;
R⁴ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups and the aromatic ring systems and heteroaromatic ring systems are each substituted by R⁵ radicals; and where one or more CH₂ groups in the alkyl, alkoxy, alkenyl and alkynyl groups may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁵ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁵ radicals may be joined to one another and may form a ring; and where the alkyl, alkoxy, alkenyl and alkynyl groups, aromatic ring systems and heteroaromatic ring systems may be substituted by one or more radicals selected from F and CN;
k is 0 or 1, where, in the case that k=1, the Z¹ and Z² groups that bind to the T¹ group in question are C and are bonded to one another via the T¹ group, and where, in the case that k=0, the T¹ group in question is absent, and the Z¹ and Z² groups in question are not bonded to one another.

2. The electronic device as claimed in claim 1, wherein T¹ is —(C═O)(NAr¹)—.

3. The electronic device as claimed in claim 1, wherein Ar¹ is the same or different at each instance and is selected from the group consisting of phenyl, biphenyl, terphenyl, quaterphenyl, triphenylene, naphthyl, fluorenyl, dibenzofuranyl, dibenzothiophenyl, carbazolyl, benzofuranyl, benzothiophenyl, triazinyl, pyrimidyl, pyridyl, quinazoline, quinoxaline and quinoline, where the groups mentioned may each be substituted by one or more R³ radicals.

4. The electronic device as claimed in claim 1, wherein Z¹ is the same or different at each instance and is selected from C and CR¹.

5. The electronic device as claimed in claim 1, wherein k is 0.

6. The electronic device as claimed in claim 1, wherein
R¹ is the same or different at each instance and is selected from H, aromatic ring systems having 6 to 40 aromatic ring atoms, and heteroaromatic ring systems having 5 to 40 aromatic ring atoms; where the aromatic ring systems and the heteroaromatic ring systems are each substituted by R⁴ radicals; and
R² is the same or different at each instance and is selected from H, aromatic ring systems having 6 to 40 aromatic ring atoms, and heteroaromatic ring systems having 5 to 40 aromatic ring atoms; where the aromatic ring systems and the heteroaromatic ring systems are each substituted by R⁴ radicals; and
R³ is the same or different at each instance and is selected from H, aromatic ring systems having 6 to 40 aromatic ring atoms, and heteroaromatic ring systems having 5 to 40 aromatic ring atoms; where the aromatic ring systems and the heteroaromatic ring systems are each substituted by R⁴ radicals; and
R⁴ is the same or different at each instance and is selected from H, D, F, CN, Si(R⁵)₃, N(R⁵)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups, the aromatic ring systems and the heteroaromatic ring systems are each substituted by R⁵ radicals; and where one or more CH₂ groups in the alkyl or alkoxy groups may be replaced by —C≡C—, R⁵C═CR⁵—, Si(R⁵)₂, C═O, C═NR⁵, —NR⁵—, —O—, —S—, —C(═O)O— or —C(═O)NR⁵—; and
R⁵ is H.

7. The electronic device as claimed in claim 1, wherein the compound containing a structural unit of formula (I) conforms to one of the following formulae: 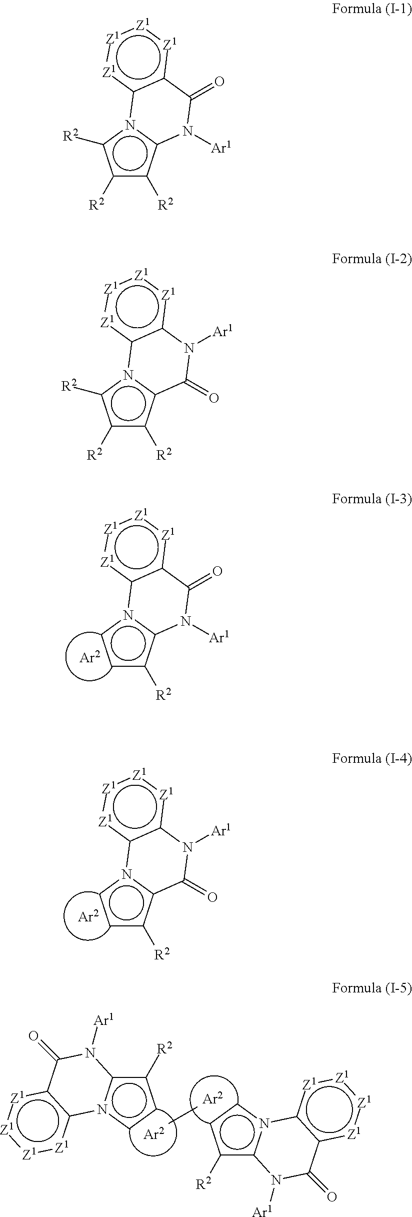 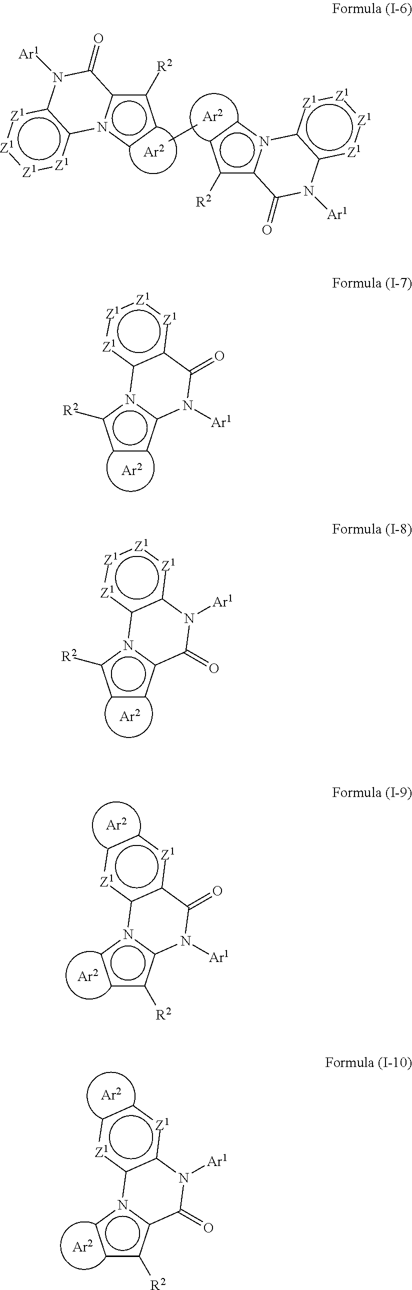 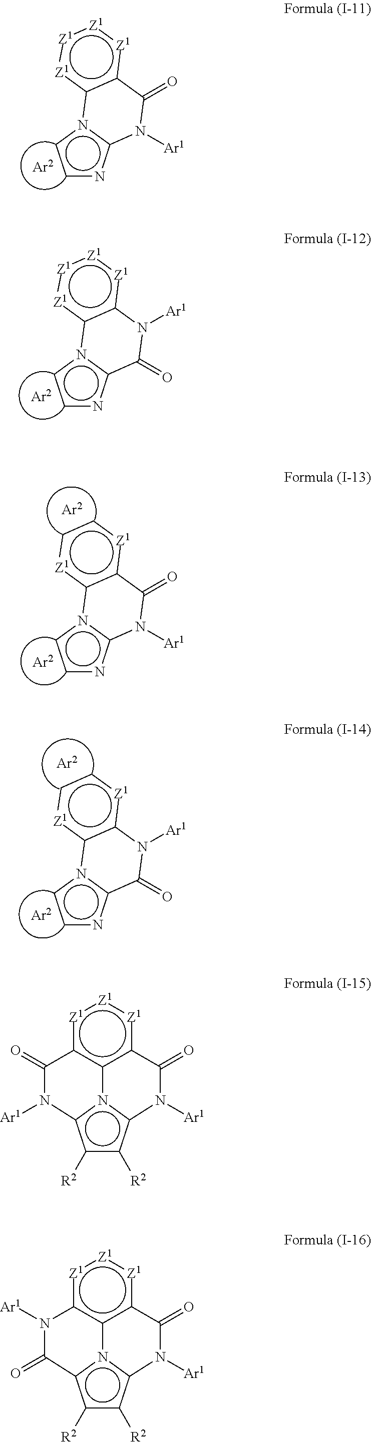 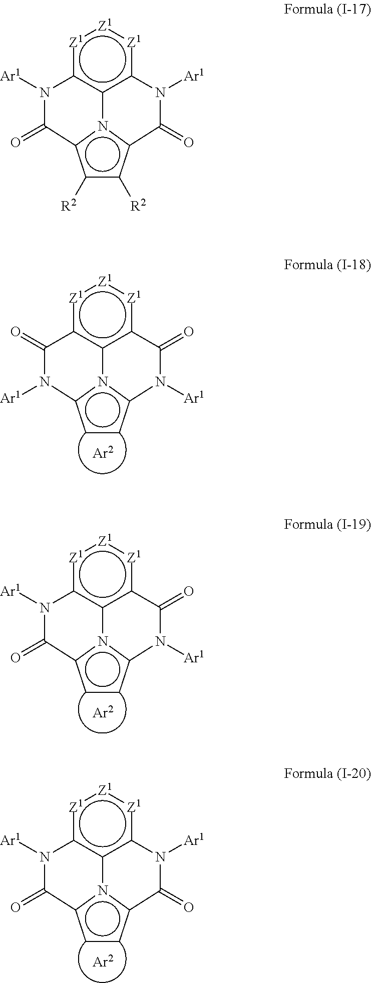
where
Ar² is selected from aromatic ring systems which have 4 to 40 aromatic ring atoms and are substituted by R^{A} radicals and heteroaromatic ring systems which have 3 to 40 aromatic ring atoms and are substituted by R^{A} radicals, where
R^{A} is the same or different at each instance and is selected from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R^{A} radicals may be joined to one another and may form a ring; where the alkyl, alkoxy, alkenyl and alkynyl groups and the aromatic ring systems and heteroaromatic ring systems are each substituted by R⁴ radicals; and where one or more CH₂ groups in the alkyl, alkoxy, alkenyl and alkynyl groups may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂; and where
Z¹ is the same or different at each instance and is CR¹ or N.

8. The electronic device as claimed in claim 7, wherein Ar² is fused-on benzene substituted by one or more R^{A} radicals.

9. The electronic device as claimed in claim 7, wherein R^{A} is the same or different at each instance and is selected from H, D, F, CN, Si(R⁴)₃, N(R⁴)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms, and heteroaromatic ring systems having 5 to 40 aromatic ring atoms, where the alkyl and alkoxy groups, the aromatic ring systems and the heteroaromatic ring systems are each substituted by R⁴ radicals.

10. The electronic device as claimed in claim 9, wherein it is an organic electroluminescent device and comprises anode, cathode and at least one emitting layer, and in that the compound containing a structural element of the formula (I) is present in an emitting layer together with at least one phosphorescent emitter, or in that the compound containing a structural element of the formula (I) is present in a layer selected from hole blocker layers, electron transport layers and electron injection layers.

11. The organic electroluminescent device as claimed in claim 10, wherein the compound containing a structural element of the formula (I) is present in an emitting layer of the device together with at least one phosphorescent emitter and at least one further compound, where the further compound is a matrix material.

12. The organic electroluminescent device as claimed in claim 11, wherein the further compound is selected from hole-transporting matrix materials, preferably carbazole compounds, biscarbazole compounds, indolocarbazole compounds and indenocarbazole compounds.

13. A compound of one of the formulae 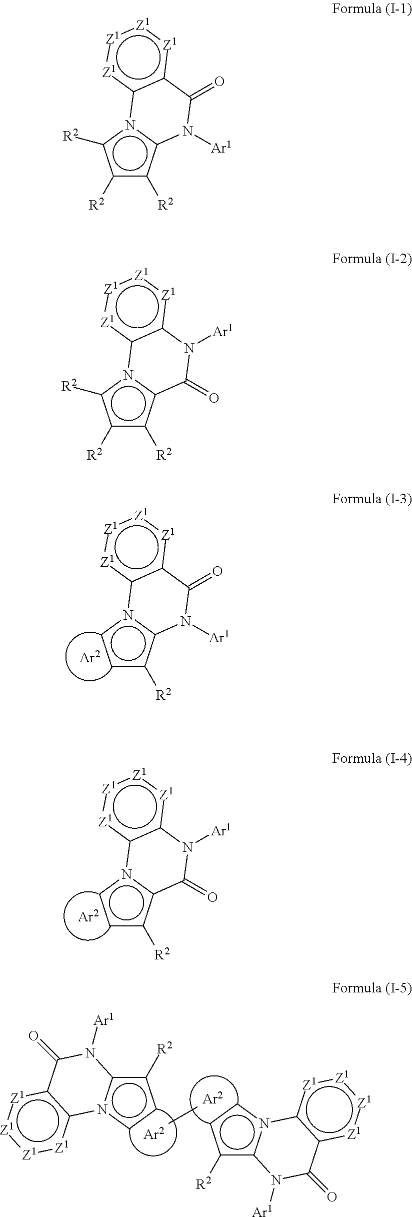 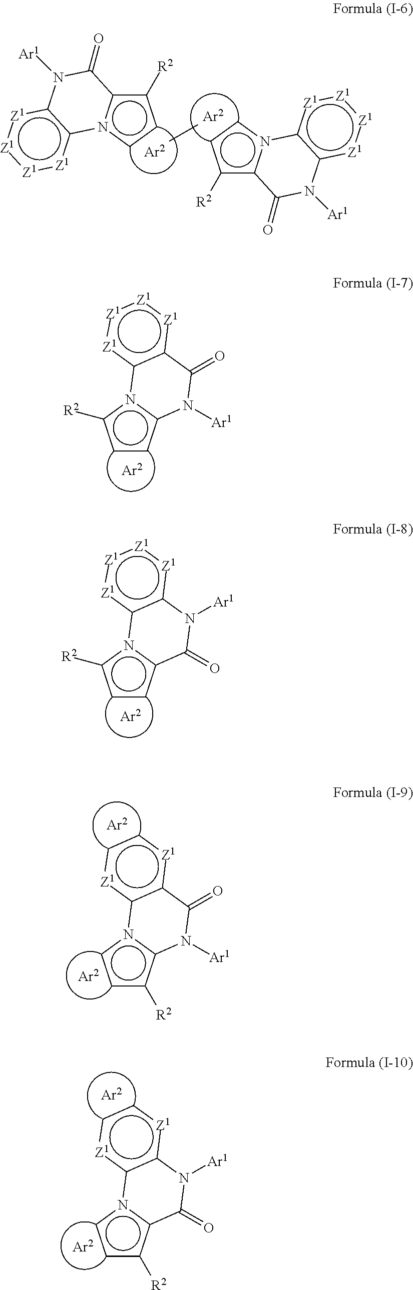 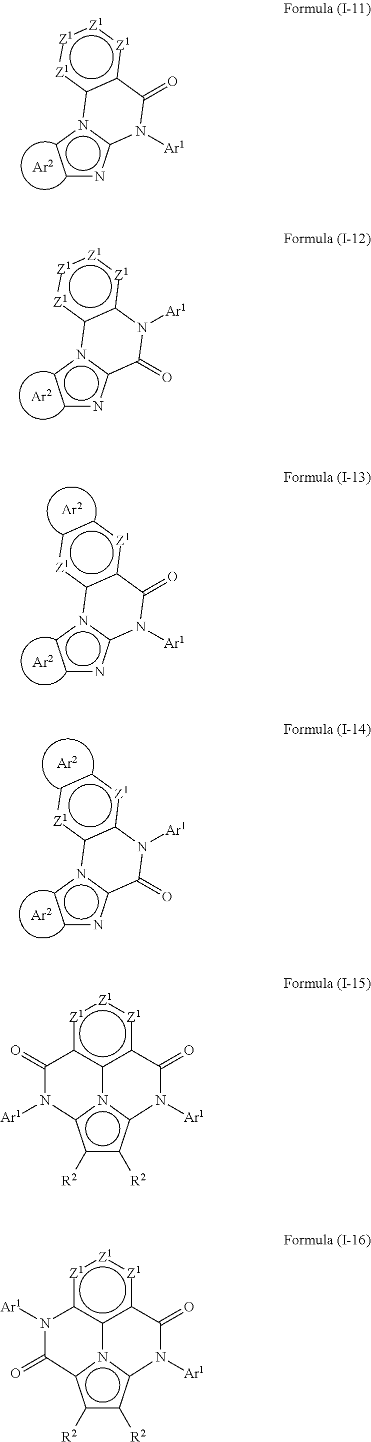 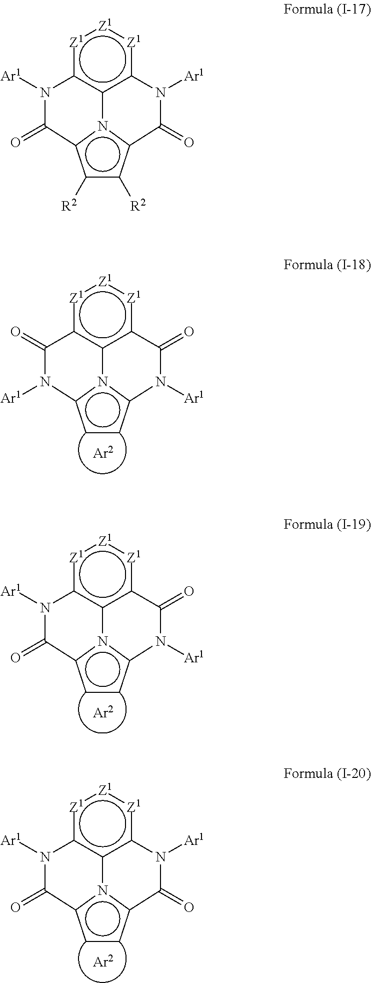
where
Ar² is selected from aromatic ring systems which have 4 to 40 aromatic ring atoms and are substituted by R^{A} radicals and heteroaromatic ring systems which have 3 to 40 aromatic ring atoms and are substituted by R^{A} radicals, where
R^{A} is the same or different at each instance and is selected from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R^{A} radicals may be joined to one another and may form a ring; where the alkyl, alkoxy, alkenyl and alkynyl groups and the aromatic ring systems and heteroaromatic ring systems are each substituted by R⁴ radicals; and where one or more CH₂ groups in the alkyl, alkoxy, alkenyl and alkynyl groups may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂; and where
Z¹ is the same or different at each instance and is CR¹ or N;
Ar¹ is selected from aromatic ring systems which have 6 to 40 aromatic ring atoms and are substituted by one or more R³ radicals, and heteroaromatic ring systems which have 5 to 40 aromatic ring atoms and are substituted by one or more R³ radicals;
R¹ is the same or different at each instance and is selected from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form a ring; where the alkyl, alkoxy, alkenyl and alkynyl groups and the aromatic ring systems and heteroaromatic ring systems are each substituted by R⁴ radicals; and where one or more CH₂ groups in the alkyl, alkoxy, alkenyl and alkynyl groups may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂;
R² is the same or different at each instance and is selected from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where the alkyl, alkoxy, alkenyl and alkynyl groups and the aromatic ring systems and heteroaromatic ring systems are each substituted by R⁴ radicals; and where one or more CH₂ groups in the alkyl, alkoxy, alkenyl and alkynyl groups may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂;
R³ is the same or different at each instance and is selected from H, D, F, Cl, Br, I, C(═O)R⁴, CN, Si(R⁴)₃, N(R⁴)₂, P(═O)(R⁴)₂, OR⁴, S(═O)R⁴, S(═O)₂R⁴, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups and the aromatic ring systems and heteroaromatic ring systems are each substituted by R⁴ radicals; and where one or more CH₂ groups in the alkyl, alkoxy, alkenyl and alkynyl groups may be replaced by —R⁴C═CR⁴—, —C≡C—, Si(R⁴)₂, C═O, C═NR⁴, —C(═O)O—, —C(═O)NR⁴—, NR⁴, P(═O)(R⁴), —O—, —S—, SO or SO₂;
R⁴ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups and the aromatic ring systems and heteroaromatic ring systems are each substituted by R⁵ radicals; and where one or more CH₂ groups in the alkyl, alkoxy, alkenyl and alkynyl groups may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁵ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁵ radicals may be joined to one another and may form a ring; and where the alkyl, alkoxy, alkenyl and alkynyl groups, aromatic ring systems and heteroaromatic ring systems may be substituted by one or more radicals selected from F and CN;
where there is at least one R¹, R², R³ or R^{A} group selected from
aromatic ring systems which have 6 to 40 aromatic ring atoms and are substituted by R⁴ radicals (radical A),
heteroaromatic ring systems which have 5 to 40 aromatic ring atoms and are substituted by R⁴ radicals (radical B),
N(R⁴)₂ (radical C).

14. An oligomer, polymer or dendrimer containing one or more compounds as claimed in claim 13, wherein the bond(s) to the polymer, oligomer or dendrimer may be localized at any desired positions substituted by R¹, R² or R³ in formula (I).

15. A formulation comprising at least one compound as claimed in claim 13, and at least one solvent.

16. A method comprising incorporating the compound as claimed in claim 13 in an electronic device.

17. A process for preparing a compound as claimed in claim 13, wherein either
a) proceeding from a compound of the formula (Int-I) or (Int-II) 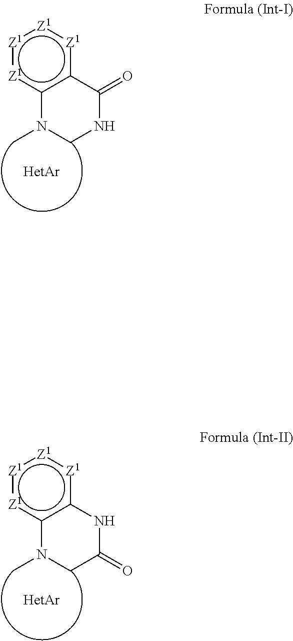
where HetAr is a heteroaromatic ring system which has 5 to 40 aromatic ring atoms and is substituted by R² radicals, and Z¹ is the same or different at each instance and is selected from CR¹ or N,
in a first step a halogen substituent is introduced, preferably Cl, Br or I, in a second step an aromatic or heteroaromatic ring system is introduced on the nitrogen atom of the lactam group in an Ullmann coupling reaction, and in a third step an amino group bearing the aromatic or heteroaromatic ring systems as substituents is introduced in the position of the halogen substituent via a Buchwald coupling, or an aromatic or heteroaromatic ring system is introduced via a Suzuki coupling; or
b) proceeding from a compound of a formula (Int-III) 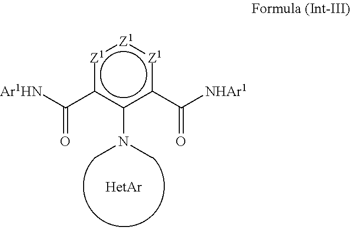
where Z¹ is the same or different at each instance and is CR¹ or N; and HetAr is a heteroaromatic ring system which has 5 to 40 aromatic ring atoms and is substituted by R² radicals; and Ar¹ is the same or different at each instance and is selected from aromatic ring systems which have 6 to 40 aromatic ring atoms and are substituted by one or more R³ radicals, and heteroaromatic ring systems which have 5 to 40 aromatic ring atoms and are substituted by one or more R³ radicals;
wherein in a first step halogen substituents, preferably Br, are introduced in the two positions vicinal to the nitrogen atom of HetAr, and in a second step the amide groups of the formula (Int-III) bind to the positions of the halogen substituents on HetAr in a metal-catalyzed coupling reaction, so as to form two lactam rings; or
c) a starting compound of a formula (Int-IV) 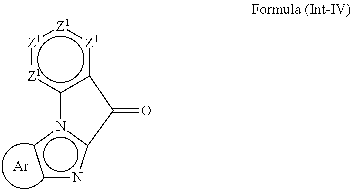
where Ar is an aromatic ring system which has 6 to 40 aromatic ring atoms and is substituted by R² radicals, and where Z¹ is the same or different at each instance and is selected from CR¹ or N,
in a first step is converted at its ketone group to the corresponding hydroxylamine derivative, this hydroxylamine derivative is then converted further in a second step in a Beckmann rearrangement, and the resultant lactam derivative is then converted in a third step at the nitrogen of the lactam unit in an Ullmann coupling.

18. A mixture comprising at least one compound as claimed in claim 13 and at least one further compound selected from matrix materials for phosphorescent emitters.
